(19) United States
(12) Patent Application Publication
(10) Pub. No.: US 2023/0383360 A1
(43) Pub. Date: Nov. 30, 2023

(54) BIOMARKERS FOR PREDICTING RESPONSIVENESS TO MEK INHIBITOR MONOTHERAPY AND COMBINATION THERAPY

(71) Applicant: D2G Oncology, Inc., Mountain View, CA (US)

(72) Inventors: Ian Paul Winters, Palo Alto, CA (US); Joseph Brian Miranda Juan, Santa Clara, CA (US); Michael Rosen, Palo Alto, CA (US); Monte Meier Winslow, Stanford, CA (US); Dmitri Alexandrovich Petrov, San Francisco, CA (US); Gregory Daniel Wall, Burlingame, CA (US)

(21) Appl. No.: 18/082,464

(22) Filed: Dec. 15, 2022

Related U.S. Application Data
(60) Provisional application No. 63/290,242, filed on Dec. 16, 2021.

(51) Int. Cl. C12Q 1/6886; A61K 31/337; A61K 45/06

(52) U.S. Cl. CPC C12Q 1/6886; A61K 31/337; A61K 45/06; C12Q 2600/106; C12Q 2600/156; C12Q 2600/158

(57) ABSTRACT

The invention provides compositions and methods comprising biomarker genes for identifying subjects that will respond to human MAP kinase kinase (MEK) inhibitor therapies, compositions and methods comprising biomarker genes for identifying subjects that will respond to MEK inhibitor/target of rapamycin complex (TORC) inhibitor combination therapies, and compositions and methods comprising biomarker genes for identifying subjects that will respond to MEK inhibitor/chemotherapy combination therapies.

### CROSS REFERENCE

This application claims the benefit of U.S. Provisional Application No. 63/290,242, filed Dec. 16, 2021, the full disclosure of which is hereby incorporated by reference herein in its entirety.

### FIELD

Provided herein, in certain aspects, are compositions and methods comprising biomarker genes for identifying subjects that will respond to human MAP kinase kinase (MEK) inhibitor therapies, compositions and methods comprising biomarker genes for identifying subjects that will respond to MEK inhibitor/target of rapamycin complex (TORC) inhibitor combination therapies, and compositions and methods comprising biomarker genes for identifying subjects that will respond to MEK inhibitor/chemotherapy combination therapies.

### BACKGROUND

Aberrant activation of the RAS-RAF-MEK-ERK1/2 pathway, one of four subfamilies of mitogen-activated protein kinases (MAPKs) that control multiple key physiological processes, occurs in more than 30% of human cancers. As part of this pathway, human MAP kinase kinase 1 (MEK1) and MEK2 have crucial roles in tumorigenesis, cell proliferation and inhibition of apoptosis and, therefore, MEK1/2 inhibition is an attractive therapeutic strategy in a number of cancers. Highly selective and potent non-ATP-competitive allosteric MEK1/2 inhibitors have been developed and assessed in numerous clinical studies over the past decade. Single-agent antitumor activity has been detected mainly in tumors that harbor mutations in genes encoding the members of the RAS and RAF protein families, such as certain melanomas. Combinations of MEK1/2 inhibitors and cytotoxic chemotherapy, and/or other targeted agents such as mechanistic target of rapamycin (mTOR) pathway inhibitors such as target of rapamycin complex 1 (TORC1) and TORC2, and more broadly phosphatidylinositol-3-kinase (PI3K) pathway inhibitors are being studied to expand the efficacy of this class of agents. Identifying predictive biomarkers and delineating de novo and acquired resistance mechanisms are essential for the future clinical development of MEK inhibitors, both in the context of monotherapy and as combination therapy with TORC inhibitors or chemotherapy. The present invention addresses this need and provides related advantages.

### SUMMARY

Compositions and methods are provided for identifying subjects that will respond to human MAP kinase kinase (MEK) inhibitor therapies as well as compositions and methods comprising biomarker genes for identifying subjects that will respond to MEK inhibitor/target of rapamycin complex (TORC) inhibitor combination therapies, and compositions and methods comprising biomarker genes for identifying subjects that will respond to MEK inhibitor/chemotherapy combination therapies.

In some embodiments, the invention provides a method for determining a genotype of one or more biomarker genes selected from ADAR, APC, ARID1A, ARID2, ASXL1, ATM, ATRX, BAP1, BRCA1, BRCA2, CDKN2A, CHD2, CIC, CMTR2, CREBBP, CUL3, DICER1, DLC1, DNMT1, DUSP4, EP300, FAT1, FBXW7, JAK1, KDM5C, KDM6A, KEAP1, KMT2C, KMT2D, KRAS, LRP1B, MET, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TREX1, TP53, TSC1, TSC2, USP15, and ZFHX3 in a biological sample from a human subject afflicted with cancer, said method comprising (a) contacting the biological sample with one or more binding agents, each binding agent specific for one of the biomarker genes, and (b) further processing the sample to determine a genotype of each of the one or more biomarker genes in the biological sample.

In some embodiments, the invention provides a method for determining a genotype of one or more biomarker genes selected from APC, ARID1A, ARID2, ASXL1, ATM, ATRX, BAP1, BRCA1, BRCA2, CDKN2A, CIC, CMTR2, CUL3, DICER1, DLC1, DUSP4, EP300, FAT1, FBXW7, KDM5C, KDM6A, KEAP1, KMT2C, KMT2D, KRAS, LRP1B, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TP53, TSC1, TSC2, USP15, and ZFHX3 in a biological sample from a human subject afflicted with cancer, said method comprising (a) contacting the biological sample with one or more binding agents, each binding agent specific for one of the biomarker genes, and (b) further processing the sample to determine a genotype of each of the one or more biomarker genes in the biological sample.

In some embodiments, the biomarker genes are selected from APC, ARID2, ATM, ATRX, BRCA2, CDKN2A, CMTR2, FBXW7, KDM6A, KEAP1, KMT2D, KRAS, MGA, MSH2, NF1, NF2, PALB2, PCNA, PTEN, PTPN11, PTPRD, RB1, RBM10, RNF43, SETD2, SMAD4, STAG2, STK11, TP53, and TSC1. In some embodiments, the biomarker genes are selected from APC, ARID2, ATM, ATRX, BRCA2, CDKN2A, CMTR2, KEAP1, KMT2D, MGA, NF1, PTEN, PTPRD, RB1, RBM10, RNF43, SETD2, SMAD4, STAG2, STK11, TP53, and TSC1.

In some embodiments, the invention provides a method of predicting resistance of tumor growth to inhibition by a therapy comprising a MEK inhibitor, said method comprising: (a) detecting in a tumor sample from a human subject afflicted with cancer a genotype of one or more biomarker genes; (b) analyzing the genotype of the one or more biomarker genes in the tumor sample; and (c) predicting resistance of tumor cell growth to inhibition by the therapy comprising a MEK inhibitor, if the tumor sample comprises (i) an inactivating KEAP1, KMT2D, or SMAD4 mutation, (ii) a decreased copy number of KEAP1, KMT2D, or SMAD4, or (iii) a decreased expression of KEAP1, KMT2D, or SMAD4 mRNA or protein.

In some embodiments, the invention provides a method of predicting resistance of tumor growth to inhibition by a therapy comprising a MEK inhibitor, said method comprising: (a) detecting in a tumor sample from a human subject afflicted with cancer a genotype of one or more biomarker genes; (b) analyzing the genotype of the one or more biomarker genes in the tumor sample; and (c) predicting resistance of tumor cell growth to inhibition by the therapy comprising a MEK inhibitor, if the tumor sample comprises (i) an inactivating ARID2, BAP1, BRCA1, CIC, KMT2D, NCOA6, or RASA1 mutation, (ii) a decreased copy number of ARID2, BAP1, BRCA1, CIC, KMT2D, NCOA6, or RASA1, or (iii) a decreased expression of ARID2, BAP1, BRCA1, CC, KMT2D, NsCOA6, or RASA1 mRNA or protein.

In some embodiments, the invention provides a method of predicting resistance of tumor growth to inhibition by a therapy comprising a MEK inhibitor, said method comprising: (a) detecting in a tumor sample from a human subject afflicted with cancer a genotype of one or more biomarker genes; (b) analyzing the genotype of the one or more biomarker genes in the tumor sample; and (c) predicting resistance of tumor cell growth to inhibition by the therapy comprising a MEK inhibitor, if the tumor sample comprises (i) an inactivating KEAP1, KMT2D, or SMAD4 mutation, (ii) a decreased copy number of KEAP1, KMT2D, or SMAD4, or (iii) a decreased expression of KEAP1, KMT2D, or SMAD4 mRNA or protein.

In some embodiments, the invention provides a method of predicting resistance of tumor growth to inhibition by a therapy comprising a MEK inhibitor, said method comprising: (a) detecting in a tumor sample from a human subject afflicted with cancer a genotype of one or more biomarker genes; (b) analyzing the genotype of the one or more biomarker genes in the tumor sample; and (c) predicting resistance of tumor cell growth to inhibition by the therapy comprising a MEK inhibitor, if the tumor sample comprises (i) an inactivating ARID2, BAP1, BRCA1, CIC, KMT2D, NCOA6, or RASA1 mutation, (ii) a decreased copy number of ARID2, BAP1, BRCA1, CIC, KMT2D, NCOA6, or RASA1, or (iii) a decreased expression of ARID2, BAP1, BRCA1, CIC, KMT2D, NCOA6, or RASA1 mRNA or protein.

In some embodiments, the invention provides a method of predicting resistance of tumor growth to inhibition by a therapy comprising a MEK inhibitor, said method comprising: (a) detecting in a tumor sample from a human subject afflicted with cancer a genotype of one or more biomarker genes; (b) analyzing the genotype of the one or more biomarker genes in the tumor sample; and (c) predicting resistance of tumor cell growth to inhibition by the therapy comprising a MEK inhibitor, if the tumor sample comprises (i) an inactivating KEAP1, KMT2D, PTEN, or SMAD4 mutation, (ii) a decreased copy number of KEAP1, KMT2D, PTEN, or SMAD4, or (iii) a decreased expression of KEAP1, KMT2D, PTEN, or SMAD4 mRNA or protein.

In some embodiments, the invention provides a method of predicting resistance of tumor growth to inhibition by a therapy comprising a MEK inhibitor, said method comprising: (a) detecting in a tumor sample from a human subject afflicted with cancer a genotype of one or more biomarker genes; (b) analyzing the genotype of the one or more biomarker genes in the tumor sample; and (c) predicting resistance of tumor cell growth to inhibition by the therapy comprising a MEK inhibitor, if the tumor sample comprises (i) an inactivating KEAP1, KMT2D, PTEN, or SMAD4 mutation, (ii) a decreased copy number of KEAP1, KMT2D, PTEN, or SMAD4, or (iii) a decreased expression of KEAP1, KMT2D, PTEN, or SMAD4 mRNA or protein.

In some embodiments, the invention provides a method of predicting resistance of tumor growth to inhibition by a therapy comprising a MEK inhibitor, said method comprising: (a) detecting in a tumor sample from a human subject afflicted with cancer a genotype of one or more biomarker genes; (b) analyzing the genotype of the one or more biomarker genes in the tumor sample; and (c) predicting resistance of tumor cell growth to inhibition by the therapy comprising a MEK inhibitor, if the tumor sample comprises (i) an inactivating ARID2, BAP1, BRCA1, CIC, KDM6A, KMT2C, KMT2D, LRP1B, NCOA6, NF2, PALB2, PTEN, RASA1, SMAD2, SMG1, SMARCA4, or TET2 mutation, (ii) a decreased copy number of ARID2, BAP1, BRCA1, CIC, KDM6A, KMT2C, KMT2D, LRP1B, NCOA6, NF2, PALB2, PTEN, RASA1, SMAD2, SMG1, SMARCA4, or TET2, or (iii) a decreased expression of ARID2, BAP1, BRCA1, CIC, KDM6A, KMT2C, KMT2D, LRP1B, NCOA6, NF2, PALB2, PTEN, RASA1, SMAD2, SMG1, SMARCA4, or TET2 mRNA or protein.

In some embodiments, the invention provides a method of predicting sensitivity of tumor growth to inhibition by a therapy comprising a MEK inhibitor, said method comprising: (a) detecting in a tumor sample from a human subject afflicted with cancer a genotype of one or more biomarker genes; (b) analyzing the genotype of the one or more biomarker genes in the tumor sample; and (c) predicting sensitivity of tumor cell growth to inhibition by the therapy comprising a MEK inhibitor, if the tumor sample comprises (i) an inactivating CDKN2A, EP300. RBM10, or SETD2 mutation, (ii) a decreased copy number of CDKN2A, EP300, RBM10, or SETD2, or (iii) decreased expression of CDKN2A, EP300, RBM10, or SETD2 mRNA or protein.

In some embodiments, the invention provides a method of predicting sensitivity of tumor growth to inhibition by a therapy comprising a MEK inhibitor, said method comprising: (a) detecting in a tumor sample from a human subject afflicted with cancer a genotype of one or more biomarker genes; (b) analyzing the genotype of the one or more biomarker genes in the tumor sample; and (c) predicting sensitivity of tumor cell growth to inhibition by the therapy comprising a MEK inhibitor, if the tumor sample comprises (i) an inactivating CDKN2A, EP300, KRAS, MGA, RB1, RBM10, SETD2, STAG2, STK11, TP53, USP15, or ZFHX3 mutation, (ii) a decreased copy number of CDKN2A, EP300, KRAS, MGA, RB1, RBM10, SETD2, STAG2, STK11, TP53, USP15, or ZFHX3, or (iii) decreased expression of CDKN2A, EP300, KRAS, MGA, RB1, RBM10, SETD2, STAG2, STK11, TP53, USP15, or ZFHX3 mRNA or protein.

In some embodiments, the one or more biomarker genes are selected from APC, ARID1A, ARID2, ASXL1, ATM, ATRX BAP1, BRCA1, BRCA2, CDKN2A, CHD2, CIC, CMTR2, CREBBP, CUL3, DICER1, DLC1, DUSP4, EP300, FAT1, FBXW7, KDM5C, KDM6A, KEAP1, KMT2C, KMT2D, KRASWT, LKB1, LRP1B, MGA, MSH2, MTAP, NCOA6, NF1, NF2, P53, PALB2, PBRM1, PCNA, PTEN, PTPN13, PTPRD, PTPRS, RASA1, RB1, RB1CC1, RBM10, RNF43, SETD2, SHP2, SMAD2, SMAD4, SMARCA4, SMG1, STAG2, TET2, TGFBR2, TSC1, TSC2, USP15, and ZFHX3.

In some embodiments, the invention provides a method of predicting sensitivity of tumor growth to inhibition by a therapy comprising a MEK inhibitor, said method comprising: (a) detecting in a tumor sample from a human subject afflicted with cancer a genotype of one or more biomarker genes; (b) analyzing the genotype of the one or more biomarker genes in the tumor sample; and (c) predicting sensitivity of tumor cell growth to inhibition by the therapy comprising a MEK inhibitor, if the tumor sample comprises (i) an inactivating APC, ARID2, ATM, ATRX, BRCA2, CDKN2A, CMTR2, MGA, NF1, PTEN, PTPRD, RB1, RBM10, RNF43, SETD2, STAG2, STK11, TP53, or TSC1 mutation, (ii) a decreased copy number of APC, ARID2, ATM, ATRX, BRCA2, CDKN2A, CMTR2, MGA, NF1, PTEN, PTPRD, RB1, RBM10, RNF43, SETD2, STAG2, STK11, TP53, or TSC1, or (iii) decreased expression of APC, ARID2, ATM, ATRX, BRCA2, CDKN2A, CMTR2, MGA, NF1, PTEN, PTPRD, RB1, RBM10, RNF43, SETD2, STAG2, STK11, TP53, or TSC1 mRNA or protein.

In some embodiments, the invention provides a method of predicting sensitivity of tumor growth to inhibition by a therapy comprising a MEK inhibitor, said method comprising: (a) detecting in a tumor sample from a human subject afflicted with cancer a genotype of one or more biomarker genes; (b) analyzing the genotype of the one or more biomarker genes in the tumor sample; and (c) predicting sensitivity of tumor cell growth to inhibition by the therapy comprising a MEK inhibitor, if the tumor sample comprises (i) an inactivating APC, ARID2, ATM, ATRX, BRCA2, CDKN2A, CMTR2, MGA, NF1, PTEN, PTPRD, RB1, RBM10, RNF43, SETD2, STAG2, STK11, TP53, or TSC1 mutation, (ii) a decreased copy number of APC, ARID2, ATM, ATRX, BRCA2, CDKN2A, CMTR2, MGA, NF1, PTEN, PTPRD, RB1, RBM10, RNF43, SETD2, STAG2, STK11, TP53, or TSC1, or (iii) decreased expression of APC, ARID2, ATM, ATRX, BRCA2, CDKN2A, CMTR2, MGA, NF1, PTEN, PTPRD, RB1, RBM10, RNF43, SETD2, STAG2, STK11, TP53, or TSC1 mRNA or protein.

In some embodiments, the invention provides a method of predicting response of tumor growth to inhibition by a therapy comprising a MEK inhibitor, said method comprising: (a) determining in a tumor sample from a human subject afflicted with cancer a genotype of one or more biomarker genes; (b) analyzing the genotype of the one or more biomarker genes in the tumor sample; and (c) predicting the response of tumor cell growth to inhibition by the therapy comprising a MEK inhibitor, if the tumor sample comprises (i) an inactivating APC, ARID2, ATM, ATRX, BRCA2, CDKN2A, CMTR2, MGA, NF1, PTEN, PTPRD, RB1, RBM10, RNF43, SETD2, STAG2, STK11, TP53, or TSC1 mutation, (ii) a decreased copy number of APC, ARID2, ATM, ATRX, BRCA2, CDKN2A, CMTR2, MGA, NF1, PTEN, PTPRD, RB1, RBM10, RNF43, SETD2, STAG2, STK11, TP53, or TSC1, (iii) decreased expression of APC, ARID2, ATM, ATRX, BRCA2, CDKN2A, CMTR2, MGA, NF1, PTEN, PTPRD, RB1, RBM10, RNF43, SETD2, STAG2, STK11, TP53, or TSC1 mRNA or protein; (iv) an inactivating KEAP1, KMT2D, or SMAD4 mutation, (v) a decreased copy number of KEAP1, KMT2D, or SMAD4, or (vi) a decreased expression of KEAP1, KMT2D, or SMAD4 mRNA or protein.

In some embodiments, the invention provides a method of predicting response of tumor growth to inhibition by a therapy comprising a MEK inhibitor, said method comprising: (a) determining in a tumor sample from a human subject afflicted with cancer a genotype of one or more biomarker genes; (b) analyzing the genotype of the one or more biomarker genes in the tumor sample; and (c) predicting the response of tumor cell growth to inhibition by the therapy comprising a MEK inhibitor, if the tumor sample comprises (i) an inactivating APC, ARID2, ATM, ATRX, BRCA2, CDKN2A, CMTR2, MGA, NF1, PTEN, PTPRD, RB1, RBM10, RNF43, SETD2, STAG2, STK11, TP53, or TSC1 mutation, (ii) a decreased copy number of APC, ARID2, ATM, ATRX, BRCA2, CDKN2A, CMTR2, MGA, NF1, PTEN, PTPRD, RB1, RBM10, RNF43, SETD2, STAG2, STK11, TP53, or TSC1, (iii) decreased expression of APC, ARID2, ATM, ATRX, BRCA2, CDKN2A, CMTR2, MGA, NF1, PTEN, PTPRD, RB1, RBM10, RNF43, SETD2, STAG2, STK11, TP53, or TSC1 mRNA or protein; (iv) an inactivating KEAP1, KMT2D, or SMAD4 mutation, (v) a decreased copy number of KEAP1, KMT2D, or SMAD4, or (vi) a decreased expression of KEAP1, KMT2D, or SMAD4 mRNA or protein.

In some embodiments, the invention provides a method of predicting response of tumor growth to inhibition by a therapy comprising a MEK inhibitor, said method comprising: (a) determining in a tumor sample from a human subject afflicted with cancer a genotype of one or more biomarker genes; (b) analyzing the genotype of the one or more biomarker genes in the tumor sample; and (c) predicting the response of tumor cell growth to inhibition by the therapy comprising a MEK inhibitor, if the tumor sample comprises (i) an inactivating CDKN2A, EP300. RBM10, or SETD2 mutation, (ii) a decreased copy number of CDKN2A, EP300, RBM10, or SETD2, (iii) decreased expression of CDKN2A, EP300, RBM10, or SETD2 mRNA or protein; (iv) an inactivating ARID2, BAP1, BRCA1, CIC, KMT2D, NCOA6, or RASA1 mutation, (v) a decreased copy number of ARID2, BAP1, BRCA1, CIC, KMT2D, NCOA6, or RASA1, or (vi) a decreased expression of ARID2, BAP1, BRCA1, CIC, KMT2D, NCOA6, or RASA1 mRNA or protein.

In some embodiments, the invention provides a method of predicting response of tumor growth to inhibition by a therapy comprising a MEK inhibitor, said method comprising: (a) determining in a tumor sample from a human subject afflicted with cancer a genotype of one or more biomarker genes; (b) analyzing the genotype of the one or more biomarker genes in the tumor sample; and (c) predicting the response of tumor cell growth to inhibition by the therapy comprising a MEK inhibitor, if the tumor sample comprises (i) an inactivating CDKN2A, EP300, KRAS, MGA, RB1, RBM10, SETD2, STAG2, STK11, TP53, USP15, or ZFHX3 mutation, (ii) a decreased copy number of CDKN2A, EP300, KRAS, MGA, RB1, RBM10, SETD2, STAG2, STK11, TP53, USP15, or ZFHX3, (iii) decreased expression of CDKN2A, EP300, KRAS, MGA, RB1, RBM10, SETD2, STAG2, STK11, TP53, USP15, or ZFHX3 mRNA or protein; (iv) an inactivating ARID2, BAP1, BRCA1, CIC, KDM6A, KMT2C, KMT2D, LRP1B, NCOA6, NF2, PALB2, PTEN, RASA1, SMAD2, SMG1, SMARCA4, or TET2 mutation, (v) a decreased copy number of ARID2, BAP1, BRCA1, CIC, KDM6A, KMT2C, KMT2D, LRP1B, NCOA6, NF2, PALB2, PTEN, RASA1, SMAD2, SMG1, SMARCA4, or TET2, or (vi) a decreased expression of ARID2, BAP1, BRCA1, CIC, KDM6A, KMT2C, KMT2D, LRP1B, NCOA6, NF2, PALB2, PTEN, RASA1, SMAD2, SMG1, SMARCA4, or TET2 mRNA or protein.

In some embodiments, the invention provides a method of treating non-small cell lung cancer (NSCLC) comprising the step of treating a subject with a MEK inhibitor when a tumor sample obtained from the subject comprises (i) an inactivating mutation in one or more of CDKN2A, EP300, RBM10, or SETD2, (ii) a decreased copy number of one or more of CDKN2A, EP300, RBM10, or SETD2, or (iii) decreased expression of CDKN2A, EP300, RBM10, or SETD2 mRNA or protein. In further embodiments, the tumor sample obtained from the subject further comprises absence of (i) an inactivating mutation in one or more of ARID2, BAP1, BRCA1, CIC, KMT2D, NCOA6, or RASA1, (ii) a decreased copy number of one or more of ARID2, BAP1. BRCA1, CIC, KMT2D, NCOA6, or RASA1, or (iii) decreased expression of ARID2, BAP1. BRCA1, CIC, KMT2D, NCOA6, or RASA1 mRNA or protein.

In some embodiments, the invention provides a method of treating non-small cell lung cancer (NSCLC) comprising the step of treating a subject with a MEK inhibitor when a tumor sample obtained from the subject comprises (i) an inactivating mutation in one or more of CDKN2A, EP300, KRAS, MGA, RB1, RBM10, SETD2, STAG2, STK11. TP53, USP15, or ZFHX3, (ii) a decreased copy number of one or more of CDKN2A, EP300, KRAS, MGA, RB1, RBM10, SETD2, STAG2, STK11, TP53, USP15, or ZFHX3, or (iii) decreased expression of CDKN2A, EP300, KRAS, MGA, RB1, RBM10, SETD2, STAG2, STK11, TP53, USP15, or ZFHX3 mRNA or protein. In further embodiments, the tumor sample obtained from the subject further comprises absence of (i) an inactivating mutation in one or more of ARID2, BAP1, BRCA1, CIC, KDM6A, KMT2C, KMT2D, LRP1B, NCOA6, NF2, PALB2, PTEN, RASA1, SMAD2, SMG1, SMARCA4, or TET2, (ii) a decreased copy number of one or more of ARID2, BAP1, BRCA1, CIC, KDM6A, KMT2C, KMT2D, LRP1B, NCOA6, NF2, PALB2, PTEN, RASA1, SMAD2, SMG1, SMARCA4, or TET2, or (iii) decreased expression of CDKN2A, EP300, KRAS, MGA, RB1, RBM10, SETD2, STAG2, STK11, TP53, USP15, or ZFHX3 mRNA or protein.

In some embodiments, the invention provides a method of predicting resistance of tumor growth to inhibition by a combination therapy comprising a MEK inhibitor and a TORC inhibitor, said method comprising: (a) detecting in a tumor sample from a human subject afflicted with cancer a genotype of one or more biomarker genes; (b) analyzing the genotype of the one or more biomarker genes in the tumor sample; and (c) predicting resistance of tumor cell growth to inhibition by the combination therapy comprising a MEK inhibitor and a TORC inhibitor, if the tumor sample comprises (i) an inactivating KMT2D or PTEN mutation, (ii) a decreased copy number of KMT2D or PTEN, or (iii) a decreased expression of KMT2D or PTEN mRNA or protein.

In some embodiments, the invention provides a method of predicting sensitivity of tumor growth to inhibition by a combination therapy comprising a MEK inhibitor and a TORC inhibitor, said method comprising: (a) detecting in a tumor sample from a human subject afflicted with cancer a genotype of one or more biomarker genes APC, ARID2, ATM, ATRX, BRCA2, CDKN2A, CMTR2, FBXW7, KDM6A, KEAP1, KMT2D, KRAS, MGA, MSH2, NF1, NF2, PALB2, PCNA, PTEN, PTPN11, PTPRD, RB1, RBM10, RNF43, SETD2, SMAD4, STAG2, STK11, TP53, and TSC1; (b) analyzing the genotype of the one or more biomarker genes in the tumor sample; and (c) predicting sensitivity of tumor cell growth to inhibition by the combination therapy comprising a MEK inhibitor and a TORC inhibitor, if the tumor sample comprises (i) an inactivating ARID2, CDKN2A, CMTR2, SETD2, STAG2, or STK11 mutation, (ii) a decreased copy number of ARID2, CDKN2A, CMTR2, SETD2, STAG2, or STK11, or (iii) decreased expression of ARID2, CDKN2A, CMTR2, SETD2, STAG2, or STK11 mRNA or protein.

In some embodiments, the invention provides a method of predicting sensitivity of tumor growth to inhibition by a combination therapy comprising a MEK inhibitor and a TORC inhibitor, said method comprising: (a) detecting in a tumor sample from a human subject afflicted with cancer a genotype of one or more biomarker genes; (b) analyzing the genotype of the one or more biomarker genes in the tumor sample; and (c) predicting sensitivity of tumor cell growth to inhibition by the combination therapy comprising a MEK inhibitor and a TORC inhibitor, if the tumor sample comprises (i) an inactivating ARID2, ATM, CDKN2A, CMTR2, KRAS, MGA, MSH2, RB1, RBM10, RNF43, SETD2, SMAD4, STAG2, or STK11 mutation, (ii) a decreased copy number of ARID2, ATM, CDKN2A, CMTR2, KRAS, MGA, MSH2, RB1, RBM10, RNF43, SETD2, SMAD4, STAG2, or STK11, or (iii) decreased expression of ARID2, ATM, CDKN2A, CMTR2, KRAS, MGA, MSH2, RB1, RBM10, RNF43, SETD2M SMAD4, STAG2, or STK11 mRNA or protein.

In some embodiments, the invention provides a method of predicting response of tumor growth to inhibition by a combination therapy comprising a MEK inhibitor and a TORC inhibitor, said method comprising: (a) determining in a tumor sample from a human subject afflicted with cancer a genotype of one or more biomarker genes; (b) analyzing the genotype of the one or more biomarker genes in the tumor sample; and (c) predicting the response of tumor cell growth to inhibition by the combination therapy comprising a MEK inhibitor and a TORC inhibitor, if the tumor sample comprises (i) an inactivating ARID2, CDKN2A, CMTR2, SETD2, STAG2, or STK11 mutation, (ii) a decreased copy number of ARID2, CDKN2A, CMTR2, SETD2, STAG2, or STK11, (iii) decreased expression of ARID2, CDKN2A, CMTR2, SETD2, STAG2, or STK11 mRNA or protein; (iv) an inactivating KMT2D or PTEN mutation, (v) a decreased copy number of KMT2D or PTEN, or (vi) a decreased expression of KMT2D or PTEN mRNA or protein.

In some embodiments, the invention provides a method of predicting response of tumor growth to inhibition by a combination therapy comprising a MEK inhibitor and a TORC inhibitor, said method comprising: (a) determining in a tumor sample from a human subject afflicted with cancer a genotype of one or more biomarker genes; (b) analyzing the genotype of the one or more biomarker genes in the tumor sample; and (c) predicting the response of tumor cell growth to inhibition by the combination therapy comprising a MEK inhibitor and a TORC inhibitor, if the tumor sample comprises (i) an inactivating ARID2, ATM, CDKN2A, CMTR2, KRAS, MGA, MSH2, RB1, RBM10, RNF43, SETD2, SMAD4, STAG2, or STK11 mutation, (ii) a decreased copy number of ARID2, ATM, CDKN2A, CMTR2, KRAS, MGA, MSH2, RB1, RBM10, RNF43, SETD2, SMAD4, STAG2, or STK11, (iii) decreased expression ARID2, ATM, CDKN2A, CMTR2, KRAS, MGA, MSH2, RB1, RBM10, RNF43, SETD2, SMAD4, STAG2, or STK11 mRNA or protein; (iv) an inactivating KMT2D or PTEN mutation, (v) a decreased copy number of KMT2D or PTEN, or (vi) a decreased expression of KMT2D or PTEN mRNA or protein.

In some embodiments, the invention provides a method of treating non-small cell lung cancer (NSCLC) comprising the step of treating a subject with a combination therapy comprising a MEK inhibitor and a TORC inhibitor when a tumor sample obtained from the subject comprises (i) an inactivating mutation in one or more of ARID2, ATM, CDKN2A, CMTR2, KRAS, MGA, MSH2, RB1, RBM10, RNF43, SETD2, SMAD4, STAG2, or STK11, (ii) a decreased copy number of one or more of ARID2, ATM, CDKN2A, CMTR2, KRAS, MGA, MSH2, RB1, RBM10, RNF43, SETD2, SMAD4, STAG2, or STK11, or (iii) a decreased expression of ARID2, ATM, CDKN2A, CMTR2, KRAS, MGA, MSH2, RB1, RBM10, RNF43, SETD2, SMAD4, STAG2, or STK11 mRNA or protein. In further embodiments, the tumor sample obtained from the subject further comprises absence of (i) an inactivating mutation in one or more of KMT2D or PTEN, (ii) a decreased copy number of one or more of KMT2D or PTEN, or (iii) a decreased expression of KMT2D or PTEN mRNA or protein.

In some embodiments, the invention provides a method of predicting resistance of tumor growth to inhibition by a combination therapy comprising a MEK inhibitor and a chemotherapy, comprising: (a) detecting in a tumor sample from a human subject afflicted with cancer a genotype of one or more biomarker genes; (b) analyzing the genotype of the one or more biomarker genes in the tumor sample; and (c) predicting resistance of tumor cell growth to inhibition by the combination therapy comprising a MEK inhibitor and a chemotherapy if the tumor sample comprises (i) an inactivating ARID2, ASXL1, ATM, BAP1, BRCA1, BRCA2, CIC, CUL3, DLC1, FAT1, KDM5C, KDM6A, KMT2C, KMT2D, LRP1B, MGA, MTAP, NCOA6, NF2, PALB2, PBRM1, PTEN, PTPN11, PTPN13, PTPRS, RASA1, RB1, RB1CC1, RNF43, SMAD2, SMAD4, SMARCA4, SMG1, TGFBR2, TP53, or TSC2 mutation, (ii) a decreased copy number of ARID2, ASXL1, ATM, BAP1, BRCA1, BRCA2, CIC, CUL3, DLC1, FAT1, KDM5C, KDM6A, KMT2C, KMT2D, LRP1B, MGA, MTAP, NCOA6, NF2, PALB2, PBRM1, PTEN, PTPN11, PTPN13, PTPRS, RASA1, RB1, RB1CC1, RNF43, SMAD2, SMAD4, SMARCA4, SMG1, TGFBR2, TP53, or TSC2, or (iii) a decreased expression of ARID2, ASXL1, ATM, BAP1, BRCA1, BRCA2, CIC, CUL3, DLC1, FAT1, KDM5C, KDM6A, KMT2C, KMT2D, LRP1B, MGA, MTAP, NCOA6, NF2, PALB2, PBRM1, PTEN, PTPN11, PTPN13, PTPRS, RASA1, RB1, RB1CC1, RNF43, SMAD2, SMAD4, SMARCA4, SMG1, TGFBR2, TP53, or TSC2 mRNA or protein. In some embodiments, the tumor sample comprises (i) an inactivating ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, or SMG1 mutation, (ii) a decreased copy number of ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, or SMG1, or (iii) a decreased expression of ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, or SMG1 mRNA or protein.

In some embodiments, the invention provides a method of predicting sensitivity of tumor growth to inhibition by a combination therapy comprising MEK inhibitor and a chemotherapy, comprising: (a) detecting in a tumor sample from a human subject afflicted with cancer a genotype of one or more biomarker genes; (b) analyzing the genotype of the one or more biomarker genes in the tumor sample; and (c) predicting sensitivity of tumor cell growth to inhibition by the combination therapy comprising a MEK inhibitor and a chemotherapy, if the tumor sample comprises (i) an inactivating APC, ARID1A, ATRX, CDKN2A, CMTR2, DUSP4, EP300, FBXW7, KEAP1, KRAS, MSH2, NF1, PTPRD, RBM10, SETD2, STAG2, STK11, TET2, TSC1, USP15, or ZFHX3 mutation, (ii) a decreased copy number of APC, ARID1A, ATRX, CDKN2A, CMTR2, DUSP4, EP300, FBXW7, KEAP1, KRAS, MSH2, NF1, PTPRD, RBM10, SETD2, STAG2, STK11, TET2, TSC1, USP15, or ZFHX3, or (iii) decreased expression of APC, ARID1A, ATRX, CDKN2A, CMTR2, DUSP4, EP300, FBXW7, KEAP1, KRAS, MSH2, NF1, PTPRD, RBM10, SETD2, STAG2, STK11, TET2, TSC1, USP15, or ZFHX3 mRNA or protein. In some embodiments, the tumor sample comprises (i) an inactivating CDKN2A, EP300, KEAP1, KRAS, RBM10, SETD2, STK11, TSC1, or USP15 mutation, (ii) a decreased copy number of CDKN2A, EP300, KEAP1, KRAS, RBM10, SETD2, STK11, TSC1, or USP15, or (iii) a decreased expression of CDKN2A, EP300, KEAP1, KRAS, RBM10, SETD2, SIK11, TSC1, or USP15 mRNA or protein.

In some embodiments, the invention provides a method of predicting response of tumor growth to inhibition by a combination therapy comprising a MEK inhibitor and a chemotherapy, comprising: (a) determining in a tumor sample from a human subject afflicted with cancer a genotype of one or more biomarker genes; (b) analyzing the genotype of the one or more biomarker genes in the tumor sample; and (c) predicting the response of tumor cell growth to inhibition by the combination therapy comprising a MEK inhibitor and a chemotherapy, if the tumor sample comprises (i) an inactivating ARID2, ASXL1, ATM, BAP1, BRCA1, BRCA2, CIC, CUL3, DLC1, FAT1, KDM5C, KDM6A, KMT2C, KMT2D, LRP1B, MGA, MTAP, NCOA6, NF2, PALB2, PBRM1, PTEN, PTPN11, PTPN13, PTPRS, RASA1, RB1, RB1CC1, RNF43, SMAD2, SMAD4, SMARCA4, SMG1, TGFBR2, TP53, or TSC2 mutation, (ii) a decreased copy number of ARID2, ASXL1, ATM, BAP1, BRCA1, BRCA2, CIC, CUL3, DLC1, FAT1, KDM5C, KDM6A, KMT2C, KMT2D, LRP1B, MGA, MTAP, NCOA6, NF2, PALB2, PBRM1, PTEN, PTPN11, PTPN13, PTPRS, RASA1, RB1, RB1CC1, RNF43, SMAD2, SMAD4, SMARCA4, SMG1, TGFBR2, TP53, or TSC2, (iii) decreased expression of ARID2, ASXL1, ATM, BAP1, BRCA1, BRCA2, CIC, CUL3, DLC1, FAT1, KDM5C, KDM6A, KMT2C, KMT2D, LRP1B, MGA, MTAP, NCOA6, NF2, PALB2, PBRM1, PTEN, PTPN11, PTPN13, PTPRS, RASA1, RB1, RB1CC1, RNF43, SMAD2, SMAD4, SMARCA4, SMG1, TGFBR2, TP53, or TSC2 mRNA or protein; (iv) an inactivating APC, ARID1A, ATRX, CDKN2A, CMTR2, DUSP4, EP300, FBXW7, KEAP1, KRAS, MSH2, NF1, PTPRD, RBM10, SETD2, STAG2, STK11, TET2, TSC1, USP15, or ZFHX3 mutation, (v) a decreased copy number of APC, ARID1A, ATRX CDKN2A, CMTR2, DUSP4, EP300, FBXW7, KEAP1, KRAS, MSH2, NF1, PTPRD, RBM10, SETD2, STAG2, STK11, TET2, TSC1, USP15, or ZFHX3, or (vi) a decreased expression of APC, ARID1A, ATRX, CDKN2A, CMTR2, DUSP4, EP300, FBXW7, KEAP1, KRAS, MSH2, NF1, PTPRD, RBM10, SETD2, STAG2, STK11, TET2, TSC1, USP15, or ZFHX3 mRNA or protein.

In some embodiments, the invention provides a method of predicting response of tumor growth to inhibition by a combination therapy comprising a MEK inhibitor and a chemotherapy, comprising: (a) determining in a tumor sample from a human subject afflicted with cancer a genotype of one or more biomarker genes; (b) analyzing the genotype of the one or more biomarker genes in the tumor sample; and (c) predicting the response of tumor cell growth to inhibition by the combination therapy comprising a MEK inhibitor and a chemotherapy, if the tumor sample comprises (i) an inactivating ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, or SMG1 mutation, (ii) a decreased copy number of ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, or SMG1, (iii) decreased expression of ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, or SMG1 mRNA or protein; (iv) an inactivating CDKN2A, EP300, KEAP1, KRAS, RBM10, SETD2, STK11, TSC1, or USP15 mutation, (v) a decreased copy number of CDKN2A, EP300, KEAP1, KRAS, RBM10, SETD2, STK11, TSC1, or USP15, or (vi) a decreased expression of CDKN2A, EP300, KEAP1, KRAS, RBM10, SETD2, STK11, TSC1, or USP15 mRNA or protein.

In some embodiments, the invention provides a method of treating non-small cell lung cancer (NSCLC) comprising the step of treating a subject with a combination therapy comprising a combination therapy comprising MEK inhibitor and a chemotherapy when a tumor sample obtained from the subject comprises (i) an inactivating mutation in one or more of CDKN2A, EP300, KEAP1, KRAS, RBM10, SETD2, STK11, TSC1, or USP15, (ii) a decreased copy number of one or more of CDKN2A, EP300, KEAP1, KRAS, RBM10, SETD2, STK11, TSC1, or USP15, or (iii) a decreased expression of CDKN2A, EP300, KEAP1, KRAS, RBM10, SETD2, STK11, TSC1, or USP15 mRNA or protein. In further embodiments, the tumor sample obtained from the subject further comprises absence of (i) an inactivating mutation in one or more of ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, or SMG1, (ii) a decreased copy number of one or more of ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, or SMG1, or (iii) a decreased expression of ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, or SMG1 mRNA or protein

In some embodiments, the methods of the invention further comprise administering chemotherapy to the subject. In some embodiment, the chemotherapy comprises a chemotherapeutic agent belonging to the class comprising taxanes. In some embodiments, the chemotherapeutic agent is paclitaxel or docetaxel. In some embodiment, the chemotherapy comprises a chemotherapeutic agent belonging to class comprising platinum-based chemotherapeutic agents. In some embodiments, the chemotherapeutic agent is carboplatin. In some embodiment, the chemotherapy comprises a chemotherapeutic agent belonging to class comprising folate antimetabolites. In some embodiments, the chemotherapeutic agent is pemetrexed. In some embodiments, the invention provides a composition comprising one or more isolated biomarker genes selected from ADAR, APC, ARID1A, ARID2, ASXL1, ATM, ATRX, BAP1, BRCA1, BRCA2, CDKN2A, CHD2, CIC, CMTR2, CREBBP, CUL3, DICER1, DLC1, DNMT1, DUSP4, EP300, FAT1, FBXW7, JAK1, KDM5C, KDM6A, KEAP1, KMT2C, KMT2D, KRAS, LRP1B, MET, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TREX1, TP53, TSC1, TSC2, USP15, and ZFHX3. In some embodiments, the invention provides a composition comprising one or more isolated biomarker genes selected from APC, ARID2, ATM, ATRX, BRCA2, CDKN2A, CMTR2, FBXW7, KDM6A, KEAP1, KMT2D, KRAS, MGA, MSH2, NF1, NF2, PALB2, PCNA, PTEN, PTPN11, PTPRD, RB1, RBM10, RNF43, SETD2, SMAD4, STAG2, STK11, TP53, and TSC1. In some embodiments, the invention provides a composition comprising one or more isolated biomarker genes selected from APC, ARID2, ATM, ATRX, BRCA2, CDKN2A, CMTR2, KEAP1, KMT2D, MGA, NF1, PTEN, PTPRD, RB1, RBM10, RNF43, SETD2, SMAD4, STAG2, STK11, TP53, and TSC1.

In some embodiments, the invention provides a composition comprising one or more isolated biomarker genes selected from APC, ARID1A, ARID2, ASXL1, ATM, ATRX, BAP1, BRCA1, BRCA2, CDKN2A, CIC, CMTR2, CUL3, DICER1, DLC1, DUSP4, EP300, FAT1, FBXW7, KDM5C, KDM6A, KEAP1, KMT2C, KMT2D, KRAS, LRP1B, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TP53, TSC1, TSC2, USP15, and ZFHX3.

In some embodiments, the invention provides a composition comprising one or more isolated biomarker genes selected from ARID2, BAP1, BRCA1, CIC, KDM6A, KMT2C, KMT2D, LRP1B, NCOA6, NF2, PALB2, PTEN, RASA1, SMAD2, SMG1, SMARCA4, and TET2. In some embodiments, the invention provides a composition comprising one or more isolated biomarker genes selected from ARID2, BAP1, BRCA1, CIC, KMT2D, NCOA6, and RASA1.

In some embodiments, the invention provides a composition comprising one or more isolated biomarker genes selected from CDKN2A, EP300, KRAS, MGA, RB1, RBM10, SETD2, STAG2, STK1, TP53, USP15, and ZFHX3. In some embodiments, the invention provides a composition comprising one or more isolated biomarker genes selected from CDKN2A, EP300. RBM10, and SETD2.

In some embodiments, the invention provides a method of detecting one or more isolated biomarker genes selected from ADAR, APC, ARID1A, ARID2, ASXL1, ATM, ATRX, BAP1, BRCA1, BRCA2, CDKN2A, CHD2, CIC, CMTR2, CREBBP, CUL3, DICER1, DLC1, DNMT1, DUSP4, EP300, FAT1, FBXW7, JAK1, KDM5C, KDM6A, KEAP1, KMT2C, KMT2D, KRAS, LRP1B, MET, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TREX1, TP53, TSC1, TSC2, USP15, and ZFHX3 in a human subject, said method comprising: a) obtaining a biological sample from a subject; and b) detecting whether the one or more isolated biomarker genes are present in the biological sample by contacting the biological sample with a binding agent and detecting binding between the one or more isolated biomarker genes and the binding agent.

In some embodiments, the invention provides a method of detecting one or more isolated biomarker genes selected from APC, ARID2, ATM, ATRX, BRCA2, CDKN2A, CMTR2, FBXW7, KDM6A, KEAP1, KMT2D, KRAS, MGA, MSH2, NF1, NF2, PALB2, PCNA, PTEN, PTPN11, PTPRD, RB1, RBM10, RNF43, SETD2, SMAD4, STAG2, STK11, TP53, and TSC1 in a human subject, said method comprising: a) obtaining a biological sample from the human subject; and b) detecting whether the one or more isolated biomarker genes are present in the biological sample by contacting the biological sample with a binding agent and detecting binding between the one or more isolated biomarker genes and the binding agent.

In some embodiments, the invention provides a method of detecting one or more isolated biomarker genes selected from APC, ARID2, ATM, ATRX, BRCA2, CDKN2A, CMTR2, KEAP1, KMT2D, MGA, NF1, PTEN, PTPRD, RB1, RBM10, RNF43, SETD2, SMAD4, STAG2, STK11, TP53, and TSC1 in a human subject, said method comprising: a) obtaining a biological sample from the human subject; and b) detecting whether the one or more isolated biomarker genes are present in the biological sample by contacting the biological sample with a binding agent and detecting binding between the one or more isolated biomarker genes and the binding agent.

In some embodiments, the invention provides a method of detecting one or more isolated biomarker genes selected from APC, ARID2, ATM, ATRX, BRCA2, CDKN2A, CMTR2, MGA, NF1, PTEN, PTPRD, RB1, RBM10, RNF43, SETD2, STAG2, STK11, TP53, and TSC1 in a human subject, said method comprising: a) obtaining a biological sample from the human subject; and b) detecting whether the one or more isolated biomarker genes are present in the biological sample by contacting the biological sample with a binding agent and detecting binding between the one or more isolated biomarker genes and the binding agent.

In some embodiments, the invention provides a method of detecting one or more isolated biomarker genes selected from ARID2, CDKN2A, CMTR2, SETD2, STAG2, and STK11 in a human subject, said method comprising: a) obtaining a biological sample from the human subject; and b) detecting whether the one or more isolated biomarker genes are present in the biological sample by contacting the biological sample with a binding agent and detecting binding between the one or more isolated biomarker genes and the binding agent. In some embodiments, the one or more isolated biomarker genes are selected from ARID2, ATM, CDKN2A, CMTR2, KRAS, MGA, MSH2, RB1, RBM10, RNF43, SETD2, SMAD4, STAG2, and STK11.

In some embodiments, the invention provides a method of detecting one or more isolated biomarker genes selected from KEAP1, KMT2D, and SMAD4 in a human subject, said method comprising: a) obtaining a biological sample from the human subject; and b) detecting whether the one or more isolated biomarker genes are present in the biological sample by contacting the biological sample with a binding agent and detecting binding between the one or more isolated biomarker genes and the binding agent.

In some embodiments, the invention provides a method of detecting one or more isolated biomarker genes selected from KEAP1, KMT2D, PTEN and SMAD4 in a human subject, said method comprising: a) obtaining a biological sample from the human subject; and b) detecting whether the one or more isolated biomarker genes are present in the biological sample by contacting the biological sample with a binding agent and detecting binding between the one or more isolated biomarker genes and the binding agent.

In some embodiments, the invention provides a method of detecting one or more isolated biomarker genes selected from KMT2D and PTEN in a human subject, said method comprising: a) obtaining a biological sample from the human subject; and b) detecting whether the one or more isolated biomarker genes are present in the biological sample by contacting the biological sample with a binding agent and detecting binding between the one or more isolated biomarker genes and the binding agent.

In some embodiments, the invention provides a method of detecting one or more isolated biomarker genes selected from APC, ARID1A, ARID2, ASXL1, ATM, ATRX, BAP1, BRCA1, BRCA2, CDKN2A, CIC, CMTR2, CUL3, DICER1, DLC1, DUSP4, EP300, FAT1, FBXW7, KDM5C, KDM6A, KEAP1, KMT2C, KMT2D, KRAS, LRP1B, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TP53, TSC1, TSC2, USP15, and ZFHX3 in a human subject, said method comprising: a) obtaining a biological sample from the human subject; and b) detecting whether the one or more isolated biomarker genes are present in the biological sample by contacting the biological sample with a binding agent and detecting binding between the one or more isolated biomarker genes and the binding agent.

In some embodiments, the invention provides a method of detecting one or more isolated biomarker genes selected from ARID2, ASXL1, ATM, BAP1, BRCA1, BRCA2, CIC, CUL3, DLC1, FAT1, KDM5C, KDM6A, KMT2C, KMT2D, LRP1B, MGA, MTAP, NCOA6, NF2, PALB2, PBRM1, PTEN, PTPN11, PTPN13, PTPRS, RASA1, RB1, RB1CC1, RNF43, SMAD2, SMAD4, SMARCA4, SMG, TGFBR2, TP53, and TSC2 in a human subject, said method comprising: a) obtaining a biological sample from the human subject; and b) detecting whether the one or more isolated biomarker genes are present in the biological sample by contacting the biological sample with a binding agent and detecting binding between the one or more isolated biomarker genes and the binding agent.

In some embodiments, the invention provides a method of detecting one or more isolated biomarker genes selected from ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, or SMG1 in a human subject, said method comprising: a) obtaining a biological sample from the human subject; and b) detecting whether the one or more isolated biomarker genes are present in the biological sample by contacting the biological sample with a binding agent and detecting binding between the one or more isolated biomarker genes and the binding agent.

In some embodiments, the invention provides a method of detecting one or more isolated biomarker genes selected from APC, ARID1A, ATRX, CDKN2A, CMTR2, DUSP4, EP300, FBXW7, KEAP1, KRAS, MSH2, NF1, PTPRD, RBM10, SETD2, STAG2, STK11, TET2, TSC1, USP15, and ZFHX3 in a human subject, said method comprising: a) obtaining a biological sample from the human subject; and b) detecting whether the one or more isolated biomarker genes are present in the biological sample by contacting the biological sample with a binding agent and detecting binding between the one or more isolated biomarker genes and the binding agent. In some embodiments, the one or more isolated biomarker genes are selected from CDKN2A, EP300, KEAP1, KRAS, RBM10, SETD2, SIK11, TSC1, and USP15.

In some embodiments, the invention provides a method of detecting one or more isolated biomarker genes selected from ARID2, BAP1, BRCA1, CIC, KDM6A, KMT2C, KMT2D, LRP1B, NCOA6, NF2, PALB2, PTEN, RASA1, SMAD2, SMG1, SMARCA4, and TET2 in a human subject, said method comprising: a) obtaining a biological sample from the human subject; and b) detecting whether the one or more isolated biomarker genes are present in the biological sample by contacting the biological sample with a binding agent and detecting binding between the one or more isolated biomarker genes and the binding agent.

In some embodiments, the invention provides a method of detecting one or more isolated biomarker genes selected from ARID2, BAP1, BRCA1, CIC, KMT2D, NCOA6, and RASA1 in a human subject, said method comprising: a) obtaining a biological sample from the human subject; and b) detecting whether the one or more isolated biomarker genes are present in the biological sample by contacting the biological sample with a binding agent and detecting binding between the one or more isolated biomarker genes and the binding agent.

In some embodiments, the invention provides a method of detecting one or more isolated biomarker genes selected from ARID2, BAP1, BRCA1, CIC, KDM6A, KMT2C, KMT2D, LRP1B, NCOA6, NF2, PALB2, PTEN, RASA1, SMAD2, SMG1, SMARCA4, and TET2 in a human subject, said method comprising: a) obtaining a biological sample from the human subject; and b) detecting whether the one or more isolated biomarker genes are present in the biological sample by contacting the biological sample with a binding agent and detecting binding between the one or more isolated biomarker genes and the binding agent.

In some embodiments, the invention provides a method of detecting one or more isolated biomarker genes selected from CDKN2A, EP300, KRAS, MGA, RB1, RBM10, SETD2, STAG2, STK11, TP53, USP15, and ZFHX3 in a human subject, said method comprising: a) obtaining a biological sample from the human subject; and b) detecting whether the one or more isolated biomarker genes are present in the biological sample by contacting the biological sample with a binding agent and detecting binding between the one or more isolated biomarker genes and the binding age

In some embodiments, the invention provides a method of detecting one or more isolated biomarker genes selected from CDKN2A, EP300, RBM10, and SETD2 in a human subject, said method comprising: a) obtaining a biological sample from the human subject; and b) detecting whether the one or more isolated biomarker genes are present in the biological sample by contacting the biological sample with a binding agent and detecting binding between the one or more isolated biomarker genes and the binding age

In some embodiments, the invention provides a method of detecting one or more isolated biomarker genes selected from CDKN2A, EP300, RBM10, and SETD2 in a human subject, said method comprising: a) obtaining a biological sample from the human subject; and b) detecting whether the one or more isolated biomarker genes are present in the biological sample by contacting the biological sample with a binding agent and detecting binding between the one or more isolated biomarker genes and the binding agent.

In some embodiments, the methods of the invention comprise administering a MEK inhibitor monotherapy. In some embodiments, the methods of the invention comprise administering a combination therapy comprising a MEK inhibitor and a TORC inhibitor. In some embodiments, the methods of the invention comprise administering a combination therapy comprising a MEK inhibitor and a chemotherapy. In some embodiments a MEK monotherapy, or combination therapy comprising a MEK inhibitor and a TORC inhibitor, or combination therapy comprising a MEK inhibitor and a chemotherapy is administered when a tumor sample obtained from the subject comprises one or more of the biomarker gene profiles provided by the present invention.

In some embodiments, the methods comprise administering a MEK monotherapy, or combination therapy comprising a MEK inhibitor and a TORC inhibitor, or combination therapy comprising a MEK inhibitor and a chemotherapy to a subject if the predicted response to the therapy is sensitivity of tumor growth to inhibition by a therapy comprising a MEK inhibitor. In some embodiments, the methods further comprise administering a MEK monotherapy, or combination therapy comprising a MEK inhibitor and a TORC inhibitor, or combination therapy comprising a MEK inhibitor and a chemotherapy to a subject if the predicted response to the therapy is sensitivity of tumor growth to inhibition and absence of resistance of tumor growth to inhibition by a therapy comprising a MEK inhibitor.

In some embodiments, the methods comprise selecting a subject for treatment with a MEK monotherapy, or combination therapy comprising a MEK inhibitor and a TORC inhibitor, or combination therapy comprising a MEK inhibitor and a chemotherapy to a subject if the predicted response to the therapy is sensitivity of tumor growth to inhibition by a therapy comprising a MEK inhibitor. In some embodiments, the methods further comprise selecting a subject for treatment with a administering a MEK monotherapy, or combination therapy comprising a MEK inhibitor and a TORC inhibitor, or combination therapy comprising a MEK inhibitor and a chemotherapy to a subject if the predicted response to the therapy is sensitivity of tumor growth to inhibition and absence of resistance of tumor growth to inhibition by a therapy comprising a MEK inhibitor.

In some embodiments, the invention provides methods for selecting a subject for treatment with a MEK monotherapy if it is likely that the subject will respond to the MEK monotherapy, wherein the likelihood of response is determined by performing any of the disclosed methods for predicting resistance, sensitivity, or response of tumor growth to inhibition by the therapy on a biological sample obtained from the subject. In some embodiments, the determination is based on results of an existing biological sample of the subject.

In some embodiments, the invention provides methods for selecting a subject for treatment with a combination therapy if it is likely that the subject will respond to the combination therapy comprising a MEK inhibitor and a TORC inhibitor, wherein the likelihood of response is determined by performing any of the disclosed methods for predicting resistance, sensitivity, or response of tumor growth to inhibition by the therapy on a biological sample obtained from the subject. In some embodiments, the determination is based on results of an existing biological sample of the subject.

In some embodiments, the invention provides methods for selecting a subject for treatment with a combination therapy comprising a MEK inhibitor and a chemotherapy if it is likely that the subject will respond to the combination therapy comprising a MEK inhibitor and a chemotherapy, wherein the likelihood of response is determined by performing any of the disclosed methods for predicting resistance, sensitivity, or response of tumor growth to inhibition by the therapy on a biological sample obtained from the subject. In some embodiments, the determination is based on results of an existing biological sample of the subject.

In some embodiments, the invention provides a method of enriching a prospective patient population for subjects likely to respond to a MEK inhibitor therapy comprising performing the disclosed methods of predicting response of tumor growth to inhibition on a biological sample obtained from one or more subjects within the patient population.

In some embodiments, the invention provides a method of enriching a prospective patient population for subjects likely to respond to a combination therapy comprising a MEK inhibitor and a TORC inhibitor comprising performing the disclosed methods of predicting response of tumor growth to inhibition on a biological sample obtained from one or more subjects within the patient population.

In some embodiments, the invention provides a method of enriching a prospective patient population for subjects likely to respond to a combination therapy comprising a MEK inhibitor and a chemotherapy comprising performing the disclosed methods of predicting response of tumor growth to inhibition on a biological sample obtained from one or more subjects within the patient population.

### BRIEF DESCRIPTION OF THE DRAWINGS

FIG. 1A-FIG. 1JJ show bootstrap confidence intervals for RTN_{score} for each of the biomarker genes tested in studies ST-0003, ST-0007, OMI-0006 and OMI-0007 as described in Example 1. FIG. 1A shows bootstrap confidence intervals for RTN_{score} for Apc in ST-0003, ST-0007, and OMI-0007. FIG. 1B shows bootstrap confidence intervals for RTN_{score} for Arid2 in ST-0003, ST-0007, and OMI-0007. FIG. 1C shows bootstrap confidence intervals for RTN_{score} for Atm in ST-0003, ST-0007, and OMI-0007. FIG. 1D shows bootstrap confidence intervals for RTN_{score} for Atrx in ST-0003, ST-0007, and OMI-0007. FIG. 1E shows bootstrap confidence intervals for RTN_{score} for Brca2 in ST-0003, ST-0007, and OMI-0007. FIG. 1F shows bootstrap confidence intervals for RTN_{score} for Cdkn2a in ST-0003, ST-0007, and OMI-0007. FIG. 1G shows bootstrap confidence intervals for RTN_{score} for Cmtr2 in ST-0003, ST-0007, and OMI-0007. FIG. 1H shows bootstrap confidence intervals for RTN_{score} for Fbxw7 in OMI-0007. FIG. 1I shows bootstrap confidence intervals for RTN_{score} for Kdm6a in OMI-0007. FIG. 1J shows bootstrap confidence intervals for RTN_{score} for Keap1 in ST-0003, ST-0007, and OMI-0007. FIG. 1K shows bootstrap confidence intervals for RTN_{score} for Kmt2d in ST-0003, ST-0007, and OMI-0007. FIG. 1L shows bootstrap confidence intervals for RTN_{score} for KrasWT in OMI-0007. FIG. 1M shows bootstrap confidence intervals for RTN_{score} for Lkb1 in ST-0003, ST-0007, and OMI-0007. FIG. 1N shows bootstrap confidence intervals for RTN_{score} for Mga in ST-0003, ST-0007, and OMI-0007. FIG. 1O shows bootstrap confidence intervals for RTN_{score} for Msh2 in OMI-0007. FIG. 1P shows bootstrap confidence intervals for RTN_{score} for Nf1 in ST-0003, ST-0007, and OMI-0007. FIG. 1Q shows bootstrap confidence intervals for RTN_{score} for Nf2 in OMI-0007. FIG. 1R shows bootstrap confidence intervals for RTN_{score} for p53 in ST-0003, ST-0007, and OMI-0007. FIG. 1S shows bootstrap confidence intervals for RTN_{score} for Palb2 in OMI-0007. FIG. 1T shows bootstrap confidence intervals for RTN_{score} for Pcna in OMI-0007. FIG. 1U shows bootstrap confidence intervals for RTN_{score} for Pten in ST-0003, ST-0007, and OMI-0007. FIG. 1V shows bootstrap confidence intervals for RTN_{score} for Ptprd in ST-0003, ST-0007, and OMI-0007. FIG. 1W shows bootstrap confidence intervals for RTN_{score} for Rb1 in ST-0003, ST-0007, and OMI-0007. FIG. 1X shows bootstrap confidence intervals for RTN_{score} for Rbm10 in ST-0003, ST-0007, and OMI-0007. FIG. 1Y shows bootstrap confidence intervals for RTN_{score} for Rnf43 in ST-0003, ST-0007, and OMI-0007. FIG. 1Z shows bootstrap confidence intervals for RTN_{score} for Setd2 in ST-0003, ST-0007, and OMI-0007. FIG. 1AA shows bootstrap confidence intervals for RTN_{score} for Shp2 in OMI-0007. FIG. 1BB shows bootstrap confidence intervals for RTN_{score} for Smad4 in ST-0003, ST-0007, and OMI-0007. FIG. 1CC shows bootstrap confidence intervals for RTN_{score} for Stag2 in ST-0003, ST-0007, and OMI-0007. FIG. 1DD shows bootstrap confidence intervals for RTN_{score} for TSC1 in ST-0003, ST-0007, and OMI-0007. FIG. 1EE shows bootstrap confidence intervals for RTN_{score} for Apc, Arid2, Atm, Atrx and Brca2 in OMI-0006. FIG. 1FF shows bootstrap confidence intervals for RTN_{score} for Cdkna2, Cmtr2, Fbxw7, Kdm6a and Keap1 in OMI-0006. FIG. 1GG shows bootstrap confidence intervals for RTN_{score} for Kmt2d, KrasWT, Lkb1, Mga and Msh2 in OMI-0006. FIG. 1HH shows bootstrap confidence intervals for RTN_{score} for Nf1, Nf2, p53, Palb2 and Pcna in OMI-0006. FIG. 1II shows bootstrap confidence intervals for RTN_{score} for Pten, Ptprd, Rb1, Rbm10 and Rnf43 in OMI-0006. FIG. 1JJ shows bootstrap confidence intervals for RTN_{score} for Setd2, Shp2, Smad4, Stag2 and Tsc1 in OMI-0006.

FIG. 2 shows a biomarker heatmap showing the study of pharmacogenomic interactions (PGx) of MEKi with inactivation of tumor suppressor genes. Relative tumor number (RTN)>0 indicates drug resistance, and RTN=1 corresponds to 2× change in tumor number (larger than each cutoff) relative to change in untreated vs. treated for oncogene-only tumors, while RTN=−1 corresponds to 0.5× change and drug sensitivity. *: p<0.05 and +: p<0.2. Both p-values are two-tailed and based on fraction of bootstraps with RTN scores great or less than 0. Missing cells in heatmap correspond to genotypes that were not assayed in their particular study.

FIG. 3 shows a table depicting benefits of MEKi/TORCi combination therapy in 30 distinct genotypes. Columns B, C, D, E, and G represent average total neoplastic cell count for mice given each respective therapy relative to vehicle controls. Columns F and H represent the fold improvement in efficacy above what one would expect from the combined product of the efficacy from each monotherapy arm.

FIG. 4A-FIG. 4C shows analysis of 1000 bootstrap resamplings of mice for which the median neoplastic cell count for each tumor genotype in each study group was computed. FIG. 4A shows the distribution of shrinkages, defined as the ratio of neoplastic cell counts of each drug group relative to the control group, for each drug group in FIG. 4B shows the effect of TORCi/MEKi combination therapies relative to monotherapies, the distribution over bootstraps of the ratio of TORCi/trametinib shrinkages is shown relative to trametinib monotherapy shrinkages, and FIG. 4C shows the product of the corresponding TORCi/trametinib monotherapy shrinkages.

FIG. 5 shows a biomarker heatmap showing the study of pharmacogenomic interactions (PGx) of MEKi/chemotherapy combination treatment with inactivation of tumor suppressor genes. Relative tumor number (RTN)>0 indicates drug resistance, and RTN=1 corresponds to 2× change in tumor number (larger than each cutoff) relative to change in untreated vs. treated for oncogene-only tumors, while RTN=−1 corresponds to 0.5× change and drug sensitivity. *: p<0.05 and +: p<0.2. Both p-values are two-tailed and based on fraction of bootstraps with RTN scores great or less than 0.

FIG. 6 shows a bar graph depicting aggregated RTN scores and their corresponding 95% confidence intervals for MEK monotherapy. Shading indicates the respective classification memberships, which was defined as follows. Resistant or Sensitive: Family-wise error rate (FWER) less or equal to 0.05 and absolute value of RTN score greater than 0.1. Extended: false discovery rate (FDR) less than or equal to 0.1 and absolute value of RTN score greater than 0.08.

FIG. 7 shows a bar graph depicting aggregated RTN scores and their corresponding 95% confidence intervals for combination therapy comprising a MEK inhibitor and a TORC inhibitor. Shading indicates the respective classification memberships, which was defined as follows. Resistant or Sensitive: Family-wise error rate (FWER) less or equal to 0.05 and absolute value of RTN score greater than 0.1. Extended: false discovery rate (FDR) less than or equal to 0.1 and absolute value of RTN score greater than 0.08.

FIG. 8 shows a bar graph depicting aggregated RTN scores and their corresponding 95% confidence intervals for combination therapy comprising a MEK inhibitor and a chemotherapy. Shading indicates the respective classification memberships, which was defined as follows. Resistant or Sensitive: Family-wise error rate (FWER) less or equal to 0.05 and absolute value of RTN score greater than 0.1. Extended: false discovery rate (FDR) less than or equal to 0.1 and absolute value of RTN score greater than 0.08.

### DETAILED DESCRIPTION

The present invention is based, in part, on the surprising discovery that the genotype of particular biomarker genes can be used to predict a human subject's response to a MEK inhibitor therapy. In one embodiment, the genotype is predictive of sensitivity to a MEK inhibitor therapy. In one embodiment, the genotype is predictive of resistance to a MEK inhibitor therapy. The inventions disclosed herein provide new and advantageous methods for determining whether a human subject afflicted with cancer is a candidate for a MEK inhibitor therapy.

The present invention is further based on the surprising discovery that the genotype of particular biomarker genes can be used to predict a human subject's response to a combination therapy comprising a MEK inhibitor and a TORC inhibitor. In one embodiment, the genotype is predictive of sensitivity to a combination therapy comprising a MEK inhibitor and a TORC inhibitor. In one embodiment, the genotype is predictive of resistance to a combination therapy comprising a MEK inhibitor and a TORC inhibitor. The inventions disclosed herein provide new and advantageous methods for determining whether a human subject afflicted with cancer is a candidate for a combination therapy comprising a MEK inhibitor and a TORC inhibitor.

The present invention is further based on the surprising discovery that the genotype of particular biomarker genes can be used to predict a human subject's response to a combination therapy comprising a MEK inhibitor and a chemotherapy. In one embodiment, the genotype is predictive of sensitivity to a combination therapy comprising a MEK inhibitor and a chemotherapy. In one embodiment, the genotype is predictive of resistance to a combination therapy comprising a MEK inhibitor and a chemotherapy. The inventions disclosed herein provide new and advantageous methods for determining whether a human subject afflicted with cancer is a candidate for a combination therapy comprising a MEK inhibitor and a chemotherapy.

In one embodiment, the invention provides a method comprising: (a) determining the genotype of one or more biomarker genes in a biological sample from a human subject; (b) identifying whether the subject is sensitive to a therapy comprising a MEK inhibitor based on the genotype. In one embodiment, the invention provides a method comprising: (a) determining the genotype of one or more biomarker genes in a biological sample from a human subject; (b) identifying whether the subject is resistant to a therapy comprising a MEK inhibitor based on the genotype. In some embodiment, the methods further comprise treating a subject comprising administering to the subject a MEK inhibitor if the subject is identified as sensitive to a therapy comprising a MEK inhibitor.

In one embodiment, the invention provides a method comprising: (a) determining the genotype of one or more biomarker genes in a biological sample from a human subject; (b) identifying whether the subject is sensitive to a combination therapy comprising a MEK inhibitor and a TORC inhibitor based on the genotype. In one embodiment, the invention provides a method comprising: (a) determining the genotype of one or more biomarker genes in a biological sample from a human subject; (b) identifying whether the subject is resistant to a combination therapy comprising a MEK inhibitor and a TORC inhibitor based on the genotype. In some embodiment, the methods further comprise treating a subject comprising administering to the subject a combination therapy comprising a MEK inhibitor and a TORC inhibitor if the subject is identified as sensitive to a combination therapy comprising a MEK inhibitor and a TORC inhibitor.

In one embodiment, the invention provides a method comprising: (a) determining the genotype of one or more biomarker genes in a biological sample from a human subject; (b) identifying whether the subject is sensitive to a combination therapy comprising a MEK inhibitor and a chemotherapy based on the genotype. In one embodiment, the invention provides a method comprising: (a) determining the genotype of one or more biomarker genes in a biological sample from a human subject; (b) identifying whether the subject is resistant to a combination therapy comprising a MEK inhibitor and a chemotherapy based on the genotype. In some embodiment, the methods further comprise treating a subject comprising administering to the subject a combination therapy comprising a MEK inhibitor and a chemotherapy if the subject is identified as sensitive to a combination therapy comprising a MEK inhibitor and a chemotherapy.

In one embodiment, the biomarker genes useful in the methods of the invention comprise one or more of ADAR, APC, ARID1A, ARID2, ASXL1, ATM, ATRX, BAP1, BRCA1, BRCA2, CDKN2A, CHD2, CIC, CMTR2, CREBBP, CUL3, DICER1, DLC1, DNMT1, DUSP4, EP300, FAT1, FBXW7, JAK1, KDM5C, KDM6A, KEAP1, KMT2C, KMT2D, KRAS, LRP1B, MET, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TREX1, TP53, TSC1, TSC2, USP15, and ZFHX3. In one embodiment, the biomarker genes useful in the methods of the invention comprise two or more of ADAR, APC, ARID1A, ARID2, ASXL1, ATM, ATRX, BAP1, BRCA1, BRCA2, CDKN2A, CHD2, CIC, CMTR2, CREBBP, CUL3, DICER1, DLC1, DNMT1, DUSP4, EP300, FAT1, FBXW7, JAK1, KDM5C, KDM6A, KEAP1, KMT2C, KMT2D, KRAS, LRP1B, MET, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TREX1, TP53, TSC1, TSC2, USP15, and ZFHX3. In one embodiment, the biomarker genes useful in the methods of the invention comprise three or more of ADAR, APC, ARID1A, ARID2, ASXL1, ATM, ATRX, BAP1, BRCA1, BRCA2, CDKN2A, CHD2, CIC, CMTR2, CREBBP, CUL3, DICER1, DLC1, DNMT1, DUSP4, EP300, FAT1, FBXW7, JAK1, KDM5C, KDM6A, KEAP1, KMT2C, KMT2D, KRAS, LRP1B, MET, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TREX1, TP53, TSC1, TSC2, USP15, and ZFHX3.

In one embodiment, the biomarker genes useful in the methods of the invention comprise one or more of APC, ARID2, ATM, ATRX, BRCA2, CDKN2A, CMTR2, FBXW7, KDM6A, KEAP1, KMT2D, KRAS, MGA, MSH2, NF1, NF2, PALB2, PCNA, PTEN, PTPN11, PTPRD, RB1, RBM10, RNF43, SETD2, SMAD4, STAG2, STK11, TP53, and TSC1. In one embodiment, the biomarker genes useful in the methods of the invention comprise two or more of APC, ARID2, ATM, ATRX, BRCA2, CDKN2A, CMTR2, FBXW7, KDM6A, KEAP1, KMT2D, KRAS, MGA, MSH2, NF1, NF2, PALB2, PCNA, PTEN, PTPN11, PTPRD, RB1, RBM10, RNF43, SETD2, SMAD4, STAG2, STK11, TP53, and TSC1. In one embodiment, the biomarker genes useful in the methods of the invention comprise three or more of APC, ARID2, ATM, ATRX, BRCA2, CDKN2A, CMTR2, FBXW7, KDM6A, KEAP1, KMT2D, KRAS, MGA, MSH2, NF1, NF2, PALB2, PCNA, PTEN, PTPN11, PTPRD, RB1, RBM10, RNF43, SETD2, SMAD4, STAG2, STK11, TP53, and TSC1.

In one embodiment, the biomarker genes useful in the methods of the invention comprise one or more of APC, ARID2, ATM, ATRX, BRCA2, CDKN2A, CMTR2, KEAP1, KMT2D, MGA, NF1, PTEN, PTPRD, RB1, RBM10, RNF43, SETD2, SMAD4, STAG2, STK11, TP53, and TSC1. In one embodiment, the biomarker genes useful in the methods of the invention comprise two or more of APC, ARID2, ATM, ATRX, BRCA2, CDKN2A, CMTR2, KEAP1, KMT2D, MGA, NF1, PTEN, PTPRD, RB1, RBM10, RNF43, SETD2, SMAD4, STAG2, STK11, TP53, and TSC1. In one embodiment, the biomarker genes useful in the methods of the invention comprise three or more of APC, ARID2, ATM, ATRX, BRCA2, CDKN2A, CMTR2, KEAP1, KMT2D, MGA, NF1, PTEN, PTPRD, RB1, RBM10, RNF43, SETD2, SMAD4, STAG2, STK11, TP53, and TSC1.

In one embodiment, the biomarker genes useful in the methods of the invention comprise one or more of CDKN2A, EP300, RBM10, and SETD2, In one embodiment, the biomarker genes useful in the methods of the invention comprise two or more of CDKN2A, EP300, RBM10, and SETD2, In one embodiment, the biomarker genes useful in the methods of the invention comprise three or more of CDKN2A, EP300, RBM10, and SETD2.

In one embodiment, the biomarker genes useful in the methods of the invention comprise one or more of CDKN2A, EP300, KRAS, MGA, RB1, RBM10, SETD2, STAG2, STK11, TP53, USP15, and ZFHX3. In one embodiment, the biomarker genes useful in the methods of the invention comprise two or more of CDKN2A, EP300, KRAS, MGA, RB1, RBM10, SETD2, STAG2, STK11, TP53, USP15, and ZFHX3. In one embodiment, the biomarker genes useful in the methods of the invention comprise three or more of CDKN2A, EP300, KRAS, MGA, RB1, RBM10, SETD2, STAG2, STK11, TP53, USP15, and ZFHX3.

In one embodiment, the biomarker genes useful in the methods of the invention comprise one or more of APC, ARID2, ATM, ATRX, BRCA2, CDKN2A, CMTR2, MGA, NF1, PTEN, PTPRD, RB1, RBM10, RNF43, SETD2, STAG2, STK11, TP53, and TSC1. In one embodiment, the biomarker genes useful in the methods of the invention comprise two or more of APC, ARID2, ATM, ATRX, BRCA2, CDKN2A, CMTR2, MGA, NF1, PTEN, PTPRD, RB1, RBM10, RNF43, SETD2, STAG2, STK11, TP53, and TSC1. In one embodiment, the biomarker genes useful in the methods of the invention comprise three or more of APC, ARID2, ATM, ATRX, BRCA2, CDKN2A, CMTR2, MGA, NF1, PTEN, PTPRD, RB1, RBM10, RNF43, SETD2, STAG2, STK11, TP53, and TSC1.

In one embodiment, the biomarker genes useful in the methods of the invention comprise one or more of ARID2, CDKN2A, CMTR2, SETD2, STAG2, and STK11. In one embodiment, the biomarker genes useful in the methods of the invention comprise two or more of ARID2, CDKN2A, CMTR2, SETD2, STAG2, and STK11. In one embodiment, the biomarker genes useful in the methods of the invention comprise three or more of ARID2, CDKN2A, CMTR2, SETD2, STAG2, and STK11.

In one embodiment, the biomarker genes useful in the methods of the invention comprise one or more of ARID2, ATM, CDKN2A, CMTR2, KRAS, MGA, MSH2, RB1, RBM10, RNF43, SETD2, SMAD4, STAG2, and STK11. In one embodiment, the biomarker genes useful in the methods of the invention comprise two or more of ARID2, ATM, CDKN2A, CMTR2, KRAS, MGA, MSH2, RB1, RBM10, RNF43, SETD2, SMAD4, STAG2, and STK11. In one embodiment, the biomarker genes useful in the methods of the invention comprise three or more, four or more, five or more, six or more, seven or more of ARID2, ATM, CDKN2A, CMTR2, KRAS, MGA, MSH2, RB1, RBM10, RNF43, SETD2, SMAD4, STAG2, and STK11.

In one embodiment, the biomarker genes useful in the methods of the invention comprise one or more of KEAP1, KMT2D, and SMAD4. In one embodiment, the biomarker genes useful in the methods of the invention comprise two or more of KEAP1, KMT2D, and SMAD4. In one embodiment, the biomarker genes useful in the methods of the invention comprise one or more of KEAP1, KMT2D, PTEN and SMAD4. In one embodiment, the biomarker genes useful in the methods of the invention comprise two or more of KEAP1, KMT2D, PTEN and SMAD4. In one embodiment, the biomarker genes useful in the methods of the invention comprise three or more of KEAP1, KMT2D, PTEN and SMAD4. In some embodiments, the biomarker genes useful in the methods of the invention comprise one or more of KEAP1, KMT2D, PTEN and SMAD4 and are resistance biomarker genes

In one embodiment, the biomarker genes useful in the methods of the invention comprise one or more of ARID2, BAP1, BRCA1, CIC, KMT2D, NCOA6, and RASA1. In one embodiment, the biomarker genes useful in the methods of the invention comprise two or more of ARID2, BAP1, BRCA1, CIC, KMT2D, NCOA6, and RASA1. In one embodiment, the biomarker genes useful in the methods of the invention comprise three or more of ARID2, BAP1, BRCA1, CIC, KMT2D, NCOA6, and RASA1.

In one embodiment, the biomarker genes useful in the methods of the invention comprise one or more of ARID2, BAP1, BRCA1, CIC, KDM6A, KMT2C, KMT2D, LRP1B, NCOA6, NF2, PALB2, PTEN, RASA1, SMAD2, SMG1, SMARCA4, and TET2. In one embodiment, the biomarker genes useful in the methods of the invention comprise two or more of ARID2, BAP1, BRCA1, CIC, KDM6A, KMT2C, KMT2D, LRP1B, NCOA6, NF2, PALB2. PTEN, RASA1, SMAD2, SMG1, SMARCA4, and TET2. In one embodiment, the biomarker genes useful in the methods of the invention comprise three or more, four or more, five or more, six or more, seven or more of ARID2, BAP1, BRCA1, CIC, KDM6A, KMT2C, KMT2D, LRP1B, NCOA6, NF2, PALB2, PTEN, RASA1, SMAD2, SMG1, SMARCA4, and TET2.

In one embodiment, the biomarker genes useful in the methods of the invention comprise one or more of KMT2D and PTEN.

In one embodiment, the biomarker genes useful in the methods of the invention comprise one or more of APC, ARID1A, ARID2, ASXL1, ATM, ATRX, BAP1, BRCA1, BRCA2, CDKN2A, CIC, CMTR2, CUL3, DICER1, DLC1, DUSP4, EP300, FAT1, FBXW7, KDM5C, KDM6A, KEAP1, KMT2C, KMT2D, KRAS, LRP1B, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TP53, TSC1, TSC2, USP15, and ZFHX3.

In one embodiment, the biomarker genes useful in the methods of the invention comprise one or more of ARID2, ASXL1, ATM, BAP1, BRCA1, BRCA2, CIC, CUL3, DLC1, FAT1, KDM5C, KDM6A, KMT2C, KMT2D, LRP1B, MGA, MTAP, NCOA6, NF2, PALB2, PBRM1, PTEN, PTPN11, PTPN13, PTPRS, RASA1, RB1, RB1CC1, RNF43, SMAD2, SMAD4, SMARCA4, SMG1, TGFBR2, TP53, and TSC2. In one embodiment, the biomarker genes useful in the methods of the invention comprise one or more of ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, or SMG1.

In one embodiment, the biomarker genes useful in the methods of the invention comprise one or more of APC, ARID1A, ATRX, CDKN2A, CMTR2, DUSP4, EP300, FBXW7, KEAP1, KRAS, MSH2, NF1, PTPRD, RBM10, SETD2, STAG2, STK11, TET2, TSC1, USP15, and ZFHX3. In one embodiment, the biomarker genes useful in the methods of the invention comprise one or more of CDKN2A, EP300, KEAP1, KRAS, RBM10, SETD2, SIK11, TSC1, and USP15.

In one embodiment, the invention provides a composition comprising one or more isolated biomarker genes selected from ADAR, APC, ARID1A, ARID2, ASXL1, ATM, ATRX, BAP1, BRCA1, BRCA2, CDKN2A, CHD2, CIC, CMTR2, CREBBP, CUL3, DICER1, DLC1, DNMT1, DUSP4, EP300, FAT1, FBXW7, JAK1, KDM5C, KDM6A, KEAP1, KMT2C, KMT2D, KRAS, LRP1B, MET, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TREX1, TP53, TSC1, TSC2, USP15, and ZFHX3. In one embodiment, the invention provides a composition comprising two or more isolated biomarker genes selected from ADAR, APC, ARID1A, ARID2, ASXL1, ATM, ATRX, BAP1, BRCA1, BRCA2, CDKN2A, CHD2, CIC, CMTR2, CREBBP, CUL3, DICER1, DLC1, DNMT1, DUSP4, EP300, FAT1, FBXW7, JAK1, KDM5C, KDM6A, KEAP1, KMT2C, KMT2D, KRAS, LRP1B, MET, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TREX1, TP53, TSC1, TSC2, USP15, and ZFHX3. In one embodiment, the invention provides a composition comprising three or more isolated biomarker genes selected from ADAR, APC, ARID1A, ARID2, ASXL1, ATM, ATRX, BAP1, BRCA1, BRCA2, CDKN2A, CHD2, CIC, CMTR2, CREBBP, CUL3, DICER1, DLC1, DNMT1, DUSP4, EP300, FAT1, FBXW7, JAK1, KDM5C, KDM6A, KEAP1, KMT2C, KMT2D, KRAS, LRP1B, MET, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TREX1, TP53, TSC1, TSC2, USP15, and ZFHX3.

In one embodiment, the invention provides a composition comprising one or more isolated biomarker genes selected from APC, ARID2, ATM, ATRX, BRCA2, CDKN2A, CMTR2, FBXW7, KDM6A, KEAP1, KMT2D, KRAS, MGA, MSH2, NF1, NF2, PALB2, PCNA, PTEN, PTPN11, PTPRD, RB1, RBM10, RNF43, SETD2, SMAD4, STAG2, STK11, TP53, and TSC1. In one embodiment, the invention provides a composition comprising two or more isolated biomarker genes selected from APC, ARID2, ATM, ATRX, BRCA2, CDKN2A, CMTR2, FBXW7, KDM6A, KEAP1, KMT2D, KRAS, MGA, MSH2, NF1, NF2, PALB2, PCNA, PTEN, PTPN11, PTPRD, RB1, RBM10, RNF43, SETD2, SMAD4, STAG2, STK11, TP53, and TSC1. In one embodiment, the invention provides a composition comprising three or more isolated biomarker genes selected from APC, ARID2, ATM, ATRX, BRCA2, CDKN2A, CMTR2, FBXW7, KDM6A, KEAP1, KMT2D, KRAS, MGA, MSH2, NF1, NF2, PALB2, PCNA, PTEN, PTPN11, PTPRD, RB1, RBM10, RNF43, SETD2, SMAD4, STAG2, STK11, TP53, and TSC1.

In one embodiment, the invention provides a composition comprising one or more isolated biomarker genes selected from APC, ARID2, ATM, ATRX, BRCA2, CDKN2A, CMTR2, KEAP1, KMT2D, MGA, NF1, PTEN, PTPRD, RB1, RBM10, RNF43, SETD2, SMAD4, STAG2, STK11, TP53, and TSC1. In one embodiment, the invention provides a composition comprising two or more isolated biomarker genes selected from APC, ARID2, ATM, ATRX, BRCA2, CDKN2A, CMTR2, KEAP1, KMT2D, MGA, NF1, PTEN, PTPRD, RB1, RBM10, RNF43, SETD2, SMAD4, STAG2, STK11, TP53, and TSC1. In one embodiment, the invention provides a composition comprising three or more isolated biomarker genes selected from APC, ARID2, ATM, ATRX, BRCA2, CDKN2A, CMTR2, KEAP1, KMT2D, MGA, NF1, PTEN, PTPRD, RB1, RBM10, RNF43, SETD2, SMAD4, STAG2, STK11, TP53, and TSC1.

In one embodiment, the invention provides a composition comprising one or more isolated biomarker genes selected from APC, ARID2, ATM, ATRX, BRCA2, CDKN2A, CMTR2, MGA, NF1, PTEN, PTPRD, RB1, RBM10, RNF43, SETD2, STAG2, STK11, TP53, and TSC1. In one embodiment, the invention provides a composition comprising two or more isolated biomarker genes selected from APC, ARID2, ATM, ATRX, BRCA2, CDKN2A, CMTR2, MGA, NF1, PTEN, PTPRD, RB1, RBM10, RNF43, SETD2, STAG2, STK11, TP53, and TSC1. In one embodiment, the invention provides a composition comprising three or more isolated biomarker genes selected from APC, ARID2, ATM, ATRX, BRCA2, CDKN2A, CMTR2, MGA, NF1, PTEN, PTPRD, RB1, RBM10, RNF43, SETD2, STAG2, STK11, TP53, and TSC1.

In one embodiment, the invention provides a composition comprising one or more isolated biomarker genes selected from CDKN2A, EP300, KRAS, MGA, RB1, RBM10, SETD2, STAG2, STK11, TP53, USP15, and ZFHX3. In one embodiment, the invention provides a composition comprising two or more isolated biomarker genes selected from CDKN2A, EP300, KRAS, MGA, RB1, RBM10, SETD2, STAG2, STK11, TP53, USP15, and ZFHX3. In one embodiment, the invention provides a composition comprising three or more isolated biomarker genes selected from CDKN2A, EP300, KRAS, MGA, RB1, RBM10, SETD2, STAG2, SIK11, TP53, USP15, and ZFHX3.

In one embodiment, the invention provides a composition comprising one or more isolated biomarker genes selected from CDKN2A, EP300, RBM10, and SETD2, In one embodiment, the invention provides a composition comprising two or more isolated biomarker genes selected from CDKN2A, EP300, RBM10, and SETD2, In one embodiment, the invention provides a composition comprising three or more isolated biomarker genes selected from CDKN2A, EP300, RBM10, and SETD2.

In one embodiment, the invention provides a composition comprising one or more isolated biomarker genes selected from ARID2, CDKN2A, CMTR2, SETD2, STAG2, and STK11. In one embodiment, the invention provides a composition comprising two or more isolated biomarker genes selected from ARID2, CDKN2A, CMTR2, SETD2, STAG2, and STK11. In one embodiment, the invention provides a composition comprising three or more isolated biomarker genes selected from ARID2, CDKN2A, CMTR2, SETD2, STAG2, and STK11.

In one embodiment, the invention provides a composition comprising one or more isolated biomarker genes selected from ARID2, ATM, CDKN2A, CMTR2, KRAS, MGA, MSH2, RB1, RBM10, RNF43, SETD2, SMAD4, STAG2, and STK11. In one embodiment, the invention provides a composition comprising two or more isolated biomarker genes selected from ARID2, ATM, CDKN2A, CMTR2, KRAS, MGA, MSH2, RB1, RBM10, RNF43, SETD2, SMAD4, STAG2, and STK11. In one embodiment, the invention provides a composition comprising three or more isolated biomarker genes selected from ARID2, ATM, CDKN2A, CMTR2, KRAS, MGA, MSH2, RB1, RBM10, RNF43, SETD2, SMAD4, STAG2, and STK11.

In one embodiment, the invention provides a composition comprising one or more isolated biomarker genes selected from ARID2, BAP1, BRCA1, CIC, KDM6A, KMT2C, KMT2D, LRP1B, NCOA6, NF2, PALB2, PTEN, RASA1, SMAD2, SMG1, SMARCA4, and TET2. In one embodiment, the invention provides a composition comprising two or more isolated biomarker genes selected from ARID2, BAP1, BRCA1, CIC, KDM6A, KMT2C, KMT2D, LRP1B, NCOA6, NF2, PALB2, PTEN, RASA1, SMAD2, SMG1, SMARCA4, and TET2. In one embodiment, the invention provides a composition comprising three or more, four or more, five or more, six or more, seven or more isolated biomarker genes selected from ARID2, BAP1, BRCA1, CIC, KDM6A, KMT2C, KMT2D, LRP1B, NCOA6, NF2, PALB2, PTEN, RASA1, SMAD2, SMG1, SMARCA4, and TET2.

In one embodiment, the invention provides a composition comprising one or more isolated biomarker genes selected from ARID2, BAP1, BRCA1, CIC, KMT2D, NCOA6, and RASA1. In one embodiment, the invention provides a composition comprising two or more isolated biomarker genes selected from ARID2, BAP1, BRCA1, CIC, KMT2D, NCOA6, and RASA1. In one embodiment, the invention provides a composition comprising three or more, four or more, five or more, six or more isolated biomarker genes selected from ARID2, BAP1, BRCA1, CIC, KMT2D, NCOA6, and RASA1.

In one embodiment, the invention provides a composition comprising one or more isolated biomarker genes selected from KEAP1, KMT2D, and SMAD4. In one embodiment, the invention provides a composition comprising two or more isolated biomarker genes selected from KEAP1, KMT2D, and SMAD4.

In one embodiment, the invention provides a composition comprising one or more isolated biomarker genes selected from KEAP1, KMT2D, PTEN and SMAD4. In one embodiment, the invention provides a composition comprising two or more isolated biomarker genes selected from KEAP1, KMT2D, PTEN and SMAD4. In one embodiment, the invention provides a composition comprising three or more isolated biomarker genes selected from KEAP1, KMT2D, PTEN and SMAD4. In some embodiments, the invention provides a composition comprising one or more isolated biomarker genes selected from KEAP1, KMT2D, PTEN and SMAD4.

In one embodiment, the invention provides a composition comprising one or more isolated biomarker genes selected from KMT2D and PTEN.

In one embodiment, the invention provides a composition comprising one or more isolated biomarker genes selected from APC, ARID1A, ARID2, ASXL1, ATM, ATRX, BAP1, BRCA1, BRCA2, CDKN2A, CIC, CMTR2, CUL3, DICER1, DLC1, DUSP4, EP300, FAT1, FBXW7, KDM5C, KDM6A, KEAP1, KMT2C, KMT2D, KRAS, LRP1B, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TP53, TSC1, TSC2, USP15, and ZFHX3.

In one embodiment, the invention provides a composition comprising two or more isolated biomarker genes selected from APC, ARID1A, ARID2, ASXL1, ATM, ATRX, BAP1, BRCA1, BRCA2, CDKN2A, CIC, CMTR2, CUL3, DICER1, DLC1, DUSP4, EP300, FAT1, FBXW7, KDM5C, KDM6A, KEAP1, KMT2C, KMT2D, KRAS, LRP1B, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TP53, TSC1, TSC2, USP15, and ZFHX3.

In one embodiment, the invention provides a composition comprising three or more isolated biomarker genes selected from APC, ARID1A, ARID2, ASXL1, ATM, ATRX, BAP1, BRCA1, BRCA2, CDKN2A, CIC, CMTR2, CUL3, DICER1, DLC1, DUSP4, EP300, FAT1, FBXW7, KDM5C, KDM6A, KEAP1, KMT2C, KMT2D, KRAS, LRP1B, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TP53, TSC1, TSC2, USP15, and ZFHX3.

In one embodiment, the invention provides a composition comprising four or more isolated biomarker genes selected from APC, ARID1A, ARID2, ASXL1, ATM, ATRX, BAP1, BRCA1, BRCA2, CDKN2A, CIC, CMTR2, CUL3, DICER1, DLC1, DUSP4, EP300, FAT1, FBXW7, KDM5C, KDM6A, KEAP1, KMT2C, KMT2D, KRAS, LRP1B, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TP53, TSC1, TSC2, USP15, and ZFHX3. In one embodiment, the invention provides a composition comprising five or more isolated biomarker genes selected from APC, ARID1A, ARID2, ASXL1, ATM, ATRX, BAP1, BRCA1, BRCA2, CDKN2A, CIC, CMTR2, CUL3, DICER1, DLC1, DUSP4, EP300, FAT1, FBXW7, KDM5C, KDM6A, KEAP1, KMT2C, KMT2D, KRAS, LRP1B, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TP53, TSC1, TSC2, USP15, and ZFHX3.

In one embodiment, the invention provides a composition comprising one or more isolated biomarker genes selected from ARID2, ASXL1, ATM, BAP1, BRCA1, BRCA2, CIC, CUL3, DLC1, FAT1, KDM5C, KDM6A, KMT2C, KMT2D, LRP1B, MGA, MTAP, NCOA6, NF2, PALB2, PBRM1, PTEN, PTPN11, PTPN13, PTPRS, RASA1, RB1, RB1CC1, RNF43, SMAD2, SMAD4, SMARCA4, SMG1, TGFBR2, TP53, and TSC2. In one embodiment, the invention provides a composition comprising two or more isolated biomarker genes selected from ARID2, ASXL1, ATM, BAP1, BRCA1, BRCA2, CIC, CUL3, DLC1, FAT1, KDM5C, KDM6A, KMT2C, KMT2D, LRP1B, MGA, MTAP, NCOA6, NF2, PALB2, PBRM1, PTEN, PTPN11, PTPN13, PTPRS, RASA1, RB1, RB1CC1, RNF43, SMAD2, SMAD4, SMARCA4, SMG1, TGFBR2, TP53, and TSC2. In one embodiment, the invention provides a composition comprising three or more isolated biomarker genes selected from ARID2, ASXL1, ATM, BAP1, BRCA1, BRCA2, CIC, CUL3, DLC1, FAT1, KDM5C, KDM6A, KMT2C, KMT2D, LRP1B, MGA, MTAP, NCOA6, NF2, PALB2, PBRM1, PTEN, PTPN11, PTPN13, PTPRS, RASA1, RB1, RB1CC1, RNF43, SMAD2, SMAD4, SMARCA4, SMG, TGFBR2, TP53, and TSC2. In one embodiment, the invention provides a composition comprising four or more isolated biomarker genes selected from ARID2, ASXL1, ATM, BAP1, BRCA1, BRCA2, CIC, CUL3, DLC1, FAT1, KDM5C, KDM6A, KMT2C, KMT2D, LRP1B, MGA, MTAP, NCOA6, NF2, PALB2, PBRM1, PTEN, PTPN11, PTPN13, PTPRS, RASA1, RB1, RB1CC1, RNF43, SMAD2, SMAD4, SMARCA4, SMG, TGFBR2, TP53, and TSC2. In one embodiment, the invention provides a composition comprising five or more isolated biomarker genes selected from ARID2, ASXL1, ATM, BAP1, BRCA1, BRCA2, CIC, CUL3, DLC1, FAT1, KDM5C, KDM6A, KMT2C, KMT2D, LRP1B, MGA, MTAP, NCOA6, NF2, PALB2, PBRM1, PTEN, PTPN11, PTPN13, PTPRS, RASA1, RB1, RB1CC1, RNF43, SMAD2, SMAD4, SMARCA4, SMG1, TGFBR2, TP53, and TSC2.

In one embodiment, the invention provides a composition comprising one or more isolated biomarker genes selected from ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, and SMG1. In one embodiment, the invention provides a composition comprising two or more isolated biomarker genes selected from ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, and SMG1. In one embodiment, the invention provides a composition comprising three or more isolated biomarker genes selected from ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, and SMG1. In one embodiment, the invention provides a composition comprising four or more isolated biomarker genes selected from ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, and SMG1. In one embodiment, the invention provides a composition comprising five or more isolated biomarker genes selected from ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, and SMG1.

In one embodiment, the invention provides a composition comprising one or more isolated biomarker genes selected from APC, ARID1A, ATRX, CDKN2A, CMTR2, DUSP4, EP300, FBXW7, KEAP1, KRAS, MSH2, NF1, PTPRD, RBM10, SETD2, STAG2, STK11, TET2, TSC1, USP15, and ZFHX3. In one embodiment, the invention provides a composition comprising two or more isolated biomarker genes selected from APC, ARID1A, ATRX, CDKN2A, CMTR2, DUSP4, EP300, FBXW7, KEAP1, KRAS, MSH2, NF1, PTPRD, RBM10, SETD2, STAG2, STK11, TET2, TSC1, USP15, and ZFHX3. In one embodiment, the invention provides a composition comprising three or more isolated biomarker genes selected from APC, ARID1A, ATRX, CDKN2A, CMTR2, DUSP4, EP300, FBXW7, KEAP1, KRAS, MSH2, NF1, PTPRD, RBM10, SETD2, STAG2, STK11, TET2, TSC1, USP15, and ZFHX3. In one embodiment, the invention provides a composition comprising four or more isolated biomarker genes selected from APC, ARID1A, ATRX, CDKN2A, CMTR2, DUSP4, EP300, FBXW7, KEAP1, KRAS, MSH2, NF1, PTPRD, RBM10, SETD2, STAG2, STK11, TET2, TSC1, USP15, and ZFHX3. In one embodiment, the invention provides a composition comprising five or more isolated biomarker genes selected from APC, ARID1A, ATRX, CDKN2A, CMTR2, DUSP4, EP300, FBXW7, KEAP1, KRAS, MSH2, NF1, PTPRD, RBM10, SETD2, STAG2, STK11, TET2, TSC1, USP15, and ZFHX3.

In one embodiment, the invention provides a composition comprising one or more isolated biomarker genes selected from CDKN2A, EP300, KEAP1, KRAS, RBM10, SETD2, STK11, TSC1, and USP15. In one embodiment, the invention provides a composition comprising two or more isolated biomarker genes selected from CDKN2A, EP300, KEAP1, KRAS, RBM10, SETD2, STK11, TSC1, and USP15. In one embodiment, the invention provides a composition comprising three or more isolated biomarker genes selected from CDKN2A, EP300, KEAP1, KRAS, RBM10, SETD2, STK11, TSC1, and USP15. In one embodiment, the invention provides a composition comprising four or more isolated biomarker genes selected from CDKN2A, EP300, KEAP1, KRAS, RBM10, SETD2, STK11, TSC1, and USP15. In one embodiment, the invention provides a composition comprising five or more isolated biomarker genes selected from CDKN2A, EP300, KEAP1, KRAS, RBM10, SETD2, STK11, TSC1, and USP15.

In one embodiment, the invention provides a method of detecting one or more isolated biomarker genes selected from ADAR, APC, ARID1A, ARID2, ASXL1, ATM, ATRX, BAP1, BRCA1, BRCA2, CDKN2A, CHD2, CIC, CMTR2, CREBBP, CUL3, DICER1, DLC1, DNMT1, DUSP4, EP300, FAT1, FBXW7, JAK1, KDM5C, KDM6A, KEAP1, KMT2C, KMT2D, KRAS, LRP1B, MET, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TREX1, TP53, TSC1, TSC2, USP15, and ZFHX3 in a human subject, the method comprising: detecting whether the one or more isolated biomarker genes are present in a biological sample of the human subject by contacting the biological sample with a binding agent and detecting binding between the one or more isolated biomarker genes and the binding agent. In certain embodiments, the method further comprises obtaining a biological sample from the human subject.

In one embodiment, the invention provides a method of detecting one or more isolated biomarker genes selected from APC, ARID2, ATM, ATRX, BRCA2, CDKN2A, CMTR2, FBXW7, KDM6A, KEAP1, KMT2D, KRAS, MGA, MSH2, NF1, NF2, PALB2, PCNA, PTEN, PTPN11, PTPRD, RB1, RBM10, RNF43, SETD2, SMAD4, STAG2, STK11, TP53, and TSC1 in a human subject, the method comprising: detecting whether the one or more isolated biomarker genes are present in a biological sample of the human subject by contacting the biological sample with a binding agent and detecting binding between the one or more isolated biomarker genes and the binding agent. In certain embodiments, the method further comprises obtaining a biological sample from the human subject.

In one embodiment, the invention provides a method of detecting one or more isolated biomarker genes selected from APC, ARID2, ATM, ATRX, BRCA2, CDKN2A, CMTR2, KEAP1, KMT2D, MGA, NF1, PTEN, PTPRD, RB1, RBM10, RNF43, SETD2, SMAD4, STAG2, STK11, TP53, and TSC1 in a human subject, the method comprising: detecting whether the one or more isolated biomarker genes are present in a biological sample of the human subject by contacting the biological sample with a binding agent and detecting binding between the one or more isolated biomarker genes and the binding agent. In certain embodiments, the method further comprises obtaining a biological sample from the human subject.

In one embodiment, the invention provides a method of detecting one or more isolated biomarker genes selected from CDKN2A, EP300, KRAS, MGA, RB1, RBM10, SETD2, STAG2, STK11, TP53, USP15, and ZFHX3 in a human subject, the method comprising: detecting whether the one or more isolated biomarker genes are present in a biological sample of the human subject by contacting the biological sample with a binding agent and detecting binding between the one or more isolated biomarker genes and the binding agent. In certain embodiments, the method further comprises obtaining a biological sample from the human subject.

In one embodiment, the invention provides a method of detecting one or more isolated biomarker genes selected from CDKN2A, EP300, RBM10, and SETD2 in a human subject, the method comprising: detecting whether the one or more isolated biomarker genes are present in a biological sample of the human subject by contacting the biological sample with a binding agent and detecting binding between the one or more isolated biomarker genes and the binding agent. In certain embodiments, the method further comprises obtaining a biological sample from the human subject.

In one embodiment, the invention provides a method of detecting one or more isolated biomarker genes selected from APC, ARID2, ATM, ATRX, BRCA2, CDKN2A, CMTR2, MGA, NF1, PTEN, PTPRD, RB1, RBM10, RNF43, SETD2, STAG2, STK11, TP53, and TSC1 in a human subject, the method comprising: detecting whether the one or more isolated biomarker genes are present in the biological sample by contacting a biological sample of the human subject with a binding agent and detecting binding between the one or more isolated biomarker genes and the binding agent. In certain embodiments, the method further comprises obtaining a biological sample from the human subject.

In one embodiment, the invention provides a method of detecting one or more isolated biomarker genes selected from ARID2, CDKN2A, CMTR2, SETD2, STAG2, and STK11 in a human subject, the method comprising: detecting whether the one or more isolated biomarker genes are present in the biological sample by contacting a biological sample of the human subject with a binding agent and detecting binding between the one or more isolated biomarker genes and the binding agent. In certain embodiments, the method further comprises obtaining a biological sample from the human subject.

In one embodiment, the invention provides a method of detecting one or more isolated biomarker genes selected from ARID2, A7M, CDKN2A, CMTR2, KRAS, MGA, MSH2, RB1, RBM10, RNF43, SETD2, SMAD4, STAG2, and STK11 in a human subject, the method comprising: detecting whether the one or more isolated biomarker genes are present in a biological sample of the human subject by contacting the biological sample with a binding agent and detecting binding between the one or more isolated biomarker genes and the binding agent. In certain embodiments, the method further comprises obtaining a biological sample from the human subject.

In one embodiment, the invention provides a method of detecting one or more isolated biomarker genes selected from ARID2, BAP1, BRCA1, CIC, KDM6A, KMT2C, KMT2D, LRP1B, NCOA6, NF2, PALB2, PTEN, RASA1, SMAD2, SMG1, SMARCA4, and TET2 in a human subject, the method comprising: detecting whether the one or more isolated biomarker genes are present in a biological sample of the human subject by contacting the biological sample with a binding agent and detecting binding between the one or more isolated biomarker genes and the binding agent. In certain embodiments, the method further comprises obtaining a biological sample from the human subject.

In one embodiment, the invention provides a method of detecting one or more isolated biomarker genes selected from ARID2, BAP1, BRCA1, CIC, KMT2D, NCOA6, and RASA1 in a human subject, the method comprising: detecting whether the one or more isolated biomarker genes are present in a biological sample of the human subject by contacting the biological sample with a binding agent and detecting binding between the one or more isolated biomarker genes and the binding agent. In certain embodiments, the method further comprises obtaining a biological sample from the human subject.

In one embodiment, the invention provides a method of detecting one or more isolated biomarker genes selected from KEAP1, KMT2D, and SMAD4 in a human subject, the method comprising: detecting whether the one or more isolated biomarker genes are present in a biological sample of the human subject by contacting the biological sample with a binding agent and detecting binding between the one or more isolated biomarker genes and the binding agent. In certain embodiments, the method further comprises obtaining a biological sample from the human subject.

In one embodiment, the invention provides a method of detecting one or more isolated biomarker genes selected from KEAP1, KMT2D, PTEN and SMAD4 in a human subject, the method comprising: detecting whether the one or more isolated biomarker genes are present in a biological sample of the human subject by contacting the biological sample with a binding agent and detecting binding between the one or more isolated biomarker genes and the binding agent. In certain embodiments, the method further comprises obtaining a biological sample from the human subject.

In one embodiment, the invention provides a method of detecting one or more isolated biomarker genes selected from KMT2D and PTEN in a human subject, the method comprising: detecting whether the one or more isolated biomarker genes are present in a biological sample of the human subject by contacting the biological sample with a binding agent and detecting binding between the one or more isolated biomarker genes and the binding agent. In certain embodiments, the method further comprises obtaining a biological sample from the human subject.

In one embodiment, the invention provides a method of detecting one or more isolated biomarker genes selected from APC, ARID1A, ARID2, ASXL1, ATM, ATRX BAP1, BRCA1, BRCA2, CDKN2A, CIC, CMTR2, CUL3, DICER1, DLC1, DUSP4, EP300, FAT1, FBXW7, KDM5C, KDM6A, KEAP1, KMT2C, KMT2D, KRAS, LRP1B, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TP53, TSC1, TSC2, USP15, and ZFHX3 in a human subject, the method comprising: detecting whether the one or more isolated biomarker genes are present in a biological sample of the human subject by contacting the biological sample with a binding agent and detecting binding between the one or more isolated biomarker genes and the binding agent. In certain embodiments, the method further comprises obtaining a biological sample from the human subject.

In one embodiment, the invention provides a method of detecting one or more isolated biomarker genes selected from ARID2, ASXL1, ATM, BAP1, BRCA1, BRCA2, CIC, CUL3, DLC1, FAT1, KDM5C, KDM6A, KMT2C, KMT2D, LRP1B, MGA, MTAP, NCOA6, NF2, PALB2, PBRM1, PTEN, PTPN11, PTPN13, PTPRS, RASA1, RB1, RB1CC1, RNF43, SMAD2, SMAD4, SMARCA4, SMG, TGFBR2, TP53, and TSC2 in a human subject, the method comprising: detecting whether the one or more isolated biomarker genes are present in a biological sample of the human subject by contacting the biological sample with a binding agent and detecting binding between the one or more isolated biomarker genes and the binding agent. In certain embodiments, the method further comprises obtaining a biological sample from the human subject.

In one embodiment, the invention provides a method of detecting one or more isolated biomarker genes selected from ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, and SMG1 in a human subject, the method comprising: detecting whether the one or more isolated biomarker genes are present in a biological sample of the human subject by contacting the biological sample with a binding agent and detecting binding between the one or more isolated biomarker genes and the binding agent. In certain embodiments, the method further comprises obtaining a biological sample from the human subject.

In one embodiment, the invention provides a method of detecting one or more isolated biomarker genes selected from APC, ARID1A, ATRX, CDKN2A, CMTR2, DUSP4, EP300, FBXW7, KEAP1, KRAS, MSH2, NF1, PTPRD, RBM10, SETD2, STAG2, STK11, TET2, TSC1, USP15, and ZFHX3 in a human subject, the method comprising: detecting whether the one or more isolated biomarker genes are present in a biological sample of the human subject by contacting the biological sample with a binding agent and detecting binding between the one or more isolated biomarker genes and the binding agent. In certain embodiments, the method further comprises obtaining a biological sample from the human subject. In some embodiments, the one or more isolated biomarker genes are selected from CDKN2A, EP300, KEAP1, KRAS. RBM10, SETD2, STK11, TSC1, and USP15.

In some embodiments of the methods of detecting one or more isolated biomarker genes, the binding agent comprises a reagent capable of determining the genotype by detecting, for example, a polypeptide or nucleic acid that encodes the biomarker gene or fragments thereof. In some embodiments, a binding agent can be a sequencing reagent. In some embodiments, a binding agent can be a probe and/or primer, for sequencing a biomarker gene or portion thereof. In some embodiments, a binding agent can be an antibody or an antigen-binding fragment thereof. In some embodiments, a binding agent comprises a label. In embodiments comprising detection of more than one isolated biomarker gene, the reagents can be referred to, for example, a “first reagent” or “first binding agent” specific for biomarker gene KMT2D, a “second reagent” or “second binding agent” specific for biomarker gene PTEN, and so forth.

It must be noted that as used herein and in the appended claims, the singular forms “a,” “an,” and “the” include plural reference unless the context clearly dictates otherwise.

It should also be understood that the terms “about,” “approximately,” “generally,” “substantially,” and like terms, used herein when referring to a dimension or characteristic of a component of embodiments provided herein, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (for example, rounding, measurement or other systematic errors, manufacturing tolerances, etc.), would not vary the least significant digit. Unless otherwise stated, any numerical values, such as a concentration or a concentration range described herein, are to be understood as being modified in all instances by the term “about.”

Unless otherwise indicated, the term “at least” preceding a series of elements is to be understood to refer to every element in the series and any one or any and all combinations of the elements.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition.

As used herein, the term “subject” refers to any organism, preferably a mammal, for whom diagnosis, prognosis, or therapy is desired. Mammalian subjects include humans, domestic animals, farm animals, sports animals, and zoo animals including, for example, humans, non-human primates, dogs, cats, guinea pigs, rabbits, rats, mice, horses, cattle, and so on. In some embodiments, the subject is human. In one embodiment, the subject has been diagnosed with cancer. In some aspects, the subject is afflicted with cancer and has been diagnosed with a need for treatment for cancer.

The terms “inhibit,” “block,” and “suppress” are used interchangeably and refer to any statistically significant decrease in a biological activity, including full blocking of the activity. An “inhibitor” is an active agent that inhibits, blocks, or suppresses biological activity in vitro or in vivo. Inhibitors include but are not limited to small molecule compounds; nucleic acids, such as siRNA and shRNA; polypeptides, such as antibodies or antigen-binding fragments thereof, dominant-negative polypeptides, and inhibitory peptides; and oligonucleotide or peptide aptamers.

As used herein, the terms “chemotherapy,” ‘chemotherapeutic” or “chemotherapeutic agent” refers to traditional or standard chemotherapy, which is understood in the art as a systemic therapy with a chemical agent that is used to treat cancer by directly killing rapidly dividing cells or by stopping cell division. Chemotherapy is distinct from therapies used to treat cancer in different ways, including targeted therapy, hormone therapy, and immunotherapy. Generally, chemotherapeutics prevent cancer cells from multiplying by: (1) interfering with the cell's ability to replicate DNA and (2) inducing cell death and/or apoptosis in the cancer cells. Chemotherapeutics fall into several different classes including, for example and without limitation, antimetabolites (purine analogs, purine antagonists, pyrimidine antagonists, antifolates, ribonucleotide reductase inhibitors), alkylating agents (platinum-based agents, hydrazine, oxazaphosphorines, nitrogen-mustards), mitotic spindle inhibitors (taxanes, vinca alkaloids), topoisomerase-1 inhibitors, and topoisomerase-2 inhibitors.

Chemotherapeutics include, for example and without limitation, Abiraterone acetate, Altretamine, Belinostat, Bendamustine, Bleomycin, Bortezomib, Brentuximab vedotin, Busulfan, Cabazitaxel, Capecitabine, Carboplatin, Carmustine, Ceritinib, Chlorambucil, Cisplatin, Cladribine, Crizotinib, Cyclophosphamide, Cytarabine (Ara-C), Dabrafenib, Dacarbazine, Dactinomycin, Dasatinib, Daunorubicin, DaunoXome (liposomal daunorubicin), DepoCyt (liposomal cytarabine), Docetaxel, Doxil (liposomal doxorubicin), Doxorubicin, Epirubicin, Eribulin mesylate, Erlotinib, Estramustine, Etoposide, Everolimus, Floxuridine, Fludarabine, Fluorouracil, Gefitinib, Gemcitabine, Gliadel wafers, Hydroxyurea, Ibrutinib, Idarubicin, Idelalisib, Ifosfamide, Imatinib, Ipilimumab, Irinotecan, Ixabepilone, Lanreotide, Lapatinib, Lenalidomide, Lenvatinib, Lomustine, Mechlorethamine, Melphalan, Mercaptopurine, Methotrexate, Mitomycin, Mitoxantrone, Nilotinib, Olaparib, Oxaliplatin, Paclitaxel, Palbociclib, Pazopanib, Panobinostat, PEG-asparaginase, Peginterferon alfa-2b, Pemetrexed, Pentostatin, Pralatrexate, Procarbazine, Romidepsin, Ruxolitinib, Sipuleucel-T, Sorafenib, Streptozocin, Sunitinib, Temozolomide, Temsirolimus, Teniposide, Thalidomide, Thioguanine, Thiotepa, Topotecan, Tositumomab, Trametinib, Valrubicin, Vandetanib, Vemurafenib, Vinblastine, Vincristine, and Vinorelbine.

In one embodiment, the methods of the invention further comprise administering chemotherapy to the subject. In some embodiment, the chemotherapy comprises a chemotherapeutic agent belonging to the class comprising taxanes. In some embodiments, the chemotherapeutic agent is paclitaxel or docetaxel. In some embodiment, the chemotherapy comprises a chemotherapeutic agent belonging to class comprising platinum-based chemotherapeutic agents. In some embodiments, the chemotherapeutic agent is carboplatin. In some embodiment, the chemotherapy comprises a chemotherapeutic agent belonging to class comprising folate antimetabolites. In some embodiments, the chemotherapeutic agent is pemetrexed.

In one embodiment, the chemotherapeutic is a small molecule. In another embodiment, the chemotherapeutic is conjugated to a polypeptide. In another embodiment, the chemotherapeutic is conjugated to a polypeptide analog. In another embodiment, the chemotherapeutic is conjugated to a pepetidomimetic. In another embodiment, the chemotherapeutic is conjugated to an aptamer. In another embodiment, the chemotherapeutic is conjugated to a nanoparticle.

Any compound chemical agent that binds to and specifically kills rapidly growing cells can be utilized in accordance with the present disclosure.

“MEK inhibitor” or “MEKi” refers to any active agent that antagonizes the activity of a MEK protein, reduces its production or activity in a cell. As such, the term encompasses inhibitors of either MEK1, MEK2 and dual inhibitors of MEK1/2. A MEKi generally targets the Ras/Raf/MEK/ERK signaling pathway, inhibiting cell proliferation and inducing apoptosis. Blockage of the pathway with a MEKi inhibitors can confer clinical benefits for treatment of cancers with RAS/RAF dysfunction.

Any suitable MEK inhibitor can be used in the methods described herein. Exemplary MEK inhibitors include, for example, Trametinib (GSK1120212, JTP-74057) (NOVARTIS), Cobimetinib (GDC-0973, XL518) (Genentech, Inc.), CI-1040, PD-0325901, Selumetinib (ARRY-142886; AZD6244) (ASTRAZENECA), Binimetinib (MEK162, ARRY-438162) (Array Biopharma Inc.), AZD-8330 (ARRY-424704), TAK-733, GDC-0623 (RG 7421) (Genentech, Inc.), Refametinib (RDEA-119, BAY-869766) (Bayer AG), Pimasertib (AS703026) (Merck KGaA), R04987655 (CH4987655), CH5126766 (RO5126766)(Chugai Pharmaceutical Co., Roche), WX-554, HL-085 (Shanghai Kechow Pharma, Inc.), CinQ-03, G-573, Mirdametinib (PD-0325901) (Spring Works Therapeutics), PD184161, PD318088, PD98059, R05068760, U0126, E6201 (Eisai Co Ltd./Strategia Theraputics), SHR7390 (Hengrui Medicine), TQ-B3234 (Chiatai Tianqing), CS-3006 (CStone Pharmaceuticals), FCN-159 (Fosun Pharma) and SL327. (Cheng and Tian, Molecules 22, 1551 (2017); doi:10.3390/molecules2210155. Han, J., Liu, Y., Yang, S. et al. MEK inhibitors for the treatment of non-small cell lung cancer. J Hematol Oncol 14, 1 (2021); doi:10.1186/s13045-020-01025-7).

“mTOR pathway inhibitor” refers to any active agent that antagonizes the activity, reduces the production or activity in a cell of mTOR protein kinase, which is the catalytic subunit of two distinct protein complexes, mTORC1 and mTORC2, also interchangeably referred to herein as TORC1 and TORC2, respectively. As such, the terms “TORC inhibitor” or “TORCi” encompasses inhibitors of either mTORC1, mTORC2 and dual inhibitors of mTORC1/2. An mTORCi generally targets the phosphoinositide 3-kinase (PI3K)/protein kinase B (AKT)/mammalian target of rapamycin (mTOR) signaling pathway, a central regulator of cellular growth, proliferation and survival. Dysregulation of PI3K/AKT/mTOR activity is frequently observed in human cancers. As part of the mammalian target of rapamycin complex 1 (mTORC1) and 2 (mTORC2), mTOR is a key intracellular point of convergence for several pathways, thus representing an important therapeutic target.

Although described in the context of MEK below, any general description of suitable inhibitors throughout this application applies equally to TORC inhibitors.

Any suitable mTOR inhibitor can be used in the methods described herein. Exemplary mTOR inhibitors include, for example, MLN0128 (INK128, Sapanisertib, TAK-228, PP242 (Tokinib), AZD2014 (Vistusertib) and its analog AZD8055, Voxtalisib (SAR24540; XL765) and Gedatolisib (PKI-587; PF05212384).

In one embodiment, the MEK inhibitor is a small molecule. In another embodiment, the MEK inhibitor is a polypeptide. In another embodiment, the MEK inhibitor is a polypeptide analog. In another embodiment, the MEK inhibitor is a peptidomimetic. In another embodiment, the MEK inhibitor is an aptamer.

In another embodiment, a MEK inhibitor is an antibody, or an antigen-binding fragment thereof. For example, the antibody, or antigen-binding fragment thereof, can be a humanized antibody, a recombinant antibody, a diabody, a chimerized or chimeric antibody, a monoclonal antibody, a deimmunized antibody, a fully human antibody, a single chain antibody, an Fᵥ fragment, an F_{d} fragment, a Fab fragment, a Fab′ fragment, or an F(ab′)₂ fragment.

Any compound which binds to and inhibits, or otherwise inhibits the activity, function and/or the expression of a MEK protein or its receptor can be utilized in accordance with the present disclosure. For example, an inhibitor of a MEK protein can be, for example, a small molecule, a nucleic acid or a nucleic acid analog, a peptidomimetic, or a macromolecule that is not a nucleic acid or a protein. Accordingly, compounds which can be utilized as MEK inhibitors include, but are not limited to, proteins, protein fragments, peptides, small molecules, RNA aptamers, L-RNA aptamers, spiegelmers, antisense compounds, serine protease inhibitors, molecules which can be utilized in RNA interference (RNAi) such as double stranded RNA including small interfering RNA (siRNA), locked nucleic acid (LNA) inhibitors, peptide nucleic acid (PNA) inhibitors, etc.

A MEK inhibitor of can also be, for example, a small molecule, a polypeptide analog, a nucleic acid, or a nucleic acid analog.

“Small molecule” as used herein, is meant to refer to an agent, which has a molecular weight preferably of less than about 6 kDa and most preferably less than about 2.5 kDa. Many pharmaceutical companies have extensive libraries of chemical and/or biological mixtures comprising arrays of small molecules, often fungal, bacterial, or algal extracts, which can be screened with any of the assays of the application. It is within the scope of this application that such a library can be used to screen for agents that bind to a target antigen of interest (for example, a MEK protein). There are numerous commercially available compound libraries, such as the Chembridge DIVERSet® screening library. Libraries are also available from academic and governmental entities, such as the National Cancer Institute's. Developmental Therapeutics Program (DTP). Rational drug design can also be employed and can be achieved based on known compounds, for example, a known inhibitor of a MEK protein (for example, an antibody, or antigen-binding fragment thereof, that binds to a MEK protein).

In one embodiment, the MEK inhibitor is an antibody or an antigen-binding fragment thereof, which binds to a MEK protein.

As used herein, the term “antibody” is used in a broad sense and includes immunoglobulin or antibody molecules including human, humanized, composite and chimeric, single-chain, bi-specific and multi-specific antibodies and antibody fragments, in particular, antigen-binding fragments, that are monoclonal or polyclonal. In general, antibodies are proteins or peptide chains that exhibit binding specificity to a specific antigen. Antibody structures are well known. Immunoglobulins can be assigned to five major classes (specifically, IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4. Accordingly, the antibodies provided herein can be of any of the five major classes or corresponding sub-classes. In specific embodiments, the antibodies provided herein are IgG1, IgG2, IgG3 or IgG4. Antibody light chains of vertebrate species can be assigned to one of two clearly distinct types, namely kappa and lambda, based on the amino acid sequences of their constant domains.

In one embodiment, the MEK inhibitor is a nucleic acid inhibitor. Nucleic acid inhibitors can be used to bind to and inhibit a target antigen of interest. The nucleic acid antagonist can be, for example, an aptamer or a small interfering RNA (siRNA). Aptamers are short oligonucleotide sequences that can be used to recognize and specifically bind almost any molecule, including cell surface proteins. The systematic evolution of ligands by exponential enrichment (SELEX) process is powerful and can be used to readily identify such aptamers. Aptamers can be made for a wide range of proteins of importance for therapy and diagnostics, such as growth factors and cell surface antigens. These oligonucleotides bind their targets affinities and specificities similar to those of antibodies.

By introducing a certain nucleic acid modality to the desired tissue of the subject, MEK gene expression can be downregulated, augmented or corrected. Small interfering RNA (siRNA), microRNA (miRNA) and inhibitory antisense oligonucleotides (ASOs) are representative molecules used to trigger gene inhibition, whereas plasmid DNA, messenger RNA (mRNA), small activating RNA (saRNA), splicing-modulatory ASOs and CRISPR (clustered regularly interspaced short palindromic repeats)/Cas (CRISPR-associated protein) systems are usually employed to increase or correct target gene expression.

In one embodiment, the MEK inhibitor is a non-antibody scaffold protein. These proteins are, generally, obtained through combinatorial chemistry-based adaptation of pre-existing antigen-binding proteins. For example, the binding site of human transferrin for human transferrin receptor can be modified using combinatorial chemistry to create a diverse library of transferrin variants, some of which have acquired affinity for different antigens. The portion of human transferrin not involved with binding the receptor remains unchanged and serves as a scaffold, like framework regions of antibodies, to present the variant binding sites. The libraries are then screened, as an antibody library is, against a target antigen of interest to identify those variants having optimal selectivity and affinity for the target antigen. Non-antibody scaffold proteins, while similar in function to antibodies, are touted as having a number of advantages as compared to antibodies, which advantages include, among other things, enhanced solubility and tissue penetration, less costly manufacture, and ease of conjugation to other molecules of interest. Hey et al. (2005) TRENDS Biotechnol 23(10):514-522.

As used herein, the terms “inhibit” or “inhibiting” or “reducing” and grammatical variations thereof refer to the decrease, limitation or blockage of, for example, a particular action, function, or interaction. For example, in the context of tumor growth, “inhibited” means terminated, reduced, delayed or prevented. Tumor growth is also “inhibited” if recurrence or metastasis of the cancer is reduced, slowed, delayed or prevented.

An “isolated” biomarker gene is one which is separated from other materials which are present in the natural source of the biomarker gene. An isolated biomarker has markedly different characteristics from its naturally occurring counterpart. A biomarker gene of the present invention can be isolated using standard molecular biology techniques and the sequence information in the database records described herein. Using all or a portion of such biomarker gene nucleic acid sequences, nucleic acid molecules of the present invention can be isolated using standard hybridization and cloning techniques (e.g., as described in Sambrook et al., ed., Molecular Cloning: A Laboratory Manual, 2nd ed., Cold Spring Harbor Laboratory Press, Cold Spring Harbor, N.Y., 1989).

The term “combination therapy” or “combination treatment” or any variation of the terms as used herein in relation to the administration of a MEK inhibitor therapy and TORC inhibitor therapy refers to the administration of the MEK and TORC inhibitors such that the individual therapies/drugs are present within a human subject at the same time. In addition to the concomitant administration of MEK and TORC inhibitors (via the same or alternative routes), simultaneous administration may include the administration of the MEK and TORC inhibitors (via the same or an alternative route) at different times. The same definition also applies to a MEK inhibitor therapy and chemotherapy. The terms “therapy” and “treatment” are used interchangeably and afforded the same meaning in the methods described herein.

As used herein, “reducing the tumor,” means reducing the size, volume, or weight of the tumor, reducing the number of metastases, reducing the size or weight of a metastasis, or combinations thereof. In some examples, a metastasis is cutaneous or subcutaneous. Thus, in some embodiments, administration of the MEK inhibitor reduces the size or volume of the tumor by at least about 10%, at least about 20%, at least about 30%, at least about 40%, at least about 50%, at least about 60%, at least about 70%, at least about 75%, at least about 80%, at least about 90%, at least about 95%, at least about 98% or at least about 99%, for example, relative to a control drug in a subject of the same genotype. In some examples, administration of the MEK inhibitor reduces the weight of the tumor by at least about 10%, at least about 20%, at least about 30%, at least about 40%, at least about 50%, at least about 60%, at least about 70%, at least about 75%, at least about 80%, at least about 90%, at least about 95%, at least about 98% or at least about 99%, for example, relative to a control drug in a subject of the same genotype. In some examples, administration of the MEK inhibitor reduces the size or volume of a metastasis by at least about 10%, at least about 20%, at least about 30%, at least about 40%, at least about 50%, at least about 60%, at least about 70%, at least about 75%, at least about 80%, at least about 90%, at least about 95%, at least about 98% or at least about 99%, for example, relative to a control drug in a subject of the same genotype. In some examples, administration of the MEK inhibitor reduces the number of metastases by at least about 10%, at least about 20%, at least about 30%, at least about 40%, at least about 50%, at least about 60%, at least about 70%, at least about 75%, at least about 80%, at least about 90%, at least about 95%, at least about 98% or at least about 99%, for example, relative to a control drug in a subject of the same genotype. In some examples, combinations of these effects are achieved.

A “biomarker gene” can be any gene having a genotype and/or expression level that can be determined, measured and/or evaluated as an indicator of a biologic process, pathogenic process, or pharmacologic response to a therapeutic intervention. A biomarker gene useful to practice the methods of the invention can be used as an indicator to determine whether a human subject having cancer will be sensitive or resistant to a therapy with a MEK inhibitor and/or for monitoring response to a therapy with a MEK protein inhibitor. In one embodiment, a biomarker gene is a tumor suppressor gene. Sensitivity or resistance to a therapy with a MEK protein inhibitor can be determined by analyzing a nucleic acid molecule (DNA, mRNA, cDNA etc.) corresponding to a biomarker gene or the protein encoded by the biomarker gene. Biomarker genes can include any gene whose genotype and/or level of expression in a tissue or cell can be used to predict response to a MEK inhibitor therapy. The detection, and in some cases the level, of one or more biomarker genes of the invention permits the classification of a human subject as sensitive or resistant to a MEK inhibitor theory. A biomarker gene useful to practice the methods of the invention can be selected from any of the biomarker gene panels and lists described herein.

In some embodiments, a biomarker gene is a tumor suppressor gene. In some embodiments, a biomarker gene is part of a pathway or otherwise related to one of the biomarker genes disclosed herein. Additional biomarker genes useful in the methods disclosed herein can be identified by those skilled in the art based on the present disclosure. See, for example, Itatani et al., Int J Mol Sci., 20(23):5822, (2019) (TGF-β signaling pathway); Manning et al., Genes Dev., 19(15):1773-1778, (2005) (PI3K-Akt pathway); Johannessen et al., Proc Natl Acad Sci, 102(24): 8573-8578, (2005) (PI3K-Akt pathway); Tsukiyama et al., Mol Cell Biol, 35:2007-2023, (2015)(Wnt signaling pathway); Zhang et al., Molecular Cancer, 17, 45 (2018)(c-Met signaling pathway); Mombach et al., BMC Genomics., 15 Suppl 7(Suppl 7):S7 (2014) (Gl/S checkpoint); Shain et al., PLoS ONE, 8(1): e55119, (2013)(SWI/SNF complex); Iyer et al., Oncogene, 23:4225-4231, (2004) (p300 and CBP); Villeneuve et al., Mol Cell., 51(1): 68-79, (2013)(USP15, KEAP1, and CUL3); Sahtoe et al., Nat Commun 7, 10292 (2016) (BAP1 and ASXL1); Harris et al., Oncogene, 24:2899-2908 (2005)(p53).

As used herein, the term “biomarker profile” means an aggregate of information derived from one or more individual biomarker genes. A biomarker profile can be based on, for example, adding two or more sensitizing mutations, adding two or more resistance mutations, adding two or more of sensitizing and resistance mutations, as well accounting for sensitizing or resistance mutations by assigning different weighted scores.

As used herein, the terms “determine,” “determine the genotype of a biomarker gene,” “determine the level of a biomarker gene,” “determine the amount of a biomarker gene,” “determine the biomarker gene level,” and the like are meant to encompass any technique that can be used to detect or measure the genotype, presence or expression level of one or more biomarker genes or any fragment thereof, and involves physical steps. Such techniques can give qualitative or quantitative results. Biomarker gene levels can be determined by detecting the entire biomarker molecule or by detecting fragments or reaction products that are characteristic of the biomarker gene. The terms determining, measuring, or taking a measurement refer to a quantitative or qualitative determination of a property of an entity, for example, quantifying the amount or concentration of a molecule or the activity level of a molecule. Any known method of detecting or measuring the level of a biomarker can be used to practice the present invention, so long as the method detects the genotype, presence, absence, or expression level of the biomarker gene.

In one embodiment, determining the genotype of a biomarker gene is performed at the nucleic acid level by performing RNA-seq, a reverse transcriptase polymerase chain reaction (RT-PCR) or a hybridization assay with oligonucleotides that are substantially complementary to portions of cDNA molecules of the at least one biomarker gene under conditions suitable for RNA-seq, RT-PCR or hybridization and obtaining expression levels of the at least one biomarker gene.

As used herein, the terms “cancer” or “tumor” refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist isolated within an animal, or can be non-tumorigenic, such as a leukemia cell. Cancers include, but are not limited to, B cell malignancies, for example, multiple myeloma, the heavy chain diseases, such as, for example, alpha chain disease, gamma chain disease, and mu chain disease, benign monoclonal gammopathy, and immunocytic amyloidosis, skin cancer, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like. Other non-limiting examples of types of cancers applicable to the methods encompassed by the present invention include human sarcomas and carcinomas, for example, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, for example, acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, the cancer is an epithelial cancer such as, but not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (for example, serous ovarian carcinoma), or breast carcinoma. The amount of a tumor in an individual is the “tumor burden” which can be measured as the number, volume, and/or weight of the tumor.

Exemplary cancers in the embodiments of the invention include skin cancer, lung cancer, pancreatic cancer, breast cancer, colorectal cancer, bladder cancer, liver cancer, kidney cancer, leukemia, and lymphoma. In some embodiments, the cancer is an advanced solid tumor. In some embodiments, the cancer is selected from the group consisting of lung cancer, pancreatic cancer, colorectal cancer, ovarian cancer, urothelial carcinoma, B cell lymphoma, chronic lymphocytic leukemia (CLL), head and neck squamous cell carcinoma (HNSCC), metastatic castration-resistant prostate cancer (mCRPC), and prolymphocytic leukemia (PLL). In some embodiments, the cancer is lung cancer. In some embodiments, the lung cancer is non-small cell lung cancer (NSCLC). In some embodiments, NSCLC is lung adenocarcinoma. In some embodiments, the cancer is pancreatic cancer. In some embodiments, the cancer is colorectal cancer.

As used herein, the term “lung cancer” refers to the collection of cancers affecting lung tissue. Non-small cell lung cancer (NSCLC) represents approximately 87% of all lung cancers. There are three main types of NSCLC: squamous cell carcinoma, large cell carcinoma, and adenocarcinoma.

The term “classifying” includes associating a sample with a response to a MEK inhibitor therapy. In certain instances, “classifying” is based on statistical evidence, empirical evidence, or both. In certain embodiments, the methods of classifying utilize a training set of samples having known genotypes. Once established, the training data set can serve as a basis, model, or template against which the features of an unknown sample are compared, in order to classify the sample.

The term “control” refers to any reference standard suitable to provide a comparison to the expression products in the test sample. A control can comprise a reference standard expression product level or genotype score from any suitable source, including but not limited to housekeeping genes, an expression product level range from normal tissue (or other previously analyzed control sample), a previously determined expression product level range within a test sample from a group of subjects, or a set of subjects with a certain outcome or receiving a certain therapy. It will be understood by those of skill in the art that such control samples and reference standard expression product levels can be used in combination as controls in the methods of the present invention. In various embodiments, the biomarker gene expression can be compared to a reference. A “reference” can be any value derived by art known methods for establishing a reference.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed protein can be within the cytoplasm of a host cell, into the extracellular milieu such as the growth medium of a cell culture or anchored to the cell membrane.

The term “gene product” as used herein, refers to RNA transcribed from a gene and to one or more proteins, polypeptides of fragments thereof that are the product of translation of the RNA transcribed from the gene, and further encompasses all naturally occurring post-transcriptional and post-translational modifications. The expressed protein can be within the cytoplasm of a host cell, into the extracellular milieu such as the growth medium of a cell culture or anchored to the cell membrane.

The terms “expression level” and “level of expression” as used herein refers to information regarding the relative or absolute level of expression of one or more biomarker genes in a cell or group of cells. The level of expression of a biomarker gene can be determined based on the level of RNA, such as mRNA, encoded by the gene. Alternatively, the level of expression can be determined based on the level of a polypeptide or fragment thereof encoded by the biomarker gene. Gene expression data can be acquired for an individual cell, or for a group of cells such as a tumor or biopsy sample. Gene expression data and gene expression levels can be stored on computer readable media, for example, the computer readable medium used in conjunction with a microarray or chip reading device. Such gene expression data can be manipulated to generate gene expression signatures.

The expression level of a biomarker gene can be determined using a reagent such as a probe, primer or antibody and/or a method performed on a biological sample, for example a tumor sample of the subject, for ascertaining or measuring quantitatively, semi-quantitatively or qualitatively the amount of a of a polypeptide or mRNA (or cDNA derived therefrom) corresponding to one or more biomarker genes. For example, a level of a biomarker gene can be determined by a number of methods including for example immunoassays including for example immunohistochemistry, ELISA, Western blot, immunoprecipitation and the like, where a detection agent such as an antibody for example, a labeled antibody, specifically binds the encoded polypeptide and permits relative or absolute ascertaining of the amount of polypeptide encoded by the biomarker gene, hybridization and PCR protocols where a probe or primer or primer set are used to ascertain the amount of nucleic acid corresponding to the biomarker gene, including for example probe based and amplification based methods including for example microarray analysis, RT-PCR such as quantitative RT-PCR (qRT-PCR), gRT-PCR, serial analysis of gene expression (SAGE), Northern Blot, digital molecular barcoding technology, for example Nanostring Counter Analysis, and TaqMan quantitative PCR assays.

Other methods of mRNA detection and quantification can be applied, such as mRNA in situ hybridization in formalin-fixed, paraffin-embedded (FFPE) tissue samples or cells, which uses probe sets for each mRNA that bind specifically to an amplification system to amplify the hybridization signals; these amplified signals can be visualized using a standard fluorescence microscope or imaging system.

TaqMan probe-based gene expression analysis (PCR-based) can also be used for measuring biomarker gene expression levels in tissue samples, including mRNA levels in FFPE samples. TaqMan probe-based assays utilize a probe that hybridizes specifically to the mRNA target. This probe contains a quencher dye and a reporter dye (fluorescent molecule) attached to each end, and fluorescence is emitted only when specific hybridization to the mRNA target occurs. During the amplification step, the exonuclease activity of the polymerase enzyme causes the quencher and the reporter dyes to be detached from the probe, and fluorescence emission can occur. This fluorescence emission is recorded and signals are measured by a detection system; these signal intensities are used to calculate the abundance of a given transcript (gene expression) in a sample.

As used herein, a “nucleic acid” can generally refer to a polynucleotide sequence, or fragment thereof. A nucleic acid can comprise nucleotides. A nucleic acid can be exogenous or endogenous to a cell. A nucleic acid can exist in a cell-free environment. A nucleic acid can be a gene or fragment thereof. A nucleic acid can be DNA. A nucleic acid can be RNA. A nucleic acid can comprise one or more analogs (for example, altered backbone, sugar, or nucleobase). “Nucleic acid”, “polynucleotide, “target polynucleotide”, and “target nucleic acid” can be used interchangeably.

As used herein, the term “mRNA” or sometimes refer by “mRNA transcripts” include but is not limited to pre-mRNA transcript(s), transcript processing intermediates, mature mRNA(s) ready for translation and transcripts of the gene or genes, or nucleic acids derived from the mRNA transcript(s). Transcript processing can include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

As used herein “sequencing” a nucleic acid molecule means determining the identity of at least one nucleotide in the molecule. In one embodiment, the identity of less than all of the nucleotides in a molecule are determined. In other embodiments, the identity of a majority or all of the nucleotides in the molecule is determined.

As used herein, the term “biological sample” refers to any sample obtained from a subject. A biological sample can be obtained from a subject prior to or after a diagnosis, at one or more time points prior to or following treatment or therapy, at one or more time points during which there is no treatment or therapy or can be collected from a healthy subject. The biological sample can be a tissue sample or a fluid sample. In one embodiment, the biological sample includes a tissue sample, a biopsy sample, a tumor aspirate, a bone marrow aspirate, or a blood sample (or a fraction thereof, such as blood or serum). In one embodiment, the biological sample includes a tumor cell or cancer cell, for example a circulating tumor cell present in a fluid sample, for example, blood or a fraction thereof. In one embodiment, the biological sample includes a cell free nucleic acid present in a fluid sample, for example, blood or a fraction thereof. In one embodiment, the biological sample comprises a cell lysate (or lysate fraction) or cell extract; or a solution containing one or more molecules derived from a cell or cellular material (for example a polypeptide or nucleic acid). The cell lysate can include proteins, nuclear and/or mitochondrial fractions. In other embodiments, the cell lysate includes a cytosolic fraction. In some embodiments, the cell lysate includes a nuclear/mitochondrial fraction and a cytosolic fraction.

The source of a biological sample can be solid tissue as from a fresh, frozen and/or preserved organ, tissue sample, biopsy, or aspirate; blood or any blood constituents; bodily fluids such as cerebral spinal fluid, amniotic fluid, peritoneal fluid, or interstitial fluid; or cells from any time in gestation or development of the subject. The biological sample can contain compounds that are not naturally intermixed with the tissue in nature such as preservatives, anticoagulants, buffers, fixatives, nutrients, antibiotics or the like. The biological sample can be preserved as a frozen sample or as formaldehyde- or paraformaldehyde-fixed paraffin-embedded (FFPE) tissue preparation. For example, the sample can be embedded in a matrix, for example, an FFPE block or a frozen sample. However, other tissue and sample types are amenable for use herein. In one embodiment, the other tissue and sample types can be fresh frozen tissue, wash fluids, or cell pellets, or the like. A biological sample can be a tumor sample, which contains nucleic acid molecules from a tumor or cancer. A biological sample that is a tumor sample can be DNA, for example, genomic DNA, or cDNA derived from RNA. In one embodiment, the tumor nucleic acid sample is purified or isolated (for example, it is removed from its natural state). In one embodiment, the sample is a tissue (for example, a tumor biopsy), a CTC or cell free nucleic acid.

In one embodiment, a tumor sample is isolated from a human subject. In a further embodiment, the analysis is performed on a tumor biopsy embedded in paraffin wax. In one embodiment, the sample can be a fresh frozen tissue sample. In another embodiment, the sample can be a bodily fluid obtained from the subject. The bodily fluid can be blood or fractions thereof (specifically, serum, plasma), urine, saliva, sputum, or cerebrospinal fluid (CSF). The sample can contain cellular as well as extracellular sources of nucleic acid. The extracellular sources can be cell-free nucleic acids and/or exosomes. The methods described herein, including the RT-PCR methods, are sensitive, precise and have multi-analyte capability for use with paraffin embedded samples. See, for example, Cronin et al., Am. J Pathol. 164(1):35-42 (2004).

General methods for mRNA extraction are well known in the art and are disclosed in standard textbooks of molecular biology, including Ausubel et al, ed., Current Protocols in Molecular Biology, John Wiley & Sons, New York 1987-1999. Methods for RNA extraction from paraffin embedded tissues are disclosed, for example, in Rupp and Locker (Lab Invest. 56:A67, 1987) and De Andres et al. (Biotechniques 18:42-44, 1995). In particular, RNA isolation can be performed using a purification kit, a buffer set and protease from commercial manufacturers according to the manufacturers' instructions. RNA prepared from a tumor can be isolated, for example, by cesium chloride density gradient centrifugation.

“Likely to” or “increased likelihood,” as used herein, refers to an increased probability that an event will occur. Thus, in one example, a subject that is likely to respond to a treatment with a MEK inhibitor has an increased probability of responding to a treatment with the MEK inhibitor relative to a reference subject or group of subjects.

“Unlikely to” refers to a decreased probability that an event, item, object, thing or person will occur with respect to a reference. Thus, a subject that is unlikely to respond to a treatment with a MEK inhibitor has a decreased probability of responding to a treatment with the MEK inhibitor relative to a reference subject or group of subjects.

As used herein, “genomic profiling” means sequencing a part or all of the genome of a subject, such as to identify the nucleotide sequence of one or more genes in the subject, such as to identify genomic alterations (for example, mutations) in one or more biomarker genes that would identify the subject as a candidate to receive certain drugs or other therapeutic agents. Genomic profiling can be performed by a method described herein, such as by a next-generation sequencing method, or a massively parallel sequencing method.

The term “probe” refers to any molecule which is capable of selectively binding to a specifically intended target molecule, for example, a nucleotide transcript or protein encoded by or corresponding to a marker. Probes can be either synthesized by one skilled in the art, or derived from appropriate biological preparations. For purposes of detection of the target molecule, probes can be specifically designed to be labeled, as described herein. Examples of molecules that can be utilized as probes include, but are not limited to, RNA, DNA, proteins, antibodies, and organic molecules.

As used herein, the term “genotype” refers to the alleles at one or more specific biomarker genes. The genotype of a biomarker gene can be determined by methods that include nucleic acids (RNA, cDNA, and DNA) and proteins, and variants and fragments thereof.

As used herein, the term “sensitive” in the context of a MEK inhibitor therapy, means that the MEK inhibitor therapy is more effective at reducing the tumor relative to a control drug in a subject of the same genotype.

As used herein, the term “resistant” in the context of a MEK inhibitor therapy, means that the MEK inhibitor therapy is less effective at reducing the tumor relative to a control drug in a subject of the same genotype.

As described herein, responses to a MEK inhibitor include sensitivity and resistance compared to the response to a control drug in a subject of the same genotype. Such genotype-specific therapeutic responses (GSTRs) that can be characterized based on the relative numbers of tumors above a certain size after treatment (ScoreRTN—Relative Tumor Number) and the geometric mean of tumors from the full distribution of tumor sizes (ScoreRGM—Relative Geometric Mean) as described in Li, C., Lin, W.-Y et al. Quantitative in vivo analyses reveal a complex pharmacogenomic landscape in lung adenocarcinoma. Cancer Res (2021) 81 (17): 4570-4580 (doi: 10.1158/0008-5472.CAN-21-0716).

In one embodiment, the resistance and/or sensitivity profiles of one or more biomarker genes for a MEK inhibitor, alone or in combination with a TORC inhibitor or a chemotherapy, can be compared to the corresponding resistance and/or sensitivity scores for a standard of care therapy in order to determine whether a human subject is likely to benefit from a MEK inhibitor therapy. For example, the MEK inhibitor therapy can be compared to a standard of care (SoC) therapy for a particular cancer to determine which genotypes are sensitive or resistant relative to the SoC therapy using the described herein. Then, by excluding resistant subjects and enrichment for sensitive subjects, the performance of a MEK inhibitor relative to a SoC can be improved. For example, if there are four biomarker genes predictive of resistance to a MEK inhibitor therapy and two of the four biomarker genes show a lower resistance to the MEK inhibitor therapy relative to the SoC therapy, while the other two show a higher resistance relative to the standard of care therapy, only the former two biomarker genes can be used for selecting the subject for the MEK inhibitor therapy over the standard of care therapy.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

A nucleic acid molecule corresponding to a biomarker gene of the present invention can be isolated using standard molecular biology techniques and the sequence information in the database records described herein. Using all or a portion of such nucleic acid sequences, nucleic acid molecules of the present invention can be isolated using standard hybridization and cloning techniques (for example, as described in Sambrook et al., ed., Molecular Cloning: A Laboratory Manual, 2nd ed., Cold Spring Harbor Laboratory Press, Cold Spring Harbor, N.Y., 1989).

A nucleic acid molecule of the present invention can be amplified using cDNA, mRNA, or genomic DNA as a template and appropriate oligonucleotide primers according to standard PCR amplification techniques.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

In the case of measuring protein levels to determine biomarker gene expression, any method known in the art is suitable provided it results in adequate specificity and sensitivity. For example, protein levels can be measured by binding to an antibody or antibody fragment specific for the protein and measuring the amount of antibody-bound protein. Antibodies can be labeled by radioactive, fluorescent, or other detectable reagents to facilitate detection. Methods of detection include, without limitation, enzyme-linked immunosorbent assay (ELISA) and immunoblot techniques.

In some aspects, the invention provides a method of determining a genotype of one or more biomarker genes selected from ADAR, APC, ARID1A, ARID2, ASXL1, ATM, ATRX, BAP1, BRCA1, BRCA2, CDKN2A, CHD2, CIC, CMTR2, CREBBP, CUL3, DICER1, DLC1, DNMT1, DUSP4, EP300, FAT1, FBXW7, JAK1, KDM5C, KDM6A, KEAP1, KMT2C, KMT2D, KRAS, LRP1B, MET, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TREX1, TP53, TSC1, TSC2, USP15, and ZFHX3 in a biological sample from a human subject afflicted with cancer, the method comprising (a) contacting the biological sample with one or more binding agents, each binding agent specific for one of the biomarker genes, and (b) further processing the sample to determine a genotype of each of the one or more biomarker genes in the biological sample.

In one embodiment, the one or more biomarker genes are selected from APC, ARID2, ATM, ATRX, BRCA2, CDKN2A, CMTR2, KEAP1, KMT2D, MGA, NF1, PTEN, PTPRD, RB1, RBM10, RNF43, SETD2, SMAD4, STAG2, STK11, TP53, and TSC1. In one embodiment, the one or more biomarker genes comprise APC, ARID2, ATM, ATRX, BRCA2, CDKN2A, CMTR2, FBXW7, KDM6A, KEAP1, KMT2D, KRAS, MGA, MSH2, NF1, NF2, PALB2, PCNA, PTEN, PTPN11, PTPRD, RB1, RBM10, RNF43, SETD2, SMAD4, STAG2, STK11, TP53, and TSC1.

In one embodiment, the one or more biomarker genes are selected from APC, ARID2, ATM, ATRX, BRCA2, CDKN2A, CMTR2, MGA, NF1, PTEN, PTPRD, RB1, RBM10, RNF43, SETD2, STAG2, STK11, TP53, and TSC1. In one embodiment, the one or more biomarker genes are selected from KEAP1, KMT2D, and SMAD4.

In one embodiment, the one or more biomarker genes are selected from CDKN2A, EP300, KRAS, MGA, RB1, RBM10, SETD2, STAG2, STK11, TP53, USP15, and ZFHX3. In one embodiment, the one or more biomarker genes comprise CDKN2A, EP300, RBM10, and SETD2.

In one embodiment, the one or more biomarker genes are selected from ARID2, BAP1, BRCA1, CIC, KDM6A, KMT2C, KMT2D, LRP1B, NCOA6, NF2, PALB2, PTEN, RASA1, SMAD2, SMG1, SMARCA4, and TET2. In one embodiment, the one or more biomarker genes are selected from ARID2, BAP1, BRCA1, CIC, KMT2D, NCOA6, and RASA1.

In one embodiment, the one or more biomarker genes are selected from ARID2, CDKN2A, CMTR2, SETD2, STAG2, and STK11. In one embodiment, the one or more biomarker genes are selected from ARID2, ATM, CDKN2A, CMTR2, KRAS, MGA, MSH2, RB1, RBM10, RNF43, SETD2, SMAD4, STAG2, and STK11 In one embodiment, the one or more biomarker genes comprise KMT2D and PTEN.

In some aspects, the invention provides a method of determining a genotype of one or more biomarker genes selected from APC, ARID1A, ARID2, ASXL1, ATM, ATRX, BAP1, BRCA1, BRCA2, CDKN2A, CIC, CMTR2, CUL3, DICER1, DLC1, DUSP4, EP300, FAT1, FBXW7, KDM5C, KDM6A, KEAP1, KMT2C, KMT2D, KRAS, LRP1B, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TP53, TSC1, TSC2, USP15, and ZFHX3 in a biological sample from a human subject afflicted with cancer, the method comprising (a) contacting the biological sample with one or more binding agents, each binding agent specific for one of the biomarker genes, and (b) further processing the sample to determine a genotype of each of the one or more biomarker genes in the biological sample.

In some aspects, the invention provides a method of determining a genotype of one or more biomarker genes selected from ARID2, ASXL1, ATM, BAP1, BRCA1, BRCA2, CIC, CUL3, DLC1, FAT1, KDM5C, KDM6A, KMT2C, KMT2D, LRP1B, MGA, MTAP, NCOA6, NF2, PALB2, PBRM1, PTEN, PTPN11, PTPN13, PTPRS, RASA1, RB1, RB1CC1, RNF43, SMAD2, SMAD4, SMARCA4, SMG1, TGFBR2, TP53, and TSC2 in a biological sample from a human subject afflicted with cancer, the method comprising (a) contacting the biological sample with one or more binding agents, each binding agent specific for one of the biomarker genes, and (b) further processing the sample to determine a genotype of each of the one or more biomarker genes in the biological sample. In some embodiments, the one or more biomarker genes are ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, and SMG1.

In some aspects, the invention provides a method of determining a genotype of one or more biomarker genes selected from APC, ARID1A, ATRX CDKN2A, CMTR2, DUSP4, EP300, FBXW7, KEAP1, KRAS, MSH2, NF1, PTPRD, RBM10, SETD2, STAG2, STK11, TET2, TSC1, USP15, and ZFHX3 in a biological sample from a human subject afflicted with cancer, the method comprising (a) contacting the biological sample with one or more binding agents, each binding agent specific for one of the biomarker genes, and (b) further processing the sample to determine a genotype of each of the one or more biomarker genes in the biological sample. In some embodiments, the one or more biomarker genes are selected from CDKN2A, EP300, KEAP1, KRAS, RBM10, SETD2, STK11, TSC1, and USP15.

In some embodiments, the invention provides a method of predicting resistance of tumor growth to inhibition by a therapy comprising a MEK inhibitor, the method comprising: (a) detecting in a tumor sample from a human subject afflicted with cancer a genotype of one or more biomarker genes; (b) analyzing the genotype of the one or more biomarker genes in the tumor sample; and (c) predicting resistance of tumor cell growth to inhibition by the therapy comprising a MEK inhibitor, if the tumor sample comprises (i) an inactivating ARID2, BAP1, BRCA1, CIC, KMT2D, NCOA6, or RASA1 mutation, (ii) a decreased copy number of ARID2, BAP1, BRCA1, CIC, KMT2D, NCOA6, or RASA1, or (iii) a decreased expression of ARID2, BAP1, BRCA1, CIC, KMT2D, NCOA6, or RASA1 mRNA or protein.

In some embodiments, the invention provides a method of predicting resistance of tumor growth to inhibition by a therapy comprising a MEK inhibitor, the method comprising: (a) detecting in a tumor sample from a human subject afflicted with cancer a genotype of one or more biomarker genes; (b) analyzing the genotype of the one or more biomarker genes in the tumor sample; and (c) predicting resistance of tumor cell growth to inhibition by the therapy comprising a MEK inhibitor, if the tumor sample comprises (i) an inactivating ARID2, BAP1, BRCA1, CIC, KDM6A, KMT2C, KMT2D, LRP1B, NCOA6, NF2, PALB2, PTEN, RASA1, SMAD2, SMG1, SMARCA4, or TET2 mutation, (ii) a decreased copy number of ARID2, BAP1, BRCA1, CIC, KDM6A, KMT2C, KMT2D, LRP1B, NCOA6, NF2, PALB2, PTEN, RASA1, SMAD2, SMG1, SMARCA4, or TET2, or (iii) a decreased expression of ARID2, BAP1, BRCA1, CIC, KDM6A, KMT2C, KMT2D, LRP1B, NCOA6, NF2, PALB2, PTEN, RASA1, SMAD2, SMG1, SMARCA4, or TET2 mRNA or protein.

In some embodiments, the invention provides a method of predicting sensitivity of tumor growth to inhibition by a therapy comprising a MEK inhibitor, the method comprising: (a) detecting in a tumor sample from a human subject afflicted with cancer a genotype of one or more biomarker genes; (b) analyzing the genotype of the one or more biomarker genes in the tumor sample; and (c) predicting sensitivity of tumor cell growth to inhibition by the therapy comprising a MEK inhibitor, if the tumor sample comprises (i) an inactivating CDKN2A, EP300, KRAS, MGA, RB1, RBM10, SETD2, STAG2, STK11, TP53, USP15, or ZFHX3 mutation, (ii) a decreased copy number of CDKN2A, EP300, KRAS, MGA, RB1, RBM10, SETD2, STAG2, STK11, TP53, USP15, or ZFHX3, or (iii) decreased expression of CDKN2A, EP300, KRAS, MGA, RB1, RBM10, SETD2, STAG2, STK11, TP53, USP15, or ZFHX3 mRNA or protein.

In some embodiments, the invention provides a method of predicting sensitivity of tumor growth to inhibition by a therapy comprising a MEK inhibitor, the method comprising: (a) detecting in a tumor sample from a human subject afflicted with cancer a genotype of one or more biomarker genes; (b) analyzing the genotype of the one or more biomarker genes in the tumor sample; and (c) predicting sensitivity of tumor cell growth to inhibition by the therapy comprising a MEK inhibitor, if the tumor sample comprises (i) an inactivating CDKN2A, EP300, RBM10, or SETD2 mutation, (ii) a decreased copy number of CDKN2A, EP300, RBM10, or SETD2, or (iii) decreased expression of CDKN2A, EP300, RBM10, or SETD2 mRNA or protein.

In some embodiments, the invention provides a method of predicting response of tumor growth to inhibition by a therapy comprising a MEK inhibitor, the method comprising: (a) determining in a tumor sample from a human subject afflicted with cancer a genotype of one or more biomarker genes; (b) analyzing the genotype of the one or more biomarker genes in the tumor sample; and (c) predicting the response of tumor cell growth to inhibition by the therapy comprising a MEK inhibitor, if the tumor sample comprises (i) an inactivating CDKN2A, EP300. KRAS, MGA, RB1, RBM10, SETD2, STAG2, STK11, TP53, USP15, or ZFHX3 mutation, (ii) a decreased copy number of CDKN2A, EP300, KRAS, MGA, RB1, RBM10, SETD2, STAG2, STK11, TP53, USP15, or ZFHX3, (iii) decreased expression of CDKN2A, EP300, KRAS, MGA, RB1, RBM10, SETD2, STAG2, STK11, TP53, USP15, or ZFHX3 mRNA or protein; (iv) an inactivating ARID2, BAP1, BRCA1, CIC, KDM6A, KMT2C, KMT2D, LRP1B, NCOA6, NF2, PALB2, PTEN, RASA1, SMAD2, SMG1, SMARCA4, or TET2, (v) a decreased copy number of ARID2, BAP1, BRCA1, CIC, KDM6A, KMT2C, KMT2D, LRP1B, NCOA6, NF2, PALB2, PTEN, RASA1, SMAD2, SMG1, SMARCA4, or TET2, or (vi) a decreased expression of ARID2, BAP1, BRCA1, CIC, KDM6A, KMT2C, KMT2D, LRP1B, NCOA6, NF2, PALB2, PTEN, RASA1, SMAD2, SMG1, SMARCA4, or TET2 mRNA or protein.

In some embodiments, the invention provides a method of predicting response of tumor growth to inhibition by a therapy comprising a MEK inhibitor, the method comprising: (a) determining in a tumor sample from a human subject afflicted with cancer a genotype of one or more biomarker genes; (b) analyzing the genotype of the one or more biomarker genes in the tumor sample; and (c) predicting the response of tumor cell growth to inhibition by the therapy comprising a MEK inhibitor, if the tumor sample comprises (i) an inactivating CDKN2A, EP300, RBM10, or SETD2 mutation, (ii) a decreased copy number of CDKN2A, EP300, RBM10, or SETD2, (iii) decreased expression of CDKN2A, EP300, RBM10, or SETD2 mRNA or protein; (iv) an inactivating ARID2, BAP1, BRCA1, CIC, KMT2D, NCOA6, or RASA1 mutation, (v) a decreased copy number of ARID2, BAP1, BRCA1, CIC, KMT2D, NCOA6, or RASA1, or (vi) a decreased expression of ARID2, BAP1, BRCA1, CIC, KMT2D, NCOA6, or RASA1 mRNA or protein.

In the methods described above and throughout this application, the biomarker genes can be selected from any suitable list known or curated by one skilled in the art, for example, ADAR, APC, ARID1A, ARID2, ASXL1, ATM, ATRX, BAP1, BRCA1, BRCA2, CDKN2A, CHD2, CIC, CMTR2, CREBBP, CUL3, DICER1, DLC1, DNMT1, DUSP4, EP300, FAT1, FBXW7, JAK1, KDM5C, KDM6A, KEAP1, KMT2C, KMT2D, KRAS, LRP1B, MET, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TREX1, TP53, TSC1, TSC2, USP15, and ZFHX3.

In some embodiments, the invention provides a method of treating non-small cell lung cancer (NSCLC) comprising treating a subject with a MEK inhibitor when a tumor sample obtained from the subject comprises (i) an inactivating mutation in one or more of CDKN2A, EP300, RBM10, or SETD2, (ii) a decreased copy number of one or more of CDKN2A, EP300, RBM10, or SETD2, or (iii) decreased expression of CDKN2A, EP300, RBM10, or SETD2 mRNA or protein. In further embodiments, the tumor sample obtained from the subject further comprises absence of (i) an inactivating mutation in one or more of ARID2, BAP1, BRCA1, CIC, KMT2D, NCOA6, or RASA1, (ii) a decreased copy number of one or more of ARID2, BAP1. BRCA1, CIC, KMT2D, NCOA6, or RASA1, or (iii) decreased expression of ARID2, BAP1. BRCA1, CIC, KMT2D, NCOA6, or RASA1 mRNA or protein.

In some embodiments, the invention provides a method of treating non-small cell lung cancer (NSCLC) comprising treating a subject with a MEK inhibitor when a tumor sample obtained from the subject comprises (i) an inactivating mutation in one or more of CDKN2A, EP300, KRAS, MGA, RB1, RBM10, SETD2, STAG2, STK11, TP53, USP15, or ZFHX3, (ii) a decreased copy number of one or more of CDKN2A, EP300, KRAS, MGA, RB1, RBM10, SETD2, STAG2, STK11, TP53, USP15, or ZFHX3, or (iii) decreased expression of CDKN2A, EP300, KRAS, MGA, RB1, RBM10, SETD2, STAG2, STK11, TP53, USP15, or ZFHX3 mRNA or protein. In further embodiments, the tumor sample obtained from the subject further comprises absence of (i) an inactivating mutation in one or more of ARID2, BAP1, BRCA1, CIC, KDM6A, KMT2C, KMT2D, LRP1B, NCOA6, NF2, PALB2, PTEN, RASA1, SMAD2, SMG1, SMARCA4, or TET2, (ii) a decreased copy number of one or more of ARID2, BAP1, BRCA1, CIC, KDM6A, KMT2C, KMT2D, LRP1B, NCOA6, NF2, PALB2, PTEN, RASA1, SMAD2, SMG1, SMARCA4, or TET2, or (iii) decreased expression of CDKN2A, EP300, KRAS, MGA, RB1, RBM10, SETD2, STAG2, STK11, 7P53, USP15, or ZFHX3 mRNA or protein.

In some embodiments, the invention provides a method of treating non-small cell lung cancer (NSCLC) comprising treating a subject with a combination therapy comprising a MEK inhibitor and a TORC inhibitor when a tumor sample obtained from the subject comprises (i) an inactivating mutation in one or more of ARID2, ATM, CDKN2A, CMTR2, KRAS, MGA, MSH2, RB1, RBM10, RNF43, SETD2, SMAD4, STAG2, or STK11, (ii) a decreased copy number of one or more of ARID2, ATM, CDKN2A, CMTR2, KRAS, MGA, MSH2, RB1, RBM10, RNF43, SETD2, SMAD4, STAG2, or STK11, or (iii) a decreased expression of ARID2, ATM, CDKN2A, CMTR2, KRAS, MGA, MSH2, RB1, RBM10, RNF43, SETD2, SMAD4, STAG2, or STK11 mRNA or protein. In further embodiments, the tumor sample obtained from the subject further comprises absence of (i) an inactivating mutation in one or more of KMT2D or PTEN, (ii) a decreased copy number of one or more of KMT2D or PTEN, or (iii) a decreased expression of KMT2D or PTEN mRNA or protein.

In some embodiments, the invention provides a method of treating non-small cell lung cancer (NSCLC) comprising treating a subject with a combination therapy comprising a MEK inhibitor and a TORC inhibitor when a tumor sample obtained from the subject comprises (i) an inactivating mutation in one or more of ARID2, CDKN2A, CMTR2, SETD2, STAG2, or STK11, (ii) a decreased copy number of one or more of ARID2, CDKN2A, CMTR2, SETD2, STAG2, or STK11, or (iii) a decreased expression of ARID2, CDKN2A, CMTR2, SETD2, STAG2, or STK11 mRNA or protein. In further embodiments, the tumor sample obtained from the subject further comprises absence of (i) an inactivating mutation in one or more of KMT2D or PTEN, (ii) a decreased copy number of one or more of KMT2D or PTEN, or (iii) a decreased expression of KMT2D or PTEN mRNA or protein.

In some embodiments, the invention provides a method of treating non-small cell lung cancer (NSCLC) comprising treating a subject with a combination therapy comprising a combination therapy comprising MEK inhibitor and a chemotherapy when a tumor sample obtained from the subject comprises (i) an inactivating mutation in one or more of CDKN2A, EP300, KEAP1, KRAS, RBM10, SETD2, SIK11, TSC1, or USP15, (ii) a decreased copy number of one or more of CDKN2A, EP300, KEAP1, KRAS, RBM10, SETD2, STK11, TSC1, or USP15, or (iii) a decreased expression of CDKN2A, EP300, KEAP1, KRAS, RBM10, SETD2, STK11, TSC1, or USP15 mRNA or protein. In further embodiments, the tumor sample obtained from the subject further comprises absence of (i) an inactivating mutation in one or more of ARID2, ASAI, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, or SMG1, (ii) a decreased copy number of one or more of ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, or SMG1, or (iii) a decreased expression of ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, or SMG1 mRNA or protein.

Accordingly, in some embodiments of the methods disclosed herein, a biological sample of the subject has previously been tested for a mutation in one or more genes. In some embodiments, the one or more genes comprise a driver gene. In some embodiments, the driver gene is an oncogene. In some embodiments, the driver oncogene can be, for example and without limitation, Kirsten rat sarcoma viral oncogene homolog (KRAS), epidermal growth factor receptor (EGFR), anaplastic lymphoma kinase (ALK), c-ROS oncogene 1 (ROS1), BRAF, rearranged during transfection gene (RET), MET and human epidermal growth factor receptor 2 (HER2). In some embodiments, the biological sample of the subject has previously been tested for KRAS mutant variants, for example, G12C, G12D, or G12V. In some embodiments, the biological sample of the subject has previously been tested for prevalence of immune biomarkers such as programmed cell death ligand 1 (PD-L1). In some embodiments, a biological sample of the subject has previously been tested for a mutation in one or more tumor suppressor genes. In some embodiments, a biological sample of the subject has been previously tested with a multigene panel that interrogates for mutations in several genes at once. In some embodiments, a biological sample of the subject has been previously tested with a targeted single variant test, a single gene test, or has been analyzed by whole exome sequencing or whole genome sequencing. In some embodiments the subjects have a known driver mutation or other genetic profile.

The present invention provides, in part, methods for accurately classifying a human subject afflicted with cancer as sensitive to therapy with a MEK inhibitor. The present invention provides, in part, methods for accurately classifying a human subject afflicted with cancer as sensitive to a combination therapy with a MEK inhibitor and a TORC inhibitor. The present invention also provides methods for accurately classifying a human subject afflicted with cancer as sensitive to a combination therapy with a MEK inhibitor and a chemotherapy.

In some embodiments, the invention provides methods for selecting a subject for treatment with a MEK monotherapy if it is likely that the subject will respond to the MEK monotherapy, wherein the likelihood of response is determined by performing any of the disclosed methods for predicting resistance, sensitivity, or response of tumor growth to inhibition by the therapy on a biological sample obtained from the subject. In some embodiments, the determination is based on results of an existing biological sample of the subject.

In some embodiments, the invention provides methods for selecting a subject for treatment with a combination therapy if it is likely that the subject will respond to the combination therapy comprising a MEK inhibitor and a TORC inhibitor, wherein the likelihood of response is determined by performing any of the disclosed methods for predicting resistance, sensitivity, or response of tumor growth to inhibition by the therapy on a biological sample obtained from the subject. In some embodiments, the determination is based on results of an existing biological sample of the subject.

In some embodiments, the invention provides methods for selecting a subject for treatment with a combination therapy comprising a MEK inhibitor and a chemotherapy if it is likely that the subject will respond to the combination therapy comprising a MEK inhibitor and a chemotherapy, wherein the likelihood of response is determined by performing any of the disclosed methods for predicting resistance, sensitivity, or response of tumor growth to inhibition by the therapy on a biological sample obtained from the subject. In some embodiments, the determination is based on results of an existing biological sample of the subject.

In some embodiments, the methods comprise administering to a subject a MEK monotherapy, or combination therapy comprising a MEK inhibitor and a TORC inhibitor, or a combination therapy comprising a MEK inhibitor and a chemotherapy if the predicted response of the subject to the therapy is sensitivity of tumor growth to inhibition by a therapy comprising a MEK inhibitor. In some embodiments, the methods further comprise administering to a subject a MEK monotherapy, or a combination therapy comprising a MEK inhibitor and a TORC inhibitor, or a combination therapy comprising a MEK inhibitor and a chemotherapy if the predicted response of the subject to the therapy is sensitivity of tumor growth to inhibition and absence of resistance of tumor growth to inhibition by a therapy comprising a MEK inhibitor.

In some embodiments, the methods comprise selecting a subject for treatment with a MEK monotherapy, or a combination therapy comprising a MEK inhibitor and a TORC inhibitor, or a combination therapy comprising a MEK inhibitor and a chemotherapy to a subject if the predicted response to the therapy of the subject is sensitivity of tumor growth to inhibition by a therapy comprising a MEK inhibitor. In some embodiments, the methods further comprise administering to a subject a MEK monotherapy, or a combination therapy comprising a MEK inhibitor and a TORC inhibitor, or a combination therapy comprising a MEK inhibitor and a chemotherapy if the predicted response of the subject to the therapy is sensitivity of tumor growth to inhibition and absence of resistance of tumor growth to inhibition by a therapy comprising a MEK inhibitor.

The present invention provides, in part, methods for enriching a patient population for subjects sensitive to a therapy with a MEK inhibitor. The present invention provides, in part, methods for enriching a patient population for subjects sensitive to a combination therapy with a MEK inhibitor and a TORC inhibitor. The present invention also provides methods for enriching a patient population for subjects sensitive to a combination therapy with a MEK inhibitor and a chemotherapy. Any of the methods herein can be applied on a population level to enrich for subjects sensitive to a therapy. As used herein, the term “prospective patient population” describes a population of human subjects previously diagnosed with cancer. In some embodiments the cancer is lung cancer. In some embodiments, the lung cancer is non-small cell lung cancer (NSCLC). The term, “enriching” or “enrichment” as used herein in reference to a prospective patient population refers to stratification of the population to identify those patients who are most likely to respond to a particular therapy.

The present invention provides, in part, methods for accurately classifying a human subject afflicted with cancer as resistant to a therapy with a MEK inhibitor. The present invention provides, in part, methods for accurately classifying a human subject afflicted with cancer as resistant to a combination therapy with a MEK inhibitor and a TORC inhibitor. The present invention also provides methods for accurately classifying a human subject afflicted with cancer as resistant to a combination therapy with a MEK inhibitor and a chemotherapy.

The methods comprise obtaining a tumor sample from the subject and determining a genotype of one or more biomarker genes. In one embodiment, the biological sample (for example, tumor sample) contains polypeptides encoded by the one or more biomarker genes. Alternatively, the biological sample can contain mRNA molecules or genomic DNA corresponding to the one or more biomarker genes. In some embodiments, the methods involve obtaining a tumor sample from the subject and contacting the tumor sample with a reagent capable of determining the genotype by detecting, for example, a polypeptide or nucleic acid that encodes the biomarker gene or fragments thereof.

The methods of the invention detect mRNA, polypeptide, genomic DNA, or fragments thereof, in a biological sample in vitro as well as in vivo. For example, in vitro techniques for detection of mRNA or a fragment thereof include Northern hybridizations and in situ hybridizations. In vitro techniques for detection of polypeptide include enzyme linked immunosorbent assays (ELISAs), Western blots, immunoprecipitations and immunofluorescence. In vitro techniques for detection of biomarker genomic DNA or a fragment thereof include Southern hybridizations. Furthermore, in vivo techniques for detection of one or more polypeptides or fragments thereof include labeled antibodies. For example, the antibody can be labeled with a radioactive marker whose presence and location in a human subject can be detected by standard imaging techniques.

In some embodiments, the genotype, presence or level of at least one, two, three, four, five, six, seven, eight, nine, ten, fifty, sixty, or more biomarker genes of the invention is determined in the tumor sample. In some embodiments, methods of the invention employ a statistical algorithm and/or empirical data (for example, the presence or level of one or biomarker genes described herein). In certain instances, a single learning statistical classifier system can be used to classify a sample. The use of a single learning statistical classifier system typically classifies the sample accurately with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Other suitable statistical algorithms are well known to those of skill in the art. For example, learning statistical classifier systems include a machine learning algorithmic technique capable of adapting to complex data sets (for example, panel of markers of interest) and making decisions based upon such data sets. In some embodiments, a single learning statistical classifier system such as a classification tree (for example, random forest) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (for example, decision/classification trees such as random forests, classification and regression trees (C&RT), boosted trees, etc.), Probably Approximately Correct (PAC) learning, connectionist learning (for example, neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (for example, passive learning in a known environment such as naive learning, adaptive dynamic learning, and temporal difference learning, passive learning in an unknown environment, active learning in an unknown environment, learning action-value functions, applications of reinforcement learning, and genetic algorithms and evolutionary programming. Other learning statistical classifier systems include support vector machines (for example, Kernel methods), multivariate adaptive regression splines (MARS), Levenberg-Marquardt algorithms, Gauss-Newton algorithms, mixtures of Gaussians, gradient descent algorithms, and learning vector quantization (LVQ). In certain embodiments, the method of the present invention further comprises sending the cancer classification results to a clinician, for example, an oncologist or hematologist.

In one embodiment, determining the genotype of a biomarker gene comprises genomic profiling to directly determine the genotype of the one or more biomarker genes. In one embodiment, genomic profiling comprises contacting the biological sample with reagents, including, probes and/or primers, for sequencing a biomarker gene or portion thereof. In one embodiment, probes or primers can be designed to detect a mutation in a biomarker gene. In one embodiment, the mutation is an inactivating mutation. In one embodiment, the mutation results in decreased gene expression.

In one embodiment, the methods comprise directly determining the genotype of a biomarker gene by genomic profiling to detect the presence or absence of a genetic alteration characterized by at least one alteration affecting the integrity of a gene encoding one or more biomarker polypeptides, or the mis-expression of the biomarker (for example, mutations and/or splice variants). For example, such genetic alterations can be detected by ascertaining the existence of at least one of a deletion of one or more nucleotides from one or more biomarker genes; an addition of one or more nucleotides to one or more biomarker genes; a substitution of one or more nucleotides of one or more biomarker genes; a chromosomal rearrangement of one or more biomarker genes; an alteration in the level of a mRNA transcript of one or more biomarker genes; aberrant modification of one or more biomarker genes; such as of the methylation pattern of the genomic DNA; the presence of a non-wild type splicing pattern of a messenger RNA transcript of one or more biomarker genes; a non-wild type level of one or more biomarker polypeptides; allelic loss of one or more biomarker genes, and inappropriate post-translational modification of one or more biomarker polypeptides. As described herein, there are a large number of assays known in the art which can be used for detecting alterations in one or more biomarker genes.

In certain embodiments, detection of the alteration involves the use of a probe/primer in a polymerase chain reaction (PCR), such as anchor PCR or RACE PCR, or, alternatively, in a ligation chain reaction (LCR) (see, for example, Landegran et al. (1988) Science 241:1077-1080; and Nakazawa et al. (1994)Proc. Natl. Acad. Sci. USA 91:360-364), the latter of which can be particularly useful for detecting point mutations in one or more biomarker genes (see Abravaya et al. (1995) Nucleic Acids Res. 23:675-682). This method can include the steps of collecting a sample of cells from the human subject, isolating nucleic acid from the cells of the sample, contacting the nucleic acid sample with one or more primers which specifically hybridize to one or more biomarker genes of the invention, or fragments thereof, under conditions such that hybridization and amplification of the biomarker gene (if present) occurs, and detecting the presence or absence of an amplification product, or detecting the size of the amplification product and comparing the length to a control sample. It is anticipated that PCR and/or LCR can be desirable to use as a preliminary amplification step in conjunction with any of the techniques used for detecting mutations described herein.

Alternative amplification methods include self-sustained sequence replication (Guatelli, J. C. et al. (1990)Proc. Natl. Acad. Sci. USA 87:1874-1878), transcriptional amplification system (Kwoh, D. Y. et al. (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in one or more biomarker genes of the invention, or a fragment thereof, from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes can be used to score for the presence of specific mutations by development or loss of a ribozyme cleavage site.

In other embodiments, genetic mutations in one or more biomarker genes of the invention, or a fragment thereof, can be identified by hybridizing a nucleic acid to high density arrays containing hundreds or thousands of oligonucleotide probes (Cronin, M. T. et al. (1996) Hum. Mutat. 7:244-255; Kozal, M. J. et al. (1996) Nat. Med. 2:753-759).

In yet another embodiment, any of a variety of sequencing methods known in the art can be used to directly sequence one or more biomarker genes of the invention, or a fragment thereof, and detect mutations by comparing the sequence of the sample biomarker gene with the corresponding wild-type (control) sequence. Examples of sequencing reactions include next-generation sequencing to determine the nucleotide sequence of either individual nucleic acid molecules (for example, in single molecule sequencing) or clonally expanded proxies for individual nucleic acid molecules in a highly parallel fashion.

Next generation sequencing methods are known in the art, and are described, for example, in Metzker, M. (2010) Nature Biotechnology Reviews 11:31-46. Next generation sequencing collectively refers to several DNA/RNA sequencing technologies that vary according to the input material, length of read, and portion of the genome to be sequenced. Broadly, the 2 major next generation sequencing technologies are short-read sequencing and long-read sequencing. Short-read sequencing generally refers to reads that are shorter than 300 bp, whereas long-read sequencing refers to reads that are longer than 2.5 Kb. Short-read sequencing is a relatively inexpensive option (low costs per Gb) that has a high level of accuracy and is used more frequently in clinical practice for the detection of specific mutation hotspots. Moreover, based on the initial input material, different sequencing approaches can be used (for example, genomic DNA [DNA-seq], messenger or noncoding RNA [RNA-seq], or any nucleic or ribonucleic material obtained following the use of certain procedures).

Current next generation sequencing approaches also differ based on the extent of target enrichment and sequencing involved, with the 3 major types being whole genome sequencing (WGS), whole-exome sequencing (WES), and targeted gene panels. WGS refers to sequencing the entire genome, including coding and noncoding regions. It allows detection of several types of genetic aberrations, including single nucleotide variants and/or such structural alterations as insertions or deletions (also called indels), copy number variations involving duplications or deletions of long stretches of a chromosomal region, and rearrangements involving gross alterations in chromosomes or large chromosomal regions. WES involves sequencing only the coding regions of the genome and is limited in its ability to detect rearrangements between genes with breakpoints that frequently occur in intronic regions. RNA-based whole-transcriptome approaches can be another strategy to identify gene rearrangements.

Another next generation sequencing strategy, which is currently the most commonly used approach to cancer genotyping in clinical use, is targeted gene panels that interrogate a discrete number of genes. This approach has the advantage of being able to focus on clinically relevant targets with deeper sequencer and focused analyses. Targeted gene panels can be performed with either amplicon-based or hybrid-capture enrichment strategies and can range from small, hotspot-only panels focusing on less than fifty genes to larger, more comprehensive panels that include hundreds to greater than a thousand genes with selected intronic tiling coverage. In addition to lower costs, the advantages of targeted gene panels include greater analytic sensitivity because of the greater depth of coverage, less complex data analysis and interpretation than would be necessary for WES and WGS, and greater flexibility that allows for tailoring the testing to genomic regions relevant to cancer. Any of the known Next generation sequencing approaches can be practiced for the methods described herein and a skilled person will be able to select the best sequencing strategy to practice the methods described herein.

In one embodiment, determining the genotype of a biomarker gene comprises measuring the expression level of one or more biomarker genes. The expression level can be measured in several ways, including, but not limited to measuring the mRNA encoded by the biomarker genes; measuring the amount of protein encoded by the biomarker genes; and measuring the activity of the protein encoded by the biomarker genes. In some aspects, a genotype of a biomarker gene is determined by measuring RNA, cDNA, protein or any combination thereof. When a genotype is determined by measuring RNA, the RNA can be reverse transcribed to produce cDNA (such as by RT-PCR), and the produced cDNA expression level can be detected. The expression level of a biomarker gene can be detected by forming a complex between a nucleic acid corresponding to a biomarker gene and a labeled probe or primer. When the nucleic acid is RNA or cDNA, the RNA or cDNA can be detected by forming a complex between the RNA or cDNA and a labeled nucleic acid probe or primer. The complex between the RNA or cDNA and the labeled nucleic acid probe or primer can be a hybridization complex.

Another method of determining the genotype of a biomarker gene at the nucleic acid level is the use of an amplification method such as, for example, RT-PCR or quantitative RT-PCR (qRT-PCR). Methods for determining the level of mRNA in a sample can involve the process of nucleic acid amplification, for example, by RT-PCR, ligase chain reaction or any other nucleic acid amplification method, followed by the detection of the amplified molecules using techniques well known to those of skill in the art. Numerous different PCR or qRT-PCR protocols are known in the art and can be directly applied or adapted for use using the presently described compositions for the detection and/or quantification of expression of biomarker genes in a sample.

Isolated mRNA can be used in hybridization or amplification assays that include, but are not limited to, Southern or Northern analyses, PCR analyses and probe arrays. One method for the detection of mRNA levels involves contacting the isolated mRNA or synthesized cDNA with a nucleic acid molecule (probe) that can hybridize to the mRNA encoded by the gene being detected. The nucleic acid probe can be, for example, a cDNA, or a portion thereof, such as an oligonucleotide of at least 7, 15, 30, 50, 100, 250, or 500 nucleotides in length and sufficient to specifically hybridize under stringent conditions to the non-natural cDNA or mRNA.

As described herein, biomarker gene expression can be assessed by any of a wide variety of well-known methods for detecting expression of a transcribed molecule or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In one embodiment, activity of a particular biomarker gene is characterized by a measure of gene transcript (for example mRNA), by a measure of the quantity of translated protein, or by a measure of gene product activity. Biomarker gene expression can be monitored in a variety of ways, including by detecting mRNA levels, protein levels, or protein activity, any of which can be measured using standard techniques. Detection can involve quantification of the level of gene expression (for example, genomic DNA, cDNA, mRNA, protein, or enzyme activity), or, alternatively, can be a qualitative assessment of the level of gene expression, in particular in comparison with a control level. The type of level being detected will be clear to the skilled person from the context.

In one embodiment, detecting or determining expression levels of a biomarker gene and functionally similar homologs thereof, including a fragment or genetic alteration thereof (for example, in regulatory or promoter regions thereof) comprises detecting or determining RNA levels for the biomarker marker gene. In one embodiment, one or more cells from the subject to be tested are obtained and RNA is isolated from the cells.

General methods for mRNA extraction are well known in the art and are disclosed in standard textbooks of molecular biology, including Ausubel et al, ed., Current Protocols in Molecular Biology, John Wiley & Sons, New York 1987-1999. Methods for RNA extraction from paraffin embedded tissues are disclosed, for example, in Rupp and Locker (Lab Invest. 56:A67, 1987) and De Andres et al. (Biotechniques 18:42-44, 1995). In particular, RNA isolation can be performed using a purification kit, a buffer set and protease from commercial manufacturers according to the manufacturers' instructions. RNA prepared from a tumor can be isolated, for example, by cesium chloride density gradient centrifugation.

The population of RNA can optionally be enriched, and further be amplified. For example, where RNA is mRNA, an amplification process such as RT-PCR can be utilized to amplify the mRNA, such that a signal is detectable or detection is enhanced. Such an amplification process is beneficial particularly when the biological, tissue, or tumor sample is of a small size or volume. Various amplification and detection methods can be used. For example, it is within the scope of the present invention to reverse transcribe mRNA into cDNA followed by polymerase chain reaction (RT-PCR); or, to use a single enzyme for both steps, or reverse transcribe mRNA into cDNA followed by symmetric gap ligase chain reaction (RT-AGLCR).

Many techniques are known in the state of the art for determining absolute and relative levels of gene expression, commonly used techniques suitable for use in the present invention include Northern analysis, RNase protection assays (RPA), microarrays and PCR-based techniques, such as quantitative PCR and differential display PCR. For example, Northern blotting involves running a preparation of RNA on a denaturing agarose gel, and transferring it to a suitable support, such as activated cellulose, nitrocellulose or glass or nylon membranes. Radiolabeled cDNA or RNA is then hybridized to the preparation, washed and analyzed by autoradiography.

In situ hybridization visualization can also be employed, wherein a radioactively labeled antisense RNA probe is hybridized with a thin section of a biopsy sample, washed, cleaved with RNase and exposed to a sensitive emulsion for autoradiography. The samples can be stained with hematoxylin to demonstrate the histological composition of the sample, and dark field imaging with a suitable light filter shows the developed emulsion. Non-radioactive labels such as digoxigenin can also be used.

Alternatively, mRNA expression can be detected on a DNA array, chip or a microarray. Labeled nucleic acids of a test sample obtained from the human subject can be hybridized to a solid surface comprising biomarker DNA. Positive hybridization signal is obtained with the sample containing biomarker transcripts. In one embodiment, gene expression can be detected by microarray analysis. Differential gene expression can also be identified or confirmed using a microarray technique. The expression levels of one or more biomarker genes can be measured in either fresh or fixed tissue, using microarray technology. In this method, polynucleotide sequences of interest (including cDNAs and oligonucleotides) are plated, or arrayed, on a microchip substrate. The arrayed sequences are then hybridized with specific DNA probes from cells or tissues of interest. Fluorescently labeled cDNA probes can be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the microarray chip is scanned by a device such as, confocal laser microscopy or by another detection method. Quantitation of hybridization of each arrayed element allows for assessment of corresponding mRNA abundance. Microarray analysis can be performed by commercially available equipment, following manufacturer's protocols.

Types of probes that can be used in the methods described herein include cDNA, riboprobes, synthetic oligonucleotides and genomic probes. The type of probe used will generally be dictated by the particular situation, such as riboprobes for in situ hybridization, and cDNA for Northern blotting, for example. In one embodiment, the probe is directed to nucleotide regions unique to the RNA. The probes can be as short as is required to differentially recognize marker mRNA transcripts, and can be as short as, for example, 15 bases; however, probes of at least 17, 18, 19 or 20 or more bases can be used. In one embodiment, the primers and probes hybridize specifically under stringent conditions to a DNA fragment having the nucleotide sequence corresponding to the marker. As herein used, the term “stringent conditions” means hybridization will occur only if there is at least 95% identity in nucleotide sequences. In another embodiment, hybridization under “stringent conditions” occurs when there is at least 97% identity between the sequences.

The activity, level or presence of a protein encoded by a biomarker gene can be detected and/or quantified by detecting or quantifying the expressed polypeptide. The polypeptide can be detected and quantified by any of a number of means well known to those of skill in the art. Any method known in the art for detecting polypeptides can be used. Such methods include, but are not limited to, immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like (for example, Basic and Clinical Immunology, Sites and Terr, eds., Appleton and Lange, Norwalk, Conn. pp 217-262, 1991 which is incorporated by reference).

ELISA and RIA procedures can be conducted such that a desired protein standard is labeled (with a radioisotope such as ¹²¹I or ³⁵S, or an assayable enzyme, such as horseradish peroxidase or alkaline phosphatase), and, together with the unlabelled sample, brought into contact with the corresponding antibody, whereon a second antibody is used to bind the first, and radioactivity or the immobilized enzyme assayed (competitive assay). Alternatively, the protein in the sample is allowed to react with the corresponding immobilized antibody, radioisotope- or enzyme-labeled antibody is allowed to react with the system, and radioactivity or the enzyme assayed (ELISA-sandwich assay). Other conventional methods can also be employed as suitable.

Enzymatic and radiolabeling of a protein encoded by a biomarker gene and/or the antibodies can be effected by conventional means. It is possible to immobilize the enzyme itself on a support, but if solid-phase enzyme is required, then this is generally best achieved by binding to antibody and affixing the antibody to a support, models and systems for which are well-known in the art.

Other techniques can be used to detect protein corresponding to a biomarker gene according to a practitioner's preference based upon the present disclosure. One such technique is Western blotting (Towbin et al., Proc. Nat. Acad. Sci. USA 76:4350 (1979)), wherein a suitably treated sample is run on an SDS-PAGE gel before being transferred to a solid support, such as a nitrocellulose filter. Antibodies specific for the protein (unlabeled) are then brought into contact with the support and assayed by a secondary immunological reagent, such as labeled protein A or anti-immunoglobulin (suitable labels including ¹²¹I, horseradish peroxidase and alkaline phosphatase). Chromatographic detection can also be used.

Immunohistochemistry can be used to detect expression of a protein corresponding to a biomarker gene, for example, in a biopsy sample. A suitable antibody is brought into contact with, for example, a thin layer of cells, washed, and then contacted with a second, labeled antibody. Labeling can be by fluorescent markers, enzymes, such as peroxidase, avidin, or radiolabelling. The assay is scored visually, using microscopy. Any other art-known method can be used to detect a protein corresponding to a biomarker gene.

### EXAMPLES OF NON-LIMITING ASPECTS OF THE DISCLOSURE

Aspects, including embodiments, of the present subject matter described above may be beneficial alone or in combination, with one or more other aspects or embodiments. Without limiting the foregoing description, certain non-limiting aspects of the disclosure numbered 1-150 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. A method comprising: (a) determining a genotype of one or more biomarker genes selected from ADAR, APC, ARID1A, ARID2, ASXL1, ATM, ATRX, BAP1, BRCA1, BRCA2, CDKN2A, CHD2, CIC, CMTR2, CREBBP, CUL3, DICER1, DLC1, DNMT1, DUSP4, EP300, FAT1, FBXW7, JAK1, KDM5C, KDM6A, KEAP1, KMT2C, KMT2D, KRAS, LRP1B, MET, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TREX1, TP53, TSC1, TSC2, USP15, and ZFHX3 in a biological sample from a human subject afflicted with cancer; (b) contacting the biological sample with one or more binding agents, each binding agent specific for one of the biomarker genes, and (c) further processing the sample to determine a genotype of each of the one or more biomarker genes in the biological sample.
2. The method of 1, further comprising (c) classifying the subject as sensitive or resistant to a therapy comprising a human MAP kinase kinase (MEK) inhibitor based on the genotype of each of the one or more biomarker genes in the biological sample obtained from said subject.
3. The method of 1, wherein the biomarker genes are selected from APC, ARID2, ATM, ATRX, BRCA2, CDKN2A, CMTR2, FBXW7, KDM6A, KEAP1, KMT2D, KRAS, MGA, MSH2, NF1, NF2, PALB2, PCNA, PTEN, PTPN11, PTPRD, RB1, RBM10, RNF43, SETD2, SMAD4, STAG2, STK11, TP53, and TSC1.
4. The method of 1, wherein the biomarker genes are selected from APC, ARID2, ATM, ATRX, BRCA2, CDKN2A, CMTR2, KEAP1, KMT2D, MGA, NF1, PTEN, PTPRD, RB1, RBM10, RNF43, SETD2, SMAD4, STAG2, STK11, TP53, and TSC1.
5. The method of 1, wherein the biomarker genes are selected from APC, ARID1A, ARID2, ASXL1, ATM, ATRX, BAP1, BRCA1, BRCA2, CDKN2A, CIC, CMTR2, CUL3, DICER1, DLC1, DUSP4, EP300, FAT1, FBXW7, KDM5C, KDM6A, KEAP1, KMT2C, KMT2D, KRAS, LRP1B, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TP53, TSC1, TSC2, USP15, and ZFHX3.
6. The method of 3, wherein the biomarker genes are selected from CDKN2A, EP300, RBM10, and SETD2.
7. The method of 3, wherein the biomarker genes are selected from CDKN2A, EP300, KRAS, MGA, RB1, RBM10, SETD2, STAG2, STK11, TP53, USP15, and ZFHX3.
8. The method of 3, wherein the biomarker genes are selected from ARID2, BAP1, BRCA1, CIC, KDM6A, KMT2C, KMT2D, LRP1B, NCOA6, NF2, PALB2, PTEN, RASA1, SMAD2, SMG1, SMARCA4, and TET2.
9. The method of 3, wherein the biomarker genes are selected from ARID2, BAP1, BRCA1, CIC, KMT2D, NCOA6, and RASA1.
10. The method of 3, wherein the biomarker genes are selected from ARID2, ATM, CDKN2A, CMTR2, KRAS, MGA, MSH2, RB1, RBM10, RNF43, SETD2, SMAD4, STAG2, and STK11.
11. The method of 3, wherein the biomarker genes are selected from KMT2D and PTEN.
12. The method of 5, wherein the biomarker genes are selected from ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, and SMG1.
13. The method of 5, wherein the genes are selected from CDKN2A, EP300, KEAP1, KRAS, RBM10, SETD2, STK11, TSC1, and USP15.
14. The method of 1, wherein said biological sample has previously been determined to comprise a mutation in at least one gene.
15. The method of 1, wherein the at least one gene is an oncogene.
16. The method of 1, further comprising an initial step of obtaining a biological sample from the subject.
17. The method of 1, wherein said biological sample is a tumor sample.
18. The method of 1, wherein the genotype comprises a mutation in the one or more biomarker genes.
19. The method of 18, wherein the mutation inactivates the biomarker gene.
20. The method of 1, further comprising comparing the genotype with a reference genotype.
21. The method of 20, wherein the genotype is reported as a score.
22. The method of 1, wherein determining the genotype comprises genomic profiling.
23. The method of 1, wherein determining the genotype comprises measuring gene expression.
24. The method of 23, wherein measuring gene expression comprises detection of ribonucleic acids (RNAs) or polypeptides.
25. The method of 2, wherein the subject is classified as sensitive to the MEK inhibitor treatment.
26. The method of 2, wherein the subject is classified as resistant to the MEK inhibitor treatment.
27. The method of 1, wherein the cancer is selected from lung cancer, pancreatic cancer, and colorectal cancer.
28. The method of 27, wherein the cancer is lung cancer.
29. The method of 28, wherein the lung cancer is non-small cell lung cancer (NSCLC).
30. The method of 29, wherein the NSCLC is lung adenocarcinoma.
31. The method of 2, wherein the MEK inhibitor inhibits human MAP kinase kinase 1 (MEK1), MEK2, or MEK1/2.
32. The method of 31, wherein the MEK inhibitor comprises a small molecule.
33. The method of 32, wherein the MEK inhibitor is selected from Trametinib, Selumetinib, Pimasertib, and WX-554.
34. The method of 2, wherein the therapy further comprises an inhibitor of mammalian target of rapamycin (mTOR) kinase pathway.
35. The method of 34, wherein the mTOR pathway inhibitor is an inhibitor of mammalian target of rapamycin complex 1 (TORC1), TORC2, or TORC1/2.
36. The method of 35, wherein the TORC inhibitor is Sapanisertib or Vistusertib.
37. The method of 2, wherein the therapy further comprises a taxane.
38. The method of 37, wherein the taxane is selected from docetaxel, paclitaxel and cabazitaxel.
39. The method of 38, wherein the taxane is docetaxel.
40. The method of 1, wherein the binding agents can facilitate the genotype determination of the one or more biomarker genes.
41. The method of 40, wherein the binding agents comprise reagents capable of determining the genotype by detecting a nucleic acid encoding the biomarker gene or fragments thereof.
42. The method of 41, wherein the binding agents comprise sequencing reagents.
43. The method of 42, wherein the sequencing reagents comprise a probe or primer for sequencing the biomarker gene or a portion thereof.
44. The method of 1, wherein the binding agents comprise a reagent capable of determining the genotype by detecting a polypeptide.
45. The method of 44, wherein the binding agents comprise an antibody or an antigen-binding fragment thereof.
46. The method of 45, wherein the binding agents comprise a label.
47. The method of 2, further comprising administering to said subject a MEK inhibitor therapy.
48. The method of 1, further comprising administering to said subject a taxane therapy.
49. The method of 2, further comprising administering to said subject a combination therapy comprising a MEK inhibitor and an TORC inhibitor.
50. A method of predicting resistance of tumor growth to inhibition by a therapy comprising a MEK inhibitor, said method comprising: (a) detecting in a tumor sample from a human subject afflicted with cancer a genotype of one or more biomarker genes; (b) analyzing the genotype of the one or more biomarker genes in the tumor sample; and (c) predicting resistance of tumor cell growth to inhibition by the therapy comprising a MEK inhibitor, if the tumor sample comprises (i) an inactivating ARID2, BAP1, BRCA1, CIC, KMT2D, NCOA6, or RASA1 mutation, (ii) a decreased copy number of ARID2, BAP1, BRCA1, CIC, KMT2D, NCOA6, or RASA1, or (iii) a decreased expression of ARID2, BAP1, BRCA1, CIC, KMT2D, NCOA6, or RASA1 mRNA or protein.
51. A method of predicting resistance of tumor growth to inhibition by a therapy comprising a MEK inhibitor, said method comprising: (a) detecting in a tumor sample from a human subject afflicted with cancer a genotype of one or more biomarker genes; (b) analyzing the genotype of the one or more biomarker genes in the tumor sample; and (c) predicting resistance of tumor cell growth to inhibition by the therapy comprising a MEK inhibitor, if the tumor sample comprises (i) an inactivating ARID2, BAP1, BRCA1, CIC, KDM6A, KMT2C, KMT2D, LRP1B, NCOA6, NF2, PALB2, PTEN, RASA1, SMAD2, SMG1, SMARCA4, or TET2 mutation, (ii) a decreased copy number of ARID2, BAP1, BRCA1, CIC, KDM6A, KMT2C, KMT2D, LRP1B, NCOA6, NF2, PALB2, PTEN, RASA1, SMAD2.

SMG1, SMARCA4, or TET2, or (iii) a decreased expression of ARID2, BAP1, BRCA1, CIC, KDM6A, KMT2C, KMT2D, LRP1B, NCOA6, NF2, PALB2, PTEN, RASA1, SMAD2, SMG1, SMARCA4, or TET2 mRNA or protein.
52. A method of predicting sensitivity of tumor growth to inhibition by a therapy comprising a MEK inhibitor, said method comprising: (a) detecting in a tumor sample from a human subject afflicted with cancer a genotype of one or more biomarker genes; (b) analyzing the genotype of the one or more biomarker genes in the tumor sample; and (c) predicting sensitivity of tumor cell growth to inhibition by the therapy comprising a MEK inhibitor, if the tumor sample comprises (i) an inactivating CDKN2A, EP300, RBM10, or SETD2 mutation, (ii) a decreased copy number of CDKN2A, EP300, RBM10, or SETD2, or (iii) decreased expression of CDKN2A, EP300, RBM10, or SETD2 mRNA or protein.
53. A method of predicting sensitivity of tumor growth to inhibition by a therapy comprising a MEK inhibitor, said method comprising: (a) detecting in a tumor sample from a human subject afflicted with cancer a genotype of one or more biomarker genes; (b) analyzing the genotype of the one or more biomarker genes in the tumor sample; and (c) predicting sensitivity of tumor cell growth to inhibition by the therapy comprising a MEK inhibitor, if the tumor sample comprises (i) an inactivating CDKN2A, EP300, KRAS, MGA, RB1, RBM10, SETD2, STAG2, STK11, TP53, USP15, or ZFHX3 mutation, (ii) a decreased copy number of CDKN2A, EP300, KRAS, MGA, RB1, RBM10, SETD2, STAG2, STK11, TP53, USP15, or ZFHX3, or (iii) decreased expression of CDKN2A, EP300, KRAS, MGA, RB1, RBM10, SETD2, STAG2, STK11, TP53, USP15, or ZFHX3 mRNA or protein.
54. A method of predicting response of tumor growth to inhibition by a therapy comprising a MEK inhibitor, said method comprising: (a) determining in a tumor sample from a human subject afflicted with cancer a genotype of one or more biomarker genes; (b) analyzing the genotype of the one or more biomarker genes in the tumor sample; and (c) predicting the response of tumor cell growth to inhibition by the therapy comprising a MEK inhibitor, if the tumor sample comprises (i) an inactivating CDKN2A, EP300, KRAS, MGA, RB1, RBM10, SETD2, STAG2, STK11, TP53, USP15, or ZFHX3 mutation, (ii) a decreased copy number of CDKN2A, EP300, KRAS, MGA, RB1, RBM10, SETD2, STAG2, STK11, TP53, USP15, or ZFHX3, (iii) decreased expression of CDKN2A, EP300, KRAS, MGA, RB1, RBM10, SETD2, STAG2, STK11, TP53, USP15, or ZFHX3 mRNA or protein; (iv) an inactivating ARID2, BAP1, BRCA1, CIC, KDM6A, KMT2C, KMT2D, LRP1B, NCOA6, NF2, PALB2, PTEN, RASA1, SMAD2, SMG1, SMARCA4, or TET2 mutation, (v) a decreased copy number of ARID2, BAP1, BRCA1, CIC, KDM6A, KMT2C, KMT2D, LRP1B, NCOA6, NF2, PALB2, PTEN, RASA1, SMAD2, SMG1, SMARCA4, or TET2, or (vi) a decreased expression of ARID2, BAP1, BRCA1, CIC, KDM6A, KMT2C, KMT2D, LRP1B, NCOA6, NF2, PALB2, PTEN, RASA1, SMAD2, SMG1, SMARCA4, or TET2 mRNA or protein.
55. A method of predicting response of tumor growth to inhibition by a therapy comprising a MEK inhibitor, said method comprising: (a) determining in a tumor sample from a human subject afflicted with cancer a genotype of one or more biomarker genes; (b) analyzing the genotype of the one or more biomarker genes in the tumor sample; and (c) predicting the response of tumor cell growth to inhibition by the therapy comprising a MEK inhibitor, if the tumor sample comprises (i) an inactivating CDKN2A, EP300, RBM10, or SETD2 mutation, (ii) a decreased copy number of CDKN2A, EP300, RBM10, or SETD2, (iii) decreased expression of CDKN2A, EP300, RBM10, or SETD2 mRNA or protein; (iv) an inactivating ARID2, BAP1, BRCA1, CIC, KMT2D, NCOA6, or RASA1 mutation, (v) a decreased copy number of ARID2, BAP1, BRCA1, CIC, KMT2D, NCOA6, or RASA1, or (vi) a decreased expression of ARID2, BAP1, BRCA1, CIC, KMT2D, NCOA6, or RASA1 mRNA or protein.
56. The method of 50 to 55, wherein said biological sample has previously been determined to comprise a mutation in at least one gene.
57. The method of 56, wherein the at least one gene is an oncogene.
58. The method of any one of 50 to 55, further comprising obtaining a tumor sample from the subject.
59. The method of any one of 50 to 55, wherein the genotype comprises a mutation in the one or more biomarker genes.
60. The method of 59, wherein the mutation inactivates the biomarker gene.
61. The method of any one of 50 to 55, further comprising comparing the genotype with a reference genotype.
62. The method of 61, wherein the genotype is reported as a score.
63. The method of any one of 50 to 55, wherein determining the genotype comprises genomic profiling.
64. The method of any one of 50 to 55, wherein determining the genotype comprises measuring gene expression.
65. The method of 64, wherein measuring gene expression comprises detection of ribonucleic acids (RNAs) or polypeptides.
66. The method of 55, wherein the subject is classified as sensitive to the MEK inhibitor therapy.
67. The method of 55, wherein the subject is classified as resistant to the MEK inhibitor therapy.
68. The method of any one of 50 to 55, wherein the cancer is selected from lung cancer, pancreatic cancer, and colorectal cancer.
69. The method of 68, wherein the cancer is lung cancer.
70. The method of 69, wherein the lung cancer is non-small cell lung cancer (NSCLC).
71. The method of 70, wherein the NSCLC is lung adenocarcinoma.
72. The method of any one of 50 to 55, wherein the MEK inhibitor inhibits human MAP kinase kinase 1 (MEK1), MEK2, or MEK1/2.
73. The method of 72, wherein the MEK inhibitor comprises a small molecule.
74. The method of 73, wherein the MEK inhibitor is selected from Trametinib, Selumetinib, Pimasertib, and WX-554.
75. The method of any one of 50 to 55, wherein the therapy further comprises a taxane.
76. The method of 75, wherein the taxane is selected from docetaxel, paclitaxel and cabazitaxel.
77. The method of 76, wherein the taxane is docetaxel.
78. The method of any one of 50 to 55, wherein the genotype determination comprises one or more binding agents.
79. The method of 78, wherein the binding agents comprise reagents capable of determining the genotype by detecting a nucleic acid encoding the biomarker gene or fragments thereof.
80. The method of 79, wherein the binding agents can facilitate the genotype determination of the one or more biomarker genes.
81. The method of 80, wherein the binding agents comprise sequencing reagents.
82. The method of 81, wherein the sequencing reagents comprise a probe or primer for sequencing the biomarker gene or a portion thereof.
83. The method of any one of 50 to 55, wherein the binding agents comprise a reagent capable of determining the genotype by detecting a polypeptide.
84. The method of 83, wherein the binding agents comprise an antibody or an antigen-binding fragment thereof.
85. The method of 83, wherein the binding agents comprise a label.
86. The method of any one of 50 to 55, further comprising administering to said subject a MEK inhibitor therapy if the predicted response of the subject is sensitivity of tumor growth to inhibition by a therapy comprising a MEK inhibitor.
87. The method of any one of 50 to 55, further comprising administering to said subject a taxane therapy.
88. The method of any one of 50 to 55, further comprising administering to said subject a combination therapy comprising a MEK inhibitor and a TORC inhibitor.
89. A method of determining effectiveness of a MEK inhibitor in reducing tumor size comprising: (a) treating a first inert tumor with a control therapy; (b) treating a second inert tumor with a MEK inhibitor, wherein the first and second inert tumor comprise identical genotypes; (c) treating a first mutant tumor with the control therapy; (d) treating a second mutant tumor with the MEK inhibitor, wherein the first and second mutant tumor comprise identical genotypes; (e) comparing sizes of the first and second inert tumors after the therapy; (f) comparing sizes of the first and second mutant tumors after completion of the therapy, and (g) identifying the mutant tumor genotype as sensitive to the MEK inhibitor if the change in tumor size between the first and second inert tumors after the therapy is less than the change in tumor size between the first and second mutant tumors after the therapy.
90. A method of determining effectiveness of a MEK inhibitor in reducing tumor size comprising: (a) treating a first inert tumor with a control therapy; (b) treating a second inert tumor with a MEK inhibitor, wherein the first and second inert tumor comprise identical genotypes; (c) treating a first mutant tumor with the control therapy; (d) treating a second mutant tumor with the MEK inhibitor, wherein the first and second mutant tumor comprise identical genotypes; (e) comparing sizes of the first and second inert tumors after the therapy; (f) comparing sizes of the first and second mutant tumors after completion of the therapy, and (g) identifying the mutant tumor genotype as resistant to the MEK inhibitor if the change in tumor size between the first and second inert tumors after the therapy is greater than the change in tumor size between the first and second mutant tumors after the therapy.
91. A method of predicting resistance of tumor growth to inhibition by a combination therapy comprising a MEK inhibitor and a TORC inhibitor, said method comprising: (a) detecting in a tumor sample from a human subject afflicted with cancer a genotype of one or more biomarker genes; (b) analyzing the genotype of the one or more biomarker genes in the tumor sample; and (c) predicting resistance of tumor cell growth to inhibition by the combination therapy comprising a MEK inhibitor and a TORC inhibitor, if the tumor sample comprises (i) an inactivating KMT2D or PTEN mutation, (ii) a decreased copy number of KMT2D or PTEN, or (iii) a decreased expression of KMT2D or PTEN mRNA or protein.
92. A method of predicting sensitivity of tumor growth to inhibition by a combination therapy comprising a MEK inhibitor and a TORC inhibitor, said method comprising: (a) detecting in a tumor sample from a human subject afflicted with cancer a genotype of one or more biomarker genes; (b) analyzing the genotype of the one or more biomarker genes in the tumor sample; and (c) predicting sensitivity of tumor cell growth to inhibition by the combination therapy comprising a MEK inhibitor and a TORC inhibitor, if the tumor sample comprises (i) an inactivating ARID2, ATM, CDKN2A, CMTR2, KRAS, MGA, MSH2, RB1, RBM10, RNF43, SETD2, SMAD4, STAG2, or STK11 mutation, (ii) a decreased copy number of ARID2, ATM, CDKN2A, CMTR2, KRAS, MGA, MSH2, RB1, RBM10, RNF43, SETD2, SMAD4, STAG2, or STK11, or (iii) decreased expression of ARID2, ATM, CDKN2A, CMTR2, KRAS, MGA, MSH2, RB1, RBM10, RNF43, SETD2, SMAD4, STAG2, or STK11 mRNA or protein.
93. A method of predicting response of tumor growth to inhibition by a combination therapy comprising a MEK inhibitor and a TORC inhibitor, said method comprising: (a) determining in a tumor sample from a human subject afflicted with cancer a genotype of one or more biomarker genes; (b) analyzing the genotype of the one or more biomarker genes in the tumor sample; and (c) predicting the response of tumor cell growth to inhibition by the combination therapy comprising a MEK inhibitor and a TORC inhibitor, if the tumor sample comprises (i) an inactivating ARID2, ATM, CDKN2A, CMTR2, KRAS, MGA, MSH2, RB1, RBM10, RNF43, SETD2, SMAD4, STAG2, or STK11 mutation, (ii) a decreased copy number of ARID2, ATM, CDKN2A, CMTR2, KRAS, MGA, MSH2, RB1, RBM10, RNF43, SETD2, SMAD4, STAG2, or STK11, (iii) decreased expression of ARID2, ATM, CDKN2A, CMTR2, KRAS, MGA, MSH2, RB1, RBM10, RNF43, SETD2, SMAD4, STAG2, or STK11 mRNA or protein; (iv) an inactivating KMT2D or PTEN mutation, (v) a decreased copy number of KMT2D or PTEN, or (vi) a decreased expression of KMT2D or PTEN mRNA or protein.
94. The method any one of 91 to 93, wherein said biological sample has previously been determined to comprise a mutation in at least one gene.
95. The method of 94, wherein the at least one gene is an oncogene.
96. The method of any one of 91 to 93, further comprising obtaining a tumor sample from the subject matter.
97. The method of any one of 91-93, wherein the genotype comprises a mutation in the one or more biomarker genes.
98. The method of 97, wherein the mutation inactivates the biomarker gene.
99. The method of 91 to 93, further comprising comparing the genotype with a reference genotype.
100. The method of 99, wherein the genotype is reported as a score.
101. The method of any one of 91 to 93, wherein determining the genotype comprises genomic profiling.
102. The method of any one of 91 to 93, wherein determining the genotype comprises measuring gene expression.
103. The method of 102, wherein measuring gene expression comprises detection of ribonucleic acids (RNAs) or polypeptides.
104. The method of 95, wherein the subject is classified as sensitive to a MEK inhibitor/TORC inhibitor combination therapy.
105. The method of 95, wherein the subject is classified as resistant to a MEK inhibitor/TORC inhibitor combination therapy.
106. The method of any one of 91 to 93, wherein the cancer is selected from lung cancer, pancreatic cancer, and colorectal cancer.
107. The method of 106, wherein the cancer is lung cancer.
108. The method of 107, wherein the lung cancer is non-small cell lung cancer (NSCLC).
109. The method of 108, wherein the NSCLC is lung adenocarcinoma.
110. The method of any one of 91 to 93, wherein the MEK inhibitor inhibits human MAP kinase kinase 1 (MEK1), MEK2, or MEK1/2.
111. The method of 110, wherein the MEK inhibitor comprises a small molecule.
112. The method of 110, wherein the MEK inhibitor is selected from Trametinib, Selumetinib, Pimasertib, and WX-554.
113. The method of any one of 91 to 93, wherein the TORC inhibitor inhibits target of rapamycin complex 1 (TORC1), or TORC1/2.
114. The method of 113, wherein the TORC inhibitor comprises a small molecule.
115. The method of 114, wherein the TORC inhibitor is selected from Sapanisertib and Vistusertib.
116. The method of any one of 91 to 93, wherein the therapy further comprises a taxane.
117. The method of 116, wherein the taxane is selected from docetaxel, paclitaxel and cabazitaxel.
118. The method of 117, wherein the taxane is docetaxel.
119. The method of any one of 91 to 93, wherein the genotype determination comprises one or more binding agents.
120. The method of 119, wherein the binding agents comprise reagents capable of determining the genotype by detecting a nucleic acid encoding the biomarker gene or fragments thereof.
121. The method of 120, wherein the binding agents can facilitate the genotype determination of the one or more biomarker genes.
122. The method of 121, wherein the binding agents comprise sequencing reagents.
123. The method of 122, wherein the sequencing reagents comprise a probe or primer for sequencing the biomarker gene or a portion thereof.
124. The method of any one of 91 to 93, wherein the binding agents comprise a reagent capable of determining the genotype by detecting a polypeptide.
125. The method of 124, wherein the binding agents comprise an antibody or an antigen-binding fragment thereof.
126. The method of 119, wherein the binding agents comprise a label.
127. The method of any one of 91 to 93, further comprising administering a taxane therapy.
128. A method of determining effectiveness of a MEK inhibitor/TORC inhibitor combination in reducing tumor size comprising: (a) treating a first inert tumor with a control therapy; (b) treating a second inert tumor with a MEK inhibitor and a TORC inhibitor, wherein the first and second inert tumor comprise identical genotypes; (c) treating a first mutant tumor with the control therapy; (d) treating a second mutant tumor with the MEK inhibitor and the TORC inhibitor, wherein the first and second mutant tumor comprise identical genotypes; (e) comparing sizes of the first and second inert tumors after the therapy; (f) comparing sizes of the first and second mutant tumors after completion of the therapy, and (g) identifying the mutant tumor genotype as sensitive to the MEK inhibitor/TORC inhibitor combination if the change in tumor size between the first and second inert tumors after the therapy is less than the change in tumor size between the first and second mutant tumors after the therapy.
129. A method of determining effectiveness of a MEK inhibitor/TORC inhibitor combination in reducing tumor size comprising: (a) treating a first inert tumor with a control therapy; (b) treating a second inert tumor with a MEK inhibitor and the TORC inhibitor, wherein the first and second inert tumor comprise identical genotypes; (c) treating a first mutant tumor with the control therapy; (d) treating a second mutant tumor with the MEK inhibitor and the TORC inhibitor, wherein the first and second mutant tumor comprise identical genotypes; (e) comparing sizes of the first and second inert tumors after the therapy; (f) comparing sizes of the first and second mutant tumors after completion of the therapy, and (g) identifying the mutant tumor genotype as resistant to the MEK inhibitor/TORC inhibitor combination if the change in tumor size between the first and second inert tumors after the therapy is greater than the change in tumor size between the first and second mutant tumors after the therapy.
130. A composition comprising one or more isolated biomarker genes selected from the group comprising ADAR, APC, ARID1A, ARID2, ASXL1, ATM, ATRX, BAP1, BRCA1, BRCA2, CDKN2A, CHD2, CIC, CMTR2, CREBBP, CUL3, DICER1, DLC1, DNMT1, DUSP4, EP300, FAT1, FBXW7, JAK1, KDM5C, KDM6A, KEAP1, KMT2C, KMT2D, KRAS, LRP1B, MET, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TREX1, TP53, TSC1, TSC2, USP15, and ZFHX3.
131. The composition of 130 comprising two, three, four, five, six, seven, eight, nine, ten or more of the isolated biomarker genes.
132. A composition comprising one or more isolated biomarker genes selected from APC, ARID1A, ARID2, ASXL1, ATM, ATRX, BAP1, BRCA1, BRCA2, CDKN2A, CIC, CMTR2, CUL3, DICER1, DLC1, DUSP4, EP300, FAT1, FBXW7, KDM5C, KDM6A, KEAP1, KMT2C, KMT2D, KRAS, LRP1B, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TP53, TSC1, TSC2, USP15, and ZFHX3.
133. The composition of 132, comprising two, three, four, five, six, seven, eight, nine, ten or more of the isolated biomarker genes.
134. A composition comprising one or more isolated biomarker genes selected from APC, ARID2, ATM, ATRX, BRCA2, CDKN2A, CMTR2, FBXW7, KDM6A, KEAP1, KMT2D, KRAS, MGA, MSH2, NF1, NF2, PALB2, PCNA, PTEN, PTPN11, PTPRD, RB1, RBM10, RNF43, SETD2, SMAD4, STAG2, STK11, TP53, and TSC1.
135. The composition of 134, comprising two, three, four, five, six, seven, eight, nine, ten or more of the isolated biomarker genes.
136. A composition comprising one or more isolated biomarker genes selected from APC, ARID2, ATM, ATRX, BRCA2, CDKN2A, CMTR2, KEAP1, KMT2D, MGA, NF1, PTEN, PTPRD, RB1, RBM10, RNF43, SETD2, SMAD4, STAG2, STK11, TP53, and TSC1.
137. The composition of 136, comprising two, three, four, five, six, seven, eight, nine, ten or more of the isolated biomarker genes.
138. A composition comprising one or more isolated biomarker genes selected from CDKN2A, EP300, KRAS, MGA, RB1, RBM10, SETD2, STAG2, STK11, TP53, USP15, and ZFHX3.
139. The composition of 138, comprising two, three, four, five, six, seven, eight, nine, ten or more of the isolated biomarker genes.
140. A composition comprising one or more isolated biomarker genes selected from CDKN2A, EP300, RBM10, and SETD2
141. The composition of 140, comprising two, three, or four isolated biomarker genes.
142. A composition comprising one or more isolated biomarker genes selected from ARID2, ATM, CDKN2A, CMTR2, KRAS, MGA, MSH2, RB1, RBM10, RNF43, SETD2, SMAD4, STAG2, and STK11.
143. The composition of 142, comprising two, three, four, five, or six isolated biomarker genes.
144. A composition comprising one or more isolated biomarker genes selected from ARID2, BAP1, BRCA1, CIC, KMT2D, NCOA6, and RASA1.
145. The composition of 144, comprising two, three, four, five, six, or seven, isolated biomarker genes.
146. A composition comprising one or more isolated biomarker genes selected from ARID2, BAP1, BRCA1, CIC, KDM6A, KMT2C, KMT2D, LRP1B, NCOA6, NF2, PALB2, PTEN, RASA1, SMAD2, SMG1, SMARCA4, and TET2.
147. The composition of 146, comprising two, three, four, five, six, seven, eight, nine, ten or more of the isolated biomarker genes.
148. A composition comprising isolated biomarker genes selected from the group comprising KMT2D and PTEN.
149. The composition of 148, comprising KMT2D and PTEN.
150. A composition comprising one or more isolated biomarker genes selected from ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, and SMG1.
151. The composition of 150, comprising two, three, four, five, six, seven, eight, nine, ten or more of the isolated biomarker genes.
152. A composition comprising one or more isolated biomarker genes selected from CDKN2A, EP300, KEAP1, KRAS, RBM10, SETD2, STK11, TSC1, and USP15.
153. The composition of 152, comprising two, three, four, five, six, seven, eight, nine, ten or more of the isolated biomarker genes.
154. The composition of any one of 130 to 153 further comprising a binding agent.
155. The composition of 154, wherein the binding agent is capable of facilitating genotype determination of the biomarker gene.
156. The composition of 154, wherein the binding agent comprises a reagent capable of determining the genotype by detecting a polypeptide.
157. The composition of 155, wherein the binding agent comprises an antibody or an antigen-binding fragment thereof.
158. The composition of 154, wherein the binding agent comprises a reagent capable of determining the genotype by detecting a nucleic acid encoding the biomarker gene or fragments thereof.
159. The composition of 158, wherein the binding agent comprises a sequencing reagent.
160. The composition of 159, wherein the sequencing agent comprises a probe or primer for sequencing the biomarker gene or portion thereof.
161. The composition of 154, wherein the binding agent comprises a label.
162. A method of detecting one or more isolated biomarker genes selected from ADAR, APC, ARID1A, ARID2, ASXL1, ATM, ATRX, BAP1, BRCA1, BRCA2, CDKN2A, CHD2, CIC, CMTR2, CREBBP, CUL3, DICER1, DLC1, DNMT1, DUSP4, EP300, FAT1, FBXW7, JAK1, KDM5C, KDM6A, KEAP1, KMT2C, KMT2D, KRAS, LRP1B, MET, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TREX1, TP53, TSC1, TSC2, USP15, and ZFHX3 in a human subject, said method comprising: detecting whether the one or more isolated biomarker genes are present in a biological sample of the human subject by contacting the biological sample with a binding agent and detecting binding between the one or more isolated biomarker genes and the binding agent.
163. A method of detecting one or more isolated biomarker genes selected from APC, ARID2, ATM, ATRX, BRCA2, CDKN2A, CMTR2, FBXW7, KDM6A, KEAP1, KMT2D, KRAS, MGA, MSH2, NF1, NF2, PALB2, PCNA, PTEN, PTPN11, PTPRD, RB1, RBM10, RNF43, SETD2, SMAD4, STAG2, STK11, TP53, and TSC1 in a human subject, said method comprising: detecting whether the one or more isolated biomarker genes are present in a biological sample of the human subject by contacting the biological sample with a binding agent and detecting binding between the one or more isolated biomarker genes and the binding agent.
164. A method of detecting one or more isolated biomarker genes selected from APC, ARID2, ATM, ATRX, BRCA2, CDKN2A, CMTR2, KEAP1, KMT2D, MGA, NF1, PTEN, PTPRD, RB1, RBM10, RNF43, SETD2, SMAD4, STAG2, STK11, TP53, and TSC1 in a human subject, said method comprising: detecting whether the one or more isolated biomarker genes are present in a biological sample of the human subject by contacting the biological sample with a binding agent and detecting binding between the one or more isolated biomarker genes and the binding agent.
165. A method of detecting one or more isolated biomarker genes selected from CDKN2A, EP300, KRAS, MGA, RB1, RBM10, SETD2, STAG2, STK11, TP53, USP15, and ZFHX3 in a human subject, said method comprising: detecting whether the one or more isolated biomarker genes are present in a biological sample of the human subject by contacting the biological sample with a binding agent and detecting binding between the one or more isolated biomarker genes and the binding agent.
166. A method of detecting one or more isolated biomarker genes selected from CDKN2A, EP300, RBM10, and SETD2 in a human subject, said method comprising: detecting whether the one or more isolated biomarker genes are present in a biological sample of the human subject by contacting the biological sample with a binding agent and detecting binding between the one or more isolated biomarker genes and the binding agent.
167. A method of detecting one or more isolated biomarker genes selected from ARID2, ATM, CDKN2A, CMTR2, KRAS, MGA, MSH2, RB1, RBM10, RNF43, SETD2, SMAD4, STAG2, and STK11 in a human subject, said method comprising: detecting whether the one or more isolated biomarker genes are present in a biological sample of the human subject by contacting the biological sample with a binding agent and detecting binding between the one or more isolated biomarker genes and the binding agent.
168. A method of detecting one or more isolated biomarker genes selected from ARID2, BAP1, BRCA1, CIC, KMT2D, NCOA6, and RASA1 in a human subject, said method comprising: detecting whether the one or more isolated biomarker genes are present in a biological sample of the human subject by contacting the biological sample with a binding agent and detecting binding between the one or more isolated biomarker genes and the binding agent.
169. A method of detecting one or more isolated biomarker genes selected from ARID2, BAP1, BRCA1, CIC, KDM6A, KMT2C, KMT2D, LRP1B, NCOA6, NF2, PALB2, PTEN, RASA1, SMAD2, SMG1, SMARCA4, and TET2 in a human subject, said method comprising: detecting whether the one or more isolated biomarker genes are present in a biological sample of the human subject by contacting the biological sample with a binding agent and detecting binding between the one or more isolated biomarker genes and the binding agent.
170. A method of detecting one or more isolated biomarker genes selected from KMT2D and PTEN in a human subject, said method comprising: detecting whether the one or more isolated biomarker genes are present in a biological sample of the human subject by contacting the biological sample with a binding agent and detecting binding between the one or more isolated biomarker genes and the binding agent.
171. A method of detecting one or more isolated biomarker genes selected from APC, ARID1A, ARID2, ASXL1, ATM, ATRX, BAP1, BRCA1, BRCA2, CDKN2A, CIC, CMTR2, CUL3, DICER1, DLC1, DUSP4, EP300, FAT1, FBXW7, KDM5C, KDM6A, KEAP1, KMT2C, KMT2D, KRAS, LRP1B, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TP53, TSC1, TSC2, USP15, and ZFHX3 in a human subject, said method comprising: detecting whether the one or more isolated biomarker genes are present in a biological sample of the human subject by contacting the biological sample with a binding agent and detecting binding between the one or more isolated biomarker genes and the binding agent.
172. A method of detecting one or more isolated biomarker genes selected from ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, and SMG1 in a human subject, said method comprising: detecting whether the one or more isolated biomarker genes are present in a biological sample of the human subject by contacting the biological sample with a binding agent and detecting binding between the one or more isolated biomarker genes and the binding agent.
173. A method of detecting one or more isolated biomarker genes selected from CDKN2A, EP300, KEAP1, KRAS, RBM10, SETD2, STK11, TSC1, and USP15 in a human subject, said method comprising: detecting whether the one or more isolated biomarker genes are present in a biological sample of the human subject by contacting the biological sample with a binding agent and detecting binding between the one or more isolated biomarker genes and the binding agent.
174. The method of any one of 162 to 173, wherein the binding agent is capable of facilitating genotype determination of the biomarker gene.
175. The method of 164, wherein the binding agent comprises a reagent capable of determining the genotype by detecting a polypeptide.
176. The method of 175, wherein the binding agent comprises an antibody or an antigen-binding fragment thereof.
177. The method of 174, wherein the binding agent comprises a reagent capable of determining the genotype by detecting a nucleic acid encoding the biomarker gene or fragments thereof.
178. The method of 177, wherein the binding agent comprises a sequencing reagent.
179. The method of 178, wherein the sequencing agent comprises a probe or primer for sequencing the biomarker gene or portion thereof.
180. The method of 174, wherein the binding agent comprises a label.
181. A method of predicting resistance of tumor growth to inhibition by a combination therapy comprising a MEK inhibitor and a chemotherapy, comprising: (a) detecting in a tumor sample from a human subject afflicted with cancer a genotype of one or more biomarker genes; (b) analyzing the genotype of the one or more biomarker genes in the tumor sample; and (c) predicting resistance of tumor cell growth to inhibition by the combination therapy comprising a MEK inhibitor and a chemotherapy, if the tumor sample comprises (i) an inactivating ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, or SMG1 mutation, (ii) a decreased copy number of ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, or SMG1, or (iii) a decreased expression of ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, or SMG1 mRNA or protein.
182. A method of predicting sensitivity of tumor growth to inhibition by a combination therapy comprising a MEK inhibitor and a chemotherapy, comprising: (a) detecting in a tumor sample from a human subject afflicted with cancer a genotype of one or more biomarker genes; (b) analyzing the genotype of the one or more biomarker genes in the tumor sample; and (c) predicting sensitivity of tumor cell growth to inhibition by the combination therapy comprising a MEK inhibitor and a chemotherapy, if the tumor sample comprises (i) an inactivating APC, ARID1A, ATRX, CDKN2A, CMTR2, DUSP4, EP300, FBXW7, KEAP1, KRAS, MSH2, NF1, PTPRD, RBM10, SETD2, STAG2, STK11, TET2, TSC1, USP15, or ZFHX3 mutation, (ii) a decreased copy number of APC, ARID1A, ATRX, CDKN2A, CMTR2, DUSP4, EP300, FBXW7, KEAP1, KRAS, MSH2, NF1, PTPRD, RBM10, SETD2, STAG2, STK11, TET2, TSC1, USP15, or ZFHX3, or (iii) decreased expression of APC, ARID1A, ATRX CDKN2A, CMTR2, DUSP4, EP300, FBXW7, KEAP1, KRAS, MSH2, NF1, PTPRD, RBM10, SETD2, STAG2, STK11, TET2, TSC1, USP15, or ZFHX3 mRNA or protein.
183. A method of predicting response of tumor growth to inhibition by a combination therapy comprising a MEK inhibitor and a chemotherapy, comprising: (a) determining in a tumor sample from a human subject afflicted with cancer a genotype of one or more biomarker genes; (b) analyzing the genotype of the one or more biomarker genes in the tumor sample; and (c) predicting the response of tumor cell growth to inhibition by the combination therapy comprising a MEK inhibitor and a chemotherapy, if the tumor sample comprises (i) an inactivating ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, or SMG1 mutation, (ii) a decreased copy number of ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, or SMG1, (iii) decreased expression of ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, or SMG1 mRNA or protein; (iv) an inactivating CDKN2A, EP300, KEAP1, KRAS, RBM10, SETD2, STK11, TSC1, or USP15 mutation, (v) a decreased copy number of CDKN2A, EP300, KEAP1, KRAS, RBM10, SETD2, STK11, TSC1, or USP1S, or (vi) a decreased expression of CDKN2A, EP300, KEAP1, KRAS, RBM10, SETD2, STK1, TSC1, or USP15 mRNA or protein.
184. The method of any one of 181 to 183, wherein said biological sample has previously been determined to comprise a mutation in at least one gene.
185. The method of 184, wherein the at least one gene is an oncogene.
186. The method of any one of 181 to 183, further comprising obtaining a tumor sample from the subject.
187. The method of any one of 181 to 183, wherein the genotype comprises a mutation in the one or more biomarker genes.
188. The method of 187, wherein the mutation inactivates the biomarker gene.
189. The method of any one of 181 to 183, further comprising comparing the genotype with a reference genotype.
190. The method of 189, wherein the genotype is reported as a score.
191. The method of any one of 181 to 183, wherein determining the genotype comprises genomic profiling.
192. The method of any one of 181 to 183, wherein determining the genotype comprises measuring gene expression.
193. The method of 192, wherein measuring gene expression comprises detection of ribonucleic acids (RNAs) or polypeptides.
194. The method of 183, wherein the subject is classified as sensitive to a MEK inhibitor/chemotherapy combination therapy.
195. The method of 183, wherein the subject is classified as resistant to a MEK inhibitor/chemotherapy therapy.
196. The method of any one of 181 to 183, wherein the cancer is selected from lung cancer, pancreatic cancer, and colorectal cancer.
197. The method of 196, wherein the cancer is lung cancer.
198. The method of 197, wherein the lung cancer is non-small cell lung cancer (NSCLC).
199. The method of 198, wherein the NSCLC is lung adenocarcinoma.
200. The method of any one of 181 to 183, wherein the MEK inhibitor inhibits human MAP kinase kinase 1 (MEK1), MEK2, or MEK1/2.
201. The method of 200, wherein the MEK inhibitor comprises a small molecule.
202. The method of 201, wherein the MEK inhibitor is selected from Trametinib, Selumetinib, Pimasertib, and WX-554.
203. The method of any one of 181 to 183, further comprising administering to said subject chemotherapy.
204. The method of 203, wherein the chemotherapy comprises a chemotherapeutic agent belonging to the class taxanes.
205. The method of 204, wherein the chemotherapeutic agent is paclitaxel or docetaxel.
206. The method of any one of 181 to 183, wherein the chemotherapy comprises a chemotherapeutic agent belonging to the class platinum-based chemotherapeutic agents.
207. The method of 206, wherein the chemotherapy agent is carboplatin.
208. The method of any one of 181 to 183, wherein the chemotherapy comprises a chemotherapeutic agent belonging to the class folate antimetabolites.
209. The method of 208, wherein the chemotherapeutic agent is pemetrexed.
210. The method of any one of 181 to 183, wherein the genotype determination comprises one or more binding agents.
211. The method of 210, wherein the binding agents comprise reagents capable of determining the genotype by detecting a nucleic acid encoding the biomarker gene or fragments thereof.
212. The method of 211, wherein the binding agents are capable of facilitating the genotype determination of the one or more biomarker genes.
213. The method of 212, wherein the binding agents comprise sequencing reagents.
214. The method of 213, wherein the sequencing reagents comprise a probe or primer for sequencing the biomarker gene or a portion thereof.
215. The method of any one of 181 to 183, wherein the binding agents comprise a reagent capable of determining the genotype by detecting a polypeptide.
216. The method of 215, wherein the binding agents comprise an antibody or an antigen-binding fragment thereof.
217. The method of 210, wherein the binding agents comprise a label.
218. The method of any one of 181 to 183, further comprising administering a taxane therapy.
219. A method of determining effectiveness of a MEK inhibitor/chemotherapy combination in reducing tumor size comprising: (a) treating a first inert tumor with a control therapy; (b) treating a second inert tumor with a MEK inhibitor and a chemotherapy, wherein the first and second inert tumor comprise identical genotypes; (c) treating a first mutant tumor with the control therapy; (d) treating a second mutant tumor with the MEK inhibitor and the chemotherapy, wherein the first and second mutant tumor comprise identical genotypes; (e) comparing sizes of the first and second inert tumors after the therapy; (f) comparing sizes of the first and second mutant tumors after completion of the therapy, and (g) identifying the mutant tumor genotype as sensitive to the MEK inhibitor/chemotherapy combination if the change in tumor size between the first and second inert tumors after the therapy is less than the change in tumor size between the first and second mutant tumors after the therapy.
220. A method of determining effectiveness of a MEK inhibitor/chemotherapy combination in reducing tumor size comprising: (a) treating a first inert tumor with a control therapy; (b) treating a second inert tumor with a MEK inhibitor and the chemotherapy, wherein the first and second inert tumor comprise identical genotypes; (c) treating a first mutant tumor with the control therapy; (d) treating a second mutant tumor with the MEK inhibitor and the chemotherapy, wherein the first and second mutant tumor comprise identical genotypes; (e) comparing sizes of the first and second inert tumors after the therapy; (f) comparing sizes of the first and second mutant tumors after completion of the therapy, and (g) identifying the mutant tumor genotype as resistant to the MEK inhibitor/chemotherapy combination if the change in tumor size between the first and second inert tumors after the therapy is greater than the change in tumor size between the first and second mutant tumors after the therapy
221. A method of treating non-small cell lung cancer (NSCLC) in a subject comprising the step of treating a subject with a MEK inhibitor when a tumor sample obtained from the subject comprises (i) an inactivating mutation in one or more of CDKN2A, EP300, KRAS, MGA, RB1, RBM10, SETD2, STAG2, STK11, TP53, USP15, or ZFHX3, (ii) a decreased copy number of one or more of CDKN2A, EP300, KRAS, MGA, RB1, RBM10, SETD2, STAG2, STK11, TP53, USP15, or ZFHX3, or (iii) decreased expression of mRNA or protein in one or more of CDKN2A, EP300, KRAS, MGA, RB1, RBM10, SETD2, STAG2, STK11, TP53, USP15, or ZFHX3.
222. The method of 221, wherein the inactivating mutation is in one or more of CDKN2A, EP300, RBM10, and SETD2.
223. The method of 221, wherein the tumor sample obtained from the subject further comprises absence of (i) an inactivating mutation in one or more of ARID2, BAP1, BRCA1, CIC, KDM6A, KMT2C, KMT2D, LRP1B, NCOA6, NF2, PALB2, PTEN, RASA1, SMAD2, SMG1, SMARCA4, or TET2, (ii) a decreased copy number of one or more of ARID2. BAP1, BRCA1, CIC, KDM6A, KMT2C, KMT2D, LRP1B, NCOA6, NF2, PALB2, PTEN, RASA1, SMAD2, SMG1, SMARCA4, or TET2, or (iii) decreased expression of CDKN2A, EP300, KRAS, MGA, RB1, RBM10, SETD2, STAG2, STK11, TP53, USP15, or ZFHX3 mRNA or protein.
224. The method of 223, wherein the absence of an inactivating mutation is in one or more of ARID2, BAP1, BRCA1, CIC, KMT2D, NCOA6, or RASA1.
225. A method of treating non-small cell lung cancer (NSCLC) in a subject comprising the step of treating a subject with a MEK inhibitor and a TORC inhibitor when a tumor sample obtained from the subject comprises (i) an inactivating ARID2, ATM, CDKN2A, CMTR2, KRAS, MGA, MSH2, RB1, RBM10, RNF43, SETD2, SMAD4, STAG2, or STK11 mutation, (ii) a decreased copy number of ARID2, ATM, CDKN2A, CMTR2, KRAS, MGA, MSH2, RB1, RBM10, RNF43, SETD2, SMAD4, STAG2, or STK11, or (iii) decreased expression of ARID2, ATM, CDKN2A, CMTR2, KRAS, MGA, MSH2, RB1, RBM10, RNF43, SETD2, SMAD4, STAG2, or STK11 mRNA or protein.
226. The method of 225, wherein the tumor sample obtained from the subject further comprises absence of (i) an inactivating mutation in one or more of KMT2D or PTEN, (ii) a decreased copy number of one or more of KMT2D or PTEN, or (iii) a decreased expression of KMT2D or PTEN mRNA or protein.
227. A method of treating non-small cell lung cancer (NSCLC) in a subject comprising treating a subject with a MEK inhibitor and a chemotherapy when a tumor sample obtained from the subject comprises (i) an inactivating CDKN2A, EP300, KEAP1, KRAS, RBM10, SETD2, STK11, TSC1, or USP15 mutation, (ii) a decreased copy number of CDKN2A, EP300, KEAP1, KRAS, RBM10, SETD2, STK11, TSC1, or USP15, or (iii) decreased expression of CDKN2A, EP300, KEAP1, KRAS, RBM10, SETD2, STK11, TSC1, or USP15 mRNA or protein.
228. The method of 227, wherein the tumor sample obtained from the subject further comprises absence of (i) an inactivating mutation in one or more of ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, or SMG1, (ii) a decreased copy number of one or more of ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, or SMG1, or (iii) a decreased expression of ARID2, ASXL1, A7M, BAP1, BRCA1, CIC, KDM6A, KMT2C, KMT2D, LRP1B, NCOA6, NF2, PALB2, PTPN11, RASA1, RB1CC1, SMAD2, SMARCA4, or SMG1 mRNA or protein.
229. A method of enriching a prospective patient population for subjects likely to respond to a MEK inhibitor therapy comprising performing any of the methods of claims 50 to 55 on a biological sample obtained from one or more subjects within said patient population.
230. A method of enriching a prospective patient population for subjects likely to respond to a combination therapy comprising a MEK inhibitor and a TORC inhibitor comprising performing any of the methods of claims 91 to 93 on a biological sample obtained from on one or more subjects within said patient population
231. A method of enriching a prospective patient population for subjects likely to respond to a combination therapy comprising a MEK inhibitor and a chemotherapy comprising performing any of the methods of claims 181 to 183 on a biological sample obtained from one or more subjects within said patient population
232. A method of enriching a prospective patient population for subjects likely to respond to a MEK inhibitor therapy comprising performing any of the methods of claims 50-87 on a biological sample obtained from one or more subjects within said patient population.
233. A method of enriching a prospective patient population for subjects likely to respond to a combination therapy comprising a MEK inhibitor and a TORC inhibitor comprising performing any of the methods of claims 91 to 127 on a biological sample obtained from on one or more subjects within said patient population
234. A method of enriching a prospective patient population for subjects likely to respond to a combination therapy comprising a MEK inhibitor and a chemotherapy comprising performing any of the methods of claims 181 to 218 on a biological sample obtained from one or more subjects within said patient population
235. A method for selecting a subject for a MEK monotherapy if it is likely that the subject will respond to the MEK monotherapy, wherein said likelihood of response is determined by performing any of the methods of claims 50 to 55 on a biological sample obtained from the subject.
236. A method for selecting a subject for a combination therapy if it is likely that the subject will respond to the combination therapy comprising a MEK inhibitor and a TORC inhibitor, wherein said likelihood of response is determined by performing any of the methods of claims 91 to 93 on a biological sample obtained from the subject.
237. A method for selecting a subject for a combination therapy comprising a MEK inhibitor and a chemotherapy if it is likely that the subject will respond to the combination therapy comprising a MEK inhibitor and a chemotherapy, wherein said likelihood of response is determined by performing any of the methods of claims 181 to 183 on a biological sample obtained from the subject.

It is to be understood that the present invention is not limited to the particular methodologies, protocols, cell lines, vectors, and reagents described, as these can vary. It is also understood that the terminology used herein is for the purpose of describing particular embodiments only and is not to limit the scope of the present invention.

Various publications, articles and patents are cited or described in the background and throughout the specification; each of these references is herein incorporated by reference in its entirety. Discussion of documents, acts, materials, devices, articles and the like which has been included in the present specification is for the purpose of providing context for the invention. Such discussion is not an admission that any or all of these matters form part of the prior art with respect to any inventions disclosed or claimed. In case of conflict, the present application, including any definitions herein, will control.

Unless defined otherwise, all technical and scientific terms used herein have the same meaning as commonly understood to one of ordinary skill in the art to which this invention pertains. Otherwise, certain terms used herein have the meanings as set forth in the specification.

Particular embodiments of this invention are described herein. Those skilled in the art will recognize or be able to ascertain using no more than routine experimentation, many equivalents to the specific embodiments described herein. Such equivalents are intended to be compassed by the invention. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. A number of embodiments of the invention have been described. Nevertheless, it will be understood that various modifications can be made without departing from the spirit and scope of the invention. Accordingly, the descriptions in the Examples section are intended to illustrate but not limit the scope of invention described in the claims.

### EXAMPLES

### Example 1. Biomarkers of Responsiveness to MEK Inhibitor Therapies

This example describes the identification of tumor suppressor genes that are biomarkers of response to MEK inhibitor therapies through a method that integrates CRISPR/Cas9-based somatic genome engineering and molecular barcoding into established Cre/Lox-based genetically engineered mouse models of oncogenic Kras-driven lung cancer.

Generation of Barcoded Lenti-sgRNA/Cre Vector Pool

1. Design and Generation of sgRNAs

For studies ST-0003 and ST-0007, lentiviral vectors carrying Cre as well as an sgRNA targeting each of 22 known and putative lung adenocarcinoma tumor suppressors were generated: Apc, Arid2, Atm, Atrx, Brca2, Cdkn2a, Cmtr2, Keap1, Kmt2d, Lkb1, Mga, Nf1, p53, Pten, Ptprd, Rb1, Rbm10, Rnf43, Setd2, Smad4, Stag2, and Tsc1.

For study OMI-0007, lentiviral vectors carrying Cre as well as an sgRNA targeting each of 22 known and putative lung adenocarcinoma tumor suppressors were generated: Apc, Arid2, Atm, Atrx, Brca2, Cdkn2a, Cmtr2, Fbxw7, Kdm6a, Keap1, Kmt2d, Kras, Lkb1, Mga, Msh2, Nf1, NF2, p53, Palb2, Pcna, Pten, Ptpn11, Ptprd, Rb1, Rbm10, Rnf43, Setd2, Smad4, Stag2, and Tsc1.

For study OMI-0031, SC-0001, and SC-0002, lentiviral vectors carrying Cre as well as an sgRNA targeting each of 61 known and putative lung adenocarcinoma tumor suppressors were generated: Apc, Arid1a, Arid2, Asxl1, Atm, Atrx, Bap1, Brca1, Brca2, Cdkn2a, Chd2, Cic, Cmtr2, Crebbp, Cul3, Dicer1, Dlc1, Dusp4, Ep300, Fat1, Fbxw7, Kdm5c, Kdm6a, Keap1, Kmt2c, Kmt2d, KrasWT, Lkb1 (Stk11), Lrp1b, Mga, Msh2, Mtap, Ncoa6, Nf1, Nf2, p53, Palb2, Pbrm1, Pcna, Pten, Ptpn13, Ptprd, Ptprs, Rasa1, Rb1, Rb1cc1, Rbm10, Rnf43, Setd2, Shp2, Smad2, Smad4, Smarca4, Smg1, Stag2, Tet2, Tgfbr2, Tsc1, Tsc2, Usp15, and Zfhx3).

Vectors were also generated carrying inert guides: sgRosa26-1, sgRosa26-2, sgRosa26-3, sgNT-1, sgNT-2, and sgNT-3. All possible 20-bp sgRNAs (using an NGG PAM) targeting each tumor suppressor gene of interest were identified and scored for predicted on-target cutting efficiency using an available sgRNA design/scoring algorithm (Doench et al., Nat Biotechnol 34, 184-191 (2016). https://doi.org/10.1038/nbt.3437). For each tumor suppressor gene, we selected a unique sgRNAs predicted to be the most will produce null alleles; preference was given to sgRNAs that were previously validated in vivo (Rogers et al., Nat Methods. 2017 July; 14(7):737-742. Doi: 10.1038/nmeth.4297; Rogers et al., Nat Genet. 2018 April; 50(4):483-486. doi: 10.1038/s41588-018-0083-2; Winters et al. Nat Commun. 2017 Dec. 12; 8(1):2053. doi: 10.1038/s41467-017-01519-y. PMID: 29233960; PMCID: PMC5727199, sgRNAs with the highest predicted cutting efficiencies, as well as those targeting exons conserved in all known splice isoforms (ENSEMBL), closest to splice acceptor/splice donor sites, positioned earliest in the gene coding region, occurring upstream of annotated functional domains (InterPro; UniProt), and occurring upstream of known human lung adenocarcinoma mutation sites. Lenti-U6-sgRNA/Cre vectors containing each sgRNA were generated as previously described (Rogers et al., Nat Methods. 2017 July; 14(7):737-742. doi: 10.1038/nmeth.4297). Briefly, Q5 site-directed mutagenesis (NEB E0554S) was used to insert sgRNAs into the parental lentiviral vector containing the U6 promoter as well as PGK-Cre.

2. Barcode Diversification of Lenti-sgRNA/Cre

To enable quantification of the number of cancer cells in individual tumors in parallel using high-throughput sequencing, we diversified the Lenti-sgRNA/Cre vectors with a 46 bp multi-component barcode cassette that would be unique to each tumor by virtue of stable integration of the lentiviral vector into the initial transduced cell. This 46 bp DNA barcode cassette was comprised of a known 6-nucleotide ID specific to the vector backbone (vectorID), a 10-nucleotide ID specific to each individual sgRNA (sgID), and a 30-nucleotide random barcode containing 20 degenerate bases (random BC).

The 46 bp barcode cassette for each sgRNA was flanked by universal Illumina® TruSeq adapter sequences and synthesized as single stranded DNA oligos. Forward and reverse primers complimentary to the universal TruSeq sequences and containing 5′ tails with restriction enzyme sites (AscI and NotI) were used in a PCR reaction to generate and amplify double stranded barcode cassettes for cloning. Each Lenti-sgRNA-Cre vector and its matching insert barcode PCR product was digested with AscI and NotI.

To generate a large number of uniquely barcoded vectors, we ligated 1 μg of linear vector and 50 ng of insert with T4 DNA ligase in a 100 μl ligation reaction. After 5 hours of incubation at room temperature, ligated DNA was precipitated by centrifugation at 14 k for 12 min after adding 5 μl Glycogen (5 mg/ml) and 280 μl 100% Ethanol into the ligation reaction. The DNA pellet was washed with 80% Ethanol and air dried before being resuspended with 10 μl water. This 10 μl well-dissolved DNA was transformed into 100 μl of Sure Electrical Competent Cells using BioRad electroporation system following their manual. Electroporation-transformed cells were immediately recovered by adding into 5 ml pre-warmed SOC media. From the 5 ml cells in SOC medium, 10 μl were further diluted with LB ampicillin broth and a final dilution of 1:200K was plated on LB ampicillin plate for incubation at 37° C. The remaining cells in SOC medium were mixed gently and thoroughly before being inoculated into 100 ml LB/Ampicillin broth, shaking at 220 rpm at 37° C. overnight. The next day, colony number on LB/Ampicillin plate were counted to estimate the complexity of each library while 100 ml bacteria culture were pelleted for plasmid purification.

Eight colonies from each library were picked and PCR screened for verification of the specific sgRNA sequence and corresponding barcode sequence among these eight colonies. The final purified library plasmid for each library is again sequence verified.

Production, Purification, and Titering of Lentivirus

24 hours prior to transfection, 2.4×10⁷ 293T cells were plated on 15 cm tissue culture plate. 30 μg of pPack (packaging plasmid mix) and 15 μg of library plasmid DNA were mixed well in 1.5 ml serum free D-MEM medium before equal volume of serum free D-MEM medium containing 90 μl of LipoD293 was added. The resulted mixture was incubated at room temperature for 10-20 min before adding into 293T cells in the 15 cm plate. At 24 hours post-transfection, replace the medium containing complexes with 30 ml of fresh D-MEM medium supplemented with 10% FBS, DNase I (1 U/ml), MgCl2 (5 mM), and 20 mM HEPES, pH 7.4. The entire virus-containing medium from each plate was collected and filtered through a Nalgene 0.2 μm PES filter at 48 hours post-transfection. The viruses were further concentrated by centrifugation at 18,500 rpm, 4° C. for 2 hours and the pellet was dissolved in 500 μl PBS buffer. 50 μl virus aliquots were stored at −80° C.

To determine the titer for packaged library constructs, 1×10⁵ LSL-YFP MEF cells were transduced with 1 μl of viruses in 1 ml culture medium containing 5 μg/ml polybrene. Transduced cells were incubated for 72 hours before being collected for FACS analysis to measure the percentage of GFP cells. Control viruses were used in parallel to normalize the virus titers.

Pooling of Lenti-sgRNA/Cre Vectors

To generate a pool of barcoded Lenti-sgRNA/Cre vectors to generate multiple tumor genotypes within individual mice, barcoded Lenti-sgRNA/Cre vectors targeting 22 tumor suppressor genes (ST-0003 and ST-0007)(Apc, Arid2, Atm, Atrx, Brca2, Cdkn2a, Cmtr2, Keap1, Kmt2d, Lkb1, Mga, Nf1, p53, Pten, Ptprd, Rb1, Rbm10, Rnf43, Setd2, Smad4, Stag2, and Tsc1) or 30 tumor suppressor genes (OMI-0007) (Apc, Arid2, Atm, Atrx, Brca2, Cdkn2a, Cmtr2, Fbxw7, Kdm6a, Keap1, Kmt2d, Kras, Lkb1 (Stk11), Mga, Msh2, Nf1, NF2, p53, Palb2, Pcna, Pten, Ptpn11 (Shp2), Ptprd, Rb1, Rbm10, Rnf43, Setd2, Smad4, Stag2, and Tsc1) or 61 tumor suppressor genes (Apc, Arid1a, Arid2, Asxl1, Atm, Atrx, Bap1, Brca1, Brca2, Cdkn2a, Chd2, Cic, Cmtr2, Crebbp, Cul3, Dicer1, Dlc1, Dusp4, Ep300, Fat1, Fbxw7, Kdm5c, Kdm6a, Keap1, Kmt2c, Kmt2d, KrasWT, Lkb1 (Stk11), Lrp1b, Mga, Msh2, Mtap, Ncoa6, Nf1, Nf2, p53, Palb2, Pbrm1, Pcna, Pten, Ptpn13, Ptprd, Ptprs, Rasa1, Rb1, Rb1cc1, Rbm10, Rnf43, Setd2, Shp2, Smad2, Smad4, Smarca4, Smg1, Stag2, Tet2, Tgfbr2, Tsc1, Tsc2, Usp15, and Zfhx3) (OMI-0031, SC-0001, and SC-0002) and those containing the inert, negative control sgRNAs (sgRosa26-1, sgRosa26-2, sgRosa26-3, sgNT-1, sgNT-2, and sgNT-3) were combined such that the viruses would be at equal ratios in relation to their estimated titers. This pool was then diluted with 1×DPBS to reach a final viral titer of 180,000 FU per 60 μL.

Mice and Tumor Initiation

Kras^{LSL-G12D} (K) and H11^{LSL-Cas9} (Cas9) mice have been described (Jackson et al., Genes & Dev. 2001. 15: 3243-3248 (doi:10.1101/gad.943001); Chiou et al., Genes & Dev. 2015. 29: 1576-1585 (doi:10.1101/gad.264861.115)). Lung tumors in Kras^{LSL-G12D/+}; H11^{LSL-Cas9} (KC) mice were initiated via intratracheal delivery of 180,000 functional units (FU) of a lentivirus pool containing barcoded Lenti-U6-sgRNA/PGK-Cre vectors targeting either 22 genes (ST-0003 and ST-0007) (Apc, Arid2, Atm, Atrx, Brca2, Cdkn2a, Cmtr2, Keap1, Kmt2d, Lkb1 (Stk11), Mga, Nf1, p53, Pten, Ptprd, Rb1, Rbm10, Rnf43, Setd2, Smad4, Stag2, and Tsc1) or 30 genes (OMI-0007) (Apc, Arid2, Atm, Atrx, Brca2, Cdkn2a, Cmtr2, Fbxw7, Kdm6a, Keap1, Kmt2d, Kras, Lkb1 (Stk11), Mga, Msh2, Nf1, NF2, p53, Palb2, Pcna, Pten, Ptpn11, Ptprd, Rb1, Rbm10, Rnf43, Setd2, Smad4, Stag2, and Tsc1) or 61 tumor suppressor genes (Apc, Arid1a, Arid2, Asxl1, Atm, Atrx, Bap1, Brca1, Brca2, Cdkn2a, Chd2, Cic, Cmtr2, Crebbp, Cul3, Dicer1, Dlc1, Dusp4, Ep300, Fat1, Fbxw7, Kdm5c, Kdm6a, Keap1, Kmt2c, Kmt2d, KrasWT, Lkb1 (Stk11), Lrp1b, Mga, Msh2, Mtap, Ncoa6, Nf1, Nf2, p53, Palb2, Pbrm1, Pcna, Pten, Ptpn13, Ptprd, Ptprs, Rasa1, Rb1, Rb1cc1, Rbm10, Rnf43, Setd2, Shp2, Smad2, Smad4, Smarca4, Smg1, Stag2, Tet2, Tgfbr2, Tsc1, Tsc2, Usp15, and Zfhx3) (OMI-0031, SC-0001, and SC-0002) plus 6 negative control sgRNAs (three targeting the Rosa26 gene, which are actively cutting but functionally inert, and 3 non-cutting sgRNAs with no expected genomic target [sgNon-Targeting: sgNT]).

Drug Dosing

12 weeks post tumor initiation, mice were treated with the following:

ST-0003 (KRAS G12D/+cas9/cas9 mice)
Vehicle (n=8) delivered PO, with once daily dosing (QD), seven consecutive days a week, for three weeks until takedown at 15 weeks post induction
MEK inhibitor Trametinib (n=21) delivered PO, at 3 mg/kg with once daily dosing (QD), seven consecutive days a week, for three weeks until takedown at 15 weeks post induction

ST-0007 (KRAS G12D/+cas9/cas9 mice)
Vehicle (n=17) delivered PO, with once daily dosing (QD), seven consecutive days a week, for three weeks until takedown at 15 weeks post induction
MEK inhibitor Trametinib (n=19) delivered PO, at 3 mg/kg with once daily dosing (QD), seven consecutive days a week, for three weeks until takedown at 15 weeks post induction
MEK inhibitor Trametinib (n=20) delivered PO, at 1 mg/kg with once daily dosing (QD), seven consecutive days a week, for three weeks until takedown at 15 weeks post induction
MEK inhibitor Selumetinib (n=17) delivered PO, at 50 mg/kg with twice daily dosing (BID), seven consecutive days a week, for three weeks until takedown at 15 weeks post induction
MEK inhibitor Selumetinib (n=17) delivered PO, at 15 mg/kg with twice daily dosing (BID), seven consecutive days a week, for three weeks until takedown at 15 weeks post induction

OMI-0007 (KRAS G12D/+cas9/cas9 mice)
Vehicle (n=41) delivered PO, with once daily dosing (QD), seven consecutive days a week, for three weeks until takedown at 15 weeks post induction
MEK inhibitor Pimasertib (n=24) delivered PO, at 20 mg/kg with once daily dosing (QD), seven consecutive days a week, for three weeks until takedown at 15 weeks post induction
MEK inhibitor Pimasertib (n=26) delivered PO, at 10 mg/kg with once daily dosing (QD), seven consecutive days a week, for three weeks until takedown at 15 weeks post induction
MEK inhibitor Pimasertib (n=25) delivered PO, at 5 mg/kg with twice daily dosing (BID), seven consecutive days a week, for three weeks until takedown at 15 weeks post induction
Vehicle (n=15) delivered PO, with twice daily dosing (BID), seven consecutive days a week, for three weeks until takedown at 15 weeks post induction
MEK inhibitor Selumetinib (n=15) delivered PO, at 5 mg/kg with twice daily dosing (BID), seven consecutive days a week, for three weeks until takedown at 15 weeks post induction.

OMI-0031 (KRAS G12C/+cas9/cas9 mice)
Vehicle (n=40) delivered PO, with once daily dosing (QD), seven consecutive days a week, for three weeks until takedown at 15 weeks post induction
MEK inhibitor Trametinib (n=20) delivered PO, at 3 mg/kg with once daily dosing (QD), seven consecutive days a week, for three weeks until takedown at 15 weeks post induction.

SC-0001(KRAS G12D/+cas9/cas9 mice)
Vehicle (n=32) delivered PO, with once daily dosing (QD), seven consecutive days a week, for three weeks until takedown at 15 weeks post induction
MEK inhibitor Trametinib (n=16) delivered PO, at 3 mg/kg with once daily dosing (QD), seven consecutive days a week, for three weeks until takedown at 15 weeks post induction
MEK inhibitor Trametinib (n=14) delivered PO, at 1 mg/kg with once daily dosing (QD), seven consecutive days a week, for three weeks until takedown at 15 weeks post induction.

SC-0002 (KRAS G12D/+cas9/cas9 mice)
Vehicle (n=50) delivered PO, with once daily dosing (QD), seven consecutive days a week, for three weeks until takedown at 15 weeks post induction
MEK inhibitor Selumetinib (n=15) delivered PO, at 50 mg/kg with twice daily dosing (BID), seven consecutive days a week, for three weeks until takedown at 15 weeks post induction
MEK inhibitor Selumetinib (n=15) delivered PO, at 15 mg/kg with twice daily dosing (BID), seven consecutive days a week, for three weeks until takedown at 15 weeks post induction.

Dissection of Mouse Lungs

Bulk lung tissue was extracted from euthanized mice as previously described (Rogers et al., Nat Methods. 2017 July; 14(7):737-742. doi: 10.1038/nmeth.4297). Lung mass measurements were recorded as a proxy for overall lung tumor burden. Lungs were stored at −80° C. prior to subsequent processing.

All mouse experiments were performed in accordance with Animal Care and Use Committee guidelines.

Generation of Cell Spike-In Controls

DNA barcode cassettes comprised of known 46 bp sequences were flanked by universal Illumina® TruSeq adapter sequences and synthesized as single stranded DNA oligos. Forward and reverse primers complimentary to the universal TruSeq sequences and containing 5′ tails with restriction enzyme sites (Xba1 and BstB1) were used in a PCR reaction to generate and amplify double stranded barcode cassettes for cloning. A lentivector pRCMERP-CMV-MCS-EF1-TagR-Puro and each of the barcode insert PCR products were digested by Xba1 and BstB1 restriction enzymes.

Each digested barcode insert was cloned into linearized vector by T4 DNA ligase and transformed into OmniMax chemical competent cells (Invitrogen). Colonies from each transformation plate were screened by PCR and sequencing. One positive clone from each barcode containing construct was cultured for plasmid DNA extraction.

Virus was packaged from each of the barcoded pRCMERP constructs in 6-well plates using pPack packaging mix and LipoD293 reagent. Virus containing medium were collected at 48 hours post transfection and filtered with Nalgene 0.2 μm PES filter before being frozen down in aliquots at −80° C. Small aliquot of frozen viruses were thawed and added into HEK293 cells in 12-well plate for measuring titer by FACS analysis 72 hours after transduction.

To generate individual cell line containing each barcode construct, virus containing medium was added to HEK293 cells at MOI 0.1 in 10 cm plates. After overnight incubation, cells were recovered in fresh EMEM complete medium for 48 hours before splitting into a new plate containing 1 μg/ml puro in complete EMEM medium for puro selection.

After 3 days of puro selection, barcode-containing HEK293 cells were recovered in fresh EMEM complete medium without puro for another 3 days before being further expanded in 10 cm plates. Each established cell line was quality controlled by PCR amplification of the barcode region from genomic DNA to confirm integration of correct barcode sequences.

After cell expansion, cells from each barcoded HEK293 cell line were collected and diluted in PBS buffer containing 0.1% BSA to the desired concentrations. These cell suspensions were aliquoted and frozen down at −80° C.

Generation of dsDNA Spike-In Controls

DNA barcode cassettes comprised of 46 bp barcode cassettes and flanked by universal Illumina® TruSeq adapter sequences as well as additional buffer sequences to extend their total length to >400 bp were generated either by direct synthesis of the double-stranded DNA fragments (GeneWiz, IDT) or synthesis of single-stranded DNA oligos (GeneWiz, IDT) with overlapping complementary regions that were extended and amplified via PCR to create double-stranded DNA products that were then purified. Aliquots of these stock double-stranded DNA fragments were diluted to the desired copy numbers using DNase-free ultra-pure H2O and stored at −20° C.

Isolation of Genomic DNA from Mouse Lungs

Whole lungs were removed from freezer and allowed to thaw at room temperature. Spikeins were added to each whole lung samples. Added Qiagen Cell Lysis Buffer and proteinase K from Qiagen Gentra PureGene Tissue kit (Cat #158689) as described in manufacturer protocol. Whole lungs plus spikeins from each mouse were homogenized in the Cell Lysis buffer and Proteinase K solution using a tissue homogenizer (FastPrep-24 5G, MP Biomedicals Cat #116005500). Homogenized tissue was incubated at 55° C. overnight. To remove RNA from tissues samples, RNase A were added with additional spikeins to whole homogenized tissue. To maintain accurate representation of all tumors, DNA was extracted and alcohol precipitated from the entire lung lysate using Qiagen Gentra PureGene kit as described in manufacturer protocol. More spikeins were added to the resuspended DNA.

Preparation of sgID-BC Libraries for Sequencing

Libraries were prepared by amplifying the barcode region from 32 μg of genomic DNA per mouse. The barcode region of the integrated Lenti-sgRNA-BC/Cre vectors was PCR amplified using primer pairs that bound the universal Illumina® TruSeq adapters and contained dual unique multiplexing tags. We used a single-step PCR amplification of barcode regions, which we found to be a highly reproducible and quantitative method to determine the number of cancer cells in each tumor. We performed eight 100 μl PCR reactions per mouse (4 μg DNA per reaction) using Q5 HF HS 2× mastermix ((NEB #M0515) with the following PCR program:

| Step | Temperature (° C.) | | Time | | Cycles |
|---|---|---|---|---|---|
| Initial Denaturation | 98° | C. | 30 | seconds | |
| Denaturation | 98° | C. | 10 | seconds | 30 |
| Annealing | 63° | C. | 10 | seconds | |
| Extension | 72° | C. | 10 | seconds | |
| Final Extension | 72° | C. | 5 | minutes | |

| | | | |
|---|---|---|---|
| Hold | 4° | C. | ∞ |

PCR products were purified using SPRI beads. The concentration of purified PCR products from individual mice was determined by TapeStation (Agilent Technologies). Sets of 20-60 samples were pooled at equal ratios. Samples were sequenced on an Illumina® NextSeq and (Cellecta).

Analysis of Sequencing Data

Paired-end sequencing reads were demultiplexed via dual indexes and adapters sequences were trimmed. Paired-end alignments were constructed between mate-paired reads and library-specific databases of the expected oligonucleotide and tumor barcode insert sequences. These alignments were stringently filtered from downstream analysis if they failed to meet any of a number of quality criteria, including:
Mismatches between the two mate-pairs, which fully overlap one another, at any location.
Mismatches between the mate-paired reads and expected constant regions of the barcode or spikein to which they best align,
Any indels in alignments between mate-paired reads and the barcode or spikein to which they best align.

Following alignment, errors in paired-end reads were corrected via a simple greedy clustering algorithm:
Reads were dereplicated into read sequence/count tuples, (sᵢ, rᵢ)
These tuples were re-ordered from highest to lowest on the basis of their read abundances, {rᵢ}.
This list of tuples were traversed from i=1 . . . N, taking one of the following actions for each tuple (sᵢ, rᵢ):
   If sᵢ is not within a Hamming distance of 1 from any sⱼ with j<i, then (sᵢ, rᵢ) initiates a new cluster.
   If sⱼ is within a Hamming distance of 1 from some sⱼ with j<i, then it joins the cluster of sⱼ.
The resulting clusters are each considered to represent an error-corrected sequence equal to that of the sequence that founded the cluster and read count equal to the sum of the read counts of the dereplicated reads that are members of the cluster.

Following error correction, the read counts of each unique barcode were converted to tumor cell sizes by dividing the number of error-corrected reads of an oligonucleotide that had been spiked into the sample prior to tissue homogenization and lysis at a fixed, known concentration.

From these collections of tumor sizes across paired groups of MEK inhibitor-treated and vehicle-treated mice, the relative tumor number (RTN) metric was computed as previously described (Li, C., Lin, W.-Y et al. bioRxiv 2020.01.28.923912; doi: https://doi.org/10.1101/2020.01.28.923912).

Namely, shrinkage of inert tumors was estimated by finding the S that matches the median number of tumors in larger than a cutoff L in such paired groups after the vehicle-treated tumor sizes are multiplied by S (S<1 when MEKi works to shrink tumors). Subsequently, for each non-inert tumor genotype, the ratio of the number of tumors with this genotype larger than L in the control mice to the number of tumors larger than L*S in the treated mice was computed. The resulting ratio was divided by the same ratio computed for the inert tumors, and the log 2(⋅) of this ratio of ratios was determined. This metric, RTN_{score} is expected to be >0 for resistant genotypes and <0 for sensitive genotypes.

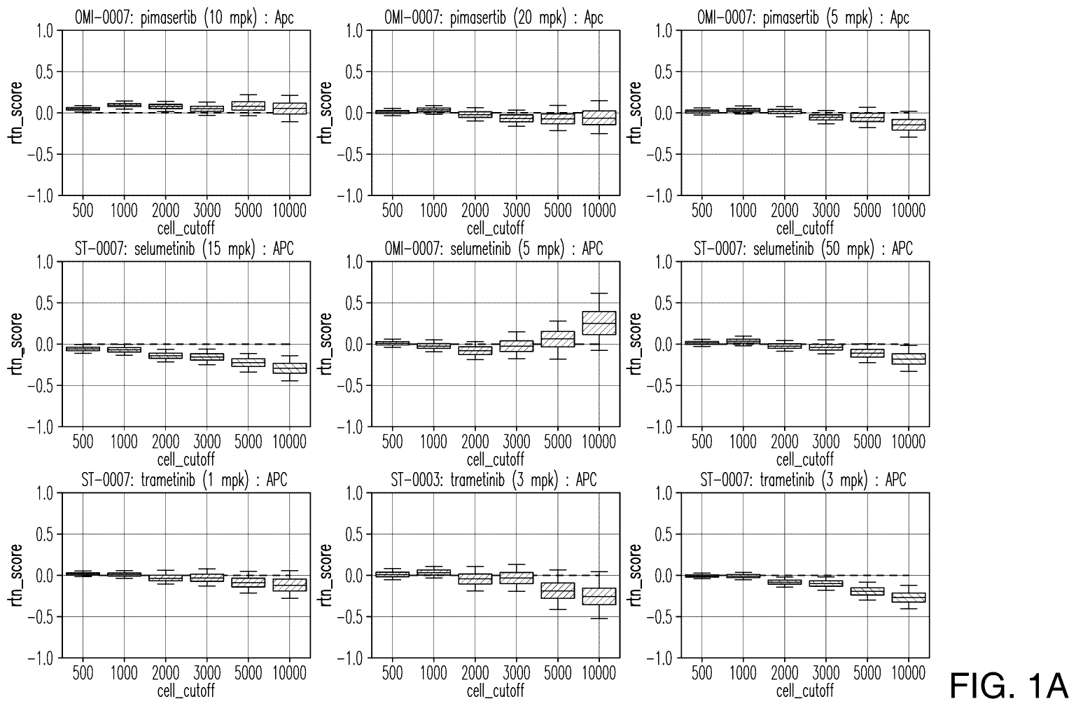

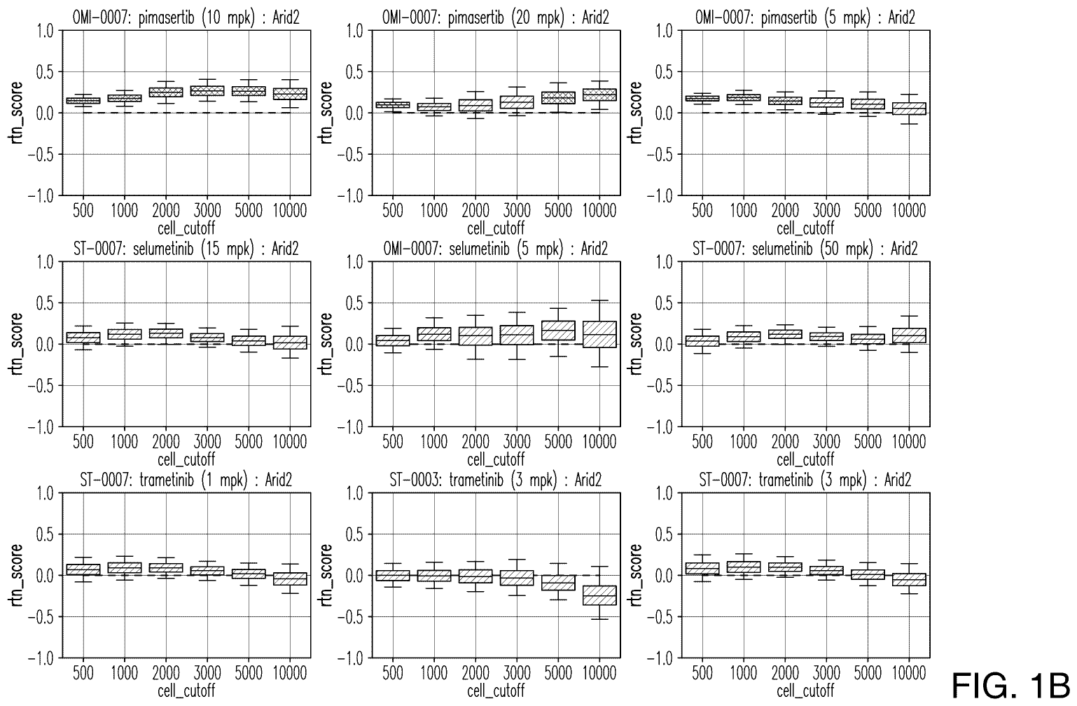

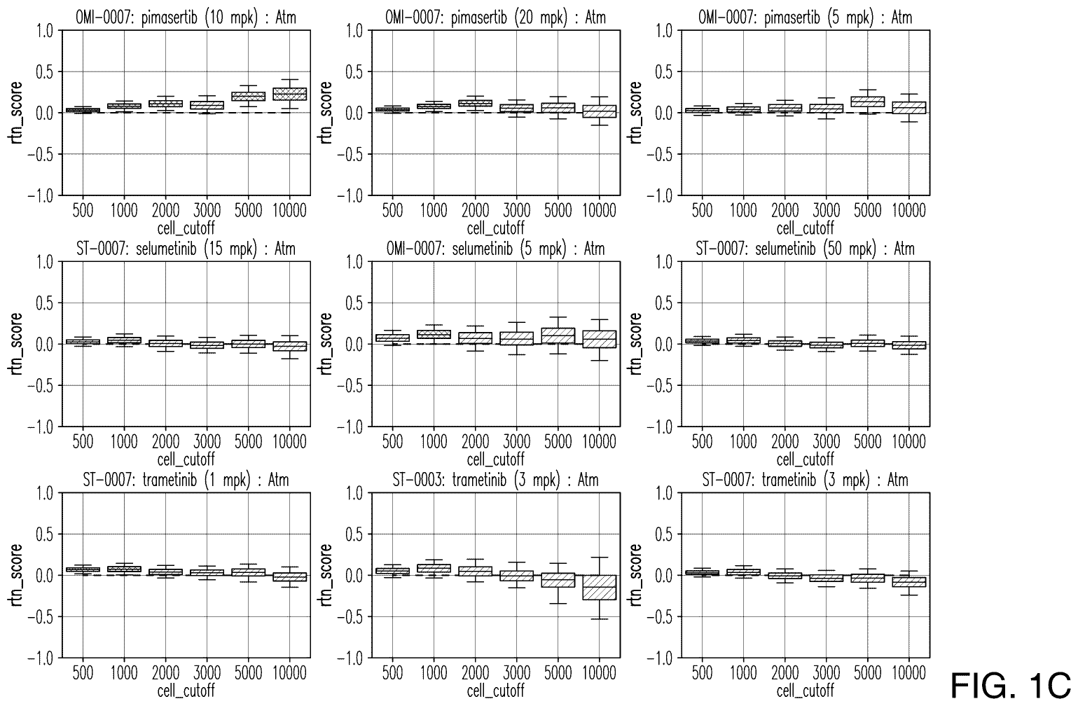

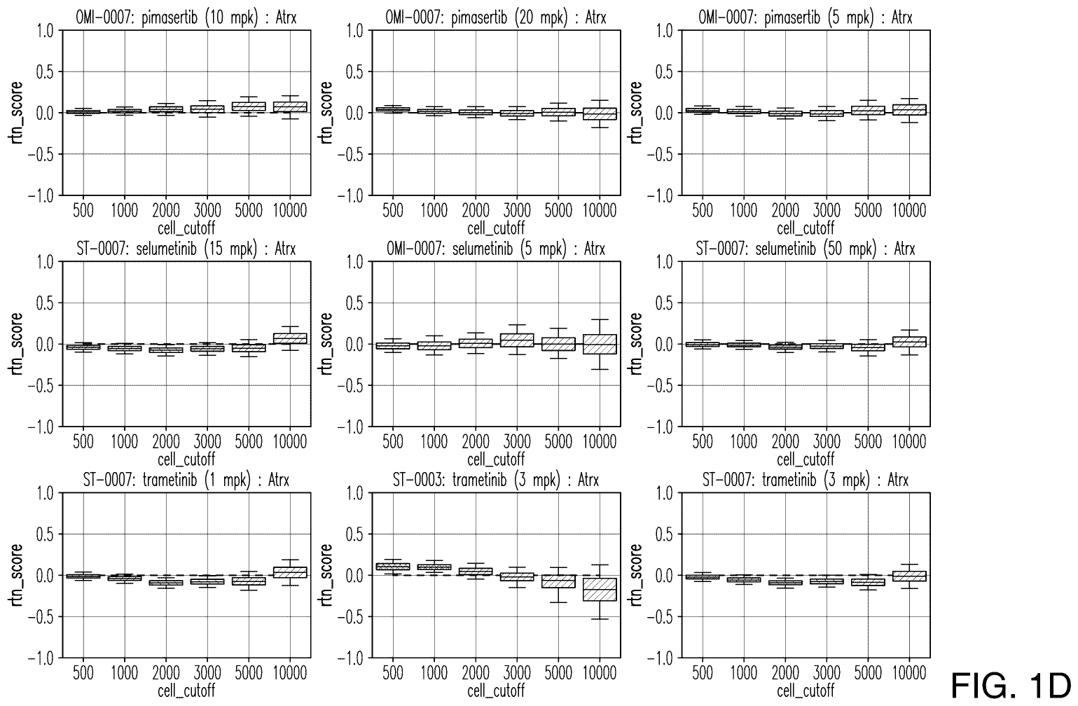

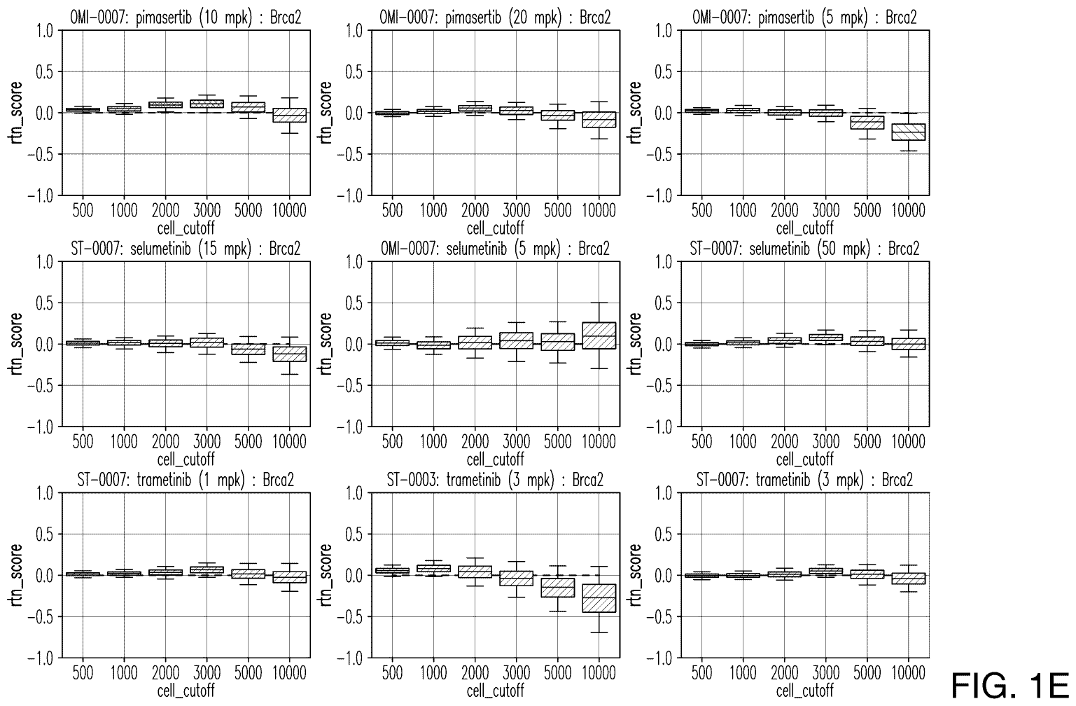

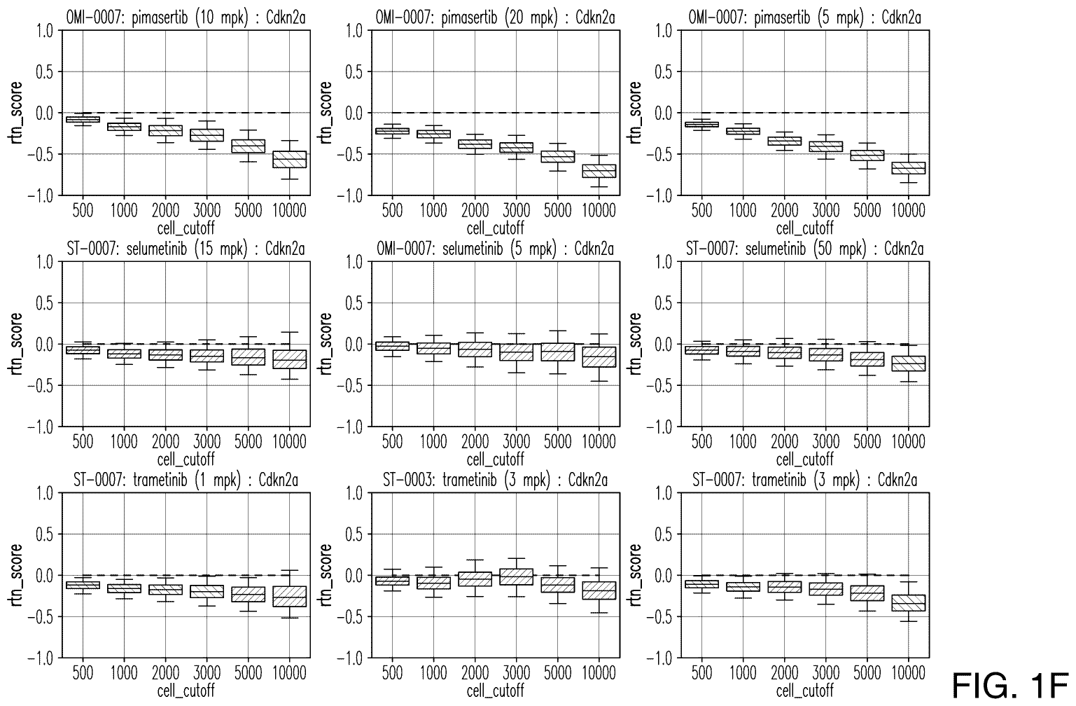

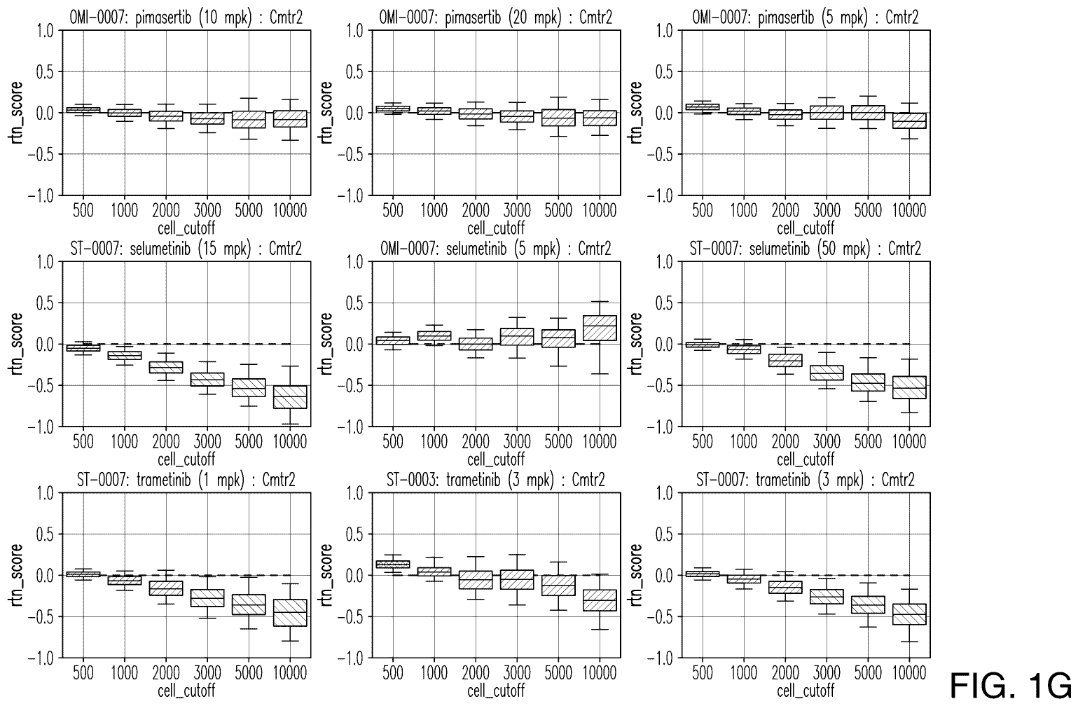

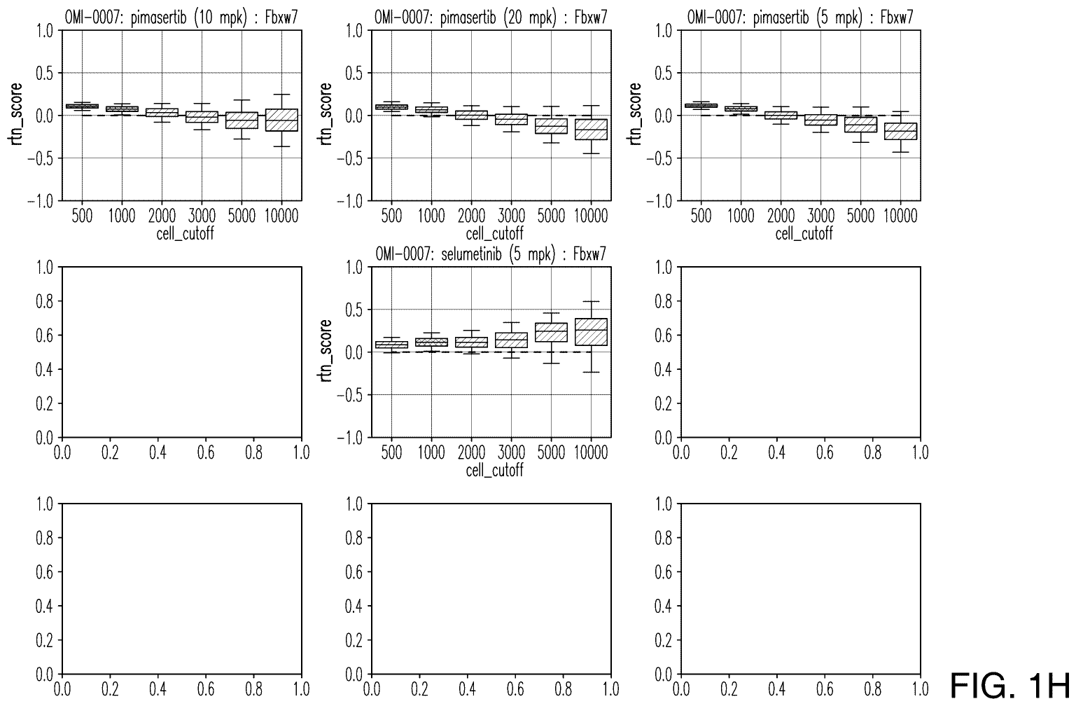

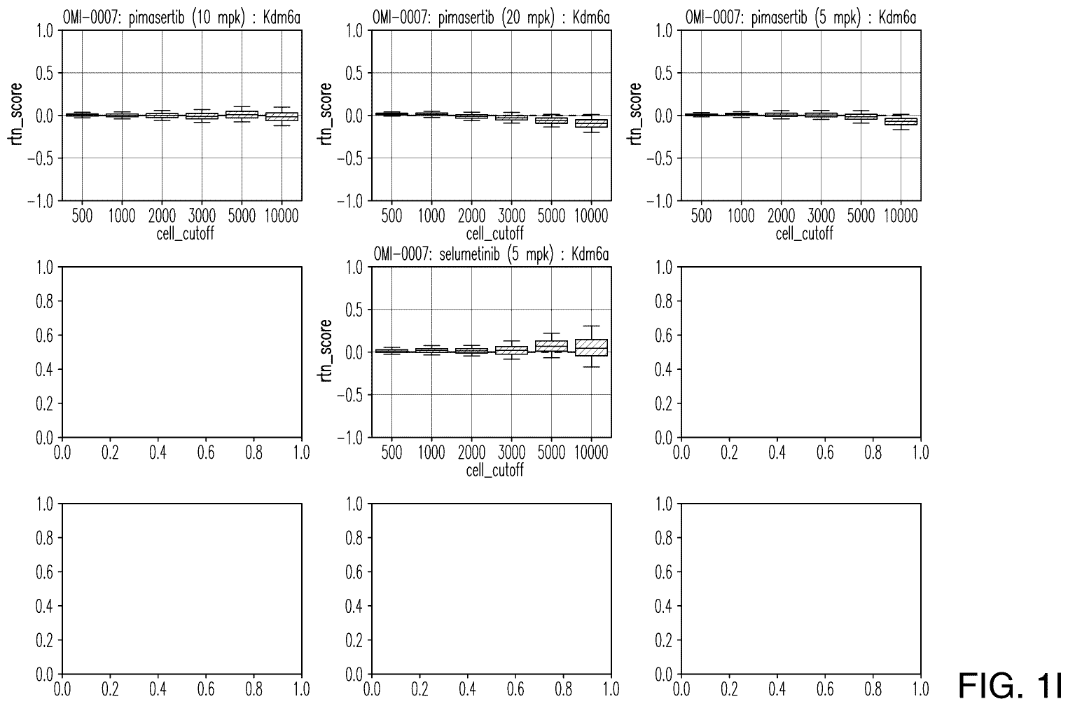

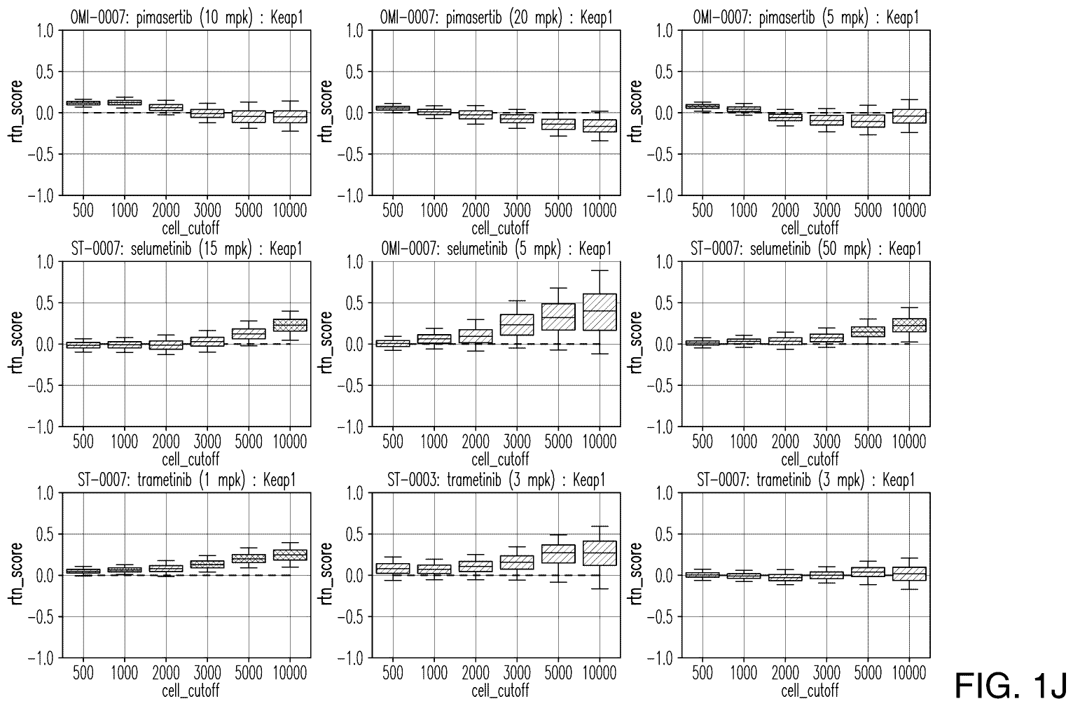

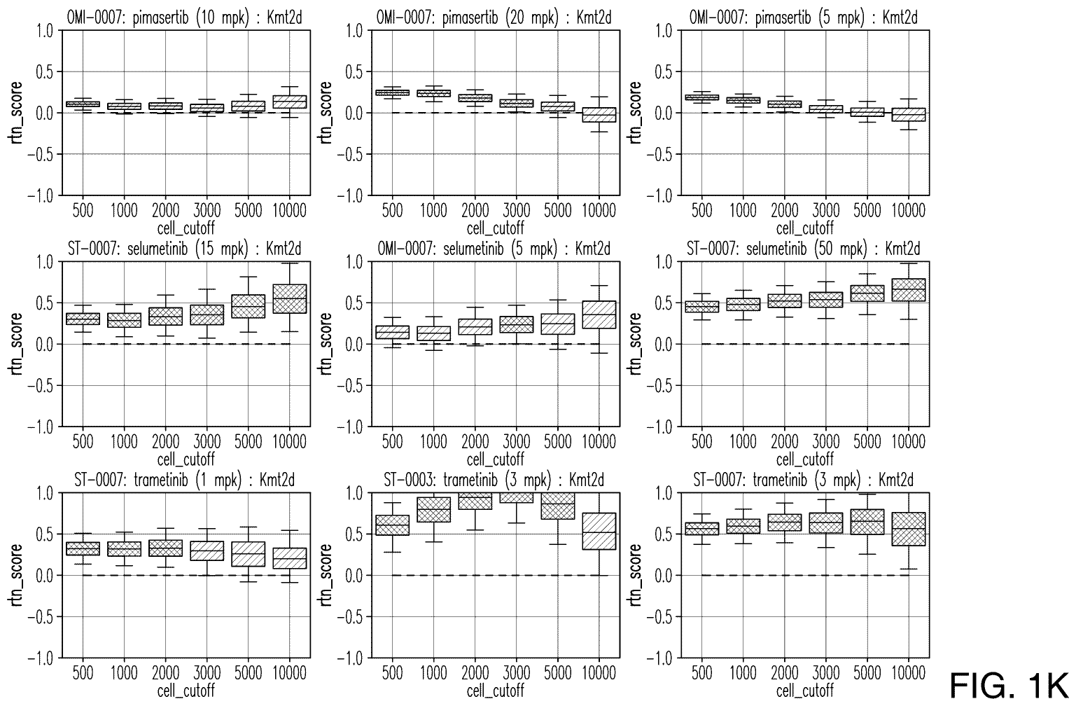

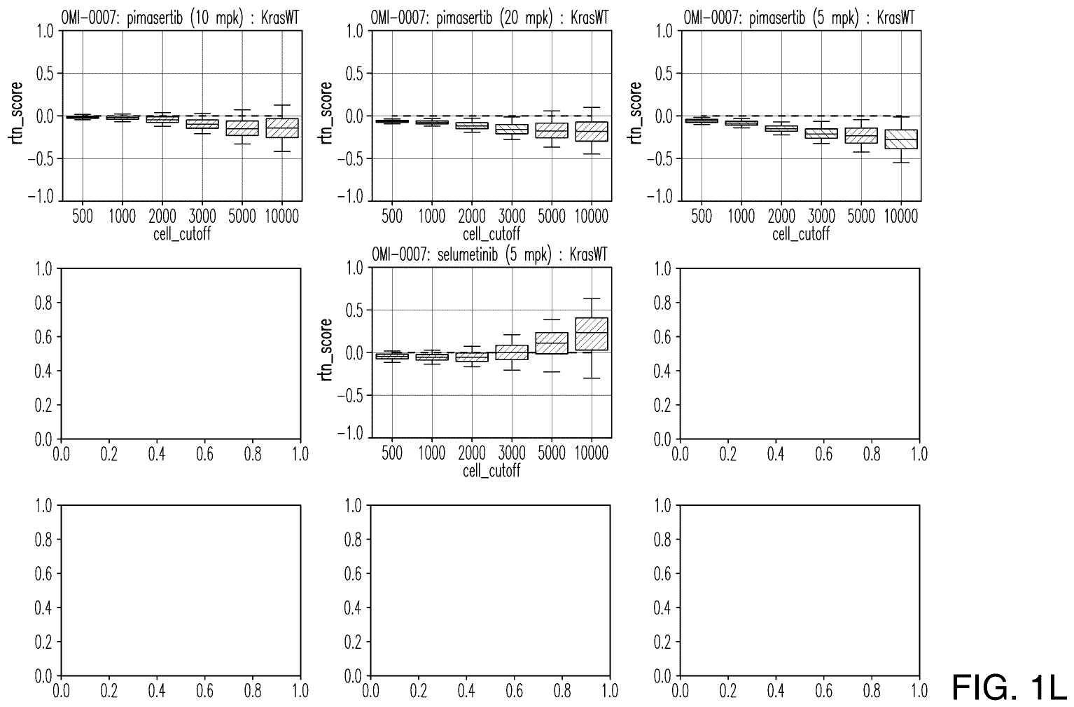

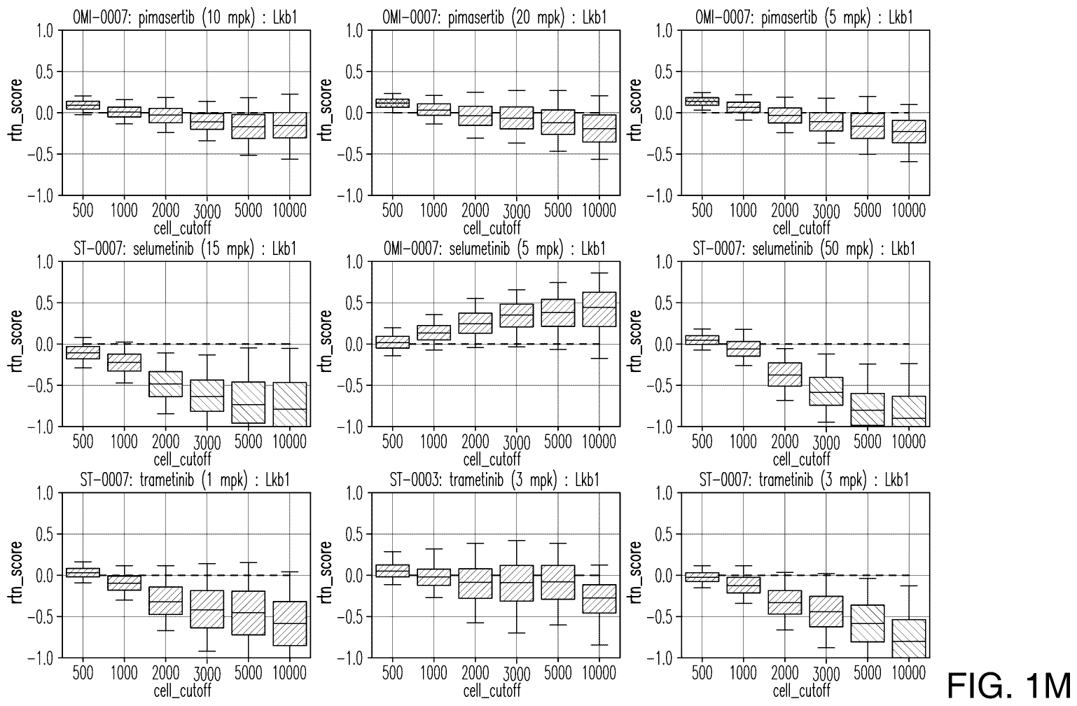

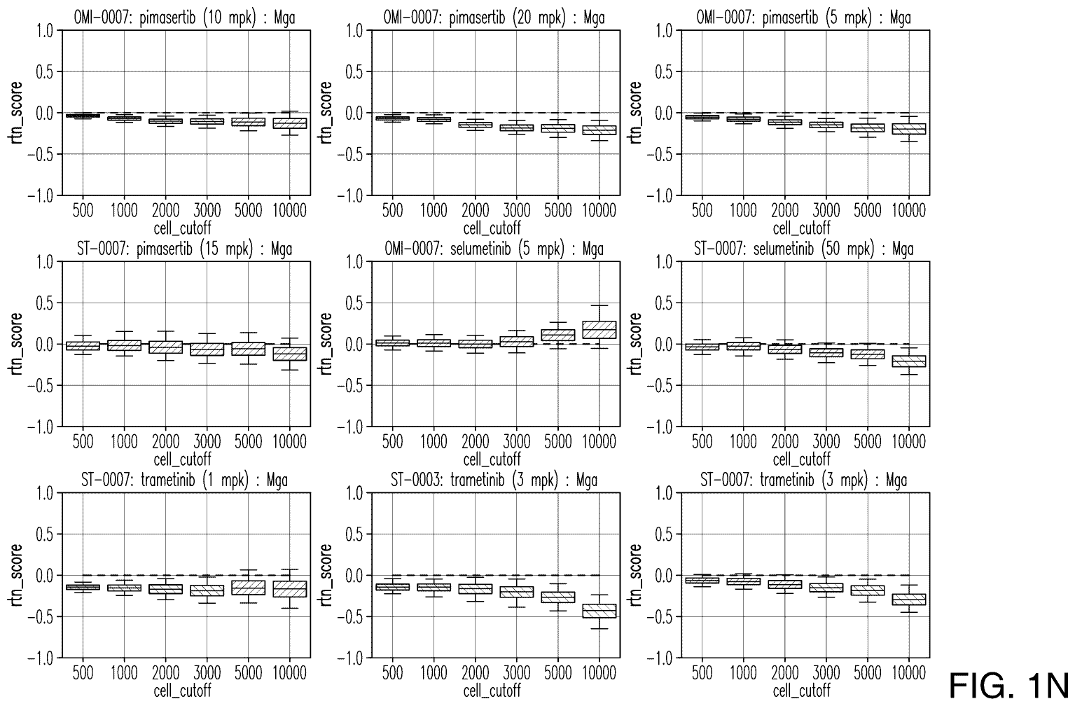

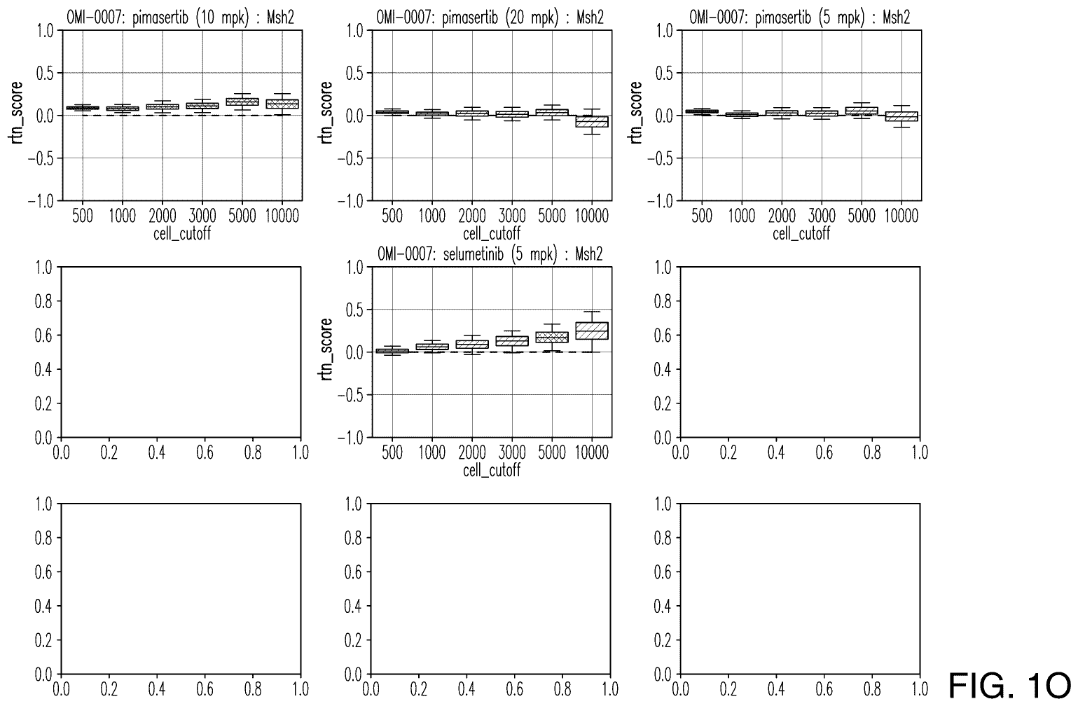

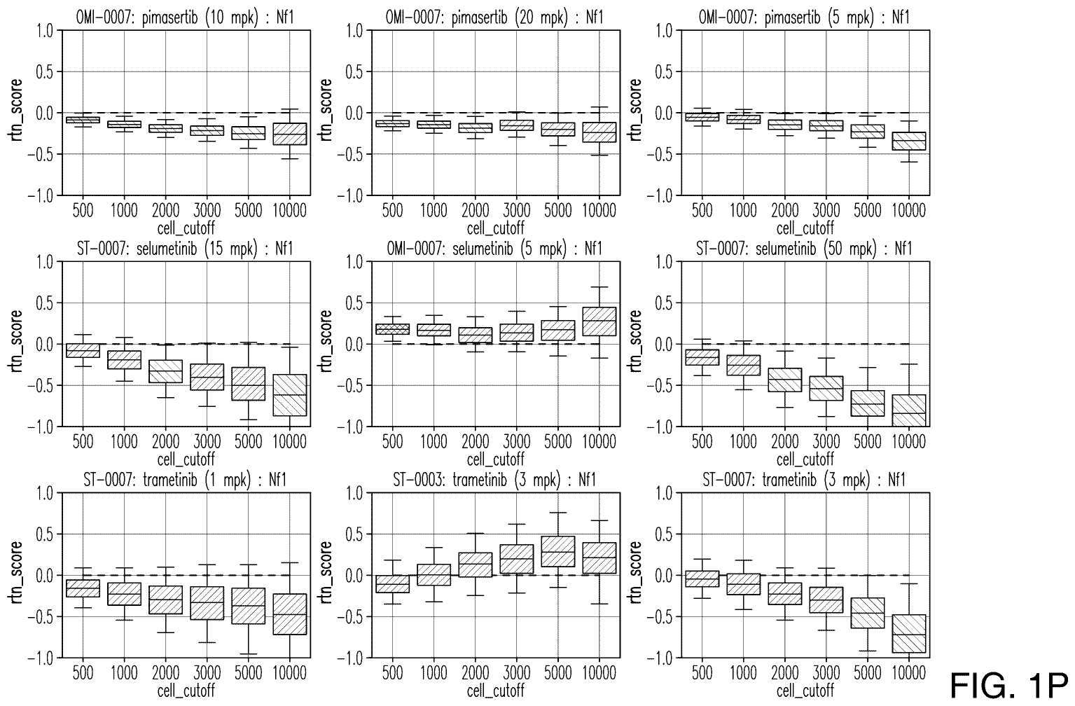

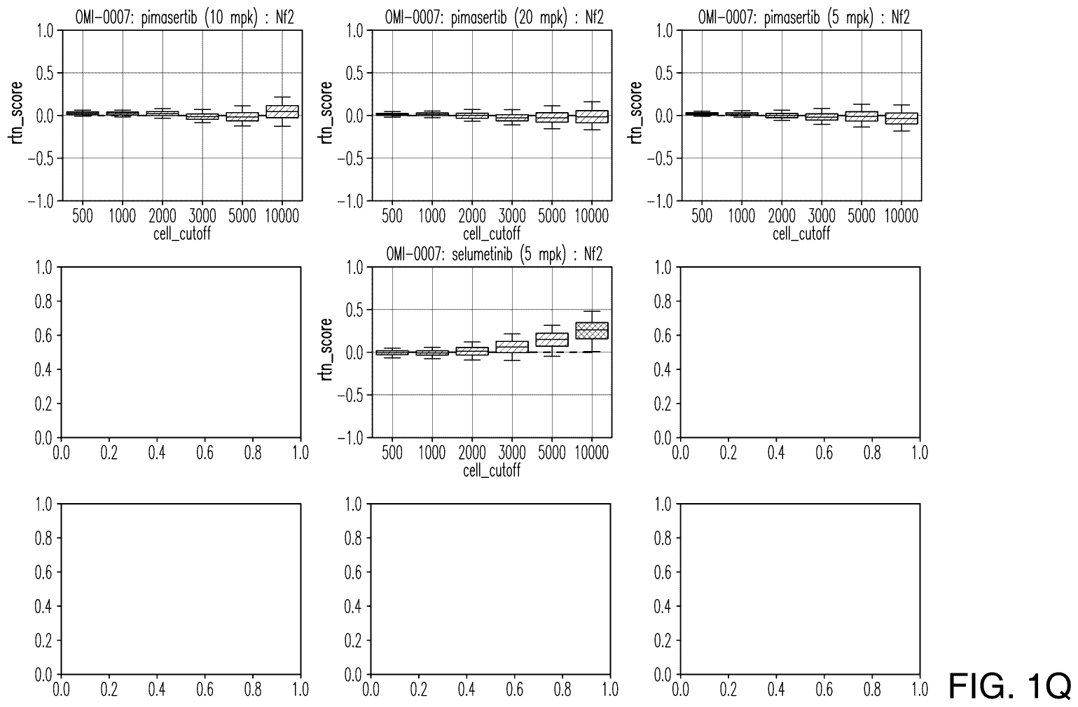

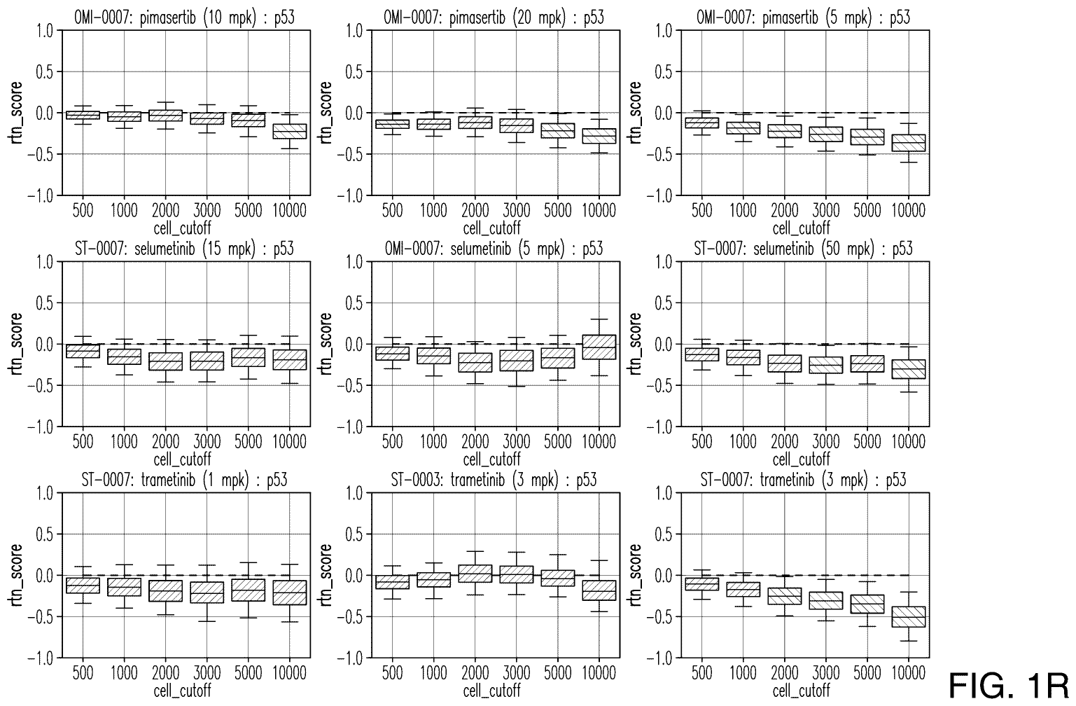

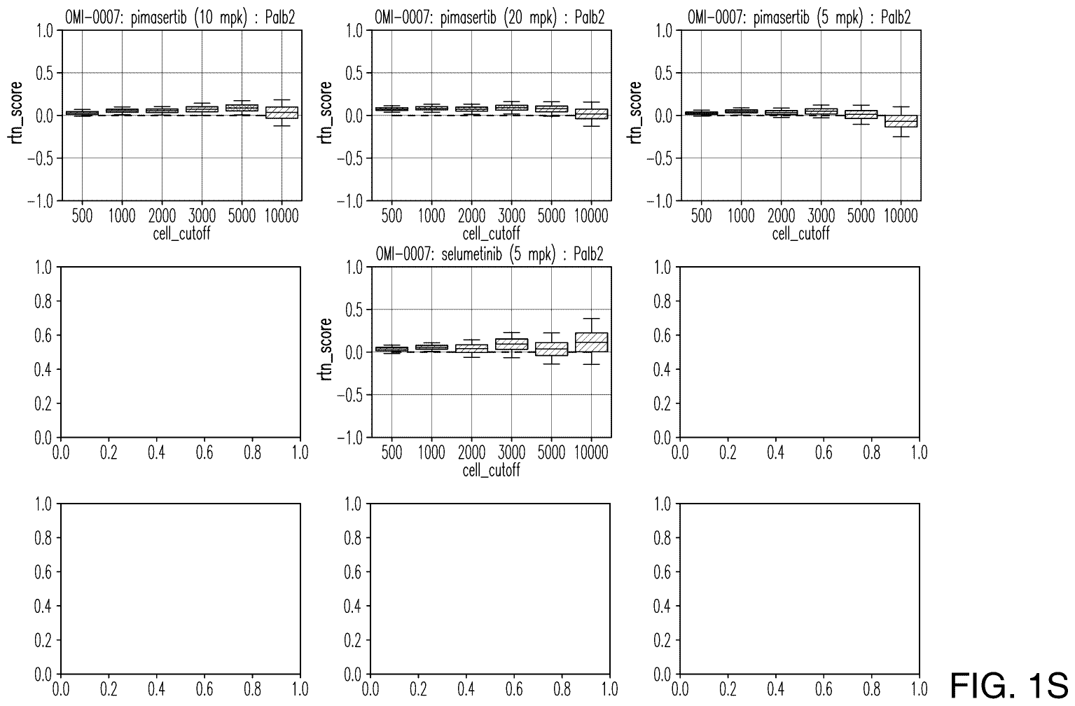

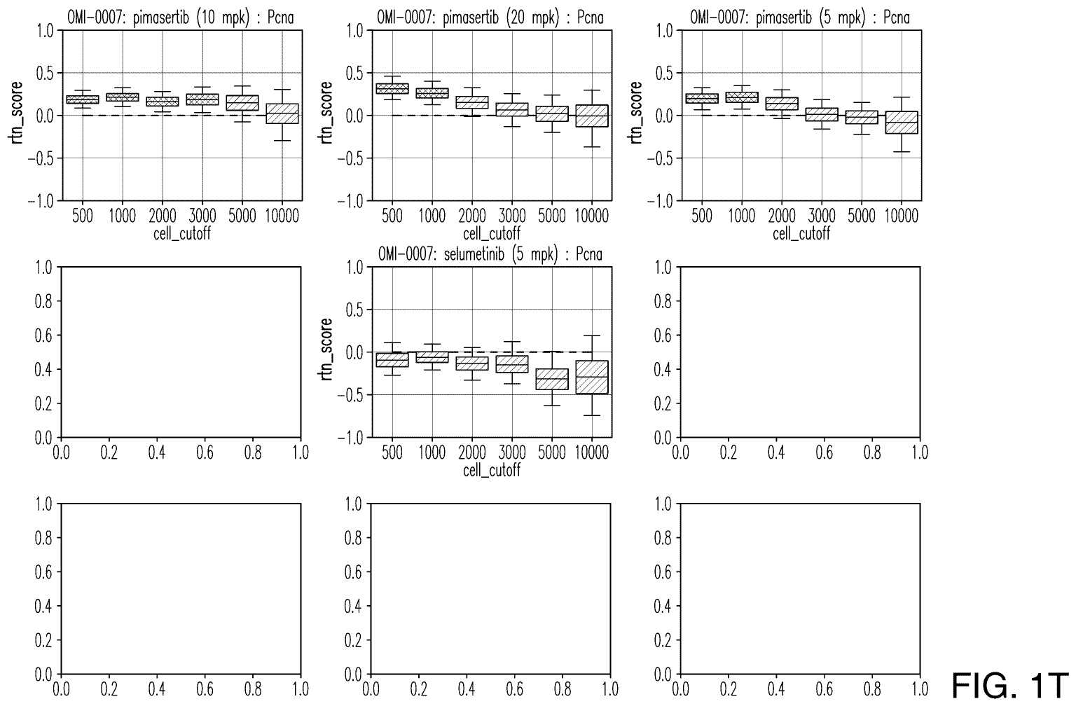

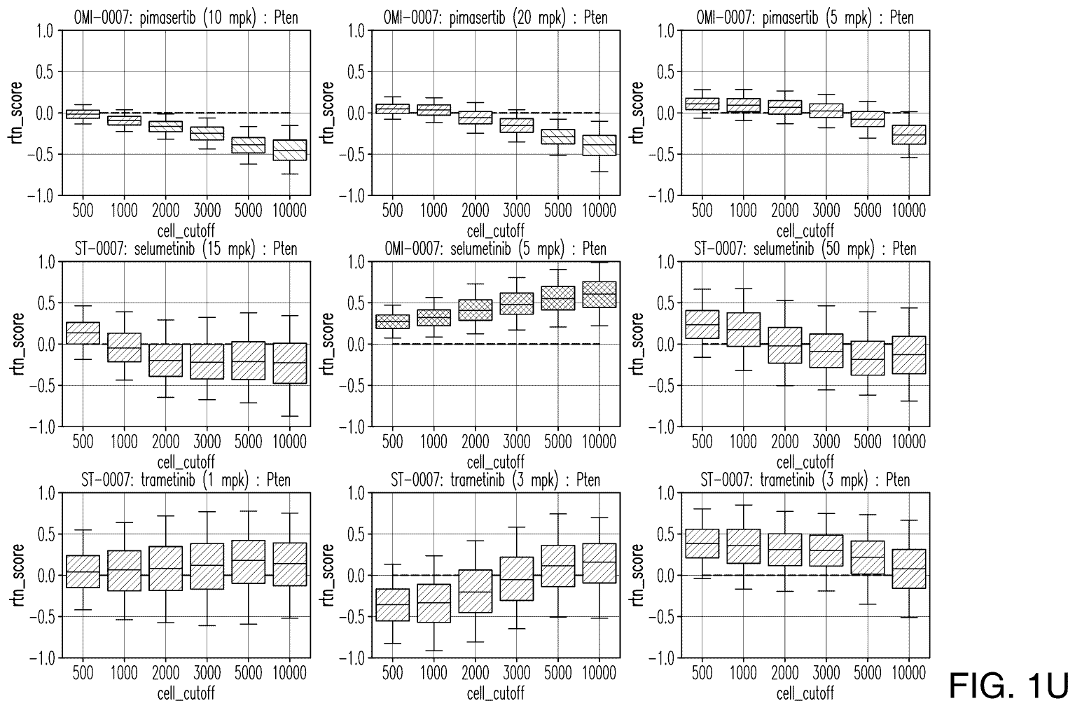

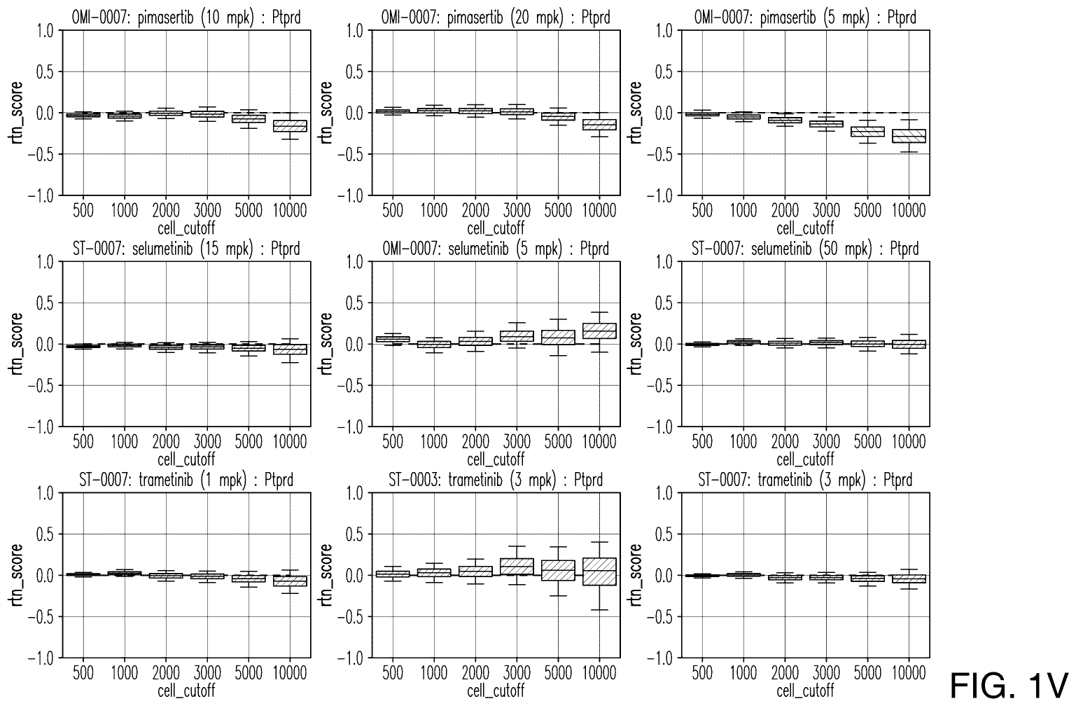

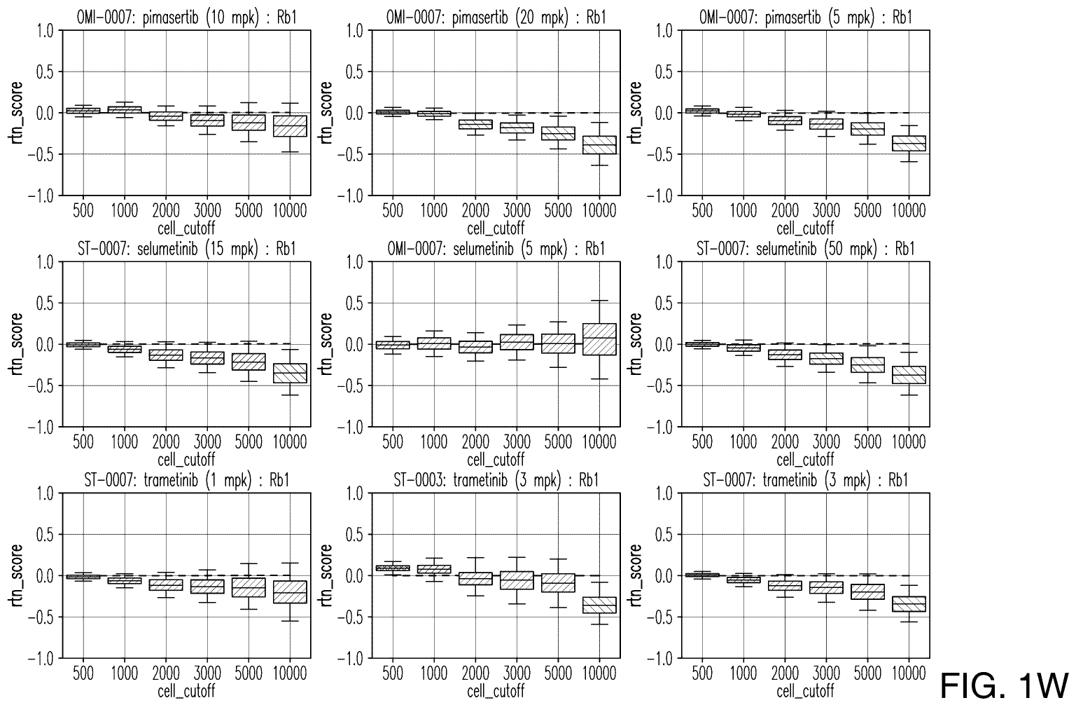

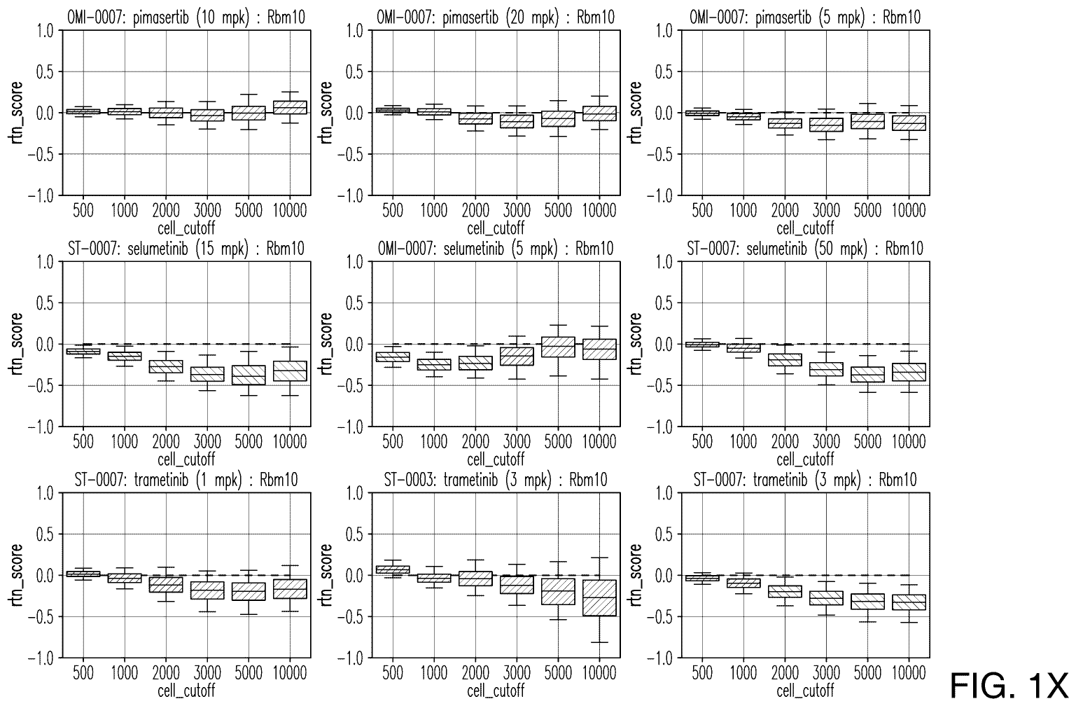

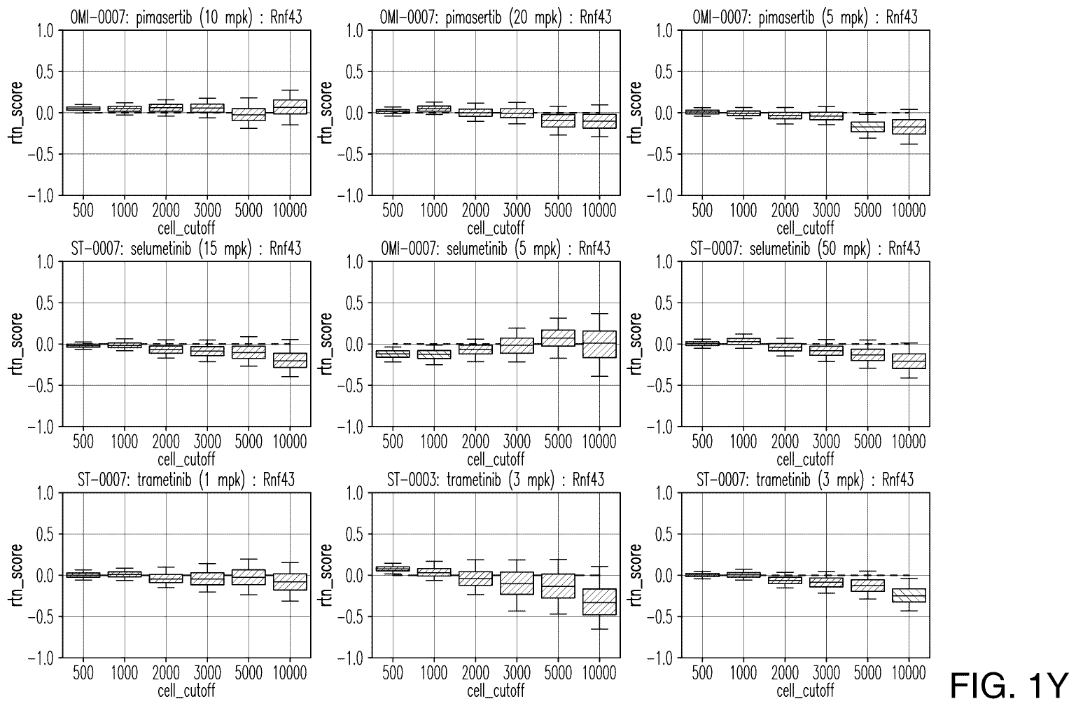

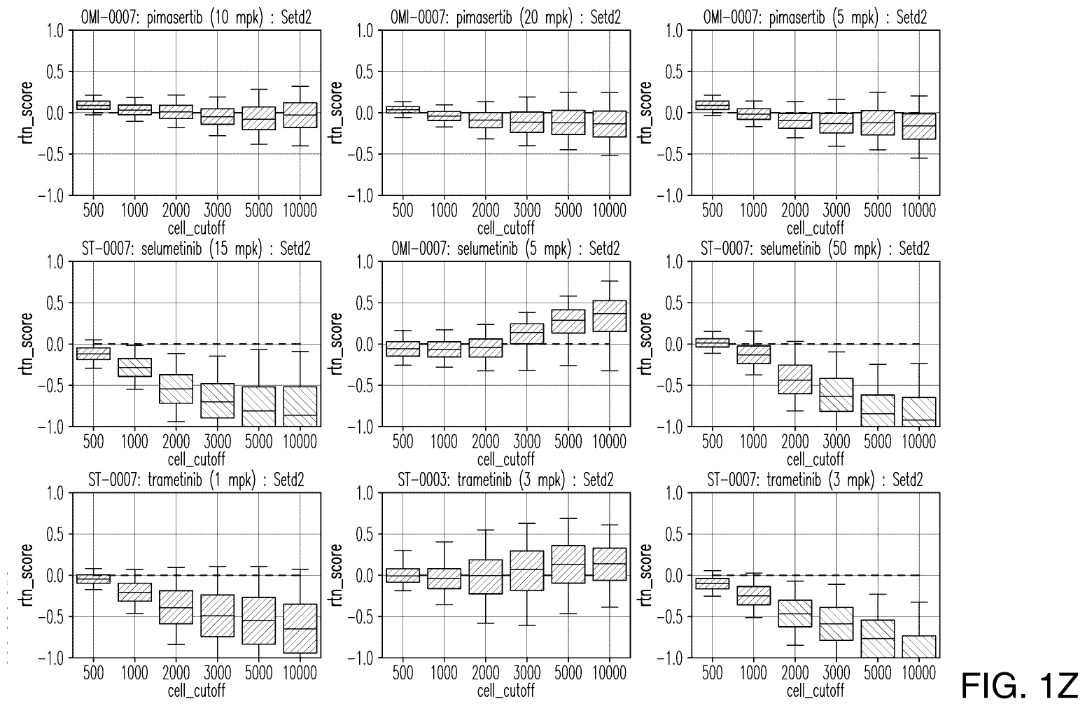

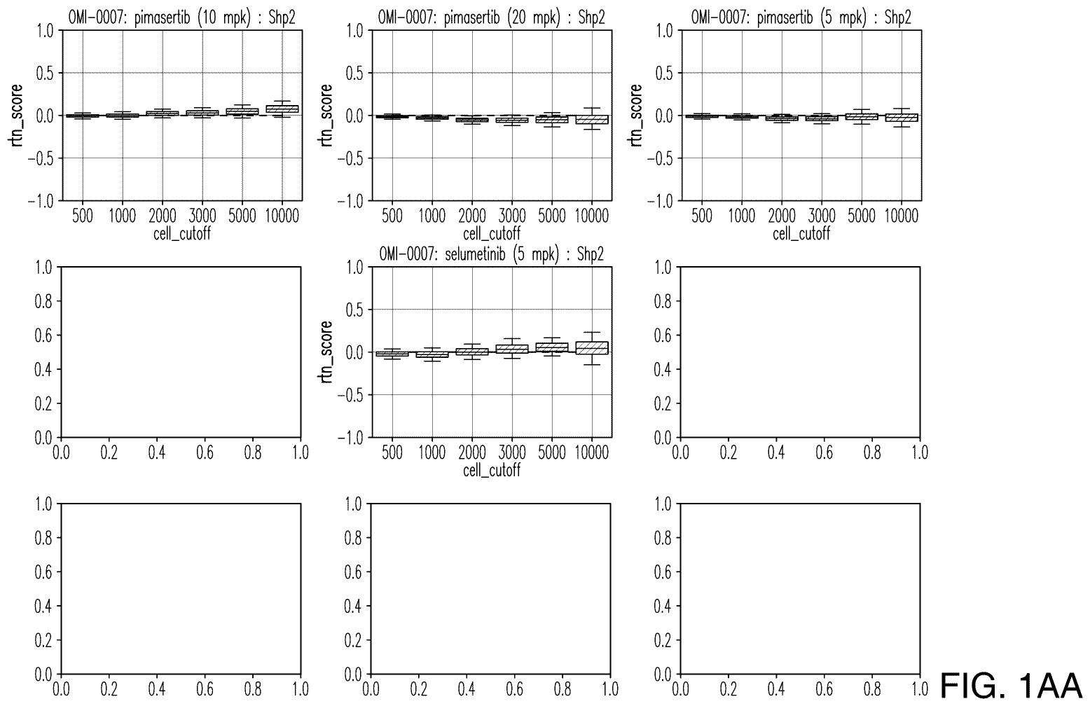

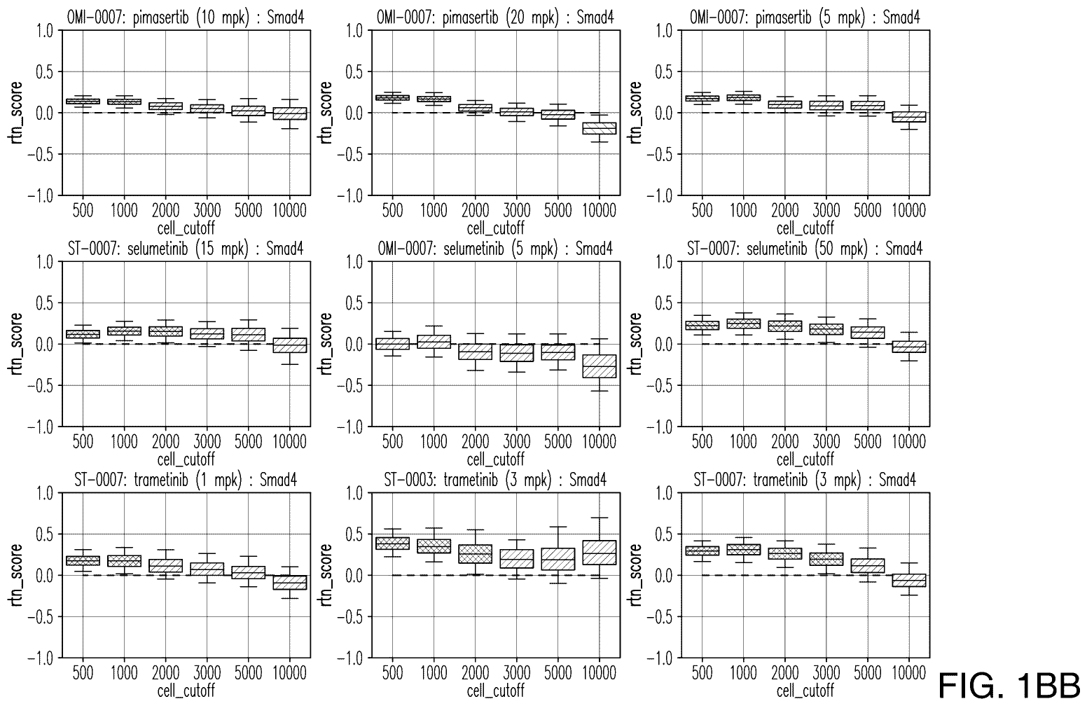

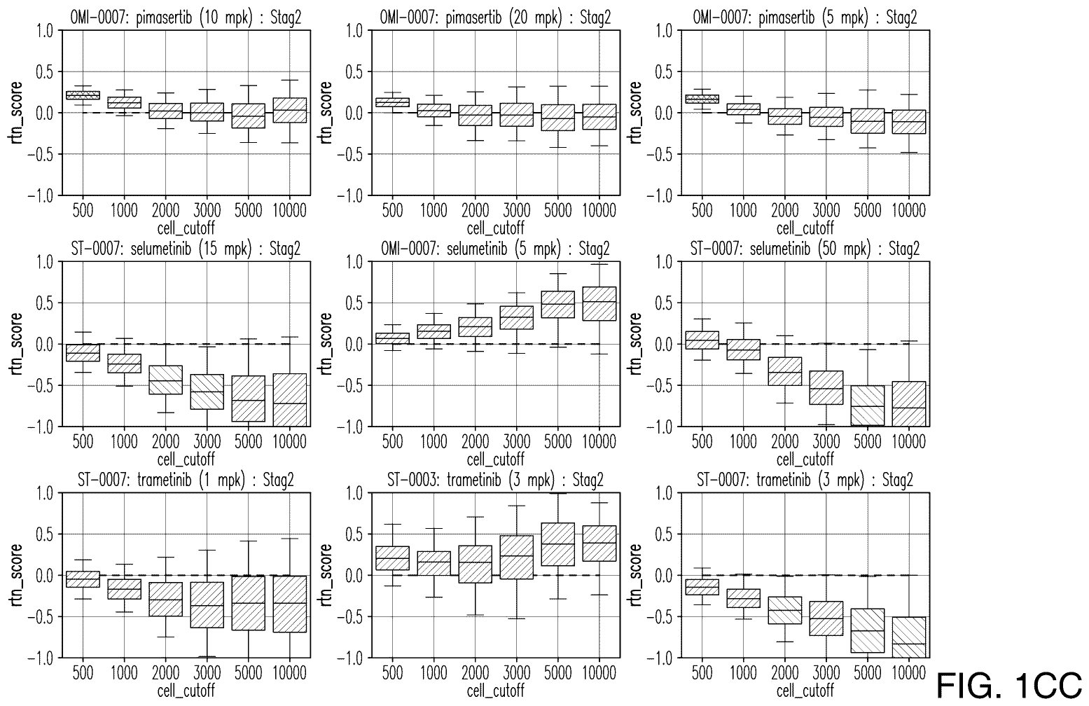

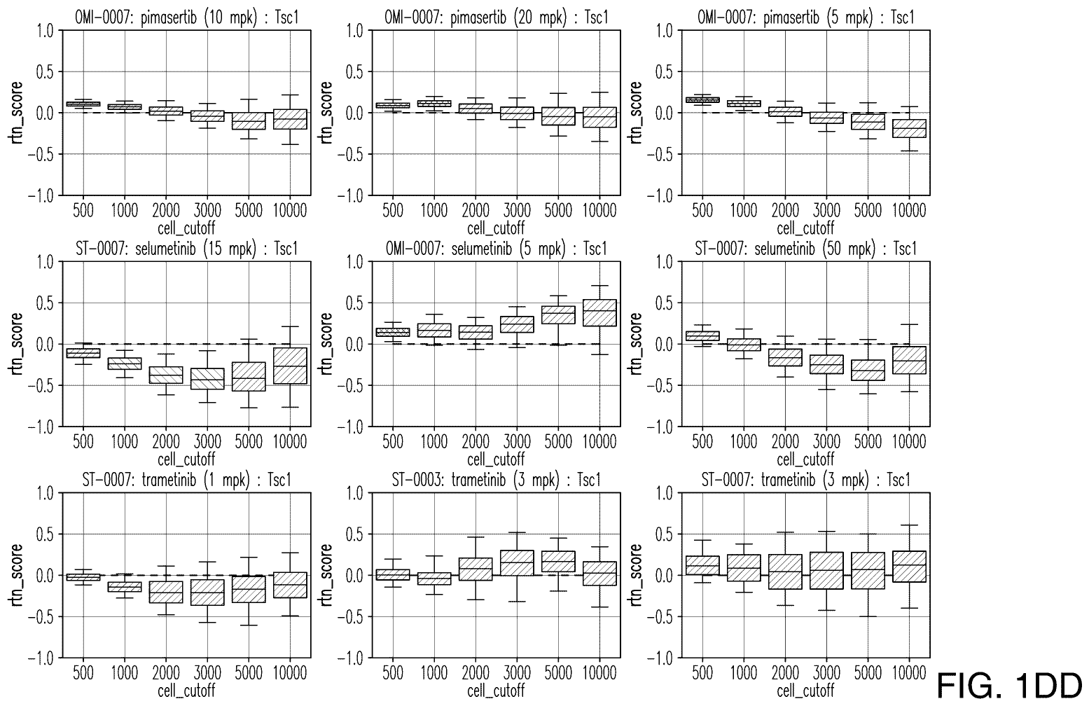

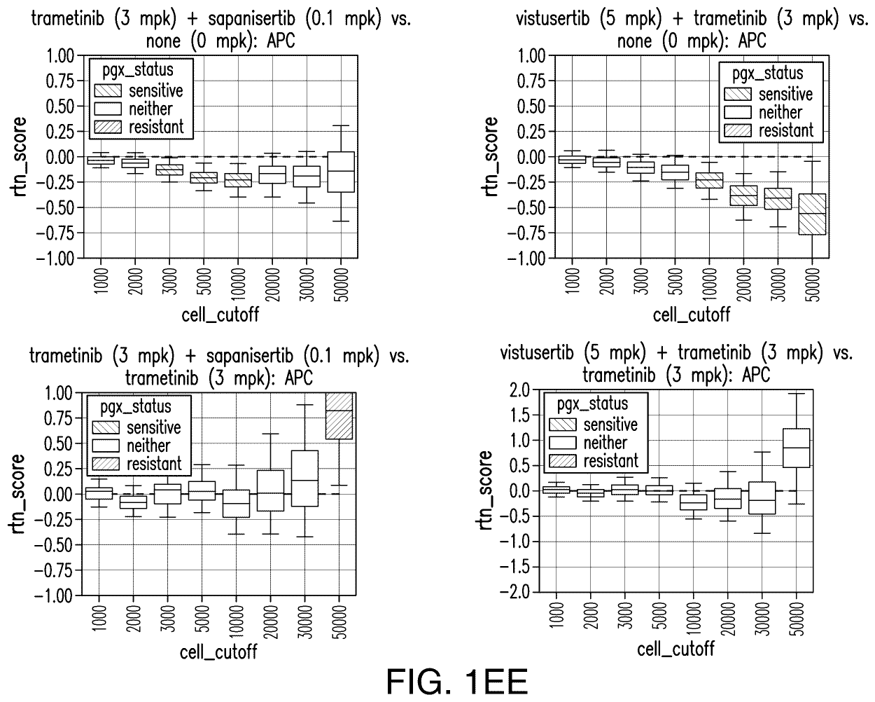

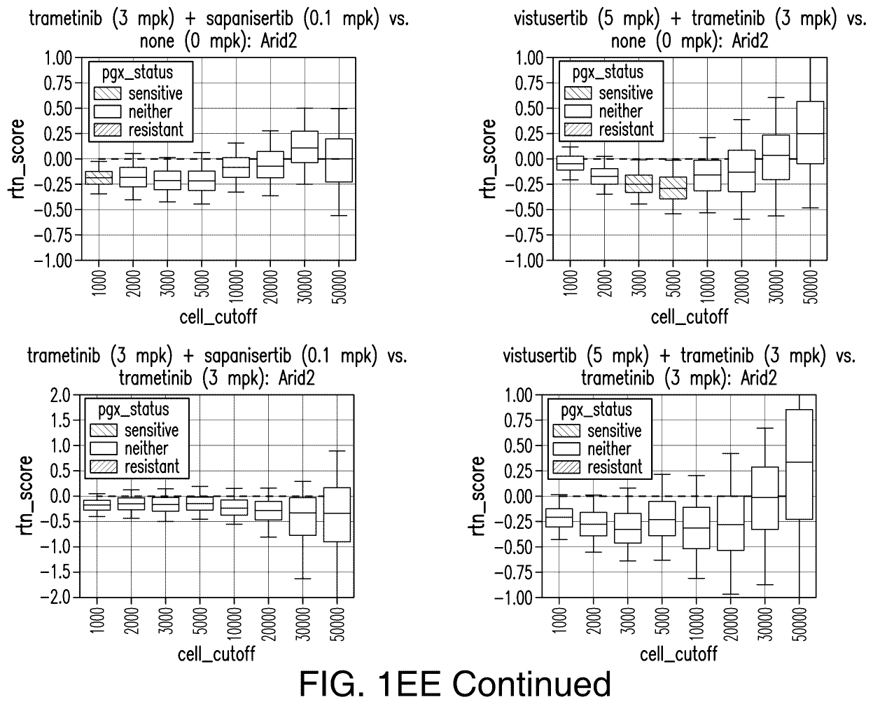

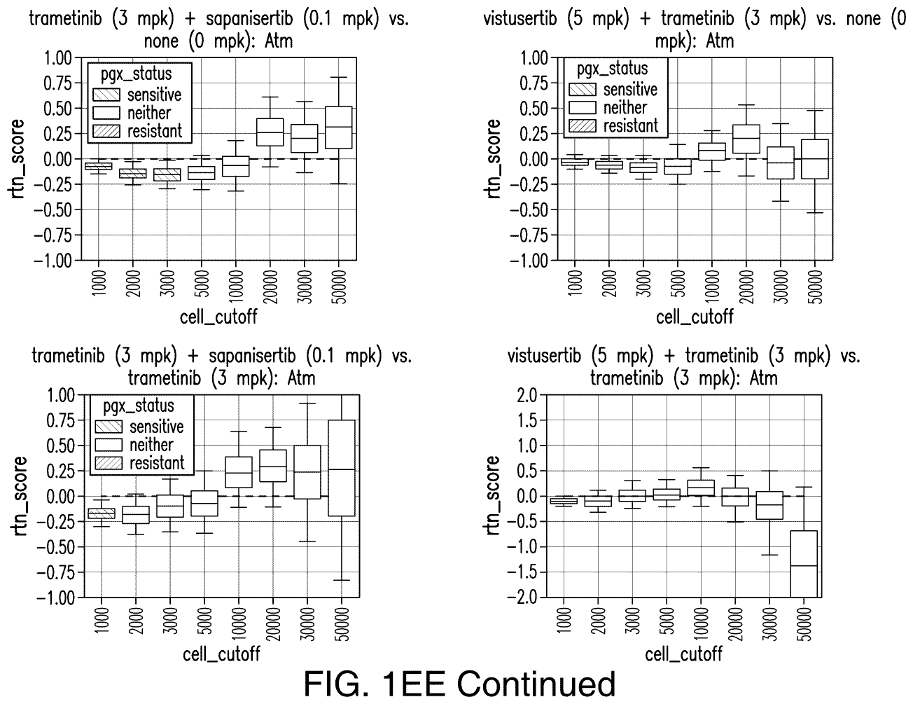

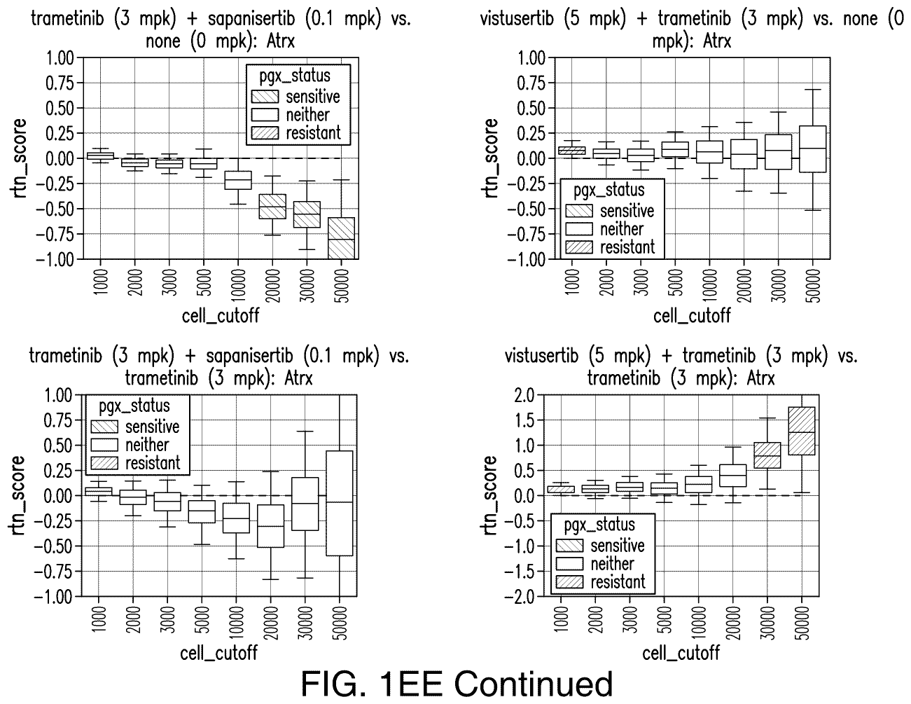

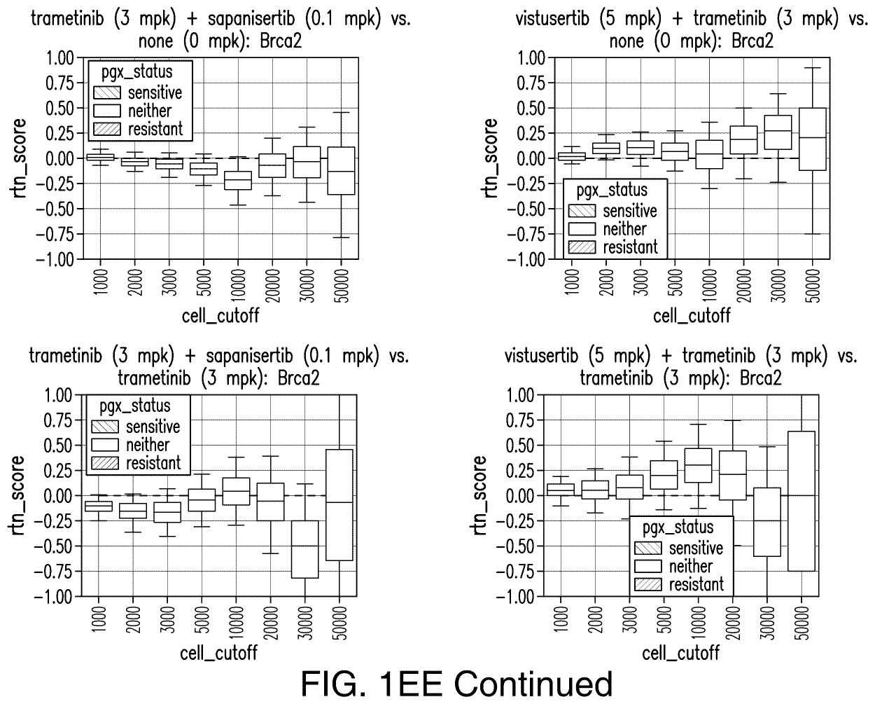

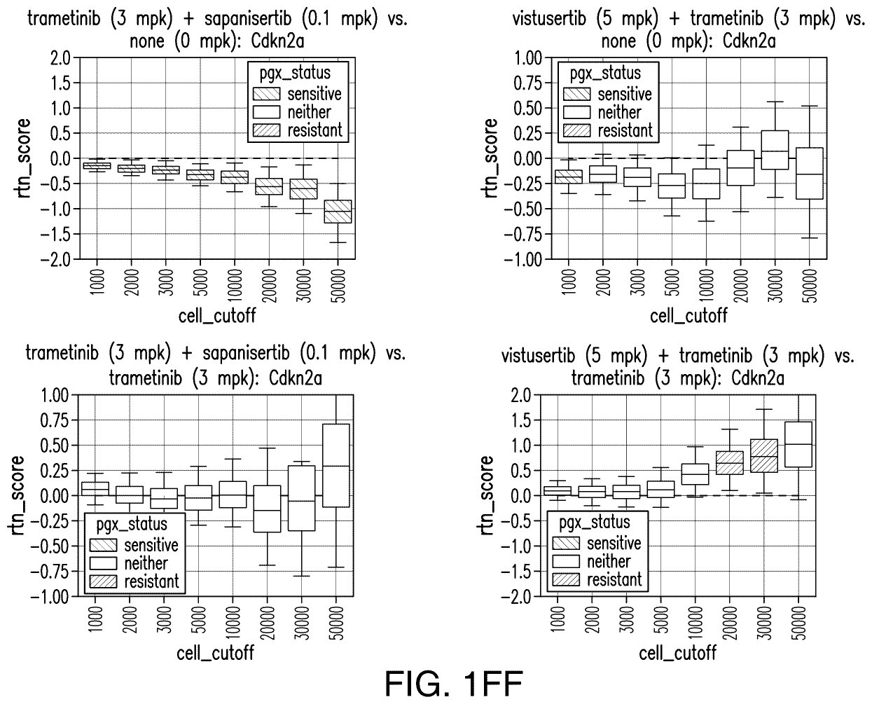

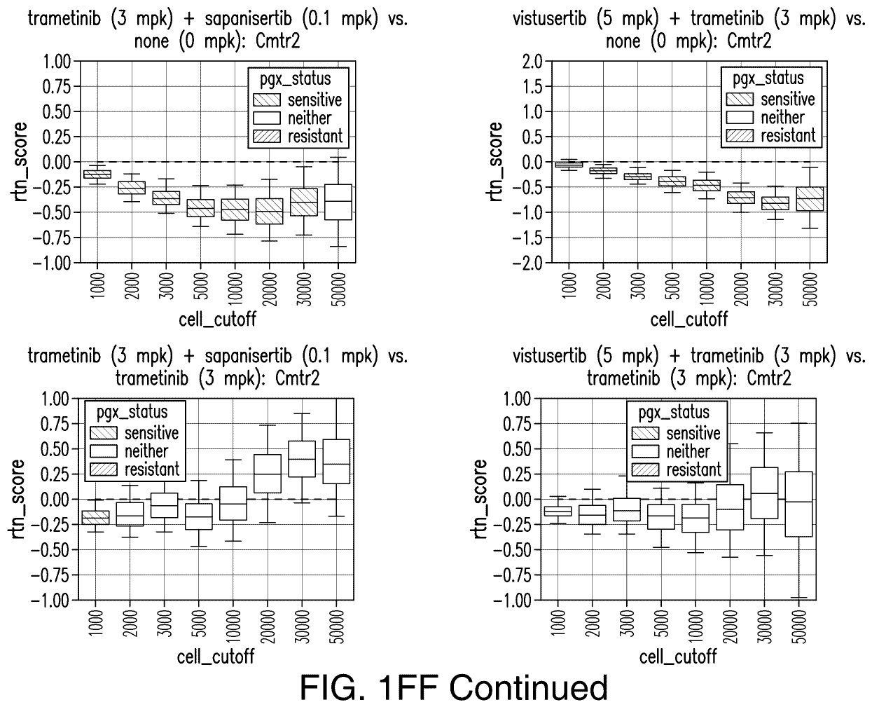

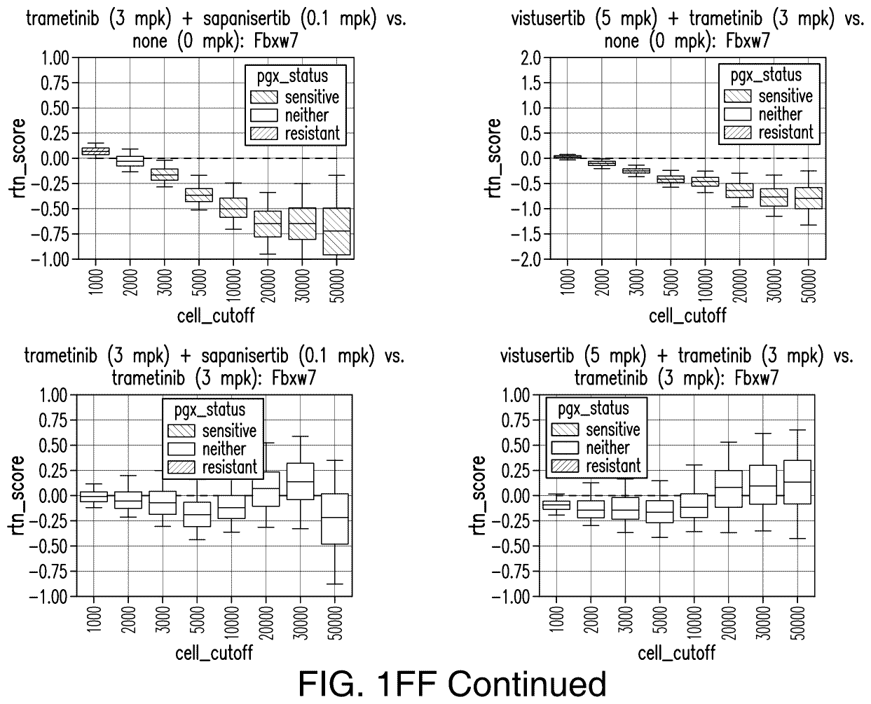

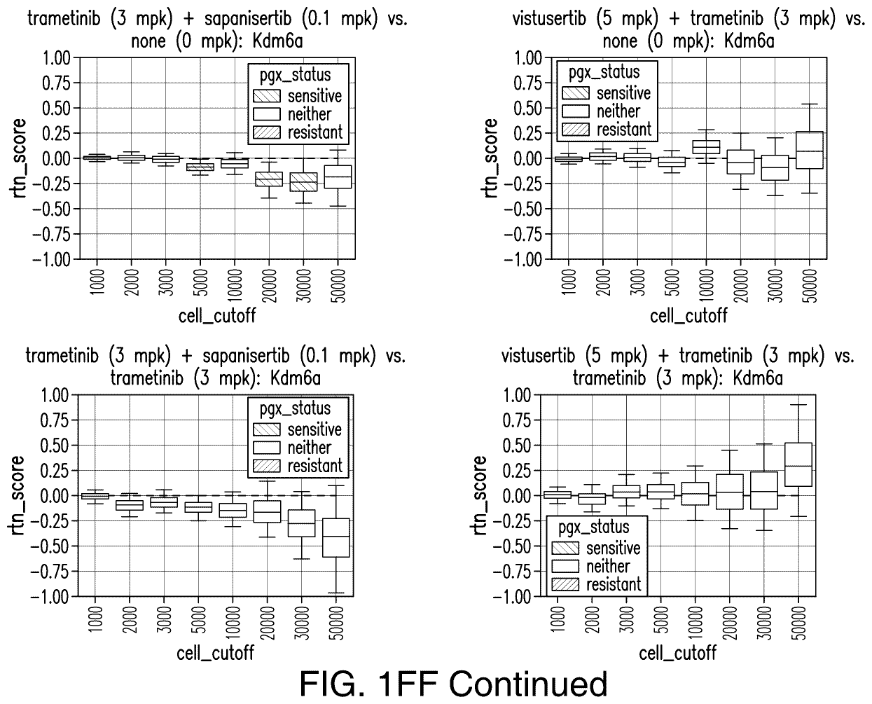

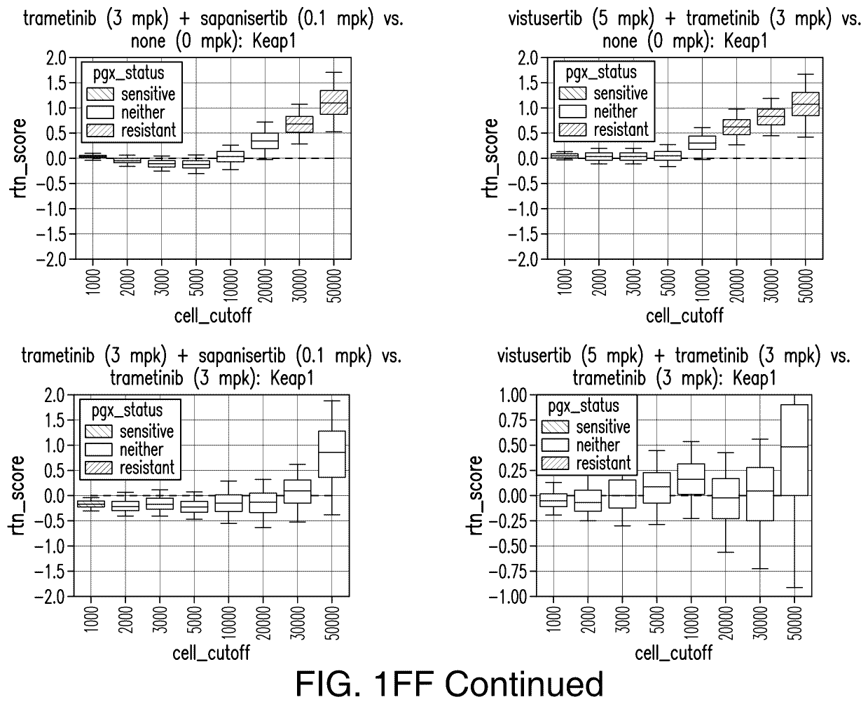

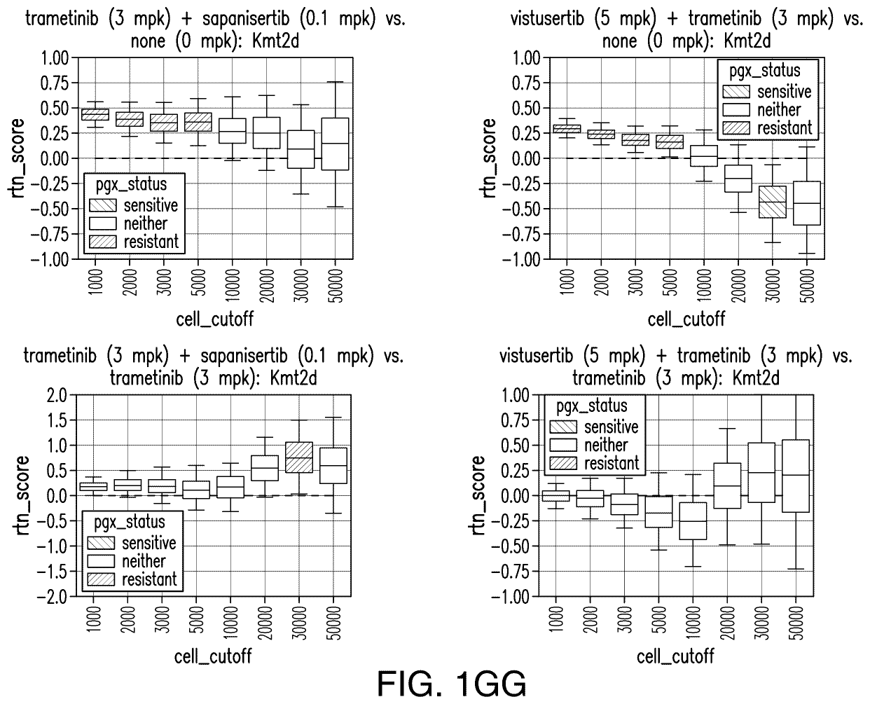

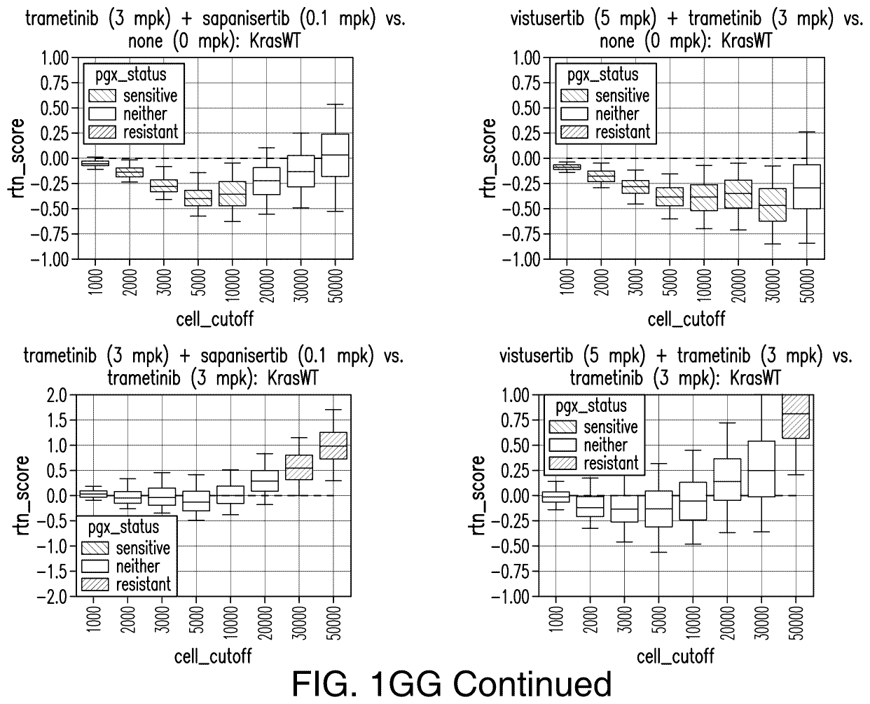

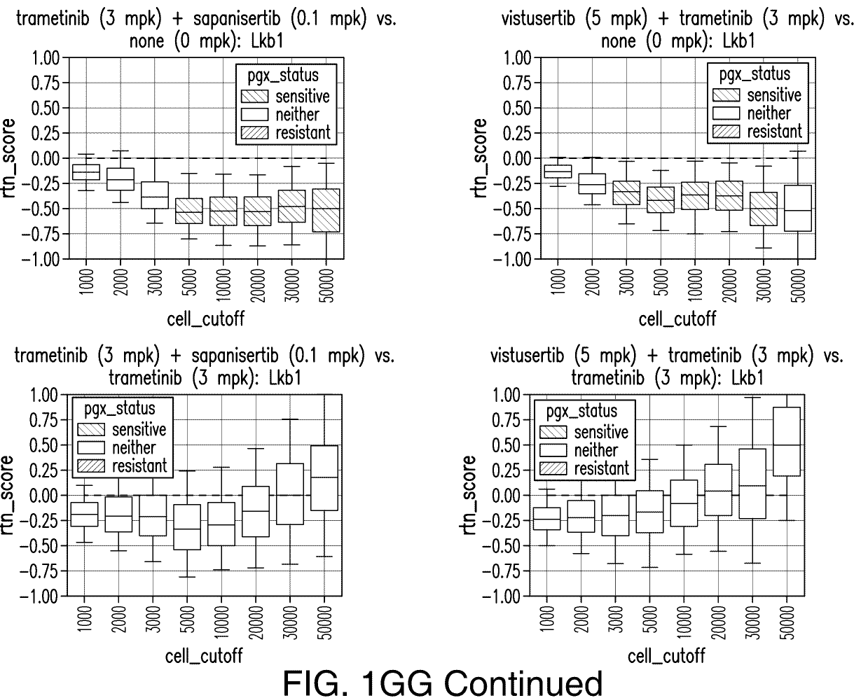

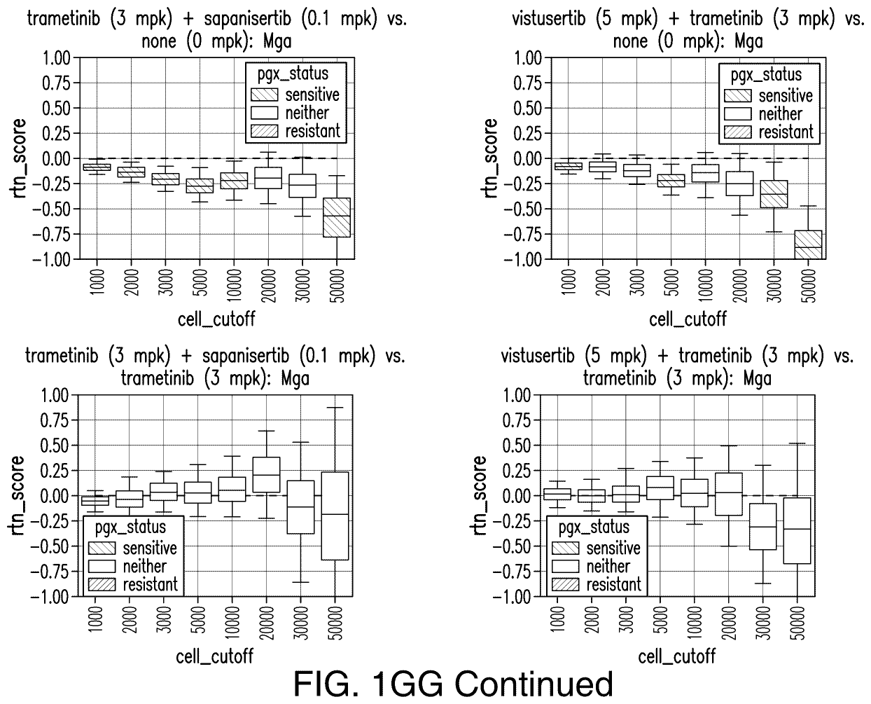

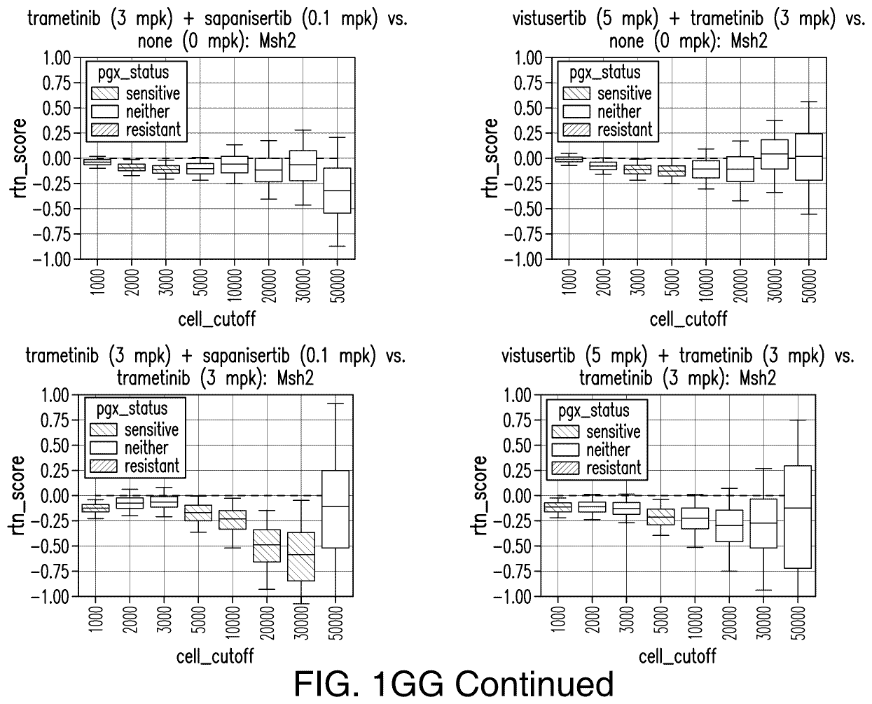

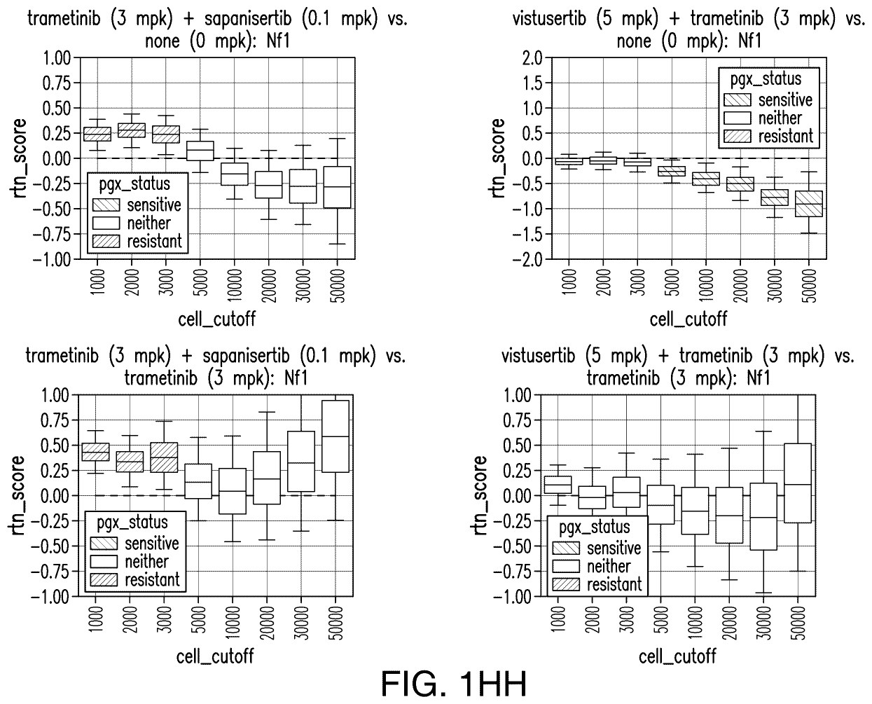

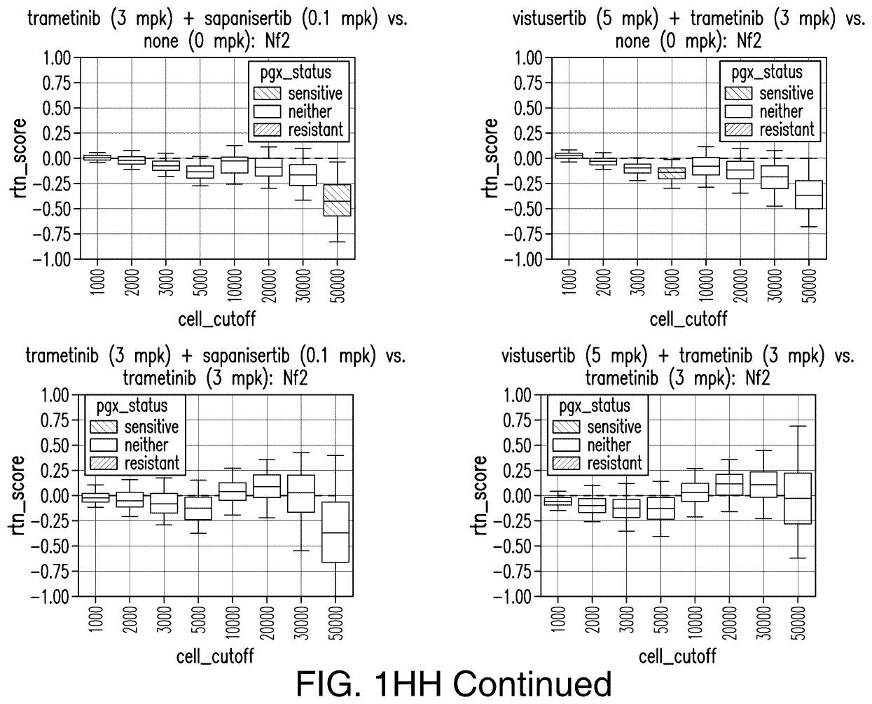

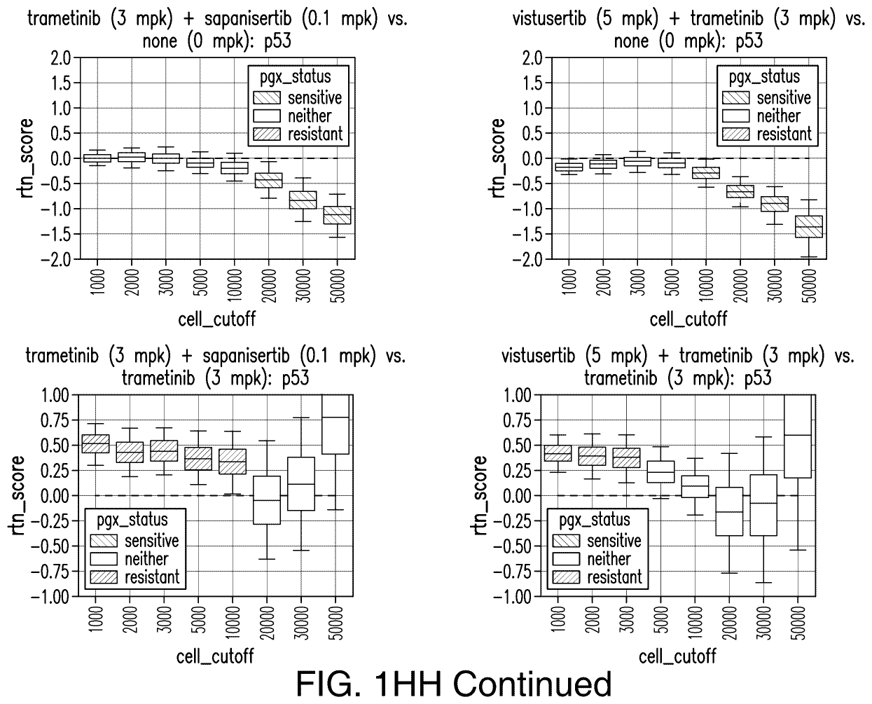

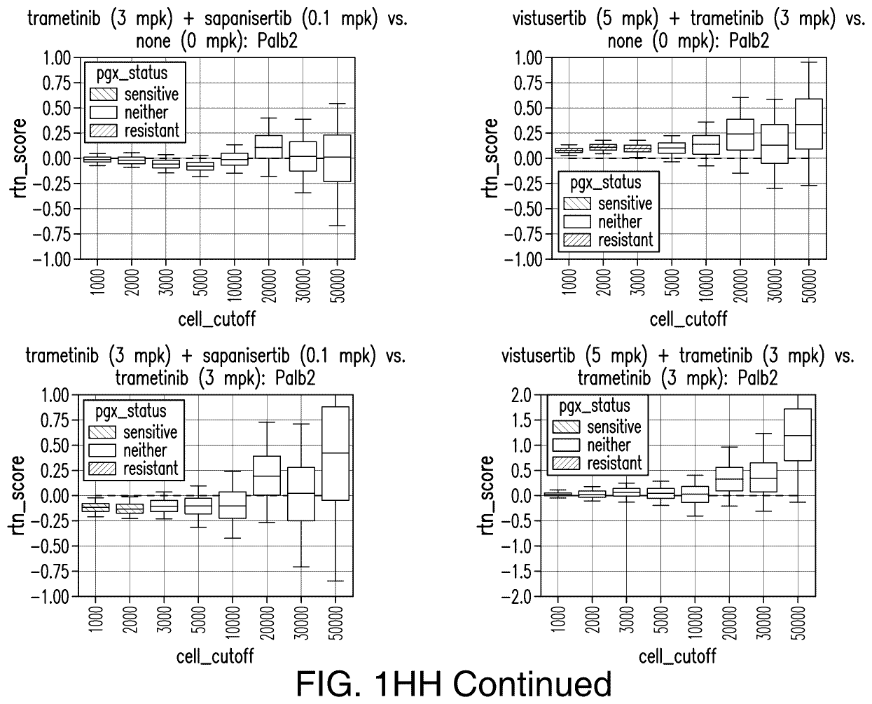

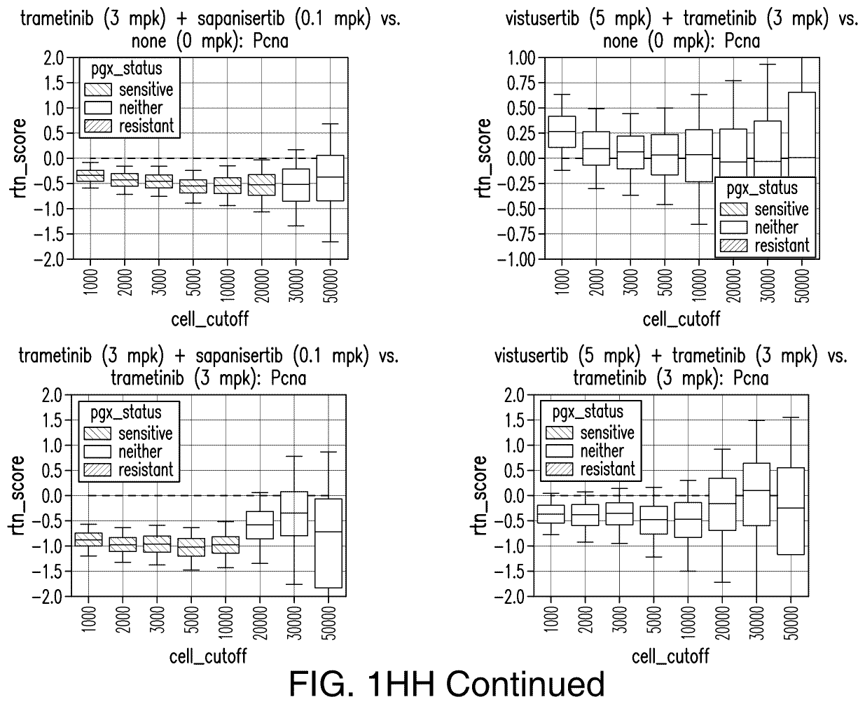

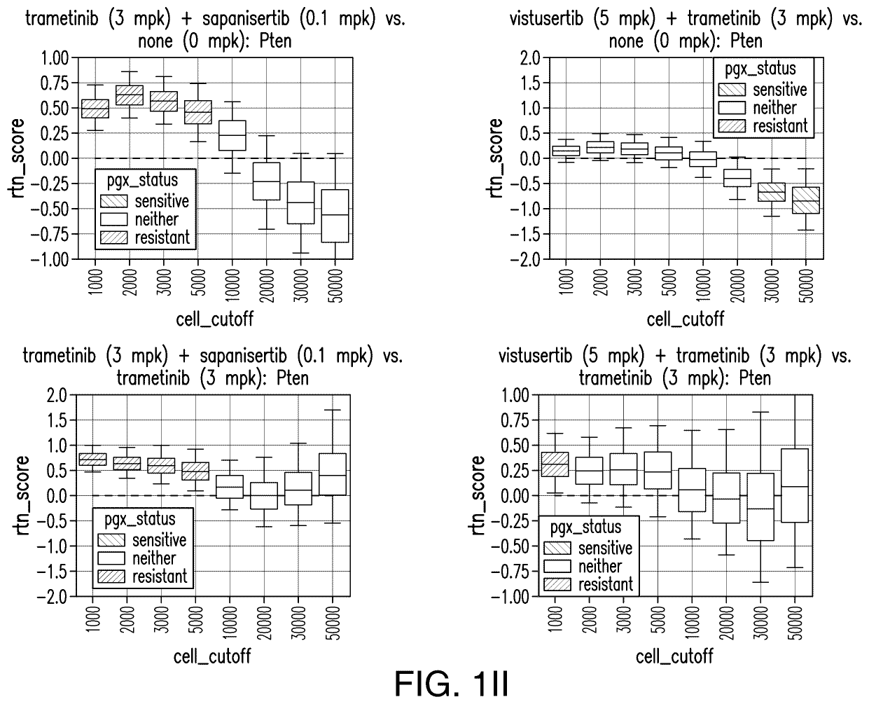

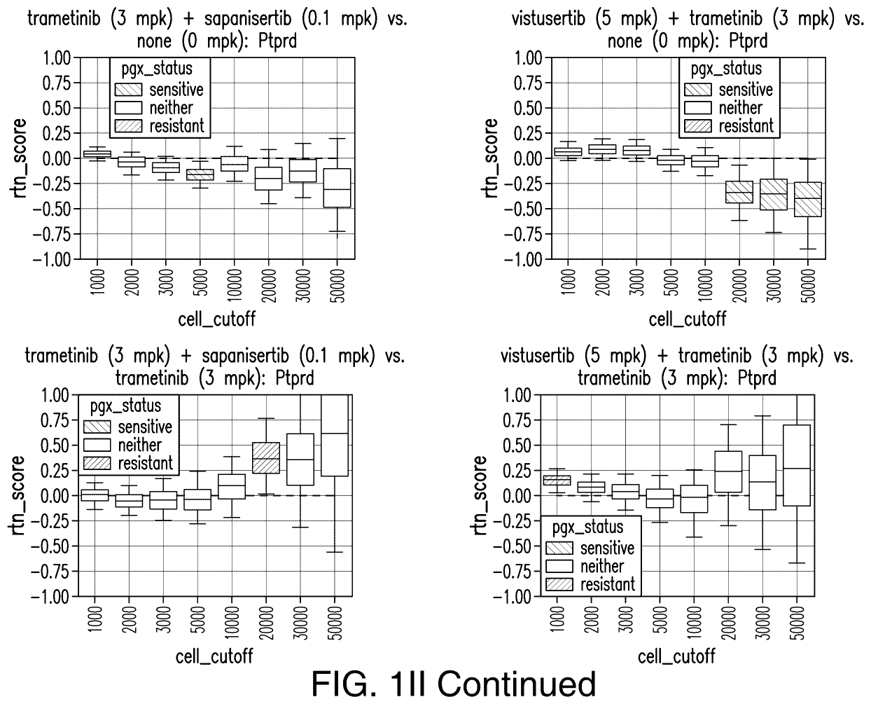

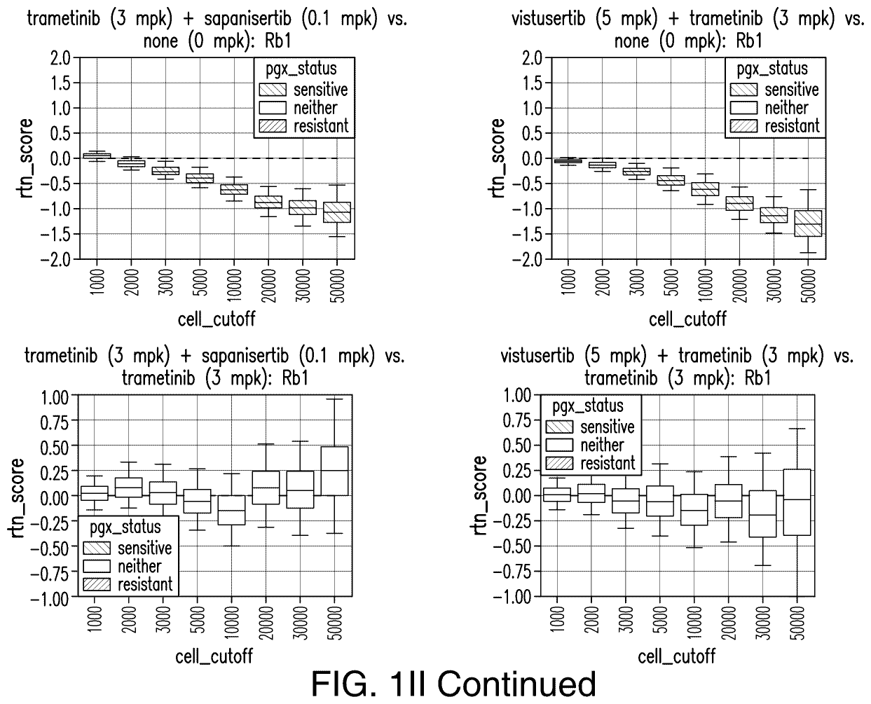

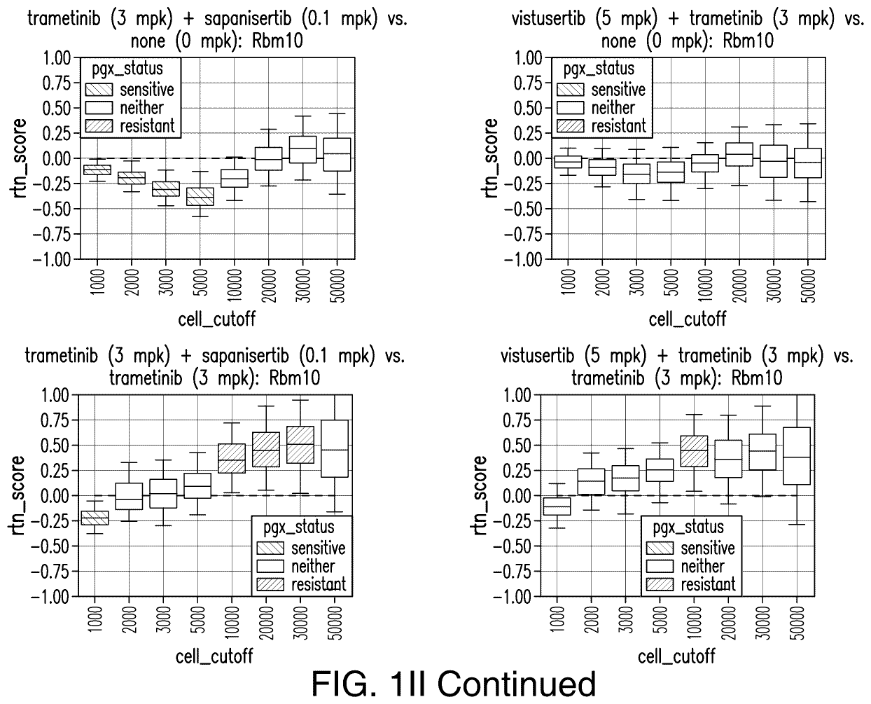

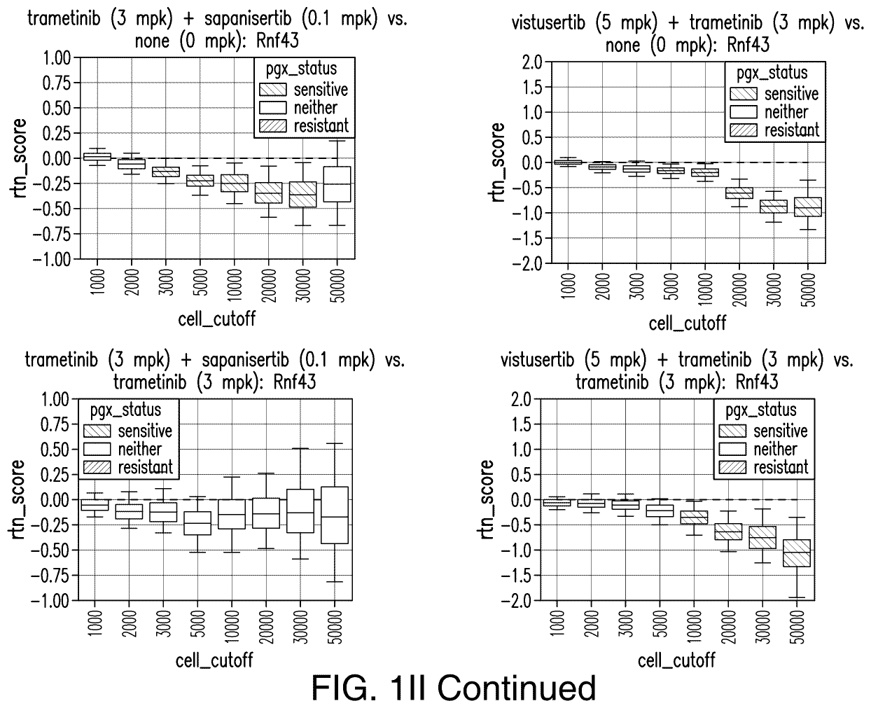

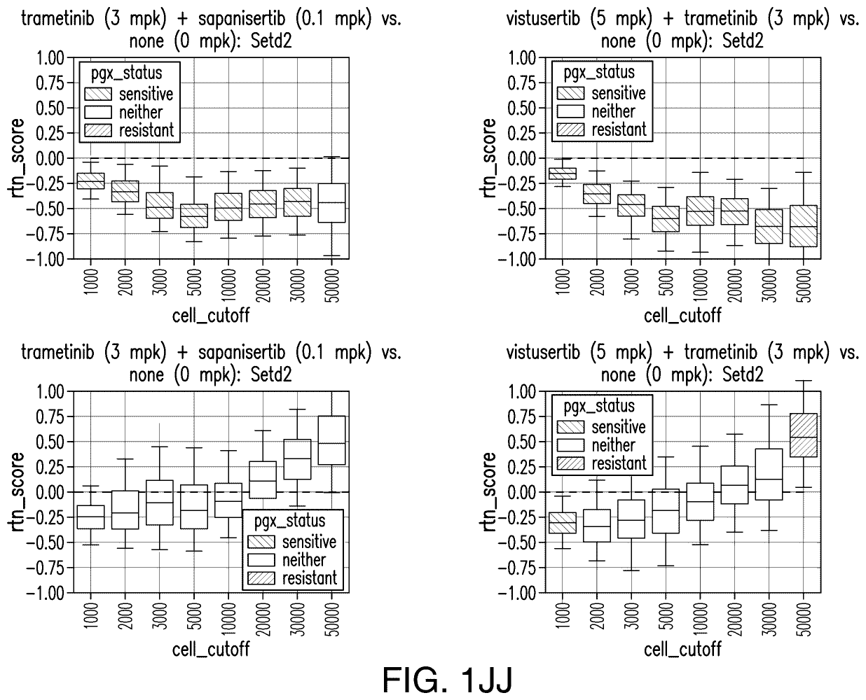

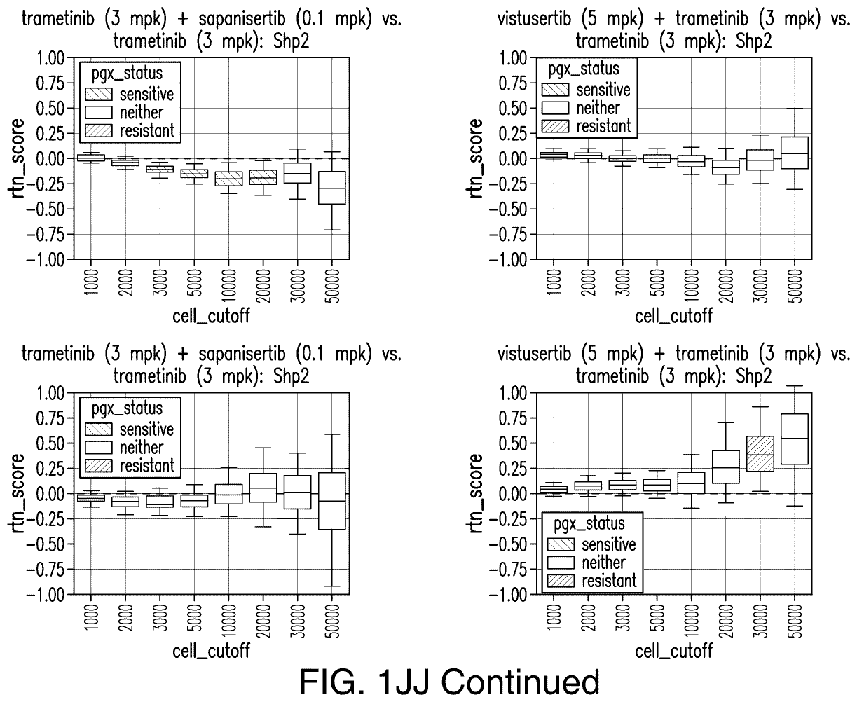

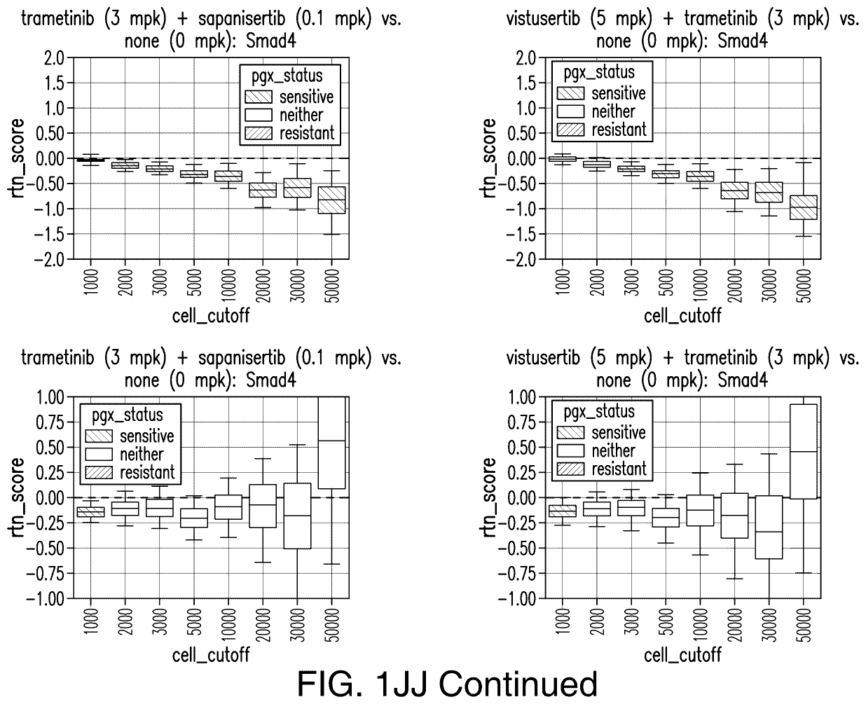

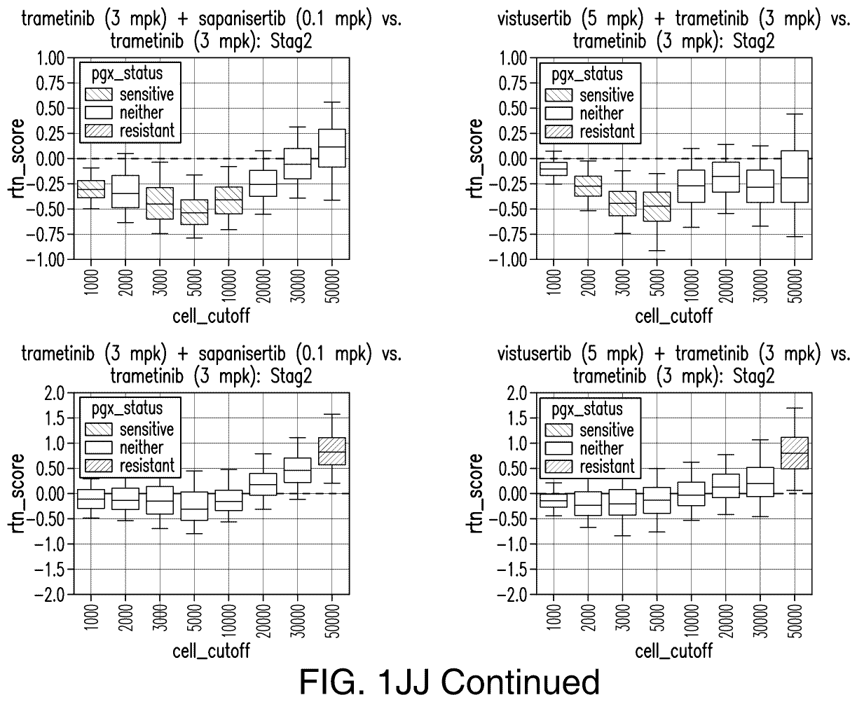

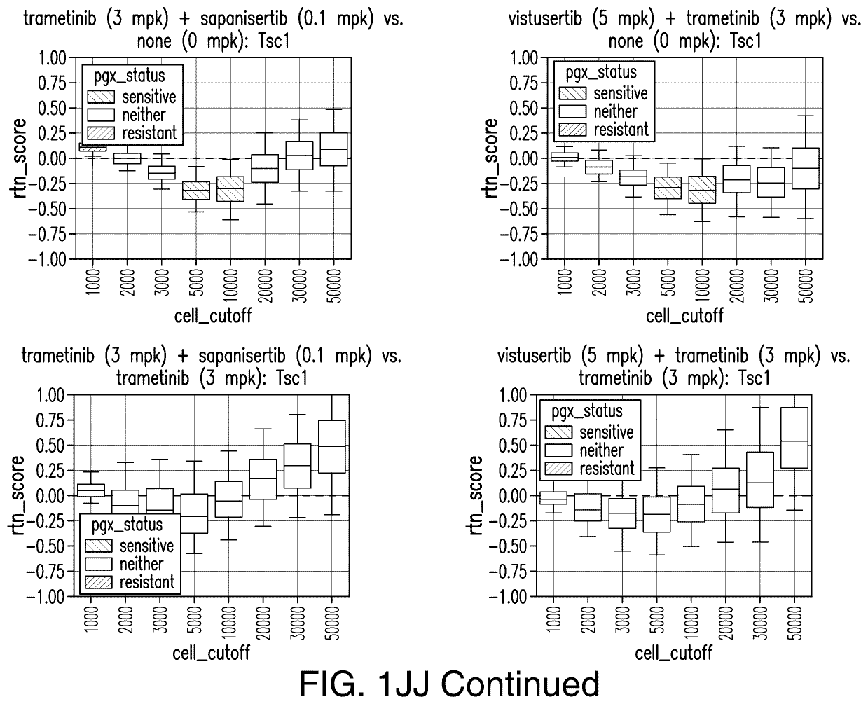

In order to generate confidence intervals for RTN_{score}, bootstrap re-samplings by (1) sampling mice with replacement from the control and therapy arms to match the original group sizes, and (2) sampling tumors (of all sizes) with replacement from each mouse were generated. For each mouse/tumor bootstrap, the RTN_{score} was re-computed. A genotype was then considered sensitive if the 95th % ile of these bootstrap RTN_{score} values fell below 0, or resistant if the 5th % ile exceeded 0. We performed this bootstrapping procedure at tumor size cutoffs ranging from L=300 cells up to L=10,000 cells. The results are shown in FIG. 1, which shows the RTN_{score} values for each twenty-two biomarker genes.

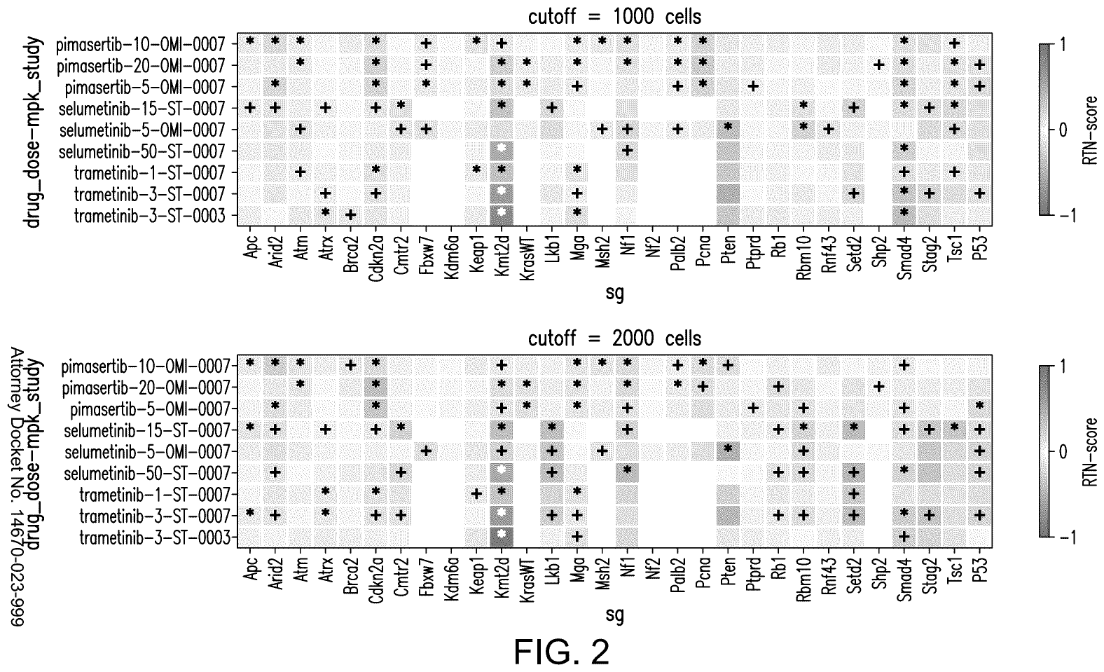

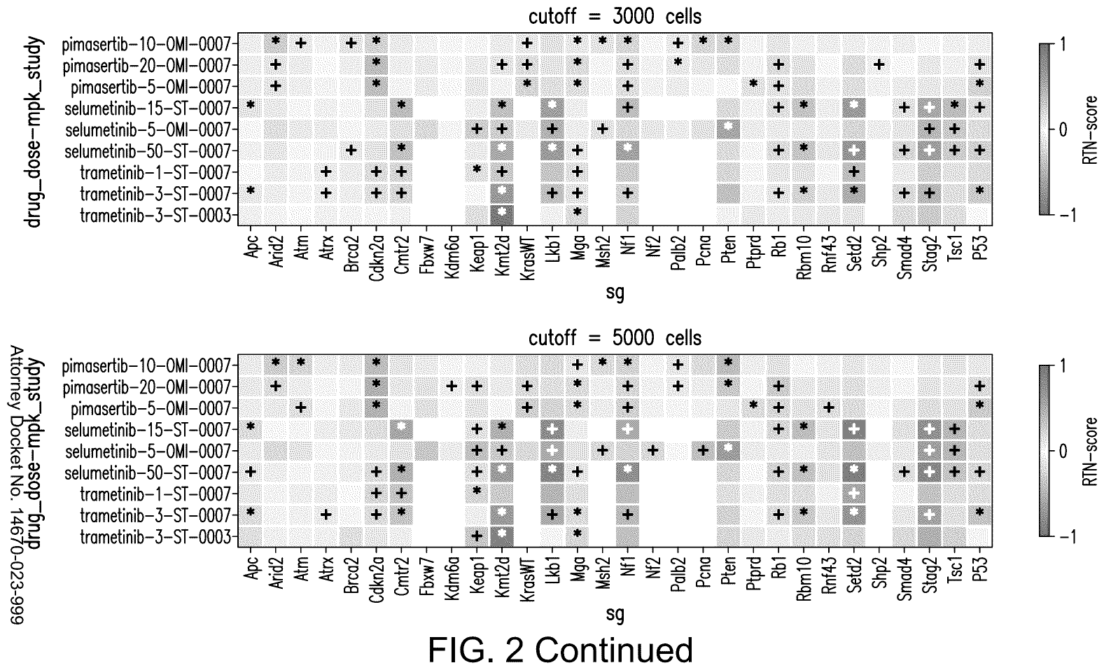

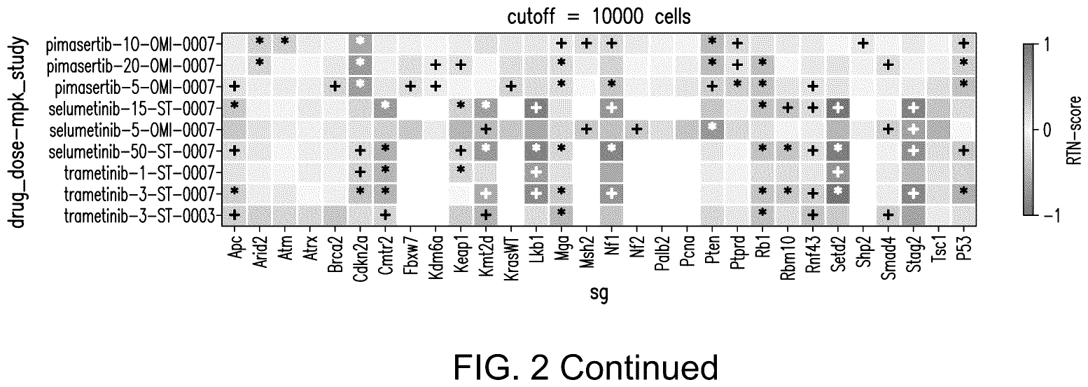

FIG. 2 shows a biomarker heatmap showing the study of pharmacogenomic interactions of MEKi with inactivation of tumor suppressor genes. Relative tumor number (RTN)>0 indicates drug resistance, and RTN=1 corresponds to 2× change in tumor number (larger than each cutoff) relative to change in untreated vs. treated for oncogene-only tumors, while RTN=−1 corresponds to 0.5× change and drug sensitivity. *: p<0.05 and +: p<0.2. Both p-values are two-tailed and based on fraction of bootstraps with RTN scores great or less than 0. Missing cells in heatmap correspond to genotypes that were not assayed in their particular study.

### Example 2. Biomarkers of Responsiveness to TORC Inhibitor Therapy and MEK/TORC Inhibitor Combination Therapies

This example describes the identification of tumor suppressor genes that are biomarkers of response to MEK/TORC inhibitor combination therapies through a method that integrates CRISPR/Cas9-based somatic genome engineering and molecular barcoding into established Cre/Lox-based genetically engineered mouse models of oncogenic Kras-driven lung cancer.

The experiments were performed according to the same protocols described in Example 1 above, with the following two differences: One, as was done for OMI-0007 described in Example 1, in addition to targeting the 22 tumor suppressor genes (Apc, Arid2, Atm, Atrx, Brca2, Cdkn2a, Cmtr2, Keap1, Kmt2d, Lkb1, Mga, Nf1, p53, Pten, Ptprd, Rb1, Rbm10, Rnf43, Setd2, Smad4, Stag2, and Tsc1) targeted in the experiments ST-0003 and ST-0007 described in Example 1, eight additional tumor suppressor genes (Fbxw7, Kdm6a, Kras, Msh2, Nf2, Palb2, Pcna, and Ptpn11) were targeted. Two, the following dosing schedule was used instead of the dosing schedule described in Example 1.

Drug Dosing

12 weeks post tumor initiation, mice were treated with the following:

OMI-0006
Vehicle (n=24) delivered PO, with once daily dosing (QD), seven consecutive days a week, for two weeks until takedown at 15 weeks post induction
TORC1/2 inhibitor Sapanisertib/TAK-228 (n=20) delivered PO, at 0.3 mg/kg with once daily dosing (QD), seven consecutive days a week, for two weeks until takedown at 14 weeks post induction
TORC1/2 inhibitor Sapanisertib/TAK-228 (n=20) delivered PO, at 0.1 mg/kg with once daily dosing (QD), seven consecutive days a week, for two weeks until takedown at 14 weeks post induction
TORC1/2 inhibitor Vistusertib (n=20) delivered PO, at 15 mg/kg with once daily dosing (QD), seven consecutive days a week, for two weeks until takedown at 14 weeks post induction
TORC1/2 inhibitor Vistusertib (n=20) delivered PO, at 5 mg/kg with once daily dosing (QD), seven consecutive days a week, for two weeks until takedown at 14 weeks post induction
MEK inhibitor Trametinib (n=16) delivered PO, at 3 mg/kg with once daily dosing (QD), seven consecutive days a week, for two weeks until takedown at 14 weeks post induction
MEK inhibitor Trametinib (n=20) delivered PO, at 3 mg/kg with once daily dosing (QD), seven consecutive days a week, and TORC1/2 inhibitor Sapanisertib/TAK-228 (n=20) delivered PO, at 0.1 mg/kg with once daily dosing (QD), seven consecutive days a week, for two weeks until takedown at 14 weeks post induction
MEK inhibitor Trametinib (n=20) delivered PO, at 3 mg/kg with once daily dosing (QD), seven consecutive days a week, and TORC1/2 inhibitor Vistusertib (n=20) delivered PO, at 5 mg/kg with once daily dosing (QD), seven consecutive days a week, for two weeks until takedown at 14 weeks post induction
MEK inhibitor Trametinib (n=21) delivered PO, at 3 mg/kg with once daily dosing (QD), seven consecutive days a week, for three weeks until takedown at 15 weeks post induction

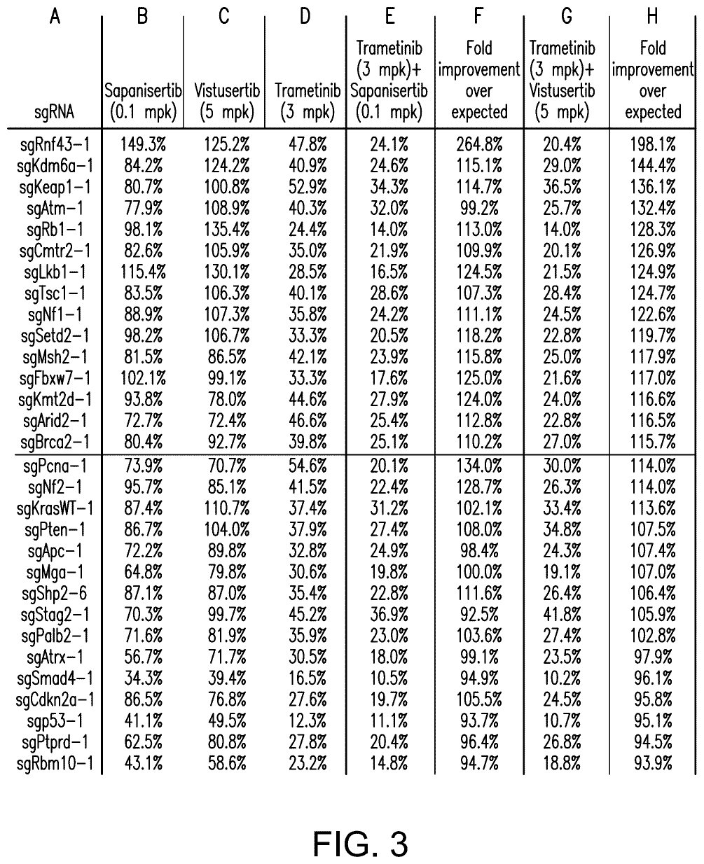

FIG. 3 shows a table depicting benefits of MEKi/TORCi combination therapy in 30 distinct genotypes. Columns B, C, D, E, and G represent average total neoplastic cell count for mice given each respective therapy relative to vehicle controls. Columns F and H represent the fold improvement in efficacy above what one would expect from the combined product of the efficacy from each monotherapy arm.

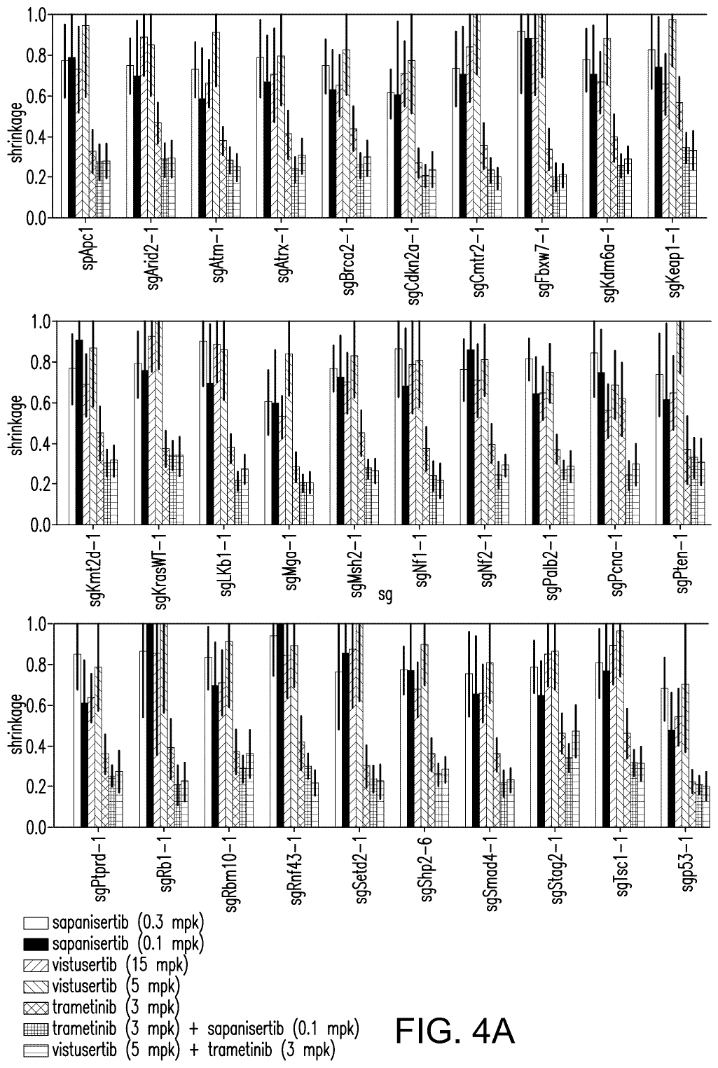

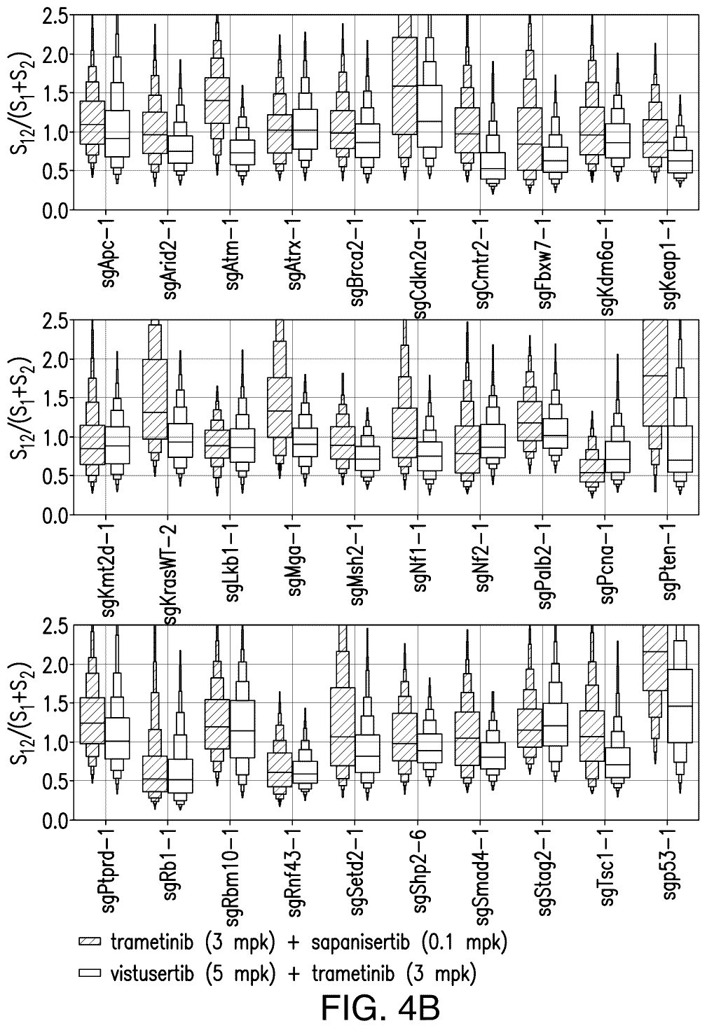

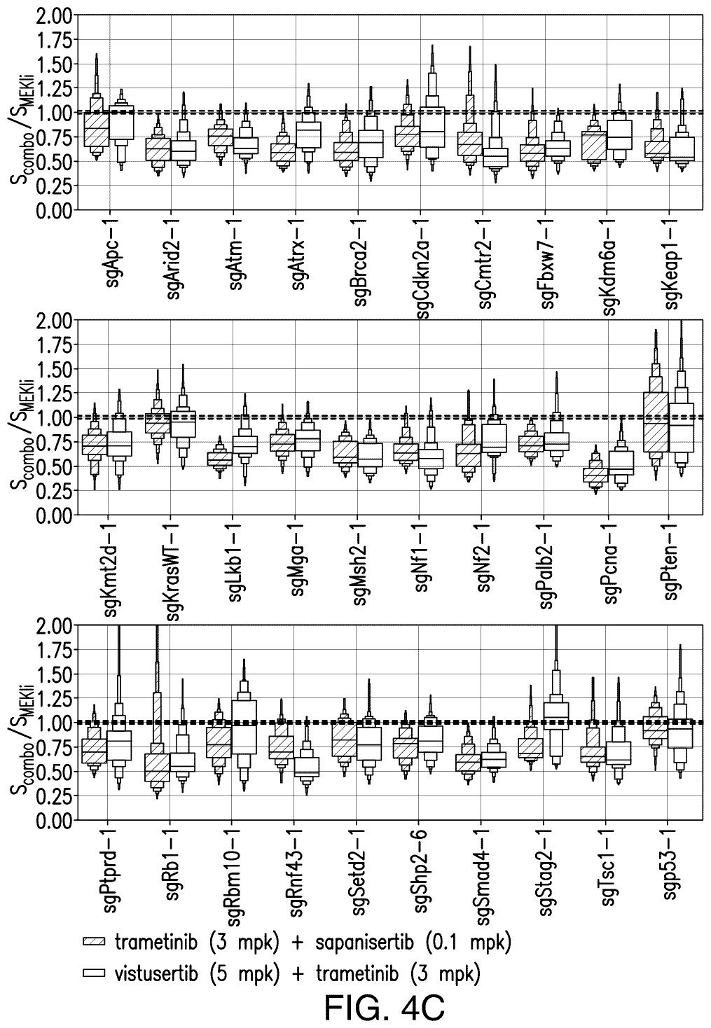

1000 bootstrap resamplings of mice were performed and the median neoplastic cell count for each tumor genotype in each study group was computed. Then, for each bootstrap, the ratio of neoplastic cell counts of each drug group relative to the control group was calculated, which is referred to as the “shrinkage.” The distribution of shrinkages for each drug group is shown in FIG. 4(A) Then, to show the effect of TORCi/MEKi combination therapies relative to monotherapies, the distribution over bootstraps of the ratio of TORCi/trametinib shrinkages is shown relative to trametinib monotherapy shrinkages (FIG. 4(B)), and the product of the corresponding TORCi/trametinib monotherapy shrinkages (FIG. 4(C)).

### Example 3. Biomarkers of Responsiveness to MEK Inhibitor/Chemotherapy Combination Therapies

This example describes the identification of tumor suppressor genes that are biomarkers of response to MEK inhibitor/chemotherapy combination therapies through a method that integrates CRISPR/Cas9-based somatic genome engineering and molecular barcoding into established Cre/Lox-based genetically engineered mouse models of oncogenic Kras-driven lung cancer.

The experiments were performed according to the same protocols described in Example 1 above, with the following two differences: Fifty-nine (59) tumor suppressor genes were targeted in this study (OMI-0015): Apc, Arid1a, Arid2, Asxl1, Atm, Atrx, Bap1, Brca1, Brca2, Cdkn2a, Cic, Cmtr2, Cul3, Dicer1, Dlc1, Dusp4, Ep300, Fat1, Fbxw7, Kdm5c, Kdm6a, Keap1, Kmt2c, Kmt2d, Kras, Lrp1b, Mga, Msh2, Mtap, Ncoa6, Nf1, Nf2, Palb2, Pbrm1, Pcna, Pten, Ptpn11, Ptpn13, Ptprd, Ptprs, Rasa1, Rb1, Rb1cc1, Rbm10, Rnf43, Setd2, Smad2, Smad4, Smarca4, Smg1, Stag2, Stk11, Tet2, Tgfbr2, Tp53, Tsc1, Tsc2, Usp15, and Zfhx3. Two, the following dosing schedule was used instead of the dosing schedule described in Example 1.

Drug Dosing

12 weeks post tumor initiation, mice were treated with the following:

OMI-0015
Vehicle (n=30) delivered PO, with once daily dosing (QD), every two days, for three weeks until takedown at 15 weeks post induction
Pimasertib (n=20) delivered PO, at 5 mg/kg with twice daily dosing (BID), seven consecutive days a week, for three weeks until takedown at 15 weeks post induction
Docetaxel (n=20) delivered IP, at 6 mg/kg with once daily dosing (QD), every 4 days, for three weeks until takedown at 15 weeks post induction and Pimasertib (n=20) delivered PO, at 5 mg/kg with twice daily dosing (BID), seven consecutive days a week, for three weeks until takedown at 15 weeks post induction
WX-554 (n=20) delivered PO, at 10 mg/kg with once daily dosing (QD), every two days, for three weeks until takedown at 15 weeks post induction
WX-554 (n=20) delivered PO, at 5 mg/kg with once daily dosing (QD), every two days, for three weeks until takedown at 15 weeks post induction
Docetaxel (n=20) delivered IP, at 6 mg/kg with once daily dosing (QD), every 4 days, for three weeks until takedown at 15 weeks post induction and WX-554 (n=20) delivered PO, at 10 mg/kg with once daily dosing (QD), every two days, for three weeks until takedown at 15 weeks post induction

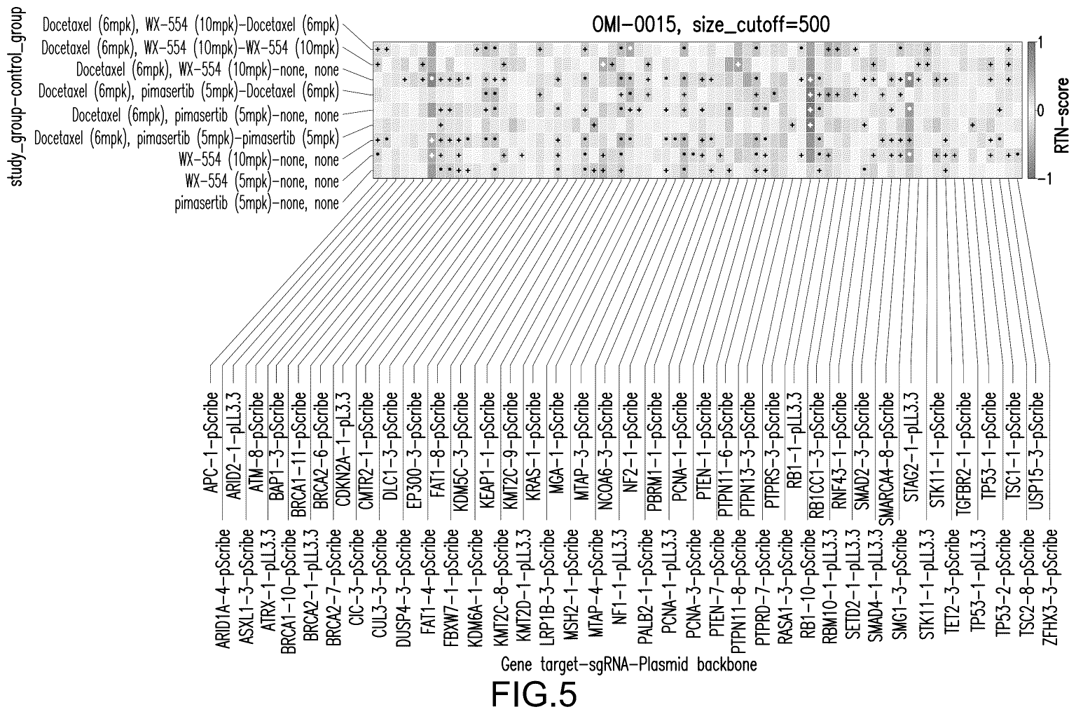

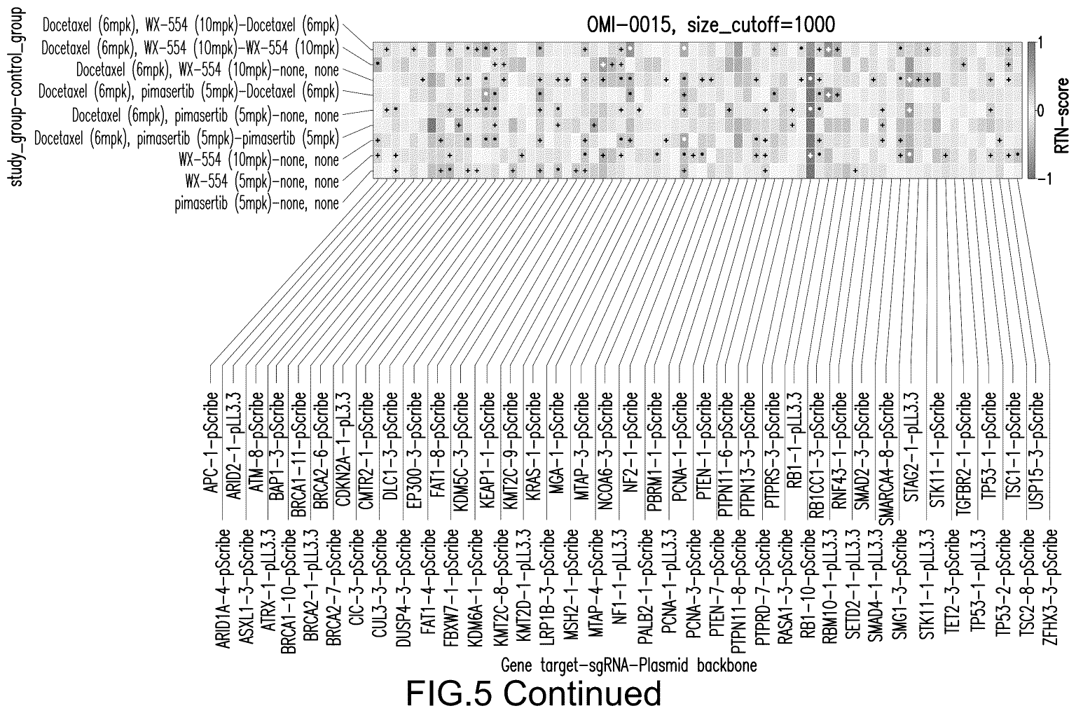

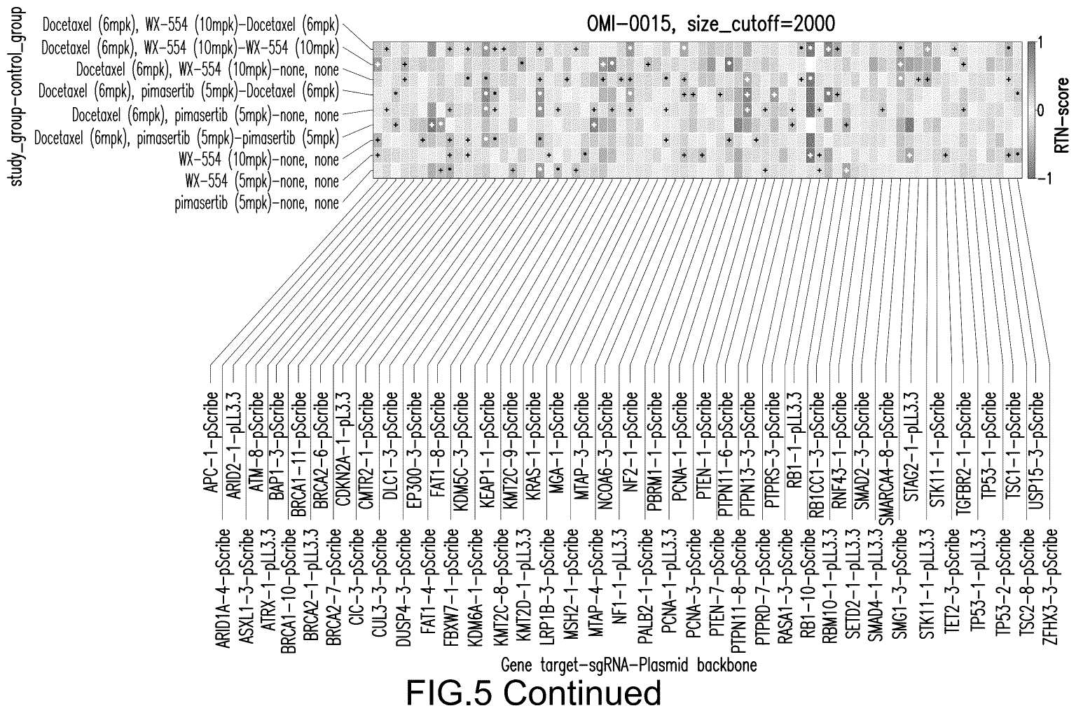

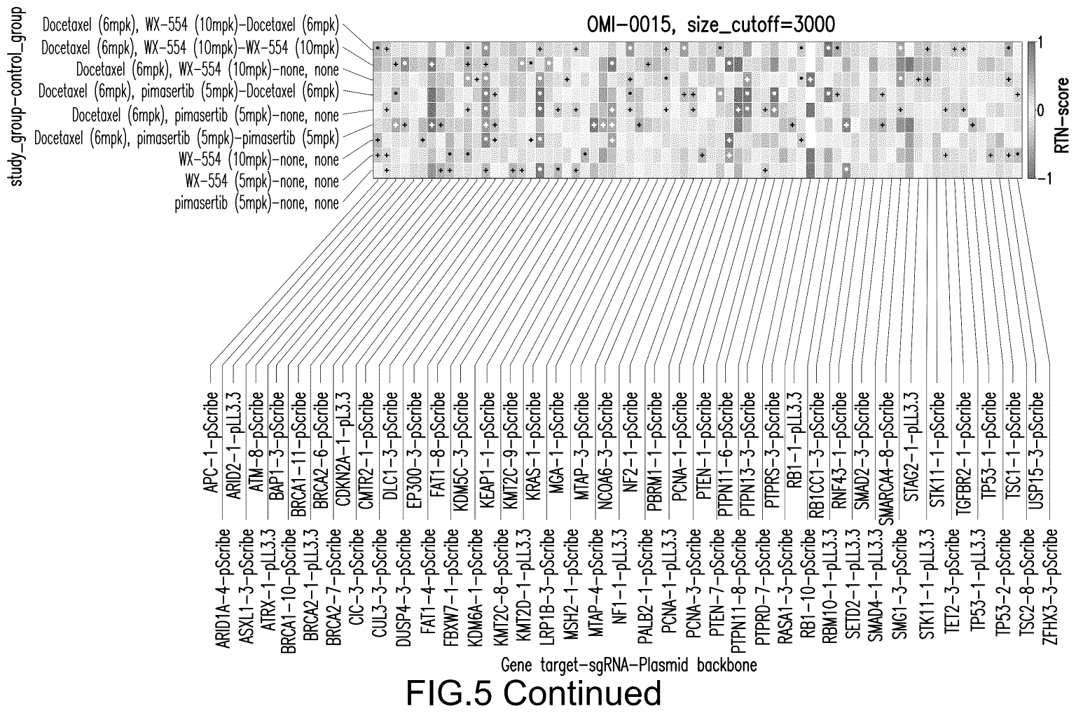

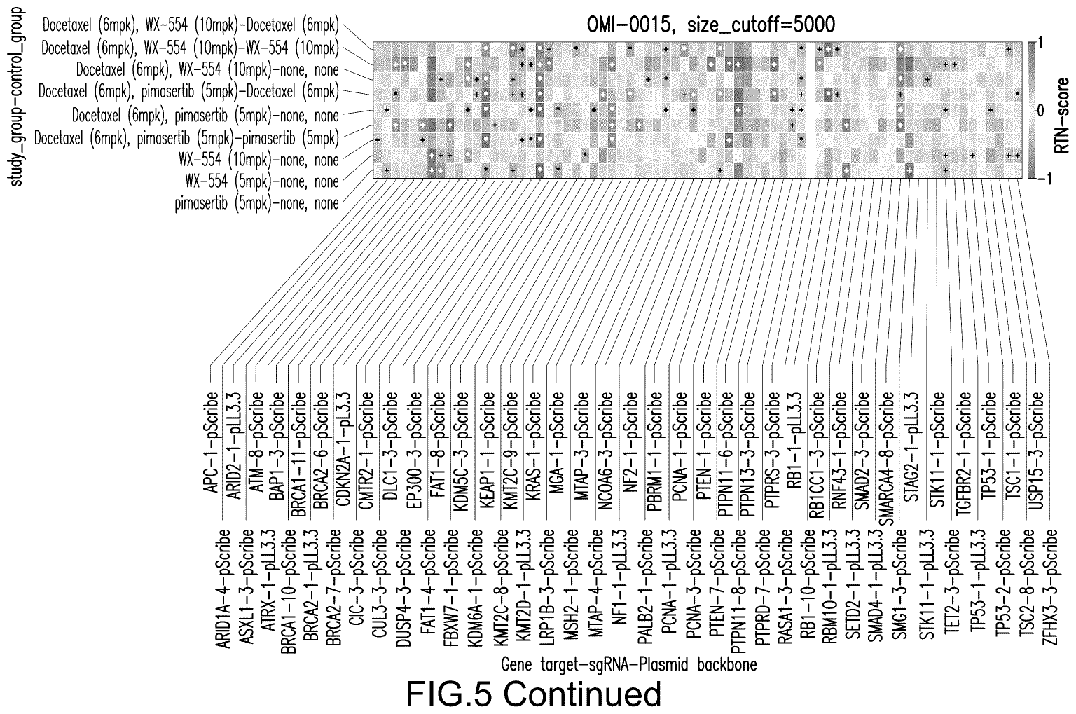

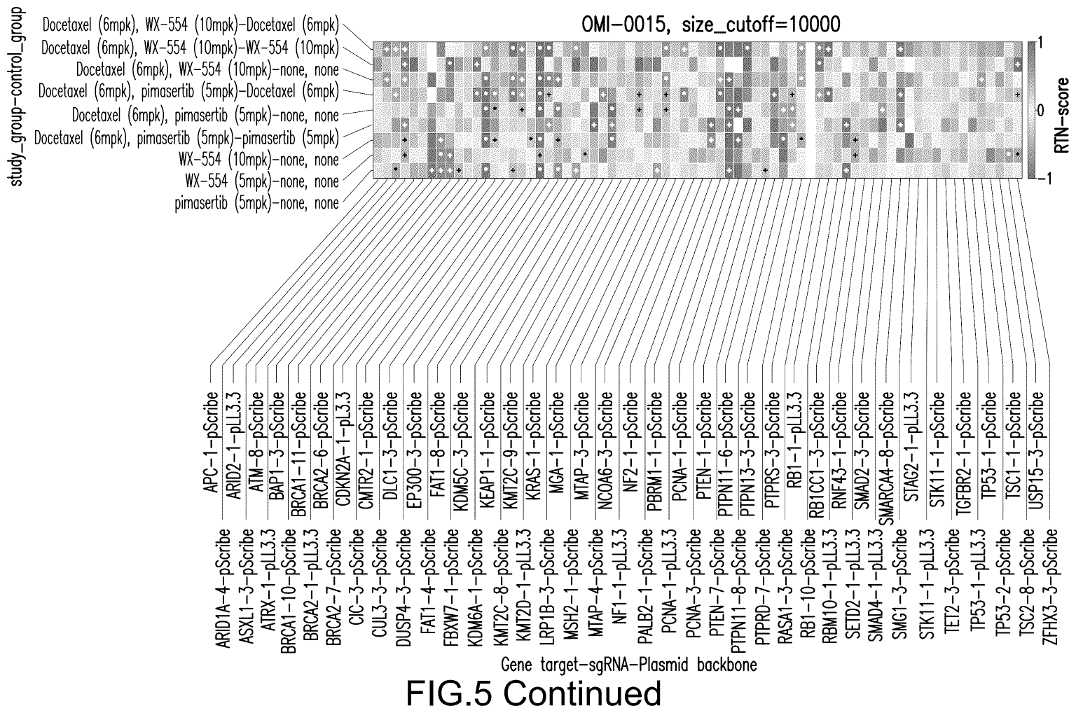

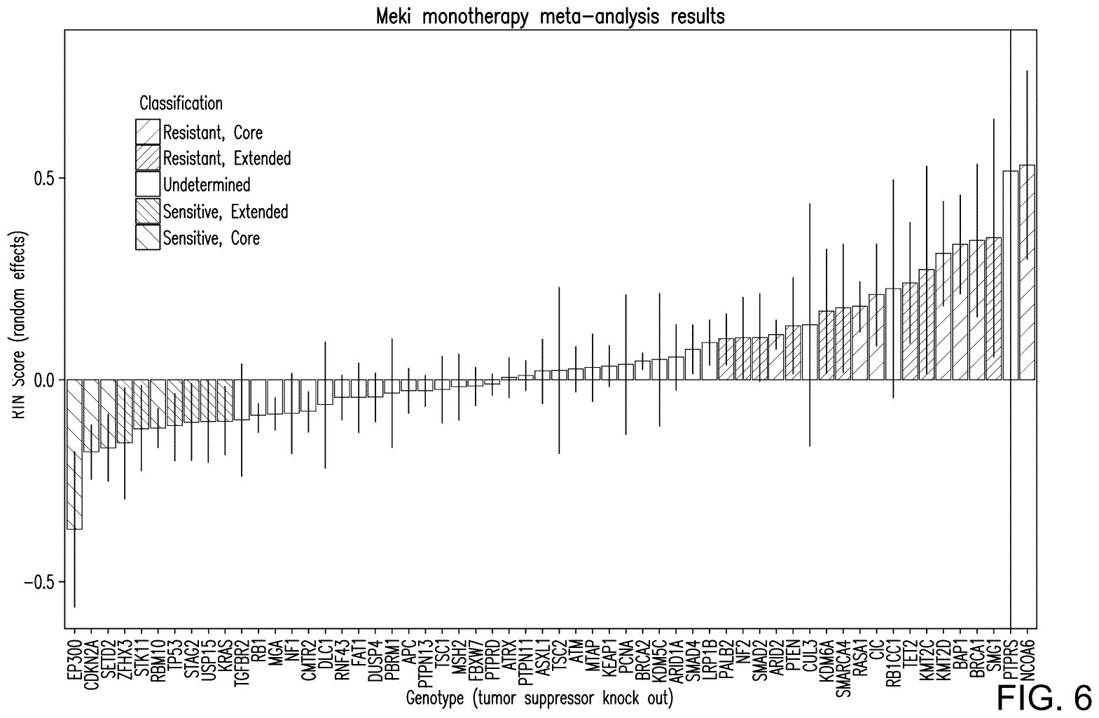

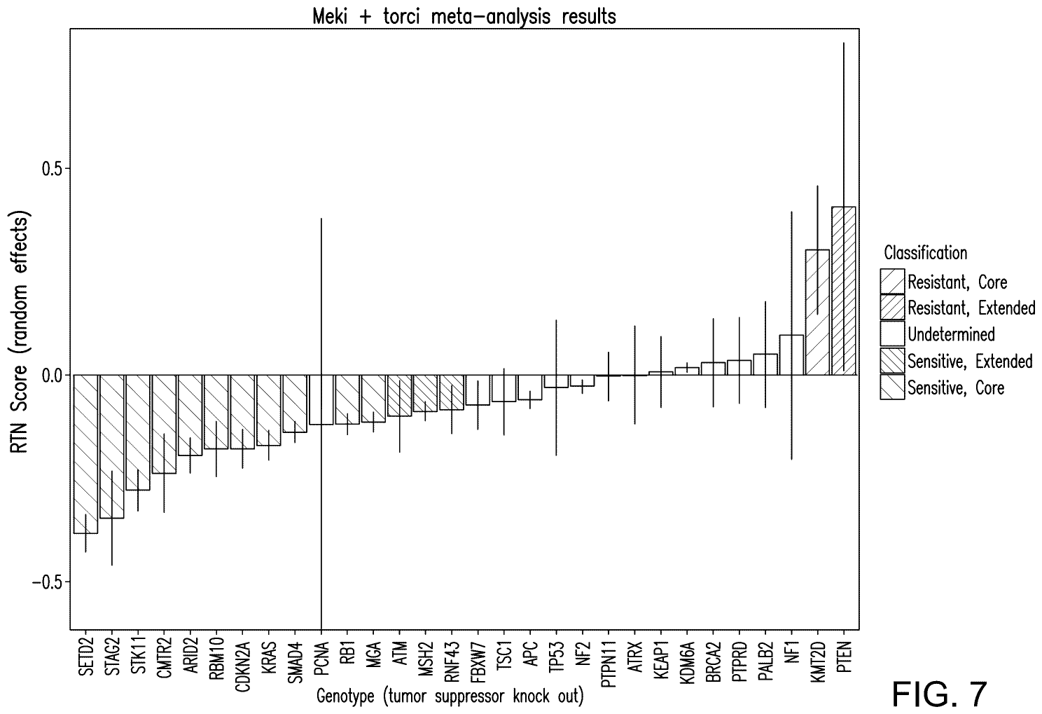

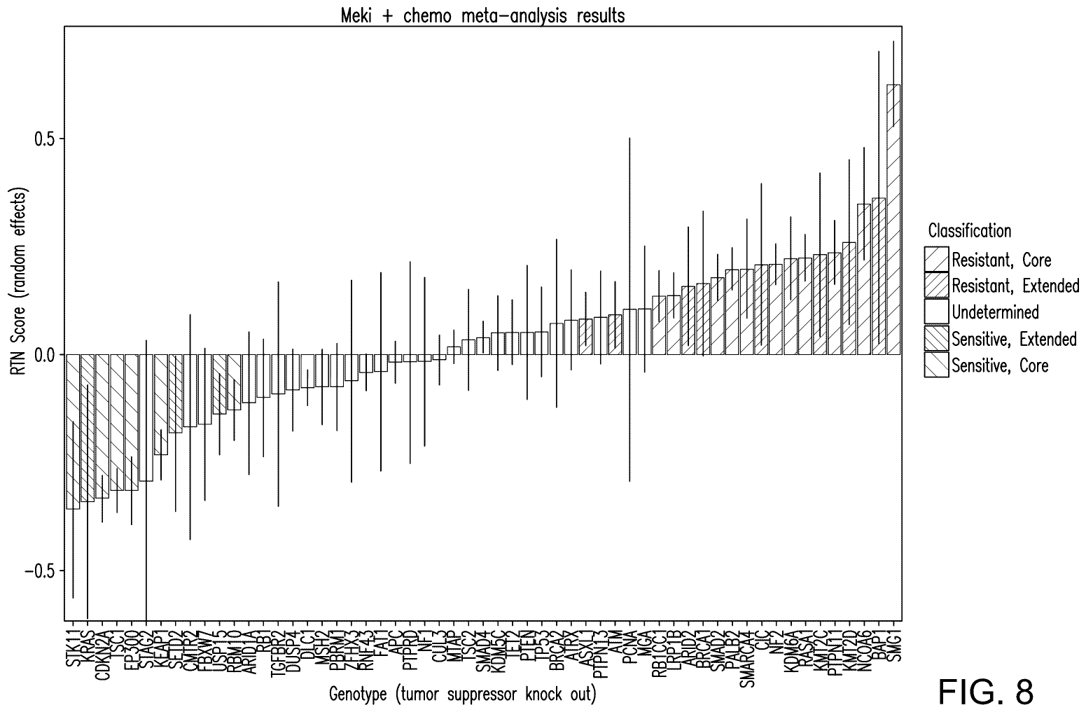

FIG. 5 shows a biomarker heatmap showing the study of pharmacogenomic interactions of MEKi/chemotherapy combination with inactivation of tumor suppressor genes. Relative tumor number (RTN)>0 indicates drug resistance, and RTN=1 corresponds to 2× change in tumor number (larger than each cutoff) relative to change in untreated vs. treated for oncogene-only tumors, while RTN=−1 corresponds to 0.5× change and drug sensitivity. *: p<0.05 and +: p<0.2. Both p-values are two-tailed and based on fraction of bootstraps with RTN scores great or less than 0.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

1. A method comprising: (a) determining a genotype of one or more biomarker genes selected from ADAR, APC, ARID1A, ARID2, ASXL1, ATM, ATRX, BAP1, BRCA1, BRCA2, CDKN2A, CHD2, CIC, CMTR2, CREBBP, CUL3, DICER1, DLC1, DNMT1, DUSP4, EP300, FAT1, FBXW7, JAK1, KDM5C, KDM6A, KEAP1, KMT2C, KMT2D, KRAS, LRP1B, MET, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TREX1, TP53, TSC1, TSC2, USP15, and ZFHX3 in a biological sample of a human subject afflicted with cancer; (b) contacting the biological sample with one or more binding agents, each binding agent specific for one of the biomarker genes, and (c) processing the sample to determine a genotype of each of the one or more biomarker genes in the biological sample.

2. The method of claim 1, further comprising (d) classifying the subject as sensitive or resistant to a therapy comprising a human MAP kinase kinase (MEK) inhibitor based on the genotype of each of the one or more biomarker genes in the biological sample.

3. The method of claim 1, wherein the biomarker genes are selected from:
(a) APC, ARID2, ATM, ATRX, BRCA2, CDKN2A, CMTR2, FBXW7, KDM6A, KEAP1, KMT2D, KRAS, MGA, MSH2, NF1, NF2, PALB2, PCNA, PTEN, PTPN11, PTPRD, RB1, RBM10, RNF43, SETD2, SMAD4, STAG2, STK11, TP53, and TSC1;
optionally wherein the biomarker genes are selected from:
(i) CDKN2A, EP300, RBM10, and SETD2;
(ii) CDKN2A, EP300, KRAS, MGA, RB1, RBM10, SETD2, STAG2, STK11, TP53, USP15, and ZFHX3;
(iii) ARID2, BAP1, BRCA1, CIC, KDM6A, KMT2C, KMT2D, LRP1B, NCOA6, NF2, PALB2, PTEN, RASA, SMAD2, SMG1, SMARCA4, and TET2;
(iv) ARID2, BAP1, BRCA1, CIC, KMT2D, NCOA6, and RASA1;
(v) ARID2, ATM, CDKN2A, CMTR2, KRAS, MGA, MSH2, RB1, RBM10, RNF43, SETD2, SMAD4, STAG2, and STK11; or
(vi) KMT2D and PTEN;
(b) APC, ARID2, ATM, ATRX, BRCA2, CDKN2A, CMTR2, KEAP1, KMT2D, MGA, NF1, PTEN, PTPRD, RB1, RBM10, RNF43, SETD2, SMAD4, STAG2, STK11, TP53, and TSC1; or
(c) APC, ARID1A, ARID2, ASXL1, ATM, ATRX, BAP1, BRCA1, BRCA2, CDKN2A, CIC, CMTR2, CUL3, DICER1, DLC1, DUSP4, EP300, FAT1, FBXW7, KDM5C, KDM6A, KEAP1, KMT2C, KMT2D, KRAS, LRP1B, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TP53, TSC1, TSC2, USP15, and ZFHX3,
optionally wherein the biomarker genes are selected from:
(i) ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, and SMG1; or
(ii) CDKN2A, EP300, KEAP1, KRAS, RBM10, SETD2, STK11, TSC1, and USP15.

4.-13. (canceled)

14. The method of claim 1, wherein said biological sample;
(a) has previously been determined to comprise a mutation in at least one gene, optionally wherein the at least one gene is an oncogene; or
(b) is a tumor sample.

15. (canceled)

16. The method of claim 1, further comprising:
(a) an initial step of obtaining a biological sample from the subject;
(b) comparing the genotype with a reference genotype, optionally wherein the genotype is reported as a score; or
(c) administering to said subject a taxane therapy.

17. (canceled)

18. The method of claim 1, wherein the genotype comprises a mutation in the one or more biomarker genes, optionally wherein the mutation inactivates the one or more biomarker genes.

19.-21. (canceled)

22. The method of claim 1, wherein determining the genotype comprises:
(a) genomic profiling; or
(b) measuring gene expression, optionally wherein measuring gene expression comprises detection of ribonucleic acids (RNAs) or polypeptides.

23.-24. (canceled)

25. The method of claim 2, wherein the subject is classified as sensitive or resistant to the MEK inhibitor therapy.

26. (canceled)

27. The method of claim 1, wherein the cancer is selected from lung cancer, pancreatic cancer, and colorectal cancer, optionally wherein the lung cancer is non-small cell lung cancer (NSCLC), further optionally wherein the NSCLC is lung adenocarcinoma.

28.-30. (canceled)

31. The method of claim 2, wherein the MEK inhibitor inhibits human MAP kinase kinase 1 (MEK1), MEK2, or MEK1/2, optionally wherein the MEK inhibitor comprises a small molecule, further optionally wherein the MEK inhibitor is selected from Trametinib, Selumetinib, Pimasertib, and WX-554.

32.-33. (canceled)

34. The method of claim 2, wherein the therapy further comprises:
(a) an inhibitor of mammalian target of rapamycin (mTOR) kinase pathway, optionally wherein the mTOR pathway inhibitor is an inhibitor of mammalian target of rapamycin complex 1 (TORC1), TORC2, or TORC1/2, and further optionally wherein the TORC inhibitor is Sapanisertib or Vistusertib; or
(b) a taxane, optionally wherein the taxane is selected from docetaxel, paclitaxel and cabazitaxel, further optionally wherein the taxane is docetaxel.

35.-39. (canceled)

40. The method of claim 1, wherein the binding agents;
(a) are capable of faciliating the genotype determination of the one or more biomarker genes, optionally wherein the binding agents comprise reagents capable of determining the genotype by detecting a nucleic acid encoding the biomarker gene or fragments thereof, further optionally wherein the binding agents comprise sequencing reagents, further optionally wherein the sequencing reagents comprise a probe or primer for sequencing the biomarker gene or a portion thereof; or
(b) comprise a reagent capable of determining the genotype by detecting a polypeptide, optionally wherein the binding agents comprise an antibody or an antigen-binding fragment thereof, further optionally wherein the binding agents comprise a label.

41.-46. (canceled)

47. The method of claim 2, further comprising administering to said subject:
(a) a MEK inhibitor therapy; or
(b) a combination therapy comprising a MEK inhibitor and an TORC inhibitor.

48.-49. (canceled)

50. A method of predicting resistance, sensitivity, or response of tumor growth to inhibition by a therapy comprising:
(1) a MEK inhibitor, said method comprising:
(A) detecting or determining a genotype of one or more biomarker genes in a tumor sample of a human subject afflicted with cancer;
(B) analyzing the genotype of the one or more biomarker genes in the tumor sample; and
(C) predicting:
(i) resistance of tumor cell growth to inhibition by the therapy, if the tumor sample comprises
(a) an inactivating mutation in one or more genes selected from ARID2, BAP1, BRCA1, CIC, KMT2D, NCOA6, and RASA1,
(b) an inactivating mutation in one or more genes selected from ARID2, BAP1, BRCA1, CIC, KDM6A, KMT2C, KMT2D, LRP1B, NCOA6, NF2, PALB2, PTEN, RASA1, SMAD2, SMG1, SMARCA4, and TET2;
(c) a decreased copy number of one or more genes selected from ARID2, BAP1, BRCA1, CIC, KMT2D, NCOA6, RASA1,
(d) a decreased copy number of one or more genes selected from ARID2, BAP1, BRCA1, CIC, KDM6A, KMT2C, KMT2D, LRP1B, NCOA6, NF2, PALB2, PTEN, RASA1, SMAD2, SMG1, SMARCA4, and TET2;
(e) a decreased expression of mRNA or protein in one or more genes selected from ARID2, BAP1, BRCA1, CIC, KMT2D, NCOA6, and RASA1; or
(f) a decreased expression of mRNA or protein in one or more genes selected from ARID2, BAP1, BRCA1, CIC, KDM6A, KMT2C, KMT2D, LRP1B, NCOA6, NF2, PALB2, PTEN, RASA1, SMAD2, SMG1, SMARCA4, and TET2, or
(ii) sensitivity of tumor cell growth to inhibition by the therapy, if the tumor sample comprises
(a) an inactivating mutation in one or more genes selected from CDKN2A, EP300, RBM10, and SETD2,
(b) an inactivating mutation in one or more genes selected from CDKN2A, EP300, KRAS, MGA, RB1, RBM10, SETD2, STAG2, STK11, TP53, USP15, and ZFHX3,
(c) a decreased copy number of one or more genes selected from CDKN2A, EP300, RBM10, and SETD2,
(d) a decreased copy number of one or more genes selected from CDKN2A, EP300, KRAS, MGA, RB1, RBM10, SETD2, STAG2, STK11, TP53, USP15, and ZFHX3,
(e) a decreased expression of mRNA or protein in one or more genes selected from CDKN2A, EP300, RBM10, and SETD2; or
(f) a decreased expression of mRNA or protein in one or more genes selected from CDKN2A, EP300, KRAS, MGA, RB1, RBM10, SETD2, STAG2, STK11, TP53, USP15, and ZFHX3; or
(iii) the response of tumor cell growth to inhibition by the therapy, if the tumor sample comprises
(a) an inactivating mutation in one or more genes selected from CDKN2A, EP300, KRAS, MGA, RB1, RBM10, SETD2, STAG2, STK11, TP53, USP15, and ZFHX3;
(b) an inactivating mutation in one or more genes selected from CDKN2A, EP300, RBM10, and SETD2;
(c) a decreased copy number of one or more genes selected from CDKN2A, EP300, KRAS, MGA, RB1, RBM10, SETD2, STAG2, STK11, TP53, USP15, and ZFHX3;
(d) a decreased copy number of one or more genes selected from CDKN2A, EP300, RBM10, and SETD2;
(e) a decreased expression of mRNA or protein in one or more genes selected from CDKN2A, EP300, KRAS, MGA, RB1, RBM10, SETD2, STAG2, STK11, TP53, USP15, and ZFHX3;
(f) a decreased expression of mRNA or protein in one or more genes selected from CDKN2A, EP300, RBM10, and SETD2;
(g) an inactivating mutation in one or more genes selected from ARID2, BAP1, BRCA1, CIC, KDM6A, KMT2C, KMT2D, LRP1B, NCOA6, NF2, PALB2, PTEN, RASA1, SMAD2, SMG1, SMARCA4, and TET2;
(h) an inactivating mutation in one or more genes selected from ARID2, BAP1, BRCA1, CIC, KMT2D, NCOA6, and RASA
(i) a decreased copy number of one or more genes selected from ARID2, BAP1, BRCA1, CIC, KDM6A, KMT2C, KMT2D, LRP1B, NCOA6, NF2, PALB2, PTEN, RASA1, SMAD2, SMG1, SMARCA4, and TET2;
(j) a decreased copy number of one or more genes selected from ARID2, BAP1, BRCA1, CIC, KMT2D, NCOA6, and RASA1;
(k) a decreased expression of mRNA or protein in one or more genes selected from ARID2, BAP1, BRCA1, CIC, KDM6A, KMT2C, KMT2D, LRP1B, NCOA6, NF2, PALB2, PTEN, RASA1, SMAD2, SMG1, SMARCA4, and TET2; or
(l) a decreased expression of mRNA or protein in one or more genes selected from ARID2, BAP1, BRCA1, CIC, KMT2D, NCOA6, and RASA1;
(2) a MEK inhibitor and a TORC inhibitor, said method comprising:
(A) detecting a genotype of one or more biomarker genes in a tumor sample of a human subject afflicted with cancer;
(B) analyzing the genotype of the one or more biomarker genes in the tumor sample; and
(C) predicting:
(i) resistance of tumor cell growth to inhibition by the combination therapy, if the tumor sample comprises
(a) an inactivating mutation in KMT2D and/or PTEN,
(b) a decreased copy number of KMT2D and/or PTEN, or
(c) a decreased expression of mRNA or protein in KMT2D and/or PTEN;
(ii) sensitivity of tumor cell growth to inhibition by the Combination therapy, if the tumor sample comprises
(a) an inactivating mutation in one or more genes selected from ARID2, ATM CDKN2A, CMTR2, KRAS, MGA, MSH2, RB1, RBM10, RNF43, SETD2, SMAD4, STAG2, and STK11,
(b) a decreased copy number of one or more genes selected from ARID2, ATM, CDKN2A, CMTR2, KRAS, MGA, MSH2, RB1, RBM10, RNF43, SETD2, SMAD4, STAG2, and STK11, or
(c) a decreased expression of mRNA or protein in one or more genes selected from ARID2, ATM, CDKN2A, CMTR2, KRAS, MGA, MSH2, RB1, RBM10, RNF43, SETD2, SMAD4, STAG2, and STK11;
(iii) response of tumor cell growth to inhibition by the combination therapy, if the tumor sample comprises
(a) an inactivating mutation in one or more genes selected from ARID2, ATM, CDKN2A, CMTR2, KRAS, MGA, MSH2, RB1, RBM10, RNF43, SETD2, SMAD4, STAG2, and STK11,
(b) a decreased copy number of one or more genes selected from ARID2, ATM, CDKN2A, CMTR2, KRAS, MGA, MSH2, RB1, RBM10, RNF43, SETD2, SMAD4, STAG2, and STK11,
(c) a decreased expression of mRNA or protein in one or more genes selected from ARID2, ATM, CDKN2A, CMTR2, KRAS, MGA, MSH2, RB1, RBM10, RNF43, SETD2, SMAD4, STAG2, and STK11;
(d) an inactivating mutation in KMT2D and/or PTEN,
(e) a decreased copy number of KMT2D and/or PTEN, or
(f) a decreased expression of mRNA or protein in KMT2D and/or PTEN; or
(3) a combination therapy comprising a MEK inhibitor and a chemotherapy, said method comprising:
(A) detecting a genotype of one or more biomarker genes in a tumor sample of a human subject afflicted with cancer;
(B) analyzing the genotype of the one or more biomarker genes in the tumor sample; and
(C) predicting:
(i) resistance of tumor cell growth to inhibition by the combination therapy, if the tumor sample comprises
(a) an inactivating ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, or SMG1 mutation,
(b) a decreased copy number of ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, or SMG1, or
(c) a decreased expression of mRNA or protein in one or more genes selected from ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, and SMG1;
(ii) sensitivity of tumor cell growth to inhibition by the combination therapy, if the tumor sample comprises:
(a) an inactivating mutation in one or more genes selected from CDKN2A, EP300, KEAP1, KRAS, RBM10, SETD2, STK11, TSC1, and USP15,
(b) a decreased copy number of one or more genes selected from CDKN2A, EP300, KEAP1, KRAS, RBM10, SETD2, STK11, TSC1, and USP15, or
(c) a decreased expression of mRNA or protein in one or more genes selected from CDKN2A, EP300, KEAP1, KRAS, RBM10, SETD2, STK11, TSC1, and USP15; or
(iii) response of tumor cell growth to inhibition by the combination therapy, if the tumor sample comprises
(a) an inactivating mutation in one or more genes selected from ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, and SMG1,
(b) a decreased copy number of one or more genes selected from ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, and SMG1,
(c) a decreased expression of mRNA or protein in one or more genes selected from ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, and SMG1;
(d) an inactivating mutation in one or more genes selected from CDKN2A, EP300, KEAP1, KRAS, RBM10, SETD2, STK11, TSC1, and USP15;
(e) a decreased copy number of one or more genes selected from CDKN2A, EP300, KEAP1, KRAS, RBM10, SETD2, STK11, TSC1, and USP15; or
(f) a decreased expression of mRNA or protein in one or more genes selected from CDKN2A, EP300, KEAP1, KRAS, RBM10, SETD2, STK11, TSC1, and USP15.

51.-55. (canceled)

56. The method of claim 50, wherein said biological sample has previously been determined to comprise a mutation in at least one gene, optionally wherein the at least one gene is an oncogene.

57. (canceled)

58. The method of claim 50, further comprising:
(a) obtaining the tumor sample from the subject; or
(b) comparing the genotype with a reference genotype, optionally wherein the genotype is reported as a score.

59. The method of claim 50, wherein the genotype comprises a mutation in the one or more biomarker genes, optionally wherein the mutation inactivates the biomarker gene.

60.-62. (canceled)

63. The method of claim 50, wherein determining the genotype comprises;
(a) genomic profiling; or
(b) measuring gene expression, optionally wherein measuring gene expression comprises detection of ribonucleic acids (RNAs) or polypeptides.

64.-65. (canceled)

66. The method of claim 55, wherein the subject is classified as sensitive or resistant to (a) a MEK inhibitor therapy, (b) a combination therapy comprising a MEK inhibitor and a TORC inhibitor, or (c) a combination therapy comprising a MEK inhibitor and a chemotherapy.

67. (canceled)

68. The method of claim 50, wherein the cancer is selected from lung cancer, pancreatic cancer, and colorectal cancer, optionally wherein the lung cancer is non-small cell lung cancer (NSCLC), further optionally wherein the NSCLC is lung adenocarcinoma.

69.-71. (canceled)

72. The method of claim 50, wherein
(a) the MEK inhibitor inhibits human MAP kinase kinase 1 (MEK1), MEK2, or MEK1/2, optionally wherein the MEK inhibitor comprises a small molecule, further optionally wherein the MEK inhibitor is selected from Trametinib, Selumetinib, Pimasertib, and WX-554; or
(b) the TORC inhibitor inhibits target of rapamycin complex 1 (TORC1), or TORC1/2, optionally wherein the TORC inhibitor comprises a small molecule, further optionally wherein the TORC inhibitor is selected from Sapanisertib and Vistusertib.

73.-74. (canceled)

75. The method of claim 50, wherein the therapy further comprises a taxane, optionally wherein the taxane is (a) selected from docetaxel, paclitaxel and cabazitaxel r (b) docetaxel.

76.-77. (canceled)

78. The method of claim 50, wherein the genotype determination comprises one or more binding agents.

79. The method of claim 78, wherein the binding agents comprise:
(a) reagents capable of determining the genotype by detecting a nucleic acid encoding the biomarker gene or fragments thereof, optionally wherein the binding agents are capable of facilitating the genotype determination of the one or more biomarker genes;
(b) a reagent capable of determining the genotype by detecting a polypeptide, optionally wherein the binding agents comprise an antibody or an antigen-binding fragment thereof;
(c) a label; or
(d) a chemotherapy, optionally wherein the chemotherapy comprises a chemotherapeutic agent belonging to the class taxanes, platinum-based chemotherapeutic agents, or folate antimetabolites.

80.-85. (canceled)

86. The method of claim 50, further comprising administering to said subject:
(a) a MEK inhibitor therapy if the predicted response of the subject is sensitivity of tumor growth to inhibition by a therapy comprising a MEK inhibitor;
(b) a taxane therapy; or
(c) a combination therapy comprising a MEK inhibitor and an TORC inhibitor.

87.-88. (canceled)

89. A method of determining effectiveness of:
(A) a MEK inhibitor in reducing tumor size comprising: (i) treating a first inert tumor with a control therapy; (ii) treating a second inert tumor with a MEK inhibitor, wherein the first and second inert tumor comprise identical genotypes; (iii) treating a first mutant tumor with the control therapy; (iv) treating a second mutant tumor with the MEK inhibitor, wherein the first and second mutant tumor comprise identical genotypes; (v) comparing sizes of the first and second inert tumors after the therapy; (vi) comparing sizes of the first and second mutant tumors after completion of the therapy, and (vii) identifying the mutant tumor genotype as (a) sensitive to the MEK inhibitor if the change in tumor size between the first and second inert tumors after the therapy is less than the change in tumor size between the first and second mutant tumors after the therapy; or (b) resistant to the MEK inhibitor if the change in tumor size between the first and second inert tumors after the therapy is greater than the change in tumor size between the first and second mutant tumors after the therapy;
(B) a combination therapy comprising a MEK inhibitor and a TORC inhibitor in reducing tumor size comprising: (i) treating a first inert tumor with a control therapy; (ii) treating a second inert tumor with a combination therapy comprising a MEK inhibitor and an TORC inhibitor, wherein the first and second inert tumor comprise identical genotypes; (iii) treating a first mutant tumor with the control therapy; (iv) treating a second mutant tumor with combination therapy, wherein the first and second mutant tumor comprise identical genotypes; (v) comparing sizes of the first and second inert tumors after the combination therapy; (vi) comparing sizes of the first and second mutant tumors after completion of the combination therapy, and (vii) identifying the mutant tumor genotype as (a) sensitive to the combination therapy if the change in tumor size between the first and second inert tumors after the therapy is less than the change in tumor size between the first and second mutant tumors after the combination therapy; or (b) resistant to the combination therapy if the change in tumor size between the first and second inert tumors after the therapy is greater than the change in tumor size between the first and second mutant tumors after the combination therapy; or
(C) a combination therapy comprising a MEK inhibitor and a chemotherapy in reducing tumor size comprising: (i) treating a first inert tumor with a control therapy; (ii) treating a second inert tumor with a combination therapy comprising a MEK inhibitor and a chemotherapy, wherein the first and second inert tumor comprise identical genotypes; (iii) treating a first mutant tumor with the control therapy; (iv) treating a second mutant tumor with the combination therapy, wherein the first and second mutant tumor comprise identical genotypes; (v) comparing sizes of the first and second inert tumors after the combination therapy; (vi) comparing sizes of the first and second mutant tumors after completion of the combination therapy, and (vii) identifying the mutant tumor genotype as (a) sensitive to the combination therapy if the change in tumor size between the first and second inert tumors after the therapy is less than the change in tumor size between the first and second mutant tumors after the combination therapy, or (b) resistant to the combination therapy if the change in tumor size between the first and second inert tumors after the combination therapy is greater than the change in tumor size between the first and second mutant tumors after the combination therapy.

90.-129. (canceled)

130. A composition comprising one or more isolated biomarker genes selected from:
(a) ADAR, APC, ARID1A, ARID2, ASXL1, ATM, ATRX, BAP1, BRCA1, BRCA2, CDKN2A, CHD2, CIC, CMTR2, CREBBP, CUL3, DICER1, DLC1, DNMT1, DUSP4, EP300, FAT1, FBXW7, JAK1, KDM5C, KDM6A, KEAP1, KMT2C, KMT2D, KRAS, LRP1B, MET, MGA, MSH2, MTAP, NCOA6, NF1, NF2, PALB2, PBRM1, PCNA, PTEN, PTPN11, PTPN13, PTPRD, PTPRS, RASA, RB1, RB1CC1, RBM10, RNF43, SETD2, SMAD2, SMAD4, SMARCA4, SMG1, STAG2, STK11, TET2, TGFBR2, TREX1, TP53, TSC1, TSC2, USP15, and ZFHX3;
(b) APC, ARID1A, ARID2, ASXL1, ATM, ATRX, BAP1, BRCA1, BRCA2, CDKN2A, CIC, CMTR2, CUL3, DICER1, DLC1, DUSP4, EP300, FAT1, FBXW7, KDM5C, KDM6A, KEAP1, KMT2C, KMT2D, KRAS, LRP1B, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TP53, TSC1, TSC2, USP15, and ZFHX3;
(c) APC, ARID2, ATM, ATRX, BRCA2, CDKN2A, CMTR2, FBXW7, KDM6A, KEAP1, KMT2D, KRAS, MGA, MSH2, NF1, NF2, PALB2, PCNA, PTEN, PTPN11, PTPRD, RB1, RBM10, RNF43, SETD2, SMAD4, STAG2, STK11, TP53, and TSC1;
(d) APC, ARID2, ATM, ATRX, BRCA2, CDKN2A, CMTR2, KEAP1, KMT2D, MGA, NF1, PTEN, PTPRD, RB1, RBM10, RNF43, SETD2, SMAD4, STAG2, STK11, TP53, and TSC1;
(e) CDKN2A, EP300, KRAS, MGA, RB1, RBM10, SETD2, STAG2, STK11, TP53, USP15, and ZFHX3;
(f) CDKN2A, EP300, RBM10, and SETD2;
(g) ARID2, ATM, CDKN2A, CMTR2, KRAS, MGA, MSH2, RB1, RBM10, RNF43, SETD2, SMAD4, STAG2, and STK11;
(h) ARID2, BAP1, BRCA1, CIC, KMT2D, NCOA6, and RASA1;
(i) ARID2, BAP1, BRCA1, CIC, KDM6A, KMT2C, KMT2D, LRP1B, NCOA6, NF2, PALB2, PTEN, RASA, SMAD2, SMG1, SMARCA4, and TET2;
(j) KMT2D and PTEN;
(k) ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, and SMG1; or
(l) CDKN2A, EP300, KEAP1, KRAS, RBM10, SETD2, STK11, TSC1, and USP15,
optionally wherein the composition comprises two, three, four, five, six, seven, eight, nine, ten or more of the isolated biomarker genes.

131-153. (canceled)

154. The composition of claim 130, further comprising a binding agent, optionally wherein the binding agent:
(a) is capable of facilitating genotype determination of the biomarker gene, further optionally wherein the binding agent comprises an antibody or an antigen-binding fragment thereof;
(b) comprises a reagent capable of determining the genotype by detecting a polypeptide or a nucleic acid encoding the biomarker gene or fragments thereof, or
(c) comprises a label.

155.-161. (canceled)

162. A method of detecting in a human subject one or more isolated biomarker genes selected from
(a) ADAR, APC, ARID1A, ARID2, ASXL1, ATM, ATRX, BAP1, BRCA1, BRCA2, CDKN2A, CHD2, CIC, CMTR2, CREBBP, CUL3, DICER1, DLC1, DNMT1, DUSP4, EP300, FAT1, FBXW7, JAK1, KDM5C, KDM6A, KEAP1, KMT2C, KMT2D, KRAS, LRP1B, MET, MGA, MSH2, MTAP, NCOA6, NF1, NF2, PALB2, PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TREX1, TP53, TSC1, TSC2, USP15, and ZFHX3,
(b) APC, ARID1, ATM, ATRX, BRCA2, CDKN2A, CMTR2, FBXW7, KDM6A, KEAP1, KMT2D, KRAS, MGA, MSH2, NF1, NF2, PALB2, PCNA, PTEN, PTPN11, PTPRD, RB1, RBM10, RNF43, SETD2, SMAD4, STAG2, STK11, TP53, and TSC1;
(c) APC, ARID2, ATM, ATRX, BRCA2, CDKN2A, CMTR2, KEAP1, KMT2D, MGA, NF1, PTEN, PTPRD, RB1, RBM10, RNF43, SETD2, SMAD4, STAG2, STK11, TP53, and TSC1;
(d) CDKN2A, EP300, KRAS, MGA, RB1, RBM10, SETD2, STAG2, STK11, TP53, USP15, and ZFHX3;
(e) CDKN2A, EP300, RBM10, and SETD2;
(f) ARID2, ATM, CDKN2A, CMTR2, KRAS, MGA, MSH2, RB1, RBM10, RNF43, SETD2, SMAD4, STAG2, and STK11;
(g) ARID2, BAP1, BRCA1, CIC, KMT2D, NCOA6, and RASA1;
(h) ARID2, BAP1, BRCA1, CIC, KDM6A, KMT2C, KMT2D, LRP1B, NCOA6, NF2, PALB2, PTEN, RASA, SMAD2, SMG1, SMARCA4, and TET2;
(i) KMT2D and PTEN;
(j) APC, ARID1A, ARID2, ASXL1, ATM ATRX BAP1, BRCA1, BRCA2, CDKN2A, CIC, CMTR2, CUL3, DICER1, DLC1, DUSP4, EP300, FAT1, FBXW7, KDM5C, KDM6A, KEAP1, KMT2C, KMT2D, KRAS, LRP1B, MGA, MSH2, MTAP, NCOA6, NF1, NF2, PALB2. PBRM1, PCNA, PTEN, PTPN11, PTPN13, PTPRD, PTPRS, RASA1, RB1, RB1CC1, RBM10, RNF43, SETD2, SMAD2, SMAD4, SMARCA4, SMG1, STAG2, STK11, TET2, TGFBR2, TP53, TSC1, TSC2, USP15, and ZFHX3,
(k) ARID2, ASXL1, ATM, BAP1, BRCA1 CIC, KDM6A, KMT2C, KMT2D, LRP1B, NCOA6, NF2, PALB2, PTPN11, RASA1, RB1CC1, SMAD2, SMARCA4, and SMG1; or
(l) CDKN2A, EP300, KEAP1, KRAS, RBM10, SETD2, STK11, TSC1, and USP15,
said method comprising: detecting whether the one or more isolated biomarker genes are present in a biological sample of the human subject by contacting the biological sample with a binding agent and detecting binding between the one or more isolated biomarker genes and the binding agent.

163.-173, (canceled)

174. The method of claim 162, wherein the binding agent:
(a) is capable off facilitate genotype determination of the biomarker gene, optionally wherein the binding agent comprises an antibody or an antigen-binding fragment thereof;
(b) comprises a reagent capable of determining the genotype by detecting a polypeptide or a nucleic acid encoding the biomarker gene or fragments thereof, or
(c) comprises a label.

175.-220. (canceled)

221. A method of treating non-small cell lung cancer (NSCLC) in a subject comprising:
(a) administering to a subject a MEK inhibitor when a tumor sample of the subject comprises (i) an inactivating mutation in one or more genes selected from CDKN2A, EP300, KRAS, MGA, RB1, RBM10, SETD2, STAG2, STK11, TP53, USP15, and ZFHX3, (ii) a decreased copy number of one or more genes selected from CDKN2A, EP300, KRAS, MGA, RB1, RBM10, SETD2, STAG2, STK11, TP53, USP15, and ZFHX3, or (iii) a decreased expression of mRNA or protein in one or more genes selected from CDKN2A, EP300, KRAS, MGA, RB1, RBM10, SETD2, STAG2, STK11, TP53, USP15, and ZFHX3,
optionally, wherein the inactivating mutation is in one or more of CDKN2A, EP300, RBM10, and SETD2;
optionally, wherein the tumor sample further comprises absence of (i) an inactivating mutation in one or more genes selected from ARID2, BAP1, BRCA1, CIC, KDM6A, KMT2C, KMT2D, LRP1B, NCOA6, NF2, PALB2, PTEN, RASA1, SMAD2, SMG1, SMARCA4, and TET2, (ii) a decreased copy number of one or more genes selected from ARID2, BAP1, BRCA1, CIC, KDM6A, KMT2C, KMT2D, LRP1B, NCOA6, NF2, PALB2, PTEN, RASA1, SMAD2, SMG1, SMARCA4, and TET2, or (iii) a decreased expression of mRNA or protein in one or more genes selected from CDKN2A, EP300, KRAS, MGA, RB1, RBM10, SETD2, STAG2, STK11, TP53, USP15, and ZFHX3, and further optionally wherein the absence of an inactivating mutation is in one or more genes selected from ARID2, BAP1, BRCA1, CIC, KMT2D, NCOA6, and RASA1;
(b) administering to a subject a MEK inhibitor and an TORC inhibitor when a tumor sample of the subject comprises (i) an inactivating mutation in one or more genes selected from ARID2, ATM, CDKN2A, CMTR2, KRAS, MGA, MSH2, RB1, RBM10, RNF43, SETD2, SMAD4, STAG2, and STK11, (ii) a decreased copy number of one or more genes selected from ARID2, ATM, CDKN2A, CMTR2, KRAS, MGA, MSH2, RB1, RBM10, RNF43, SETD2, SMAD4, STAG2, and STK11, or (iii) a decreased expression of mRNA or protein in one or more genes selected from ARID2, ATM, CDKN2A, CMTR2, KRAS, MGA, MSH2, RB1, RBM10, RNF43, SETD2, SMAD4, STAG2, and STK11,
optionally, wherein the tumor sample further comprises absence of (i) an inactivating mutation in KMT2D and/or PTEN, (ii) a decreased copy number of KMT2D and/or PTEN, or (iii) a decreased expression of mRNA or protein in KMT2D and/or PTEN; or
(c) administering to a subject a MEK inhibitor and a chemotherapy when a tumor sample of the subject comprises (i) an inactivating mutation in one or more genes selected from CDKN2A, EP300, KEAP1, KRAS, RBM10, SETD2, STK11, TSC1, and USP15, (ii) a decreased copy number of one or more genes selected from CDKN2A, EP300, KEAP1, KRAS, RBM10, SETD2, STK11, TSC1, and USP15, or (iii) a decreased expression of mRNA or protein in one or more genes selected from CDKN2A, EP300, KEAP1, KRAS, RBM10, SETD2, STK11, TSC1, and USP15,
Optionally, wherein the tumor sample further comprises absence of (i) an inactivating mutation in one or more genes selected from ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, and SMG1, (ii) a decreased copy number of one or more genes selected from ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, and SMG1, or (iii) a decreased expression of mRNA or protein in one or more genes selected from ARID2, ASXL1, ATM, BAP1, BRCA1, CIC, KDM6A, KMT2C, KMT2D, LRP1B, NCOA6, NF2, PALB2, PTPN11, RASA1, RB1CC1, SMAD2, SMARCA4, and SMG1.

222.-228. (canceled)

229. A method of enriching a prospective patient population for subjects likely to respond to a MEK inhibitor therapy, comprising performing the method of claim 50 on a biological sample of one or more subjects within said patient population.

230.-231. (canceled)
